(19) United States
(12) Patent Application Publication
(10) Pub. No.: US 2022/0396611 A1
(43) Pub. Date: Dec. 15, 2022

(54) FACTOR VIII CONSTRUCT

(71) Applicant: Freeline Therapeutics Limited, Stevenage (GB)

(72) Inventors: Azadeh KIA, Enfield (GB); Fabrizio COMPER, Stevenage (GB); Romuald CORBAU, Chiswell Green (GB); Tihomir DODEV, Enfield (GB); Jenny MCINTOSH, Epsom (GB)

(21) Appl. No.: 17/772,816

(22) PCT Filed: Oct. 30, 2020
(86) PCT No.: PCT/GB2020/052761

§ 371 Date: Apr. 28, 2022

(30) Foreign Application Priority Data
Nov. 1, 2019 (GB) 1915953.2
Nov. 1, 2019 (GB) 1915955.7
Nov. 1, 2019 (GB) 1915956.5
Dec. 6, 2019 (GB) 1917925.8
Dec. 6, 2019 (GB) 1917926.6
Dec. 6, 2019 (GB) 1917927.4
Apr. 28, 2020 (GB) 2006250.1

(51) Int. Cl. C07K 14/755; C12N 15/86

(52) U.S. Cl. CPC C07K 14/755; C12N 15/86; C12N 2710/00031; C12N 2710/00042

(57) ABSTRACT

The present invention relates to a Factor VIII (FVIII) polypeptide, a polynucleotide comprising a Factor VIII nucleotide sequence, and a recombinant AAV construct. The invention further relates to an AAV viral particle comprising the recombinant AAV construct of the invention, and a composition comprising the Factor VIII polypeptide, polynucleotide, recombinant AAV construct or AAV viral particle of the invention. The invention also relates to methods of using, and uses of, the Factor VIII polypeptide, polynucleotide, recombinant AAV construct, AAV viral particle and/or composition of the invention. The invention also relates to uses of the recombinant AAV construct of the invention for the production of AAV viral particles, and methods for producing AAV viral particles using the recombinant AAV constructs of the invention.

### FIELD OF THE INVENTION

The present invention relates to a Factor VIII (FVIII) polypeptide, a polynucleotide comprising a Factor VIII nucleotide sequence, and a recombinant AAV construct. The invention further relates to an AAV viral particle comprising the recombinant AAV construct of the invention, and a composition comprising the Factor VIII polypeptide, polynucleotide, recombinant AAV construct or AAV viral particle of the invention. The invention also relates to methods of using, and uses of, the Factor VIII polypeptide, polynucleotide, recombinant AAV construct, AAV viral particle and/or composition of the invention.

### BACKGROUND OF THE INVENTION

Haemophilia A is a bleeding disorder caused by a deficiency of blood clotting Factor VIII. It affects 1:4,000 to 1:5,000 live male births worldwide. The majority of cases are inherited as an X-linked recessive trait. Current treatment involves frequent intravenous injections (2-3 times per week) of Factor VIII protein. This treatment is highly effective at arresting bleeding but it is not curative and is extremely expensive (£150,000/patient/year), thus making it unaffordable by the majority of haemophilia A patients in the world. Gene therapy for haemophilia A offers the potential for a cure through persistent, endogenous production of Factor VIII following the transfer of a functioning copy of the Factor VIII gene to an affected patient.

Factor VIII consists of six domains, namely A1-A2-B-A3-C1-C2. Factor VIII circulates in the bloodstream in an inactive form whilst bound to von Willebrand factor. In response to injury, Factor VIII is activated (often then referred to as Factor VIIIa) and separates from von Willebrand factor. Factor VIIIa interacts with Factor IXa in the coagulation cascade. In particular, Factor FVIIIa is a cofactor for Factor IXa in the activation of Factor X.

### SUMMARY OF THE INVENTION

The present invention relates to an advantageously short Factor VIII polypeptide and an advantageously short recombinant AAV construct which can express a Factor VIII polypeptide. Recombinant AAV constructs which can express Factor VIII polypeptides are already known and generally have sizes in excess of 5000 nucleotides. Therefore they are significantly larger than the typical wild-type AAV genome size of approximately 4700 nucleotides in length. The present inventors have managed to successfully shorten the length of the Factor VIII polypeptide and the length of the Factor VIII nucleotide sequence. The present inventors have also managed to successfully shorten the length of other components of the recombinant AAV construct. By shortening the length of the Factor VIII nucleotide sequence and/or one or more other components of the recombinant AAV construct, the size of the recombinant AAV construct can be less than 4900, and preferably less than 4800, nucleotides in length and thus advantageously close to the length of a wild-type AAV genome. Shortening the length of the Factor VIII polypeptide also has further advantages, for example reducing the burdens associated with manufacturing, such as time and costs.

The present inventors have also found that some internally truncated Factor VIII polypeptides of the invention have the further advantage of maintained or increased activity of the Factor VIII polypeptide. Internally truncated Factor VIII polypeptides which lack the PACE/furin cleavage site corresponding to positions 1645 to 1648 of SEQ ID NO:1 may be expressed as single chain polypeptides and may have increased stability and/or specific activity relative to Factor VIII polypeptides which retain the PACE/furin cleavage site. Internally truncated Factor VIII polypeptides which lack residues corresponding to positions 724-740 of SEQ ID NO:1 which are N-terminal to the B domain have been found to retain Factor VIII activity and may be expressed at a higher level. Residues corresponding to positions 714-723 of SEQ ID NO:1 may promote thrombin binding and may optionally be retained in an internally truncated Factor VIII polypeptide of the invention. Internally truncated Factor VIII polypeptides which lack residues corresponding to positions 1649-1669 of SEQ ID NO:1 which are C-terminal to the B domain have also been found to retain FVIII activity and may be expressed at a higher level. Residues corresponding to positions 1670-1689 of SEQ ID NO:1 (optionally residues corresponding to positions 1670-1678 of SEQ ID NO: 1) may be required for von Willebrand factor binding, and may optionally be retained in a Factor VIII polypeptide of the invention. The inventors have therefore identified a number of internally truncated Factor VIII polypeptides which retain Factor VIII activity and are expressed well in vivo and in AAV mediated gene therapy, and which are smaller than Factor VIII polypeptides of the art.

The present inventors have found that the codon-optimisation described herein of a Factor VIII nucleotide sequence also resulted in the advantage of increased expression of the Factor VIII polypeptide. Thus, the present invention also relates to a polynucleotide sequence which encodes a Factor VIII polypeptide.

In a first aspect, the present invention provides a Factor VIII polypeptide comprising a Factor VIII amino acid sequence wherein the Factor VIII amino acid sequence comprises a modified beta domain related (BDR) region which is modified relative to wild-type BDR region, which wild-type BDR region corresponds to the region between positions 713 and 1697 of SEQ ID NO:1, wherein:
(i) the modified BDR region comprises a maximum of 88 amino acids, and the Factor VIII polypeptide has a specific activity which is higher than the specific activity of a polypeptide of SEQ ID NO:7; and/or
(ii) the modified BDR region comprises a maximum of 74 amino acids.

In a second aspect, there is provided a polynucleotide comprising a Factor VIII nucleotide sequence, wherein the Factor VIII nucleotide sequence encodes a Factor VIII polypeptide and wherein at least a portion of the Factor VIII nucleotide sequence is not wild-type.

In a third aspect, there is provided a recombinant AAV construct which comprises a polynucleotide comprising a Factor VIII nucleotide sequence, wherein the Factor VIII nucleotide sequence encodes a Factor VIII polypeptide comprising a Factor VIII amino acid sequence.

In a fourth aspect, there is provided an AAV viral particle comprising the recombinant AAV of the invention.

In a fifth aspect, there is provided a Factor FVIII polypeptide encoded by the polynucleotide or recombinant AAV construct of the invention.

In a sixth aspect, there is provided a composition comprising the Factor VIII polypeptide, polynucleotide, recombinant AAV construct or AAV viral particle of the invention.

In a seventh aspect, there is provided the Factor VIII polypeptide, polynucleotide, recombinant AAV construct, AAV viral particle or composition of the invention for use in a method of treatment.

In an eighth aspect, there is provided a method of treatment comprising administering an effective amount of the Factor VIII polypeptide, polynucleotide, recombinant AAV construct or AAV viral particle of the invention to a patient.

In a ninth aspect, there is provided a use of the Factor VIII polypeptide, polynucleotide, recombinant AAV construct, AAV viral particle or composition of the invention in the manufacture of a medicament for use in a method of treatment.

In a tenth aspect, there is provided a use of the recombinant AAV construct of the invention for producing a population of AAV viral particles.

In an eleventh aspect, there is provided a use of the recombinant AAV construct of the invention which is less than 4900 nucleotides in length for:
a) increasing the vector genome yield during AAV viral particle production when compared to the vector genome yield obtained when a comparator recombinant AAV construct is used;
b) increasing the vector genome to total particle ratio during AAV viral particle production when compared to the vector genome to total particle ratio obtained when a comparator recombinant AAV construct is used; and/or
c) decreasing the nucleic acid impurity level during AAV viral particle production when compared to the nucleic acid impurity level obtained when a comparator recombinant AAV construct is used;
   wherein the comparator recombinant AAV construct is more than 4900 nucleotides in length.

In a twelfth aspect, there is provided a method for producing a population of AAV viral particles comprising:
a) obtaining the recombinant AAV construct of the invention;
b) transfecting a host cell with the said recombinant AAV construct; and
c) culturing the host cell under conditions suitable for AAV viral particle production.

In a thirteenth aspect, there is provided a method for increasing the vector genome yield during the production of a population of AAV viral particles, comprising:
a) obtaining a recombinant AAV construct of the invention which is less than 4900 nucleotides in length;
b) transfecting a host cell with the said recombinant AAV construct; and
c) culturing the host cell under conditions suitable for AAV viral particle production;
   wherein the vector genome yield is increased when compared to the vector genome yield obtained when a comparator recombinant AAV construct of more than 4900 nucleotides in length is used.

In a fourteenth aspect, there is provided a method for increasing the vector genome to total particle ratio during the production of a population of AAV viral particles, comprising:
a) obtaining a recombinant AAV construct of the invention which is less than 4900 nucleotides in length;
b) transfecting a host cell with the said recombinant AAV construct; and
c) culturing the host cell under conditions suitable for AAV viral particle production;
   wherein the vector genome to total particle ratio is increased when compared to the vector genome to total particle ratio obtained when a comparator recombinant AAV construct of more than 4900 nucleotides in length is used.

In a fifteenth aspect, there is provided a method for decreasing the nucleic acid impurity level during the production of a population of AAV viral particles, comprising:
a) obtaining a recombinant AAV construct of the invention which is less than 4900 nucleotides in length;
b) transfecting a host cell with the said recombinant AAV construct; and
c) culturing the host cell under conditions suitable for AAV viral particle production;
   wherein the nucleic acid impurity level is decreased when compared to the nucleic acid impurity level obtained when a comparator recombinant AAV construct of more than 4900 nucleotides in length is used.

In a sixteenth aspect, there is provided a population of AAV viral particles obtained or obtainable by the methods of the invention.

### DESCRIPTION OF THE FIGURES

FIG. 1 provides a schematic diagram showing the component parts of an AAV construct of the invention. From the 5′ to the 3′ direction, the AAV construct comprises a 5′ ITR, a promoter, a nucleotide sequence encoding a signal peptide, a Factor VIII nucleotide encoding a Factor VIII polypeptide having a modified BDR region, a polyA sequence and a 3′ ITR region.

FIG. 2 provides a comparison of FVIII-SQ expression for different codon-optimised nucleotide sequences. 2A—Comparison of the FVIII-SQ expression (FVIII activity) level following Huh7 cell plasmid transfection of 20 codon-optimised (“Co ”) FVIII-SQ coding sequences, as described in Example 2. These data are representative of two separate experiments and each experiment was set in triplicate. 2B—FVIII-SQ antigen level in murine serum 4 weeks after administration with AAV8 expressing FVIII-SQ Co1, Co2, Co3 or Co19, or wild type (“wt”), coding sequences. Bars represent mean of n=5 mice.

FIG. 3 provides the relationships of the codon-optimised Factor VIII nucleotide sequences. 3A—Percentage identities between each of the codon-optimised Factor VIII nucleotide sequences. 3B —tree diagram showing the relationship between the sequences; the boxed area indicates that codon-optimised sequences 1, 2, 3 and 19 form a distinct group.

FIG. 4 provides a summary of the codon usage in a number of different codon-optimised Factor VIII nucleotide sequences encoding Factor VIII SQ polynucleotides, in comparison to a corresponding wild-type nucleotide sequence. The total number and percentage of preferred codons was calculated for each codon-optimised sequence.

FIG. 5 provides the sequences of the modified BDR (Beta Domain Related) region of a number of Factor VIII polypeptides. Polypeptide 95 shows the BDR of a Factor VIII SQ polypeptide. Polypeptide 85 shows the BDR of a Factor VIII RE polypeptide. Polypeptides 96 to 107-117 show the modified BDR regions of Factor VIII polypeptides set forth in SEQ ID NOs:9-36, respectively. These sequences form parts of SEQ ID NOs:9-36. Numbering is with respect to SEQ ID NO:1, the sequence of a full length wild-type Factor VIII polypeptide.

FIG. 6 provides the specific activity of a number of Factor VIII polypeptides having modified BDR regions. Specific activity (activity/quantity) was calculated for each Factor VIII polypeptide relative to a Factor VIII SQ.

FIG. 7 provides the level of Factor VIII-SQ polypeptide measured in blood collected from mice four weeks post injection, for polypeptides having a wild-type signal peptide, or signal peptides 5, 8 or 10.

FIG. 8 provides the results of two (8A-C and 8D-F) in vitro studies conducted using transcription regulatory elements HLP2, FRE46, FRE47, FRE48 and FRE72 in a Factor VIII expression cassette. Huh7 cells were transfected with FVIII-SQ constructs comprising a TRE of interest. The level of FVIII activity in culture supernatant was analysed at day 3 post-transfection. 8A and 8D show the FVIII activity levels (% FVIII:C, determined using the FVIII chromogenic activity assay described below); 8B and 8E show luciferase activity level from the corresponding transfected well; 8C and 8F show normalised FVIII level to the level of luciferase expression thereby showing the relative efficacy of the transcription regulatory element. HLP2 is provided for comparison purposes. The bar charts represent mean values from triplicate experiments. RLU=relative luminescence units.

FIG. 9 provides the results of in vivo studies conducted using HLP2 and FRE72 in a Factor VIII expression cassette. 6-8-week-old male C57BL/6 mice were intravenously injected with 2×10¹² vg/kg viral vector. Six mice were injected per construct. On day 28 post injection the mice were culled and blood harvested intro citrate anticoagulant. Blood and murine liver were provided for analysis. Blood was used to carry out FVIII analysis while liver biopsies were used to calculate the vector genomes per cell. 9A) shows Factor VIII antigen levels; 9B) shows estimated vector genomes per cell; 9C) shows FVIII antigen levels normalised to the vector genomes per cell thereby showing the relative efficacy of the transcription regulatory element. The bar charts represent mean values (n=6).

FIG. 10 provides a comparison of the Factor VIII activity levels in plasma from mice injected with pLIVE-SP10-FVIII co19-SQ, pLIVE-SP10-FV111-(96-106 co19), or pLIVE-comparator-FVIIIco-SQ. The comparator codon optimised FVIII SQ nucleotide sequence is an alternative codon optimised FVIII SQ nucleotide sequence set forth in SEQ ID NO:37.

FIG. 11 provides the results of an in vitro study of AAVS3 dose response of constructs which differ only by the signal peptide. Huh7 cells were transduced with AAVS3 expressing FVIIIco19-SQ coding sequences having a wild-type signal peptide or signal peptide 5. The activity of secreted FVIII was measured over a range of vector doses. MOI=multiplicity of infection.

FIG. 12 shows the results of in vitro studies into the promoter fidelity of FRE72. FRE72 promoter fidelity was assessed in cell lines from a range of different tissues; Huh7: liver. HEK293T: kidney. PANC1: pancreas. BxPC-3: pancreas. MCF7: breast. 1643: neuroblastoma. MRC-9: lung. 697: early B cell. Cells were transduced with the control vector AAVS3.CAG.GFP or AAVS3.FRE72.GFP or untreated at MOI of 1×10⁵. FIG. 12 shows three columns for each cell type; the left hand column for each cell type (grey) relates to cells transduced with AAVS3.FRE72.GFP; the central column for each cell type (black) relates to cells transduced with the control vector; and the right hand column for each cell type (white) relates to untreated cells. For the HEK293T and MCF-7 cells the left hand (“grey”) column is so small that it is not visible in FIG. 12; similarly for the HEK293T, 1643 and 697 cells the right hand (“white”) columns is so small that it is not visible in FIG. 12.

FIG. 13 shows the Wimley-White hydrophobicity scale for the free energy (AG) transition of an amino acid from an aqueous phase to a non-aqueous phase (octanol). A more negative ΔG value is associated with a more favourable transition from the aqueous phase into the non-aqueous phase, and denotes a more hydrophobic amino acid.

FIG. 14 shows the fold-change in SA (specific activity), relative to the FVIII-SQ ('95′) control lacking any substitution mutations, for several different amino-acid substitution mutation variants, including a number of alternative substituted residues for each variant. Variant 65 (11693W) exhibits an elevation in SA relative to 95.

FIG. 15 shows the fold-change in SA (specific activity), relative to the FVIII-SQ ('95′) control lacking any substitution mutations, for several different double cysteine substitution mutation variants. A number of variants exhibit an elevation in SA relative to 95.

FIG. 16 shows the Factor VIII specific activity of a series of Factor VIII polypeptides comprising substitution mutations. The effect of the substitution mutations on specific activity was assessed in ‘SQ’ and ‘96-106’ Factor VIII polypeptides, relative to a Factor VIII-SQ control which did not comprise any substitution mutations. Error bars represent the standard deviation from sample duplicates in the ELISA and activity assays.

FIG. 17 shows Factor VIII specific activity in plasma of C7BL/6 FVIII knockout mice intravenously injected with 2×10¹² vg/kg of AAV8 viral particles made from the constructs FRE72-SP5-FVIIICo19 (26-96-106)-SpA (SEQ ID NO:71); FRE72-SP5-FVIIICo19-SQ-SpA (SEQ ID NO:73); or a comparator construct comprising a codon-optimised FVIII-SQ-encoding sequence with native FVIII signal peptide, SpA and a liver-specific promoter (SEQ ID NO:72), six weeks post-injection. **P=0.0038. The left hand panel shows the specific activity of FRE72-5P5-FVIIICo19 (26-96-106)-SpA (left hand column) and the comparator construct (middle column) relative to a naïve control; the right hand panel shows the specific activity of FRE72-SP5-FVIIICo19-SQ-SpA (left hand column) and the comparator construct (right hand column).

FIG. 18 shows the vector genome yields (A), the capsid yields (B) and the vector genome to total particle ratios (C) for FRE72-5P5-FVIIICo19 (26-96-106)-SpA and the comparator FVIIIco-SQ produced in the trans-split, two plasmid manufacturing system. Vector genome yields were quantified using a transgene cassette-specific qPCR applicable for both constructs. Capsid yields were determined by Capsid-specific ELISA. The vector genome to total particle ratios were calculated based on the qPCR and ELISA results. The results are shown as fold-changes relative to the comparator FVIIIco-SQ.

FIG. 19 shows the quantification of encapsidated non-vector nucleic acid (plasmid-derived) for FRE72-5P5-FVIIICo19 (26-96-106)-SpA and the comparator FVIIIco-SQ. Virus particles were purified before analysis by a one-step affinity chromatography step using AVB Sepharose HP. Quantification of encapsidated plasmid-derived impurities is based on qPCRs specific for defined sequences of the kanamycin resistance gene (kanR) (A) or the AAV cap gene (B). The impurity levels were calculated based on the qPCR results. The results are shown as fold-changes relative to the comparator FVIIIco-SQ.

FIG. 20 shows the analysis of vector integrity for FRE72-5P5-FVIIICo19 (26-96-106)-SpA and the comparator FVIIIco-SQ by alkaline gel electrophoresis. The same number of vector genomes was applied for both constructs. A linear DNA fragment was used as loading control.

For size determination, a molecular weight marker was included in the gel electrophoresis. Gel staining was performed with SybrGold. Visualization was achieved on an imaging system with an excitation wavelength of 312 nm. The signals reflecting monomer vector genomes are highlighted by an arrow for FRE72-SP5-FVIIICo19 (26-96-106)-SpA and the comparator FVIIIco-SQ.

FIG. 21 shows the results of an in vivo study conducted to confirm the longevity of the FRE72 promoter. An AAV8 construct comprising an FVIII-SQ transgene under the transcriptional control of the FRE72 promoter was prepared and administered to wild-type mice. Blood samples were taken by tail bleeding (at days 31, 56 and 104 post-injection) and finally via cardiac puncture (at day 230 post-injection). The FVIII antigen level in each sample was measured and the data points are shown on the graph. The bars represent median values.

FIG. 22—sequence listing.

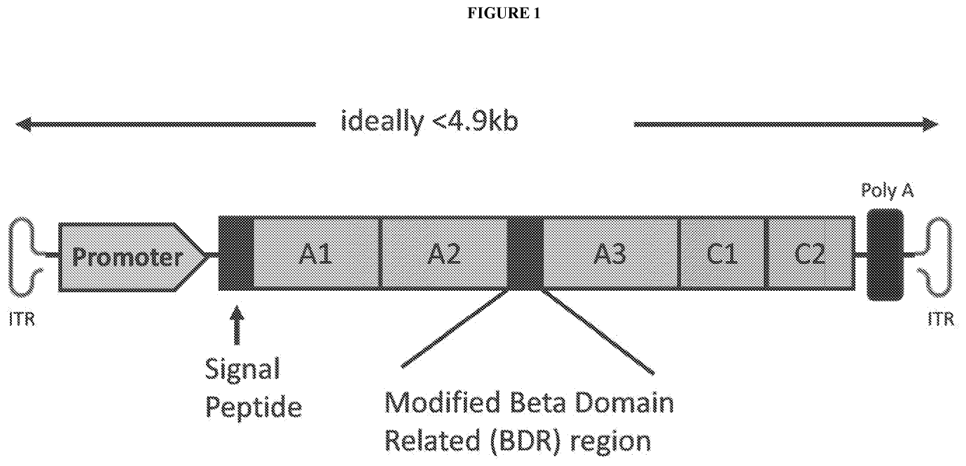

### DESCRIPTION OF THE SEQUENCE LISTING

| SEQ ID | |
|---|---|
| NO: | DESCRIPTION |
| 1 | Wild-type FVIII amino acid sequence without signal peptide |
| 2 | Wild-type FVIII nucleotide sequence without signal peptide |
| 3 | Codon optimised FVIII SQ nucleotide sequence - Co19 |
| 4 | Codon optimised FVIII SQ nucleotide sequence - Co1 |
| 5 | Codon optimised FVIII SQ nucleotide sequence - Co2 |
| 6 | Codon optimised FVIII SQ nucleotide sequence - Co3 |
| 7 | FVIII SQ amino acid sequence |
| 8 | FVIII RE amino acid sequence |
| 9 | FVIII polypeptide 96 amino acid sequence |
| 10 | FVIII polypeptide 97 amino acid sequence |
| 11 | FVIII polypeptide 98 amino acid sequence |
| 12 | FVIII polypeptide 99 amino acid sequence |
| 13 | FVIII polypeptide 100 amino acid sequence |
| 14 | FVIII polypeptide 101 amino acid sequence |
| 15 | FVIII polypeptide 102 amino acid sequence |
| 16 | FVIII polypeptide 103 amino acid sequence |
| 17 | FVIII polypeptide 104 amino acid sequence |
| 18 | FVIII polypeptide 105 amino acid sequence |
| 19 | FVIII polypeptide 106 amino acid sequence |
| 20 | FVIII polypeptide 107 amino acid sequence |
| 21 | FVIII polypeptide 108 amino acid sequence |
| 22 | FVIII polypeptide 109 amino acid sequence |
| 23 | FVIII polypeptide 110 amino acid sequence |
| 24 | FVIII polypeptide 111 amino acid sequence |
| 25 | FVIII polypeptide 112 amino acid sequence |
| 26 | FVIII polypeptide 113 amino acid sequence |
| 27 | FVIII polypeptide 114 amino acid sequence |
| 28 | FVIII polypeptide 115 amino acid sequence |
| 29 | FVIII polypeptide 116 amino acid sequence |
| 30 | FVIII polypeptide 117 amino acid sequence |
| 31 | FVIII polypeptide 96-106 amino acid sequence |
| 32 | FVIII polypeptide 99-106 amino acid sequence |
| 33 | FVIII polypeptide 96-109 amino acid sequence |
| 34 | FVIII polypeptide 99-109 amino acid sequence |
| 35 | FVIII polypeptide 96-107 amino acid sequence |
| 36 | FVIII polypeptide 107-117 amino acid sequence |
| 37 | FVIII nucleotide sequence disclosed in WO 2017/053677 |
| 38 | FVIII nucleotide sequence disclosed in WO 2017/075619 |
| 39 | Codon optimised (Co19) nucleotide sequence encoding FVIII |
| | polypeptide 96-106 |
| 40 | Codon optimised (Co1) nucleotide sequence encoding FVIII |
| | polypeptide 96-106 |
| 41 | Codon optimised (Co2) nucleotide sequence encoding FVIII |
| | polypeptide 96-106 |
| 42 | Codon optimised (Co3) nucleotide sequence encoding FVIII |
| | polypeptide 96-106 |
| 43 | Core nucleotide sequence |
| 44 | TSS |
| 45 | TSS |
| 46 | TSS |
| 47 | 3′ section of FRE49, FRE72 and FRE75 (297-335 of SEQ |
| | ID NO: 52) |
| 48 | 5′ section of HLP2 (118-162 of SEQ ID: 52) |
| 49 | 3′ section of HLP2 (243-283 of SEQ ID: 52) |
| 50 | Extended core nucleotide sequence |
| 51 | FRE72 TRE (119 bp) |
| 52 | HLP2 TRE (335 bp) |
| 53 | Amino acid sequence of native FVIII signal peptide |
| 54 | Wild-type nucleotide sequence encoding native FVIII signal |
| | peptide |
| 55 | Codon optimised (Co19) nucleotide sequence encoding FVIII |
| | signal peptide |
| 56 | Amino acid sequence of SP5 |
| 57 | Wild-type nucleotide sequence encoding SP5 |
| 58 | Amino acid sequence of SP10 |
| 59 | Wild-type nucleotide sequence encoding SP10 |
| 60 | Amino acid sequence of SP8 |
| 61 | Wild-type nucleotide sequence encoding SP8 |
| 62 | FVIII AAV construct (96-106) |
| 63 | PolyA sequence (17 bp) |
| 64 | PolyA sequence (32 bp) |
| 65 | PolyA sequence (SpA; 49 bp) |
| 66 | PolyA sequence (bGHpA; 208 bp) |
| 67 | AAV2 5′ ITR sequence |
| 68 | Amino acid sequence of AAV capsid |
| 69 | Amino acid sequence of AAV5 capsid |
| 70 | AAV2 3′ ITR sequence |
| 71 | FVIII AAV construct (26-96-106) |
| 72 | FVIII AAV construct (SEQ ID NO: 1 from WO 2017/053677) |
| 73 | FVIII AAV construct (SQ) |
| 74 | FVIII AAV construct (26-SQ) |
| 75 | Codop19 (Co19) nucleotide sequence encoding FVIII 26-96-106 |
| 76 | Codop19 (Co19) nucleotide sequence encoding FVIII 26-SQ |
| 77 | FVIII 26-96-106 amino acid sequence |
| 78 | FVIII 26-SQ amino acid sequence |
| 79 | FVIII 65-96-106 amino acid sequence |
| 80 | FVIII 26-65-96-106 amino acid sequence |
| 81 | FVIII 12SS-96-106 amino acid sequence |
| 82 | FVIII 28SS-96-106 amino acid sequence |
| 83 | FVIII 31SS-96-106 amino acid sequence |
| 84 | FVIII 65-SQ amino acid sequence |
| 85 | FVIII 26-65-SQ amino acid sequence |
| 86 | FVIII 12SS-SQ amino acid sequence |
| 87 | FVIII 28SS-SQ amino acid sequence |
| 88 | FVIII 31SS-SQ amino acid sequence |
| 89 | Amino acids 656-667 of SEQ ID NO: 1 |
| 90 | Amino acids 1823-1834 of SEQ ID NO: 1 |
| 91 | Amino acid sequence of LK03 capsid |
| 92 | Amino acid sequence of AAV6 capsid |
| 93 | The part of FVIII polypeptide SQ amino acid sequence (95) |
| | shown in FIG. 5 which contains the BDR region |
| 94 | The part of FVIII polypeptide RE amino acid sequence (85) |
| | shown in FIG. 5 which contains the BDR region |
| 95 | The part of FVIII polypeptide 96 amino acid sequence shown |
| | in FIG. 5 which contains the modified BDR region |
| 96 | The part of FVIII polypeptide 97 amino acid sequence shown |
| | in FIG. 5 which contains the modified BDR region |
| 97 | The part of FVIII polypeptide 98 amino acid sequence shown |
| | in FIG. 5 which contains the modified BDR region |
| 98 | The part of FVIII polypeptide 99 amino acid sequence shown |
| | in FIG. 5 which contains the modified BDR region |
| 99 | The part of FVIII polypeptide 100 amino acid sequence shown |
| | in FIG. 5 which contains the modified BDR region |
| 100 | The part of FVIII polypeptide 101 amino acid sequence shown |
| | in FIG. 5 which contains the modified BDR region |
| 101 | The part of FVIII polypeptide 102 amino acid sequence shown |
| | in FIG. 5 which contains the modified BDR region |
| 102 | The part of FVIII polypeptide 103 amino acid sequence shown |
| | in FIG. 5 which contains the modified BDR region |
| 103 | The part of FVIII polypeptide 104 amino acid sequence shown |
| | in FIG. 5 which contains the modified BDR region |
| 104 | The part of FVIII polypeptide 105 amino acid sequence shown |
| | in FIG. 5 which contains the modified BDR region |
| 105 | The part of FVIII polypeptide 106 amino acid sequence shown |
| | in FIG. 5 which contains the modified BDR region |
| 106 | The part of FVIII polypeptide 107 amino acid sequence shown |
| | in FIG. 5 which contains the modified BDR region |
| 107 | The part of FVIII polypeptide 108 amino acid sequence shown |
| | in FIG. 5 which contains the modified BDR region |
| 108 | The part of FVIII polypeptide 109 amino acid sequence shown |
| | in FIG. 5 which contains the modified BDR region |
| 109 | The part of FVIII polypeptide 110 amino acid sequence shown |
| | in FIG. 5 which contains the modified BDR region |
| 110 | The part of FVIII polypeptide 111 amino acid sequence shown |
| | in FIG. 5 which contains the modified BDR region |
| 111 | The part of FVIII polypeptide 112 amino acid sequence shown |
| | in FIG. 5 which contains the modified BDR region |
| 112 | The part of FVIII polypeptide 113 amino acid sequence shown |
| | in FIG. 5 which contains the modified BDR region |
| 113 | The part of FVIII polypeptide 114 amino acid sequence shown |
| | in FIG. 5 which contains the modified BDR region |
| 114 | The part of FVIII polypeptide 115 amino acid sequence shown |
| | in FIG. 5 which contains the modified BDR region |
| 115 | The part of FVIII polypeptide 116 amino acid sequence shown |
| | in FIG. 5 which contains the modified BDR region |
| 116 | The part of FVIII polypeptide 117 amino acid sequence shown |
| | in FIG. 5 which contains the modified BDR region |
| 117 | The part of FVIII polypeptide 96-106 amino acid sequence |
| | shown in FIG. 5 which contains the modified BDR region |
| 118 | The part of FVIII polypeptide 99-106 amino acid sequence |
| | shown in FIG. 5 which contains the modified BDR region |
| 119 | The part of FVIII polypeptide 96-109 amino acid sequence |
| | shown in FIG. 5 which contains the modified BDR region |
| 120 | The part of FVIII polypeptide 99-109 amino acid sequence |
| | shown in FIG. 5 which contains the modified BDR region |
| 121 | The part of FVIII polypeptide 96-107 amino acid sequence |
| | shown in FIG. 5 which contains the modified BDR region |
| 122 | The part of FVIII polypeptide 107-117 amino acid sequence |
| | shown in FIG. 5 which contains the modified BDR region |
| 123 | transcription regulatory element ten-nucleotide termination |
| | sequence ‘a’ |
| 124 | transcription regulatory element ten-nucleotide termination |
| | sequence ‘b’ |

### DETAILED DESCRIPTION

General Definitions

Unless defined otherwise, technical and scientific terms used herein have the same meaning as commonly understood by a person skilled in the art to which this invention belongs.

In general, the term “comprising” is intended to mean including but not limited to. For example, the phrase “a Factor VIII polypeptide comprising a Factor VIII amino acid sequence” should be interpreted to mean that the Factor VIII polypeptide has a Factor VIII amino acid sequence, but the Factor VIII polypeptide may contain further amino acids. Similarly, the phrase “a polynucleotide comprising a Factor VIII nucleotide sequence” refers to a polynucleotide that has a Factor VIII nucleotide sequence, but the polynucleotide may contain additional nucleotides.

In some embodiments of the invention, the word “comprising” is replaced with the phrase “consisting essentially of”. The term “consisting essentially of” means that specific further components can be present, namely those not materially affecting the essential characteristics of the subject matter.

In some embodiments of the invention, the word “comprising” is replaced with the phrase “consisting of”. The term “consisting of” is intended to be limiting. For example, the phrase “a Factor VIII polypeptide consisting of a Factor VIII amino acid sequence” should be interpreted to mean that the Factor VIII polypeptide has a Factor VIII amino acid sequence and no additional amino acids. Similarly, the phrase “a polynucleotide consisting of a Factor VIII nucleotide sequence” should be understood to mean that the polynucleotide has a Factor VIII nucleotide sequence and no additional nucleotides.

In some embodiments of the invention, the word “have” can be replaced with the word “comprise” or the phrase “consist of”.

The terms “protein” and “polypeptide” are used interchangeably herein, and are intended to refer to a polymeric chain of amino acids of any length.

The terms “nucleic acid molecule” “nucleic acid sequence”, “polynucleotide” and “nucleotide sequence” are used interchangeably herein, and are intended to refer to a polymeric chain of nucleotides of any length.

In embodiments relating to “one or more substitution mutations”, the terms “substitution mutation” and “amino acid substitution” are used interchangeably herein, and are intended to mean the substitution of one amino acid in an amino acid sequence with a different amino acid. In the phrases “substitution of” amino acid X, or “amino acid X that is (to be) substituted” amino acid X is the original or native amino acid that is present within an amino acid sequence and that is to be replaced. For example, substitution of methionine means that a native methionine amino acid is replaced by another amino acid. In the phrase “substitution with” amino acid Y, amino acid Y is the different amino acid which replaces the original or native amino acid in an amino acid sequence. For example, substitution with methionine refers to replacement of a native (non-methionine) amino acid with methionine. The standard shorthand nomenclature used to define a substitution mutation lists the original or native amino acid at a position within an amino acid sequence that is to be substituted, and the amino acid which replaces the original or native amino acid. For example, a Factor VIII amino acid sequence comprising the substitution mutation M662W refers to a Factor VIII amino acid sequence which comprises a substitution of the methionine residue at a position corresponding to position 662 with a tryptophan residue (i.e. which comprises a tryptophan residue at position 662).

In embodiments relating to “one or more substitution mutations”, the term “conservative substitution” refers to a substitution mutation in which an amino acid is substituted with another amino acid which has similar biochemical properties, such as size, charge or hydrophobicity. Amino acids may be categorised into groups on the basis of the structure of their side chains: aliphatic (glycine, alanine, valine, leucine, isoleucine); hydroxyl/sulphur-containing (serine, threonine, cysteine, methionine); cyclic (proline); aromatic (phenylalanine, tyrosine, tryptophan); basic (histidine, lysine, arginine); acidic (aspartic acid, glutamic acid); and acid amine (asparagine, glutamine). A “conservative substitution” thus refers to a substitution mutation in which an amino acid is substituted with another amino acid in the same group. Conversely, the term “non-conservative substitution” refers to a substitution in which an amino acid is substituted with another amino acid which has different biochemical properties, i.e. a substitution mutation in which an amino acid is substituted with an amino acid in another group. For example, substitution of aspartic acid with glutamic acid may be considered to be a “conservative substitution”, whilst substitution of aspartic acid with valine may be considered to be a “non-conservative” substitution.

The terms “wild-type” and “native” are used interchangeably herein, and are intended to describe something which is naturally occurring. For example, a “wild-type Factor VIII amino acid sequence” is an amino acid Factor VIII sequence which occurs in nature.

The terms “B domain” and “beta domain” are used interchangeably herein in relation to the domains of Factor VIII.

The terms “AAV viral particle” and “AAV vector” are used interchangeably herein.

The term “around” used in the context of describing the length of nucleotide or amino acid sequences indicates that a sequence may comprise or consist of a defined number of nucleotides or amino acids, plus or minus 10%, more particularly plus or minus 5%, or more particularly, plus or minus a single integer. For example, reference to an amino acid sequence of “around” 45 amino acids in length may refers to an amino acid sequence of 41-49 amino acids, more particularly 43-47 amino acids, and more particularly 44-46 amino acids in length.

In some embodiments, the term “around” used in the context of describing the length of nucleotide or amino acid sequences indicates that a sequence may comprise or consist of a defined number of nucleotides or amino acids, plus or minus 1%, more particularly plus or minus 0.1%, more particularly plus or minus 0.01%, or more particularly plus or minus a single integer.

For the purpose of this invention, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotide or amino acid residues at each position are then compared. When a position in the first sequence is occupied by the same nucleotide or amino acid as the corresponding position in the second sequence, then the nucleotides or amino acids are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence is 95% identical to SEQ ID NO: 3, SEQ ID NO: 3 would be the reference sequence. To assess whether a sequence is at least 95% identical to SEQ ID NO: 3 (an example of a reference sequence), the skilled person would carry out an alignment over the length of SEQ ID NO: 3, and identify how many positions in the test sequence were identical to those of SEQ ID NO: 3. If at least 95% of the positions are identical, the test sequence is at least 95% identical to SEQ ID NO: 3. If the sequence is shorter than SEQ ID NO: 3, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

The singular forms “a”, “an”, and “the” include plural referents unless the content clearly dictates otherwise. Thus, for example, reference to “an amino acid” includes two or more instances or versions of such amino acids.

All publications, patents and patent applications cited herein, whether supra or infra, are hereby incorporated by reference in their entirety.

A Factor VIII polypeptide comprising a Factor VIII amino acid sequence wherein the Factor VIII amino acid sequence comprises a modified beta domain related (BDR) region

The present invention provides a Factor VIII polypeptide comprising a Factor VIII amino acid sequence wherein the Factor VIII amino acid sequence comprises a BDR region which is modified relative to wild-type BDR region, which wild-type BDR region corresponds to the region between positions 713 and 1697 of SEQ ID NO: 1, wherein:
(i) the modified BDR region comprises a maximum of 88 amino acids, and the Factor VIII polypeptide has a specific activity which is higher than the specific activity of a polypeptide of SEQ ID NO: 7; and/or
(ii) the modified BDR region comprises a maximum of 74 amino acids.

The Factor VIII amino acid sequence may comprise all or part of a signal peptide, such as any of those described herein. The Factor VIII polypeptide may be a mature Factor VIII polypeptide. A “mature Factor VIII polypeptide” is a Factor VIII polypeptide which does not comprise a signal peptide. The signal peptide may have been cleaved following synthesis. The signal peptide may never have been present. For example, the Factor VIII amino acid sequence may never have comprised a signal peptide.

Wild-type Factor VIII polypeptide comprises an A1 domain, an A2 domain, a B domain, an A3 domain, a C1 domain and a C2 domain. The Factor VIII polypeptide of the invention may comprise all or part of each of the A1 domain, the A2 domain, the A3 domain, the C1 domain and/or the C2 domain. The Factor VIII polypeptide may comprise part of the B domain. The Factor VIII polypeptide may not comprise the B domain. Any or all of the domains may comprise one or more modifications, such as substitutions, insertions and/or deletions, when the Factor VIII amino acid sequence of the domain is compared to a wild-type Factor VIII amino acid sequence.

The Factor VIII polypeptide may comprise an amino acid sequence which is at least 90% identical to at least 300 amino acids of an A1 domain. The Factor VIII polypeptide may comprise an amino acid sequence which is at least 95%, at least 98%, at least 99% or 100% identical to at least 300 amino acids of an A1 domain. The Factor VIII polypeptide may comprise an amino acid sequence which is at least 90% identical to an A1 domain. The Factor VIII polypeptide may comprise an amino acid sequence which is at least 95%, at least 98%, at least 99% or 100% identical to an A1 domain. The Factor VIII polypeptide may comprise an amino acid sequence which is at least 90% identical to at least 300 amino acids of the amino acids corresponding to positions 1 to 329 of SEQ ID NO: 1. The Factor VIII polypeptide may comprise an amino acid sequence which is at least 95%, at least 98%, at least 99% or 100% identical to at least 300 amino acids of the amino acids corresponding to positions 1 to 329 of SEQ ID NO: 1. The Factor VIII polypeptide may comprise an amino acid sequence which is at least 90% identical to the amino acids corresponding to positions 1 to 329 of SEQ ID NO: 1. The Factor VIII polypeptide may comprise an amino acid sequence which is at least 95%, at least 98%, at least 99% or 100% identical to the amino acids corresponding to positions 1 to 329 of SEQ ID NO: 1.

The Factor VIII polypeptide may comprise an amino acid sequence which is at least 90% identical to at least 300 amino acids of an A2 domain. The Factor VIII polypeptide may comprise an amino acid sequence which is at least 95%, at least 96%, at least 98%, at least 99% or 100% identical to at least 300 amino acids of an A2 domain. The Factor VIII polypeptide may comprise an amino acid sequence which is at least 90% identical to an A2 domain. The Factor VIII polypeptide may comprise an amino acid sequence which is at least 95%, at least 96%, at least 98%, at least 99% or 100% identical to an A2 domain. The Factor VIII polypeptide may comprise an amino acid sequence which is at least 90% identical to at least 300 amino acids of the amino acids corresponding to positions 380 to 711 of SEQ ID NO:1. The Factor VIII polypeptide may comprise an amino acid sequence which is at least 95%, at least 98%, at least 99% or 100% identical to at least 300 amino acids of the amino acids corresponding to positions 380 to 711 of SEQ ID NO:1. The Factor VIII polypeptide may comprise an amino acid sequence which is at least 90% identical to the amino acids corresponding to positions 380 to 711 of SEQ ID NO:1. The Factor VIII polypeptide may comprise an amino acid sequence which is at least 95%, at least 98%, at least 99% or 100% identical to the amino acids corresponding to positions 380 to 711 of SEQ ID NO:1.

The Factor VIII polypeptide may comprise an amino acid sequence which is at least 90% identical to at least 300 amino acids of an A3 domain. The Factor VIII polypeptide may comprise an amino acid sequence which is at least 95%, at least 96%, at least 98%, at least 99% or 100% identical to at least 300 amino acids of an A3 domain. The Factor VIII polypeptide may comprise an amino acid sequence which is at least 90% identical to an A3 domain. The Factor VIII polypeptide may comprise an amino acid sequence which is at least 95%, at least 96%, at least 98%, at least 99% or 100% identical to an A3 domain. The Factor VIII polypeptide may comprise an amino acid sequence which is at least 90% identical to at least 300 amino acids of the amino acids corresponding to positions 1694 to 2021 of SEQ ID NO:1. The Factor VIII polypeptide may comprise an amino acid sequence which is at least 95%, at least 98%, at least 99% or 100% identical to at least 300 amino acids of the amino acids corresponding to positions 1694 to 2021 of SEQ ID NO:1. The Factor VIII polypeptide may comprise an amino acid sequence which is at least 90% identical to the amino acids corresponding to positions 1694 to 2021 of SEQ ID NO:1. The Factor VIII polypeptide may comprise an amino acid sequence which is at least 95%, at least 98%, at least 99% or 100% identical to the amino acids corresponding to positions 1694 to 2021 of SEQ ID NO:1.

The Factor VIII polypeptide may comprise an amino acid sequence which is at least 90% identical to at least 130, at least 135 or at least 140 amino acids of a C1 domain. The Factor VIII polypeptide may comprise an amino acid sequence which is at least 95%, at least 96%, at least 98%, at least 99% or 100% identical to at least 130, at least 135 or at least 140 amino acids of a C1 domain. The Factor VIII polypeptide may comprise an amino acid sequence which is at least 90% identical to a C1 domain. The Factor VIII polypeptide may comprise an amino acid sequence which is at least 95%, at least 96%, at least 98%, at least 99% or 100% identical to a C1 domain. The Factor VIII polypeptide may comprise an amino acid sequence which is at least 90% identical to at least 130, at least 135 or at least 140 amino acids of the amino acids corresponding to positions 2021 to 2169 of SEQ ID NO:1. The Factor VIII polypeptide may comprise an amino acid sequence which is at least 95%, at least 98%, at least 99% or 100% identical to at least 130, at least 135 or at least 140 amino acids of the amino acids corresponding to positions 2021 to 2169 of SEQ ID NO:1. The Factor VIII polypeptide may comprise an amino acid sequence which is at least 90% identical to the amino acids corresponding to positions 2021 to 2169 of SEQ ID NO:1. The Factor VIII polypeptide may comprise an amino acid sequence which is at least 95%, at least 98%, at least 99% or 100% identical to the amino acids corresponding to positions 2021 to 2169 of SEQ ID NO:1.

The Factor VIII polypeptide may comprise an amino acid sequence which is at least 90% identical to at least 130, at least 135 or at least 140 amino acids of a C2 domain. The Factor VIII polypeptide may comprise an amino acid sequence which is at least 95%, at least 96%, at least 98%, at least 99% or 100% identical to at least 130, at least 135 or at least 140 amino acids of a C2 domain. The Factor VIII polypeptide may comprise an amino acid sequence which is at least 90% identical to a C2 domain. The Factor VIII polypeptide may comprise an amino acid sequence which is at least 95%, at least 96%, at least 98%, at least 99% or 100% identical to a C2 domain. The Factor VIII polypeptide may comprise an amino acid sequence which is at least 90% identical to at least 130, at least 135 or at least 140 amino acids of the amino acids corresponding to positions 2174 to 2326 of SEQ ID NO:1. The Factor VIII polypeptide may comprise an amino acid sequence which is at least 95%, at least 98%, at least 99% or 100% identical to at least 130, at least 135 or at least 140 amino acids of the amino acids corresponding to positions 2174 to 2326 of SEQ ID NO:1. The Factor VIII polypeptide may comprise an amino acid sequence which is at least 90% identical to the amino acids corresponding to positions 2174 to 2326 of SEQ ID NO:1. The Factor VIII polypeptide may comprise an amino acid sequence which is at least 95%, at least 98%, at least 99% or 100% identical to the amino acids corresponding to positions 2174 to 2326 of SEQ ID NO:1.

The wild-type BDR region corresponds to the region between positions 713 and 1697 of SEQ ID NO: 1. When referring to amino acid positions, the term “between” does not include the specified positions. Thus the wild-type BDR region starts at the position that corresponds to position 714 of SEQ ID NO: 1 and ends at the position that corresponds to position 1696 of SEQ ID NO: 1. The wild-type BDR region comprises the beta domain of Factor VIII. The wild-type BDR region may be the region between positions 713 and 1697 of SEQ ID NO: 1. The wild-type BDR region may start at the position 714 of SEQ ID NO: 1 and may end at position 1696 of SEQ ID NO: 1. The wild-type BDR region may be the region from another wild-type Factor VIII amino acid sequence which corresponds to the region between positions 713 and 1697 of SEQ ID NO: 1. The wild-type BDR region may be the region from another wild-type Factor VIII amino acid sequence which corresponds to the region that starts at position 714 of SEQ ID NO: 1 and ends at position 1696 of SEQ ID NO: 1.

It is within the capabilities of the person skilled in the art to determine the region of a wild-type Factor VIII amino acid sequence which “corresponds to” the region between positions 713 and 1697 of SEQ ID NO: 1. For example, the person skilled in the art merely needs to perform a sequence alignment of the wild-type Factor VIII amino acid sequence with SEQ ID NO: 1 using a suitable alignment algorithm such as that of Needleman and Wunsch described above, and determine which region of the wild-type Factor VIII amino acid sequence aligns with the region between positions 713 and 1697 of SEQ ID NO: 1. For example, the person skilled in the art may determine which region of the wild-type Factor VIII amino acid sequence aligns with positions 714 to 1696 of SEQ ID NO: 1.

The “modified BDR region” is not identical to the wild-type BDR region. The modified BDR region is shorter than wild-type BDR region. A skilled person can readily determine whether the modified BDR region of a Factor VIII polypeptide “is modified relative to wild-type BDR region”. For example, the person skilled in the art merely needs to perform a sequence alignment of the Factor VIII amino acid sequence with a wild-type Factor VIII amino acid sequence of the Factor VIII polypeptide using a suitable alignment algorithm such as that of Needleman and Wunsch described above, determine the region of the Factor VIII amino acid sequence (modified BDR region) which aligns with the region of wild-type Factor VIII amino acid sequence (wild-type BDR region) that corresponds to the region between positions 713 and 1697 of SEQ ID NO: 1, and compare the amino acid sequence between the region in the Factor VIII amino acid sequence (modified BDR region) and the region in the wild-type Factor VIII amino acid sequence (wild-type BDR region). If there are any differences, then the region is a modified BDR region.

Since the modified BDR region of the Factor VIII polypeptide of the invention comprises a maximum of 88 amino acids, there will be one or more gap(s) in the Factor VIII amino acid sequence in the modified BDR region compared to wild-type BDR region when the Factor VIII amino acid sequence is aligned with the wild-type Factor VIII amino acid sequence. Thus, the modified BDR region of the Factor VIII polypeptide of the invention comprises one or more deletion(s) when the modified BDR region is compared to a wild-type BDR region. The modified BDR region may also have one or more modifications, such as substitutions and/or insertions, when the modified BDR region is compared to a wild-type BDR region. The modified BDR region may comprise one or more amino acids found in a wild-type BDR region. For example, the modified BDR region may comprise one or more stretches of amino acids found in a wild-type BDR region.

The modified BDR region “comprises a maximum” of a specified number of amino acids. This means that the modified BDR region comprises no more than the specified number of amino acids. The Factor VIII amino acid sequence may comprise further amino acids, but not in the modified BDR region. For example, if the modified BDR region “comprises a maximum of 88 amino acids”, the modified BDR region comprises no more than 88 amino acids. As a further example, if the modified BDR region “comprises a maximum of 74 amino acids”, the modified BDR region comprises no more than 74 amino acids.

The “Factor VIII polypeptide” is functional. A functional Factor VIII polypeptide is one which can, when activated by thrombin, form an enzymatic complex with Factor IXa, phospholipids and calcium, and the enzymatic complex can catalyse the conversion of Factor X to Factor Xa.

It is within the abilities of the person skilled in the art to determine whether a Factor VIII polypeptide is functional. The person skilled in the art merely needs to determine the specific activity of the Factor VIII polypeptide. If the specific activity of the polypeptide is at least 20% of the specific activity of a wild-type Factor VIII polypeptide such as a Factor VIII polypeptide of SEQ ID NO: 1, then it is functional. The activity of the Factor VIII polypeptide can be analysed using a chromogenic assay, such as a chromogenic assay that measures cofactor activity. For example, a suitable chromogenic assay is as follows. The

Factor VIII polypeptide is mixed with human Factor X polypeptide and Factor IXa polypeptide, thrombin, phospholipids and calcium. The thrombin activates the Factor VIII polypeptide to form Factor VIIIa polypeptide. The thrombin-activated Factor VIII polypeptide forms an enzymatic complex with Factor IXa polypeptide, phospholipids and calcium, which enzymatic complex can catalyse the conversion of Factor X polypeptide to Factor Xa polypeptide. The activity of the Factor Xa polypeptide can catalyse cleavage of a chromogenic substrate (e.g. SXa-11) to produce pNA. The level of pNA generated can be measured by determining colour development at 405 nm (e.g. measured by absorbance). Factor X polypeptide, and therefore Factor Xa polypeptide, is provided in excess. Therefore the limiting factor is Factor VIIIa polypeptide. Thus, the level of pNA generated is proportional to the amount of the Factor Xa polypeptide generated by Factor FVIIIa polypeptide in the sample, which is proportional to the activity of Factor FVIIIa polypeptide in the sample. The activity of Factor FVIIIa polypeptide in the sample is a measure of the cofactor activity of the Factor FVIII polypeptide in the sample.

For example, a suitable chromogenic assay is the BIOPHEN FVIII:C assay (Ref: 221406) manufactured by HYPHEN BioMed as used in the Examples. The activity of the Factor VIII polypeptide may be measured using the BIOPHEN FVIII:C assay.

The activity of the Factor VIII polypeptide can be analysed using a clotting assay. The assay may be a one-stage clotting assay. For example, a suitable clotting assay (a one-stage clotting assay) is as follows.

Since Factor VIII is part of the clotting cascade, a Factor VIII polypeptide that has increased activity will catalyse blood clotting more quickly than a Factor VIII polypeptide that has a lower activity. The Factor VIII polypeptide is mixed with platelet poor plasma, and incubated at 37° C. Then phospholipid and a contact activation pathway activator such as Kaolin or SynthaSIL APTT reagent are added. Calcium is then added, and the user measures the time taken for clotting to occur. Clot formation can be assessed directly by a magnetic steel ball method. Clot formation may be measured using a rotating cuvette assay in which a steel ball remains stationary in a magnetic field until the formation of fibrin strands around the ball produces movement and a change in the magnetic field can be detected. Alternatively, clot formation may be measured using a rotating steel ball assay in which a steel ball is rotated under the influence of a magnet until the formation of fibrin strands around the ball stops it rotating, which can be detected by a sensor. In either event, coagulation time may be recorded.

In patients, such as human patients, the activity of the Factor VIII polypeptide may be measured by taking a blood sample from the patient, or by performing an assay on a blood sample that has been taken from the patient.

The activity of the Factor VIII polypeptide can be analysed using a tail clip assay. A suitable tail clip assay may involve administering Factor VIII polypeptide, or polynucleotide comprising a Factor VIII nucleotide sequence in the context of a gene therapy, to mice such as knock-out mice deficient in Factor VIII. The tails of the mice are then clipped, and the time taken for the cut in the tails to clot is measured. The duration of bleeding provides a relative measure of the activity of the administered Factor VIII, for example a Factor VIII polypeptide comprising a Factor VIII amino acid sequence comprising a modified BDR region vs wild-type Factor VIII, or a Factor VIII polypeptide which is encoded by a Factor VIII nucleotide sequence wherein at least a portion of the Factor VIII nucleotide sequence is not wild-type vs wild-type Factor VIII.

For the purposes of the present application, the term “specific activity” refers to the activity (e.g. clotting activity or intrinsic enzyme activity) per unit (e.g. per jug, per IU, or per antigen level as % of level in normal human plasma) of Factor VIII polypeptide such that the activity is ‘normalised’ to take account of the amount or concentration of Factor VIII polypeptide in the sample. (Note that, typically, pooled healthy human plasma has a Factor VIII concentration of 0.2 μg/ml.) This can be done by measuring the concentration of the Factor VIII polypeptide in the sample, for example by using a standard ELISA assay, such as the assay described in Example 1, and dividing the activity by the Factor VIII concentration. A chromogenic assay may be used to measure “specific activity”. A chromogenic assay may be used to measure “specific activity” by calculating activity and dividing by the concentration of the Factor VIII polypeptide in the sample. The chromogenic assay may be any one of the chromogenic assays described herein. A clotting assay may be used to measure “specific activity”, by calculating activity and dividing by the concentration of the Factor VIII polypeptide in the sample. The clotting assay may be any one of the clotting assays described herein.

In an example of an ELISA assay, an antibody that binds to the Factor VIII polypeptide could be bound to a plate. The sample, comprising the Factor VIII polypeptide at unknown concentration, could be passed over the plate. A second detection antibody that binds to the Factor VIII polypeptide could be applied to the plate, and any excess washed off. The detection antibody that remains (i.e. is not washed off) will be bound to the Factor VIII polypeptide. The detection antibody could be linked to an enzyme such as horse radish peroxidase. The level of detection antibody that binds to the Factor VIII polypeptide on the plate could be measured by measuring the amount of the detection antibody. For example, if the detection antibody is linked to horse radish peroxidase, the horse radish peroxidase can catalyse the production of a blue reaction product from a substrate such as TMB (3,3′,5,5′-tetramethylbenzidine), and the level of the blue product can be detected by absorbance at 450 nm. The level of the blue product is proportional to the amount of detection antibody that remained after the washing step, which is proportional to the amount of the Factor VIII polypeptide in the sample. Alternatively, for example when using purified protein, the amount or concentration of Factor VIII polypeptide may be determined spectrophotometrically.

In preferred embodiments, the Factor VIII polypeptide is purified, and the specific activity is measured by a clotting assay or a chromogenic assay carried out on the purified Factor VIII polypeptide. In some embodiments, the specific activity of the Factor VIII polypeptide is measured by generating an AAV particle comprising a polynucleotide encoding the Factor VIII polypeptide, injecting mice with the AAV particle, and detecting the specific activity in plasma from the mice using a chromogenic assay. In some embodiments, the specific activity of the Factor VIII polypeptide is measured by providing cells stably expressing a polynucleotide encoding the Factor VIII polypeptide, harvesting Factor VIII polypeptide from the cells and/or culture medium, and measuring the specific activity of the Factor VIII polypeptide using a chromogenic assay.

The Factor VIII polypeptide may have a specific activity which is at least 50%, at least 60%, at least 70%, at least 75%, at least 80%, at least 85%, at least 90%, at least 95%, at least 98%, at least 99%, or at least 100% of the specific activity of a reference wild-type Factor VIII polypeptide. The “reference wild-type Factor VIII polypeptide” may be any wild-type Factor FVIII polypeptide, such as the Factor VIII polypeptide of SEQ ID NO: 1. The Factor VIII polypeptide may have a specific activity which is higher than the specific activity of a reference wild-type Factor VIII polypeptide, such as the Factor VIII polypeptide of SEQ ID NO: 1. The Factor VIII polypeptide may have a specific activity which is at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of a reference wild-type Factor VIII, such as the Factor VIII polypeptide of SEQ ID NO: 1. The Factor VIII polypeptide may have a specific activity which is between 1.2 fold and 5.5 fold, or between 1.5 fold and 5 fold, higher than the specific activity of a reference wild-type Factor VIII, such as the Factor VIII polypeptide of SEQ ID NO: 1. When referring to fold changes of activity, the term “between” includes the specified values. Thus, for example, “between 1.2 fold and 5.5 fold” includes the values 1.2 and 5.5.

The Factor VIII polypeptide may have a specific activity which is at least 50%, at least 60%, at least 70%, at least 75%, at least 80%, at least 85%, at least 90%, at least 95%, at least 98%, at least 99%, or at least 100% of the specific activity of the Factor VIII polypeptide of SEQ ID NO: 7. The Factor VIII polypeptide may have a specific activity which is higher than the specific activity of a polypeptide of SEQ ID NO: 7. The Factor VIII polypeptide may have a specific activity which is at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of the Factor VIII polypeptide of SEQ ID NO: 7. The Factor VIII polypeptide may have a specific activity which is between 1.2 fold and 5.5 fold, or between 1.5 fold and 5 fold, higher than the specific activity of the Factor VIII polypeptide of SEQ ID NO: 7. The Factor VIII polypeptide may have a specific activity which is at least 1.5 fold higher than the specific activity of the Factor VIII polypeptide of SEQ ID NO: 7. The Factor VIII polypeptide may have a specific activity which is at least 2 fold higher than the specific activity of the Factor VIII polypeptide of SEQ ID NO: 7. The Factor VIII polypeptide may have a specific activity which is between 1.5 fold and 3 fold higher than the specific activity of the Factor VIII polypeptide of SEQ ID NO: 7.

The Factor VIII polypeptide may have a specific activity which is at least 50%, at least 60%, at least 70%, at least 75%, at least 80%, at least 85%, at least 90%, at least 95%, at least 98%, at least 99%, or at least 100% of the specific activity of the Factor VIII polypeptide of SEQ ID NO: 8. The Factor VIII polypeptide may have a specific activity which is higher than the specific activity of the Factor VIII polypeptide of SEQ ID NO: 8. The Factor VIII polypeptide may have a specific activity which is at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of the Factor VIII polypeptide of SEQ ID NO: 8. The Factor VIII polypeptide may have a specific activity which is between 1.2 fold and 5.5 fold, or between 1.5 fold and 5 fold, higher than the specific activity of the Factor VIII polypeptide of SEQ ID NO: 8.

The Factor VIII polypeptide may have a specific activity which is higher than the specific activity of the Factor VIII polypeptide encoded by SEQ ID NO:37. The Factor VIII polypeptide may have a specific activity which is at least 50%, at least 60%, at least 70%, at least 75%, at least 80%, at least 85%, at least 90%, at least 95%, at least 98%, at least 99%, or at least 100% of the specific activity of the Factor VIII polypeptide encoded by SEQ ID NO: 37. The Factor VIII polypeptide may have a specific activity which is higher than the specific activity of the Factor VIII polypeptide encoded by SEQ ID NO: 37. The Factor VIII polypeptide may have a specific activity which is at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of the Factor VIII polypeptide encoded by SEQ ID NO: 37. The Factor VIII polypeptide may have a specific activity which is between 1.2 fold and 5.5 fold, or between 1.5 fold and 5 fold, higher than the specific activity of the Factor VIII polypeptide encoded by SEQ ID NO: 37. The Factor VIII polypeptide may have a specific activity which is at least 1.5 fold higher than the specific activity of the Factor VIII polypeptide encoded by SEQ ID NO: 37. The Factor VIII polypeptide may have a specific activity which is at least 2 fold higher than the specific activity of the Factor VIII polypeptide encoded by SEQ ID NO: 37. The Factor VIII polypeptide may have a specific activity which is between 1.5 fold and 3 fold higher than the specific activity of the Factor VIII polypeptide encoded by SEQ ID NO: 37.

The Factor VIII polypeptide may have a specific activity which is higher than the specific activity of the Factor VIII polypeptide encoded by SEQ ID NO:38.

The modified BDR region comprises a maximum of 88 amino acids. The modified BDR region may comprise a maximum of 87, 85, 80, 75, 70, 65, 60, 55, 50, or 45 amino acids. Preferably, the modified BDR region may comprise a maximum of 74 amino acids. The modified BDR region may comprise a maximum of 54 amino acids. The modified BDR region may comprise a maximum of 47 amino acids. The modified BDR region may comprise a maximum of 45 amino acids.

The modified BDR region may comprise at least 20, at least 25, at least 28, at least 30, at least 35, at least 40, at least 45, at least 50, at least 54, at least 55, at least 57, at least 58, at least 60, or at least 65 amino acids. The modified BDR region may comprise at least 28 amino acids. The modified BDR region may comprise at least 30 amino acids. The modified BDR region may comprise at least 45 amino acids. The modified BDR region may comprise at least 54 amino acids. The modified BDR region may comprise at least 57 amino acids. The modified BDR region may comprise at least 58 amino acids.

The modified BDR region may consist of between 20 and 89 amino acids, between 25 and 89, between 27 and 89, between 27 and 80, between 27 and 75, between 27 and 70, between 27 and 65, between 27 and 60, between 27 and 55, or between 27 and 50. The modified BDR region may consist of between 30 and 89, between 30 and 89, between 30 and 80, between 30 and 75, between 30 and 70, between 30 and 65, between 30 and 60, between 30 and 55, or between 30 and 50. The modified BDR region may consist of between 27 and 48, between 30 and 48, between 33 and 48, between 35 and 48, between 40 and 48, or between 42 and 48 amino acids. The modified BDR region may consist of between 28 and 48 amino acids. The modified BDR region may consist of between 30 and 48 amino acids. The modified BDR region may consist of around 45 amino acids. The modified BDR region may consist of 45 amino acids.

When referring to numbers of amino acids, the term “between” does not include the specified values. For example, when the modified BDR region “consists of between 20 and 89 amino acids”, the modified BDR region consists of from 21 to 88 amino acids.

The modified BDR region may consist of between 40 and 75, between 40 and 70, between 40 and 65, or between 40 and 60 amino acids. The modified BDR region may consist of between 53 and 89, between 53 and 75, between 56 and 75, between 57 and 75, or between 60 and 75 amino acids. The modified BDR region may consist of between 40 and 60 amino acids. The modified BDR region may consist of around 54 amino acids. The modified BDR region may consist of 54 amino acids.

Optionally, the Factor VIII amino acid sequence does not comprise at least 806, at least 810, at least 820, at least 835, at least 850, at least 875, or 894 amino acids corresponding to positions 746 to 1639 of SEQ ID NO: 1. Optionally, the Factor VIII amino acid sequence does not comprise at least 806 or at least 820 amino acids corresponding to positions 746 to 1639 of SEQ ID NO: 1. Optionally, the Factor VIII amino acid sequence does not comprise all the amino acids corresponding to positions 746 to 1639 of SEQ ID NO: 1. Optionally, the Factor VIII amino acid sequence does not comprise at least 820, at least 825, at least 834, at least 850, at least 875, or 908 amino acids corresponding to positions 741 to 1648 of SEQ ID NO: 1. Optionally, the Factor VIII amino acid sequence does not comprise at least 820 or at least 834 amino acids corresponding to positions 741 to 1648 of SEQ ID NO: 1. Optionally, the Factor VIII amino acid sequence does not comprise all the amino acids corresponding to positions 741 to 1648 of SEQ ID NO: 1. Optionally, the Factor VIII amino acid sequence does not comprise the beta domain.

Optionally, the Factor VIII amino acid sequence may be more than 1348 amino acids in length. Optionally, the Factor VIII amino acid sequence may be less than 1439 amino acids in length.

Optionally, the Factor VIII amino acid sequence may be at least 1349 amino acids in length.

Optionally, the Factor VIII amino acid sequence may be 1438 or fewer amino acids in length.

Optionally, the Factor VIII amino acid sequence may be at least 1357, at least 1364, at least 1366, at least 1374, at least 1376, at least 1379, at least 1384, at least 1389, or at least 1392 amino acids in length. Optionally, the Factor VIII amino acid sequence may be 1421 or fewer, 1418 or fewer, 1412 or fewer, 1409 or fewer, 1406 or fewer, 1403 or fewer, 1400 or fewer, or 1397 or fewer amino acids in length.

Optionally, the Factor VIII amino acid sequence may be between 1439 and 1348, between 1439 and 1356, between 1422 and 1375, between 1404 and 1383 or around 1394 amino acids in length. Optionally, the Factor VIII amino acid sequence may be between 1422 and 1356, between 1422 and 1367, between 1419 and 1375, between 1422 and 1376, between 1413 and 1384, between 1410 and 1385, between 1407 and 1388, between 1404 and 1389, between 1401 and 1390, between 1398 and 1391 or around 1394 amino acids in length. Optionally, the Factor VIII amino acid sequence may be around 1394 amino acids in length.

Optionally, the Factor VIII amino acid sequence may be between 1439 and 1383, between 1439 and 1402, between 1439 and 1426 or around 1438 amino acids in length. Optionally, the Factor VIII amino acid sequence may be around 1438 amino acids in length.

Optionally, the Factor VIII amino acid sequence may be around 1357, 1368, 1376, 1377, 1383, 1384, 1385, 1386, 1391, 1394, 1395, 1397, 1400, 1403, 1406, 1409, 1412, 1415, 1418 or 1421 amino acids in length. Optionally, the Factor VIII amino acid sequence may be 1357, 1368, 1376, 1377, 1383, 1384, 1385, 1386, 1391, 1394, 1395, 1397, 1400, 1403, 1406, 1409, 1412, 1415, 1418 or 1421 amino acids in length.

Optionally, the Factor VIII amino acid sequence may be around 1377, around 1378, around 1385, around 1386, around 1387, around 1389, around 1391 or around 1394 amino acids in length. Optionally, the Factor VIII amino acid sequence may be 1377, 1378, 1385, 1386, 1387, 1389, 1391 or 1394 amino acids in length. Optionally, the Factor VIII amino acid sequence may be 1394 amino acids in length.

The Factor VIII amino acid sequence may comprise at least 650, at least 700, at least 710, or 713 amino acids corresponding to positions 1 to 713 of SEQ ID NO: 1. The Factor VIII amino acid sequence may comprise all the amino acids corresponding to positions 1 to 713 of SEQ ID NO: 1. The Factor VIII amino acid sequence may comprise at least 550, at least 600, at least 620, or 636 amino acids corresponding to positions 1697 to 2332 of SEQ ID NO: 1. The Factor VIII amino acid sequence may comprise all the amino acids corresponding to positions 1697 to 2332 of SEQ ID NO: 1.

When a range of positions are provided, e.g. positions 1 to 713, the recited values are included. Therefore, in the example “positions 1 to 713 ”, 1 and 713 are included. Thus, the use of “to” is inclusive.

Optionally, the Factor VIII amino acid sequence does not comprise at least one amino acid(s) corresponding to positions 724 to 740 of SEQ ID NO: 1. Optionally, the Factor VIII amino acid sequence does not comprise at least 2, at least 3, at least 4, at least 5, at least 6, at least 7, at least 8, at least 9, at least 10, at least 11, at least 12, at least 13, at least 14, at least 15, at least 16, or 17 amino acids corresponding to positions 724 to 740 of SEQ ID NO: 1. Optionally, the Factor VIII amino acid sequence does not comprise 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, or 2 or fewer amino acids corresponding to positions 724 to 740 of SEQ ID NO: 1. Optionally, the Factor VIII amino acid sequence does not comprise between 1 and 18, between 5 and 18, between 7 and 18, between 10 and 18, between 12 and 18, or between 15 and 18 amino acids corresponding to positions 724 to 740 of SEQ ID NO 1. Optionally, the Factor VIII amino acid sequence does not comprise between 2 and 18, between 5 and 16, or between 8 and 13 amino acids corresponding to positions 724 to 740 of SEQ ID NO: 1. Optionally, the Factor VIII amino acid sequence does not comprise between 2 and 16, between 2 and 13, between 2 and 10, or between 2 and 7 amino acids corresponding to positions 724 to 740 of SEQ ID NO: 1. Optionally, the Factor VIII amino acid sequence does not comprise at least 2 contiguous amino acids corresponding to positions 724 to 740 of SEQ ID NO: 1. Optionally, the Factor VIII amino acid sequence does not comprise at least 3, at least 4, at least 5, at least 6, at least 7, at least 8, at least 9, at least 10, at least 11, at least 12, at least 13, at least 14, at least 15, at least 16, or 17 contiguous amino acids corresponding to positions 724 to 740 of SEQ ID NO: 1. Optionally, the Factor VIII amino acid sequence does not comprise amino acids corresponding to positions 739 to 740, 738 to 740, 737 to 740, 736 to 740, 735 to 740, 734 to 740, 733 to 740, 732 to 740, 731 to 740, 730 to 740, 729 to 740, 728 to 740, 727 to 740, 726 to 740, 725 to 740, or 724 to 740 of SEQ ID NO: 1. Optionally, the Factor VIII amino acid sequence does not comprise amino acids corresponding to positions 738 to 740 of SEQ ID NO: 1. Optionally, the Factor VIII amino acid sequence does not comprise amino acids corresponding to positions 735 to 740 of SEQ ID NO: 1. Optionally, the Factor VIII amino acid sequence does not comprise amino acids corresponding to positions 732 to 740 of SEQ ID NO: 1. Optionally, the Factor VIII amino acid sequence does not comprise amino acids corresponding to positions 729 to 740 of SEQ ID NO: 1. Optionally, the Factor VIII amino acid sequence does not comprise amino acids corresponding to positions 726 to 740 of SEQ ID NO: 1. Optionally, the Factor VIII amino acid sequence does not comprise amino acids corresponding to positions 724 to 740 of SEQ ID NO: 1.

The Factor VIII amino acid sequence may comprise at least one amino acid(s) corresponding to positions 724 to 740 of SEQ ID NO: 1. The Factor VIII amino acid sequence may comprise at least 2, at least 3, at least 4, at least 5, at least 6, at least 7, at least 8, at least 9, at least 10, at least 11, at least 12, at least 13, at least 14, at least 15, at least 16, or 17 amino acids corresponding to positions 724 to 740 of SEQ ID NO: 1. The Factor VIII amino acid sequence may comprise 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, or 2 or fewer amino acids corresponding to positions 724 to 740 of SEQ ID NO: 1. The Factor VIII amino acid sequence may comprise between 1 and 18, between 4 and 15, between 4 and 12, or between 4 and 9 amino acids corresponding to positions 724 to 740 of SEQ ID NO: 1.

The Factor VIII amino acid sequence may comprise at least 2 contiguous amino acids corresponding to positions 724 to 740 of SEQ ID NO: 1. The Factor VIII amino acid sequence may comprise at least 3, at least 4, at least 5, at least 6, at least 7, at least 8, at least 9, at least 10, at least 11, at least 12, at least 13, at least 14, at least 15, at least 16, or 17 contiguous amino acids corresponding to positions 724 to 740 of SEQ ID NO: 1. The Factor VIII amino acid sequence may comprise amino acids corresponding to positions 724 to 725, 724 to 726, 724 to 727, 724 to 728, 724 to 729, 724 to 730, 724 to 731, 724 to 732, 724 to 733, 724 to 734, 724 to 735, 724 to 736, 724 to 737, 724 to 738, 724 to 739, or 724 to 740 of SEQ ID NO: 1. The Factor VIII amino acid sequence may comprise amino acids corresponding to positions 724 to 725 of SEQ ID NO: 1. The Factor VIII amino acid sequence may comprise amino acids corresponding to positions 724 to 728 of SEQ ID NO: 1. The Factor VIII amino acid sequence may comprise amino acids corresponding to positions 724 to 731 of SEQ ID NO: 1. The Factor VIII amino acid sequence may comprise amino acids corresponding to positions 724 to 734 of SEQ ID NO: 1. The Factor VIII amino acid sequence may comprise amino acids corresponding to positions 724 to 737 of SEQ ID NO: 1. The Factor VIII amino acid sequence may comprise amino acids corresponding to positions 724 to 740 of SEQ ID NO: 1.

The Factor VIII amino acid sequence may comprise at least one of the tyrosine amino acids corresponding to positions 718, 719 and 723 of SEQ ID NO: 1. The Factor VIII amino acid sequence may comprise the tyrosine amino acid corresponding to position 718 of SEQ ID NO: 1. The Factor VIII amino acid sequence may comprise the tyrosine amino acid corresponding to position 719 of SEQ ID NO: 1. The Factor VIII amino acid sequence may comprise the tyrosine amino acid corresponding to position 723 of SEQ ID NO: 1. The Factor VIII amino acid sequence may comprise the tyrosine amino acids corresponding to positions 718, 719 and 723 of SEQ ID NO: 1.

The Factor VIII amino acid sequence may comprise at least one of the amino acid(s) corresponding to positions 714 to 723 of SEQ ID NO: 1. The Factor VIII amino acid sequence may comprise at least 2, at least 3, at least 4, at least 5, at least 6, at least 7, at least 8, at least 9, or 10 amino acids corresponding to positions 714 to 723 of SEQ ID NO: 1. The Factor VIII amino acid sequence may comprise 10 or fewer, 9 or fewer, 8 or fewer, 7 or fewer, 6 or fewer, 5 or fewer, 4 or fewer, 3 or fewer, or 2 or fewer amino acids corresponding to positions 714 to 723 of SEQ ID NO: 1. The Factor VIII amino acid sequence may comprise between 2 and 11, between 5 and 11, or between 7 and 11 amino acids corresponding to positions 714 to 723 of SEQ ID NO: 1. The Factor VIII amino acid sequence may comprise at least 2 contiguous amino acids corresponding to positions 714 to 723 of SEQ ID NO: 1. The Factor VIII amino acid sequence may comprise at least 3, at least 4, at least 5, at least 6, at least 7, at least 8, at least 9, or 10 contiguous amino acids corresponding to positions 714 to 723 of SEQ ID NO: 1. The Factor VIII amino acid sequence may comprise amino acids corresponding to positions 714 to 715, 714 to 716, 714 to 717, 714 to 718, 714 to 719, 714 to 720, 714 to 721, 714 to 722, or 714 to 723 of SEQ ID NO: 1. The Factor VIII amino acid sequence may comprise amino acids corresponding to positions 714 to 719 of SEQ ID NO: 1. The Factor VIII amino acid sequence may comprise amino acids corresponding to positions 714 to 722 of SEQ ID NO: 1. The Factor VIII amino acid sequence may comprise amino acids corresponding to positions 714 to 723 of SEQ ID NO: 1.

Optionally, the Factor VIII amino acid sequence does not comprise at least one amino acid(s) corresponding to positions 741 to 745 of SEQ ID NO: 1. Optionally, the Factor VIII amino acid sequence does not comprise at least 2, at least 3, at least 4, or 5 amino acids corresponding to positions 741 to 745 of SEQ ID NO: 1. Optionally, the Factor VIII amino acid sequence does not comprise 5 or fewer, 4 or fewer, 3 or fewer, or 2 or fewer amino acids corresponding to positions 741 to 745 of SEQ ID NO: 1. Optionally, the Factor VIII amino acid sequence does not comprise between 1 and 6, or between 3 and 6 amino acids corresponding to positions 741 to 745 of SEQ ID NO: 1. Optionally, the Factor VIII amino acid sequence does not comprise at least 2 contiguous amino acids corresponding to positions 741 to 745 of SEQ ID NO: 1. Optionally, the Factor VIII amino acid sequence does not comprise at least 3, at least 4, or 5 contiguous amino acids corresponding to positions 741 to 745 of SEQ ID NO: 1. Optionally, the Factor VIII amino acid sequence does not comprise amino acids corresponding to positions 744 to 745, 743 to 745, 742 to 745, or 741 to 745 of SEQ ID NO: 1. Optionally, the Factor VIII amino acid sequence does not comprise amino acids corresponding to positions 741 to 745 of SEQ ID NO: 1.

Optionally, the Factor VIII amino acid sequence does not comprise at least one amino acid(s) corresponding to positions 1640 to 1648 of SEQ ID NO: 1. Optionally, the Factor VIII amino acid sequence does not comprise at least 2, at least 3, at least 4, at least 5, at least 6, at least 7, at least 8, or 9 amino acids corresponding to positions 1640 to 1648 of SEQ ID NO: 1. Optionally, the Factor VIII amino acid sequence does not comprise 9 or fewer, 8 or fewer, 7 or fewer, 6 or fewer, 5 or fewer, 4 or fewer, 3 or fewer, or 2 or fewer amino acids corresponding to positions 1640 to 1648 of SEQ ID NO: 1. Optionally, the Factor VIII amino acid sequence does not comprise between 1 and 10, between 4 and 10, or between 6 and 10 amino acids corresponding to positions 1640 to 1648 of SEQ ID NO: 1. Optionally, the Factor VIII amino acid sequence does not comprise at least 2 contiguous amino acids corresponding to positions 1640 to 1648 of SEQ ID NO: 1. Optionally, the Factor VIII amino acid sequence does not comprise at least 3, at least 4, at least 5, at least 6, at least 7, at least 8, or 9 contiguous amino acids corresponding to positions 1640 to 1648 of SEQ ID NO: 1. Optionally, the Factor VIII amino acid sequence does not comprise amino acids corresponding to positions 1640 to 1641, 1640 to 1642, 1640 to 1643, 1640 to 1644, 1640 to 1645, 1640 to 1646, 1640 to 1647, or 1640 to 1648 of SEQ ID NO: 1. Optionally, the Factor VIII amino acid sequence does not comprise amino acids corresponding to positions 1640 to 1648 of SEQ ID NO: 1.

Optionally, the Factor VIII amino acid sequence does not comprise an amino acid sequence where the amino acid corresponding to position 1644 of SEQ ID NO: 1 is immediately juxtaposed to the amino acid corresponding to position 1649 of SEQ ID NO: 1. Thus, optionally, the amino acid sequence corresponding to positions 1645 to 1648 of SEQ ID NO: 1 is not absent (e.g. deleted) unless at least one further amino acid which is immediately juxtaposed to the amino acid sequence corresponding to positions 1645 to 1648 of SEQ ID NO: 1 (e.g. the amino acid sequence corresponding to position 1644 and/or 1649) is also absent (e.g. deleted).

Optionally, the Factor VIII amino acid sequence does not comprise different amino acids at positions corresponding to positions 1645 to 1648 of SEQ ID NO: 1. An amino acid is considered “different” if it is not identical to the amino acid in the corresponding position in a wild-type Factor VIII amino acid sequence, such as SEQ ID NO: 1. For example:
If the amino acid corresponding to position 1645 of SEQ ID NO: 1 is arginine, then it is not a different amino acid.
If the amino acid corresponding to position 1646 of SEQ ID NO: 1 is histidine, then it is not a different amino acid.
If the amino acid corresponding to position 1647 of SEQ ID NO: 1 is glutamine, then it is not a different amino acid.
If the amino acid corresponding to position 1648 of SEQ ID NO: 1 is arginine, then it is not a different amino acid.

The Factor VIII amino acid sequence may comprise amino acids corresponding to positions 1645 to 1648 of SEQ ID NO: 1. Preferably, when the Factor VIII amino acid sequence comprises amino acids corresponding to positions 1645 to 1648 of SEQ ID NO: 1:
the amino acid at the position corresponding to position 1645 of SEQ ID NO: 1 is arginine;
the amino acid at the position corresponding to position 1646 of SEQ ID NO: 1 is histidine;
the amino acid at the position corresponding to position 1647 of SEQ ID NO: 1 is glutamine; and
the amino acid at the position corresponding to position 1648 of SEQ ID NO: 1 is arginine.

Optionally, the Factor VIII amino acid sequence may comprise at least one amino acid(s) corresponding to positions 741 to 745 of SEQ ID NO:1. Optionally, the Factor VIII amino acid sequence may comprise at least 2, at least 3, at least 4 or 5 amino acids corresponding to positions 741 to 745 of SEQ ID NO:1. Optionally, the Factor VIII amino acid sequence may comprise 5 or fewer, 4 or fewer, 3 or fewer or 2 or fewer amino acids corresponding to positions 741 to 745 of SEQ ID NO:1. Optionally, the Factor VIII amino acid sequence may comprise between 1 and 6, or between 3 and 6 amino acids corresponding to positions 741 to 745 of SEQ ID NO:1. Optionally, the Factor VIII amino acid sequence may comprise at least 2 contiguous amino acids corresponding to positions 741 to 745 of SEQ ID NO:1. Optionally, the Factor VIII amino acid sequence does not comprise at least 3, at least 4 or 5 contiguous amino acids corresponding to positions 741 to 745 of SEQ ID NO:1. Optionally, the Factor VIII amino acid sequence may comprise amino acids corresponding to positions 741, 741 to 742, 741 to 743, 741 to 744, or 741 to 745 of SEQ ID NO:1. Optionally, the Factor VIII amino acid sequence may comprise amino acids corresponding to positions 741 to 745 of SEQ ID NO:1.

Optionally, the Factor VIII amino acid sequence may comprise at least one amino acid(s) corresponding to positions 1640 to 1648 of SEQ ID NO:1. Optionally, the Factor VIII amino acid sequence may comprise at least 2, at least 3, at least 4, at least 5, at least 6, at least 7, at least 8 or 9 amino acids corresponding to positions 1640 to 1648 of SEQ ID NO:1. Optionally, the Factor VIII amino acid sequence may comprise 9 or fewer, 8 or fewer, 7 or fewer, 6 or fewer, 5 or fewer, 4 or fewer, 3 or fewer or 2 or fewer amino acids corresponding to positions 1640 to 1648 of SEQ ID NO:1. Optionally, the Factor VIII amino acid sequence may comprise between 1 and 10, between 4 and 10 or between 6 and 10 amino acids corresponding to positions 1640 to 1648 of SEQ ID NO:1. Optionally, the Factor VIII amino acid sequence may comprise at least 2 contiguous amino acids corresponding to positions 1640 to 1648 of SEQ ID NO:1. Optionally, the Factor VIII amino acid sequence may comprise at least 3, at least 4, at least 5, at least 6, at least 7, at least 8 or 9 contiguous amino acids corresponding to positions 1640 to 1648 of SEQ ID NO:1. Optionally, the Factor VIII amino acid sequence may comprise amino acids corresponding to positions 1640 to 1648, 1641 to 1648, 1642 to 1648, 1643 to 1648, 1644 to 1648, 1645 to 1648, 1646 to 1648, 1647 to 1648, or 1648 of SEQ ID NO:1. Optionally, the Factor VIII amino acid sequence may comprise amino acids corresponding to positions 1640 to 1648 of SEQ ID NO:1.

Optionally, the Factor VIII amino acid sequence may comprise at least one amino acid(s) corresponding to positions 741 to 745 and 1640 to 1648 of SEQ ID NO:1. Optionally, the Factor VIII amino acid sequence may comprise at least 2, at least 3, at least 4, at least 5, at least 6, at least 7, at least 8, at least 9, at least 10, at least 11, at least 12, at least 13 or 14 amino acids corresponding to positions 741 to 745 and 1640 to 1648 of SEQ ID NO:1.

Optionally, the Factor VIII amino acid sequence may comprise 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer or 2 or fewer amino acids corresponding to positions 741 to 745 and 1640 to 1648 of SEQ ID NO:1. Optionally, the Factor VIII amino acid sequence may comprise between 1 and 15, between 4 and 15, between 7 and 15 or between 10 and 15 amino acids corresponding to positions 741 to 745 and 1640 to 1648 of SEQ ID NO:1. Optionally, the Factor VIII amino acid sequence may comprise at least 2 contiguous amino acids corresponding to positions 741 to 745 and 1640 to 1648 of SEQ ID NO:1. Optionally, the Factor VIII amino acid sequence may comprise at least 3, at least 4, at least 5, at least 6, at least 7, at least 8, at least 9, at least 10, at least 11, at least 12, at least 13 or 14 contiguous amino acids corresponding to positions 741 to 745 and 1640 to 1648 of SEQ ID NO:1. Optionally, the Factor VIII amino acid sequence may comprise amino acids corresponding to positions 741, 741 to 742, 741 to 743, 741 to 744, or 741 to 745 and/or amino acids corresponding to positions 1640 to 1648, 1641 to 1648, 1642 to 1648, 1643 to 1648, 1644 to 1648, 1645 to 1648, 1646 to 1648, 1647 to 1648, or 1648 of SEQ ID NO:1. Optionally, the Factor VIII amino acid sequence may comprise amino acids corresponding to positions 741 to 745 and 1640 to 1648 of SEQ ID NO:1. Optionally, the Factor VIII amino acid sequence may comprise the SQ linker sequence SFSQNPPVLKRHQR.

Optionally, the Factor VIII amino acid sequence does not comprise at least one amino acid(s) corresponding to positions 1649 to 1689 of SEQ ID NO:1. Optionally the Factor VIII amino acid sequence does not comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 or 41 amino acids corresponding to positions 1649 to 1689 of SEQ ID NO:1. Optionally, the Factor VIII amino acid sequence does not comprise 41 or fewer, 40 or fewer, 39 or fewer, 38 or fewer, 37 or fewer, 36 or fewer, 35 or fewer, 34 or fewer, 33 or fewer, 32 or fewer, 31 or fewer, 30 or fewer, 29 or fewer, 28 or fewer, 27 or fewer, 26 or fewer, 25 or fewer, 24 or fewer, 23 or fewer, 22 or fewer, 21 or fewer, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer amino acids corresponding to positions 1649 to 1689 of SEQ ID NO:1. Optionally, the Factor VIII amino acid sequence does not comprise between 1 and 42, between 5 and 42, between 8 and 42, between 11 and 42, between 14 and 42, between 17 and 42, between 20 and 42, between 23 and 42, between 26 and 42, between 29 and 42, between 32 and 42, between 35 and 42 or between 39 and 42 amino acids corresponding to positions 1649 to 1689 of SEQ ID NO:1. Optionally, the Factor VIII amino acid sequence does not comprise at least 2 contiguous amino acids corresponding to positions 1649 to 1689 of SEQ ID NO:1. Optionally, the Factor VIII amino acid sequence does not comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 or 41 contiguous amino acids corresponding to positions 1649 to 1689 of SEQ ID NO:1. Optionally, the Factor VIII amino acid sequence does not comprise amino acids corresponding to positions 1649 to 1670, 1649 to 1671, 1649 to 1672, 1649 to 1673, 1649 to 1674, 1649 to 1675, 1649 to 1676, 1649 to 1677, 1649 to 1678, 1649 to 1679, 1649 to 1680, 1649 to 1681, 1649 to 1682, 1649 to 1683, 1649 to 1684, 1649 to 1685, 1649 to 1686, 1649 to 1687, 1649 to 1688 or 1649 to 1689 of SEQ ID NO: 1. Optionally the Factor VIII amino acid sequence does not comprise amino acids corresponding to positions 1649 to 1670 of SEQ ID NO: 1. Optionally the Factor VIII amino acid sequence does not comprise amino acids corresponding to positions 1649 to 1673 of SEQ ID NO: 1. Optionally the Factor VIII amino acid sequence does not comprise amino acids corresponding to positions 1649 to 1676 of SEQ ID NO: 1. Optionally the Factor VIII amino acid sequence does not comprise amino acids corresponding to positions 1649 to 1679 of SEQ ID NO: 1. Optionally the Factor VIII amino acid sequence does not comprise amino acids corresponding to positions 1649 to 1682 of SEQ ID NO: 1. Optionally the Factor VIII amino acid sequence does not comprise amino acids corresponding to positions 1649 to 1685 of SEQ ID NO: 1. Optionally the Factor VIII amino acid sequence does not comprise amino acids corresponding to positions 1649 to 1686 of SEQ ID NO: 1. Optionally the Factor VIII amino acid sequence does not comprise amino acids corresponding to positions 1649 to 1687 of SEQ ID NO: 1. Optionally the Factor VIII amino acid sequence does not comprise amino acids corresponding to positions 1649 to 1688 of SEQ ID NO: 1. Optionally the Factor VIII amino acid sequence does not comprise amino acids corresponding to positions 1649 to 1689 of SEQ ID NO: 1.

Optionally, the Factor VIII amino acid sequence does not comprise at least one amino acid(s) corresponding to positions 1649 to 1669 of SEQ ID NO: 1. Optionally, the Factor VIII amino acid sequence does not comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or 21 amino acids corresponding to positions 1649 to 1669 of SEQ ID NO: 1. Optionally, the Factor VIII amino acid sequence does not comprise 21 or fewer, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, or 2 or fewer amino acids corresponding to positions 1649 to 1669 of SEQ ID NO: 1. Optionally, the Factor VIII amino acid sequence does not comprise between 1 and 22, between 5 and 22, between 8 and 22, between 11 and 22, between 14 and 22, between 17 and 22, or between 19 and 22 amino acids corresponding to positions 1649 to 1669 of SEQ ID NO: 1. Optionally, the Factor VIII amino acid sequence does not comprise at least 2 contiguous amino acids corresponding to positions 1649 to 1669 of SEQ ID NO: 1. Optionally, the Factor VIII amino acid sequence does not comprise at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or 21 contiguous amino acids corresponding to positions 1649 to 1669 of SEQ ID NO: 1. Optionally, the Factor VIII amino acid sequence does not comprise amino acids corresponding to positions 1649 to 1650, 1649 to 1651, 1649 to 1652, 1649 to 1653, 1649 to 1654, 1649 to 1655, 1649 to 1656, 1649 to 1657, 1649 to 1658, 1649 to 1659, 1649 to 1660, 1649 to 1661, 1649 to 1662, 1649 to 1663, 1649 to 1664, 1649 to 1665, 1649 to 1666, 1649 to 1667, 1649 to 1668, or 1649 to 1669 of SEQ ID NO: 1. Optionally, the Factor VIII amino acid sequence does not comprise amino acids corresponding to positions 1649 to 1651 of SEQ ID NO: 1. Optionally, the Factor VIII amino acid sequence does not comprise amino acids corresponding to positions 1649 to 1654 of SEQ ID NO: 1. Optionally, the Factor VIII amino acid sequence does not comprise amino acids corresponding to positions 1649 to 1657 of SEQ ID NO: 1. Optionally, the Factor VIII amino acid sequence does not comprise amino acids corresponding to positions 1649 to 1660 of SEQ ID NO: 1. Optionally, the Factor VIII amino acid sequence does not comprise amino acids corresponding to positions 1649 to 1663 of SEQ ID NO: 1. Optionally, the Factor VIII amino acid sequence does not comprise amino acids corresponding to positions 1649 to 1664 of SEQ ID NO: 1. Optionally, the Factor VIII amino acid sequence does not comprise amino acids corresponding to positions 1649 to 1666 of SEQ ID NO: 1. Optionally, the Factor VIII amino acid sequence does not comprise amino acids corresponding to positions 1649 to 1667 of SEQ ID NO: 1. Optionally, the Factor VIII amino acid sequence does not comprise amino acids corresponding to positions 1649 to 1669 of SEQ ID NO: 1.

The Factor VIII amino acid sequence may comprise at least one of the amino acid(s) corresponding to positions 1649 to 1689 of SEQ ID NO:1. The Factor VIII amino acid sequence may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 or 41 of the amino acids corresponding to positions 1649 to 1689 of SEQ ID NO:1. The FVIII amino acid sequence may comprise 41 or fewer, 40 or fewer, 39 or fewer, 38 or fewer, 37 or fewer, 36 or fewer, 35 or fewer, 34 or fewer, 33 or fewer, 32 or fewer, 31 or fewer, 30 or fewer, 29 or fewer, 28 or fewer, 27 or fewer, 26 or fewer, 25 or fewer, 24 or fewer, 23 or fewer, 22 or fewer, 21 or fewer, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer amino acids corresponding to positions 1649 to 1689 of SEQ ID NO:1. The FVIII amino acid sequence may comprise between 1 and 42, between 1 and 39, between 1 and 36, between 1 and 33, between 1 and 30, between 1 and 27, between 1 and 24, between 1 and 21, between 1 and 18, between 1 and 15, between 1 and 12, between 1 and 9, between 1 and 6, or between 1 and 4 amino acids corresponding to positions 1649 to 1689. The Factor VIII amino acid sequence may comprise at least 2 contiguous amino acids corresponding to positions 1649 to 1689 of SEQ ID NO:1. The Factor VIII amino acid sequence may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 or 41 contiguous amino acids corresponding to positions 1649 to 1689 of SEQ ID NO:1. The Factor VIII amino acid sequence may comprise amino acids corresponding to positions 1688 to 1689, 1687 to 1689, 1686 to 1689, 1685 to 1689, 1684 to 1689, 1683 to 1689, 1682 to 1689, 1681 to 1689, 1680 to 1689, 1679 to 1689, 1678 to 1689, 1677 to 1689, 1676 to 1689, 1675 to 1698, 1674 to 1689, 1673 to 1689, 1672 to 1689, 1671 to 1689 or 1670 to 1689 of SEQ ID NO:1. The Factor VIII amino acid sequence may comprise amino acids corresponding to positions 1673 to 1689 of SEQ ID NO:1. The Factor VIII amino acid sequence may comprise amino acids corresponding to positions 1670 to 1689 of SEQ ID NO:1. The Factor VIII amino acid sequence may comprise amino acids corresponding to positions 1649 to 1689 of SEQ ID NO:1.

The Factor VIII amino acid sequence may comprise at least one of the amino acid(s) corresponding to positions 1649 to 1669 of SEQ ID NO: 1. The Factor VIII amino acid sequence may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or 21 amino acids corresponding to positions 1649 to 1669 of SEQ ID NO: 1. The Factor VIII amino acid sequence may comprise 21 or fewer, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, or 2 or fewer amino acids corresponding to positions 1649 to 1669 of SEQ ID NO: 1. The Factor VIII amino acid sequence may comprise between 1 and 22, between 1 and 19, between 1 and 16, between 1 and 12, between 1 and 9, between 1 and 6, or between 1 and 4 amino acids corresponding to positions 1649 to 1669 of SEQ ID NO: 1. The Factor VIII amino acid sequence may comprise at least 2 contiguous amino acids corresponding to positions 1649 to 1669 of SEQ ID NO: 1. The Factor VIII amino acid sequence may comprise at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or 21 contiguous amino acids corresponding to positions 1649 to 1669 of SEQ ID NO: 1. The Factor VIII amino acid sequence may comprise amino acids corresponding to positions 1668 to 1669, 1667 to 1669, 1666 to 1669, 1665 to 1669, 1664 to 1669, 1663 to 1669, 1662 to 1669, 1661 to 1669, 1660 to 1669, 1659 to 1669, 1658 to 1669, 1657 to 1669, 1656 to 1669, 1655 to 1669, 1654 to 1669, 1653 to 1669, 1652 to 1669, 1651 to 1669, 1650 to 1669, or 1649 to 1669 of SEQ ID NO: 1. The Factor VIII amino acid sequence may comprise an amino acid corresponding to position 1664 of SEQ ID NO: 1. Preferably, the amino acid corresponding to position 1664 of SEQ ID NO: 1 is a tyrosine. The Factor VIII amino acid sequence may comprise amino acids corresponding to positions 1668 and/or 1669 of SEQ ID NO: 1. The Factor VIII amino acid sequence may comprise amino acids corresponding to positions 1668 to 1669 of SEQ ID NO: 1. The Factor VIII amino acid sequence may comprise amino acids corresponding to positions 1667 to 1669 of SEQ ID NO: 1. The Factor VIII amino acid sequence may comprise amino acids corresponding to positions 1664 to 1669 of SEQ ID NO: 1. The Factor VIII amino acid sequence may comprise amino acids corresponding to positions 1661 to 1669 of SEQ ID NO: 1. The Factor VIII amino acid sequence may comprise amino acids corresponding to positions 1658 to 1669 of SEQ ID NO: 1. The Factor VIII amino acid sequence may comprise amino acids corresponding to positions 1655 to 1669 of SEQ ID NO: 1. The Factor VIII amino acid sequence may comprise amino acids corresponding to positions 1652 to 1669 of SEQ ID NO: 1. The Factor VIII amino acid sequence may comprise amino acids corresponding to positions 1649 to 1669 of SEQ ID NO: 1.

The Factor VIII amino acid sequence may comprise at least one amino acid(s) corresponding to positions 1668 to 1678 of SEQ ID NO: 1. The Factor VIII amino acid sequence may comprise at least 2, at least 3, at least 4, at least 5, at least 6, at least 7, at least 8, at least 9, at least 10 or 11 amino acids corresponding to positions 1668 to 1678 of SEQ ID NO: 1. The

Factor VIII amino acid sequence may comprise 11 or fewer, 10 or fewer, 9 or fewer, 8 or fewer, 7 or fewer, 6 or fewer, 5 or fewer, 4 or fewer, 3 or fewer, or 2 or fewer amino acids corresponding to positions 1668 to 1678 of SEQ ID NO: 1. The Factor VIII amino acid sequence may comprise between 1 and 12, between 4 and 12, or between 7 and 12 amino acids corresponding to positions 1668 to 1678 of SEQ ID NO: 1. The Factor VIII amino acid sequence may comprise at least 2 contiguous amino acids corresponding to positions 1668 to 1678 of SEQ ID NO: 1. The Factor VIII amino acid sequence may comprise at least 3, at least 4, at least 5, at least 6, at least 7, at least 8, at least 9, at least 10, or 11 contiguous amino acids corresponding to positions 1668 to 1678 of SEQ ID NO: 1. The Factor VIII amino acid sequence may comprise amino acids corresponding to positions 1668 to 1678 of SEQ ID NO:1. The Factor VIII amino acid sequence may comprise amino acids corresponding to positions 1670 to 1678 of SEQ ID NO: 1. The Factor VIII amino acid sequence may comprise amino acids corresponding to positions 1673 to 1678 of SEQ ID NO:1. The Factor VIII polypeptide comprising the Factor VIII amino acid sequence may bind to a von Willebrand factor. The Factor VIII amino acid sequence may comprise a von Willebrand factor binding site. The binding of a von Willebrand factor can be measured by an ELISA assay. Optionally, the Factor VIII amino acid sequence comprises a von Willebrand factor binding site if it can bind to a von Willebrand factor as measured by ELISA. An exemplary assay for measuring binding of a von Willebrand factor to FVIII is as follows.

An antibody against von Willebrand factor (e.g. a rabbit anti-human von Willebrand factor antibody) may be immobilised in wells of a multi-well plate. Von Willebrand factor may be added to the wells of the multi-well plate and incubated to allow binding of von Willebrand factor to the immobilised antibodies. Unbound von Willebrand factor may be removed by washing. Factor VIII may be added to the wells of the multi-well plate and incubated to allow binding of Factor VIII to von Willebrand factor. Unbound Factor VIII may be removed by washing. Binding of Factor VIII to von Willebrand factor may be measured using an antibody against Factor VIII conjugated to peroxidase (e.g. a peroxidase-conjugated sheep-anti-human FVIII antibody) in a chromogenic assay (e.g. using tetramethylbenzidine dihydrochloride (TMB) as a chromogenic substrate). Peroxidase catalyses the conversion of TMB to a blue product which may be detected spectrophotometrically as described elsewhere herein. The level of the blue product is proportional to the amount of detection antibody that remained after the washing step, which is proportional to the amount of the Factor VIII polypeptide bound to von Willebrand factor. A standard curve may be employed using a serial dilution of a standard human plasma (SHP) standard. Optionally an assay to measure the binding of von Willebrand factor to Factor VIII may be performed in conjunction with an assay to determine the amount of von Willebrand factor present in a sample. This may be performed by immobilising von Willebrand factor as outlined above, and using an antibody against von Willebrand factor conjugated to peroxidase (e.g. a peroxidase-conjugated rabbit anti-human von Willebrand factor antibody) in a chromogenic assay.

The Factor VIII amino acid sequence may comprise an amino acid corresponding to position 1679 of SEQ ID NO: 1. The Factor VIII amino acid sequence may comprise an amino acid corresponding to position 1680 of SEQ ID NO: 1. Preferably the amino acid corresponding to position 1680 of SEQ ID NO: 1 is a tyrosine. The Factor VIII amino acid sequence may comprise amino acids corresponding to positions 1679 and 1680 of SEQ ID NO: 1. Preferably the amino acid corresponding to position 1680 is a tyrosine. Optionally, the Factor VIII amino acid sequence does not comprise an amino acid corresponding to position 1679 of SEQ ID NO: 1. Optionally, the Factor VIII amino acid sequence does not comprise an amino acid corresponding to position 1680 of SEQ ID NO: 1. Optionally, the Factor VIII amino acid sequence does not comprise amino acids corresponding to positions 1679 and 1680 of SEQ ID NO: 1.

Optionally, the Factor VIII amino acid sequence does not comprise at least one amino acid(s) corresponding to positions 1681 to 1689 of SEQ ID NO: 1. Optionally, the Factor VIII amino acid sequence does not comprise at least 2, at least 3, at least 4, at least 5, at least 6, at least 7, at least 8, or 9 amino acids corresponding to positions 1681 to 1689 of SEQ ID NO: 1. Optionally, the Factor VIII amino acid sequence does not comprise 9 or fewer, 8 or fewer, 7 or fewer, 6 or fewer, 5 or fewer, 4 or fewer, 3 or fewer, or 2 or fewer amino acids corresponding to positions 1681 to 1689 of SEQ ID NO: 1. Optionally, the Factor VIII amino acid sequence does not comprise between 1 and 10, between 4 and 10, or between 6 and 10 amino acids corresponding to positions 1681 to 1689 of SEQ ID NO: 1. Optionally, the Factor VIII amino acid sequence does not comprise at least 2 contiguous amino acids corresponding to positions 1681 to 1689 of SEQ ID NO: 1. Optionally, the Factor VIII amino acid sequence does not comprise at least 3, at least 4, at least 5, at least 6, at least 7, at least 8, or 9 contiguous amino acids corresponding to positions 1681 to 1689 of SEQ ID NO: 1. Optionally, the Factor VIII amino acid sequence does not comprise amino acids corresponding to positions 1681 to 1682, 1681 to 1683, 1681 to 1684, 1681 to 1685, 1681 to 1686, 1681 to 1687, 1681 to 1688, or 1681 to 1689 of SEQ ID NO: 1. Optionally, the Factor VIII amino acid sequence does not comprise amino acids corresponding to positions 1681 to 1683 of SEQ ID NO: 1. Optionally, the Factor VIII amino acid sequence does not comprise amino acids corresponding to positions 1681 to 1686 of SEQ ID NO: 1. Optionally, the Factor VIII amino acid sequence does not comprise amino acids corresponding to positions 1681 to 1688 of SEQ ID NO: 1. Optionally, the Factor VIII amino acid sequence does not comprise amino acids corresponding to positions 1681 to 1689 of SEQ ID NO: 1.

The Factor VIII amino acid sequence may comprise at least one of the amino acid(s) corresponding to positions 1681 to 1689 of SEQ ID NO: 1. The Factor VIII amino acid sequence may comprise at least 2, at least 3, at least 4, at least 5, at least 6, at least 7, at least 8, or 9 amino acids corresponding to positions 1681 to 1689 of SEQ ID NO: 1. The Factor VIII amino acid sequence may comprise 9 or fewer, 8 or fewer, 7 or fewer, 6 or fewer, 5 or fewer, 4 or fewer, 3 or fewer, or 2 or fewer amino acids corresponding to positions 1681 to 1689 of SEQ ID NO: 1. The Factor VIII amino acid sequence may comprise between 1 and 10, between 4 and 10, or between 6 and 10 amino acids corresponding to positions 1681 to 1689 of SEQ ID NO: 1. The Factor VIII amino acid sequence may comprise between 1 and 10, between 1 and 8, or between 1 and 5 amino acids corresponding to positions 1681 to 1689 of SEQ ID NO: 1. The Factor VIII amino acid sequence may comprise at least 2 contiguous amino acids corresponding to positions 1681 to 1689 of SEQ ID NO: 1. The Factor VIII amino acid sequence may comprise at least 3, at least 4, at least 5, at least 6, at least 7, at least 8, or 9 contiguous amino acids corresponding to positions 1681 to 1689 of SEQ ID NO: 1. The Factor VIII amino acid sequence may comprise amino acids corresponding to positions 1688 to 1689, 1687 to 1689, 1686 to 1689, 1685 to 1689, 1684 to 1689, 1683 to 1689, 1682 to 1689, or 1681 to 1689 of SEQ ID NO: 1. The Factor VIII amino acid sequence may comprise an amino acid corresponding to position 1689 of SEQ ID NO: 1. The Factor VIII amino acid sequence may comprise amino acids corresponding to positions 1687 to 1689 of SEQ ID NO: 1. The Factor VIII amino acid sequence may comprise amino acids corresponding to positions 1684 to 1689 of SEQ ID NO: 1. The Factor VIII amino acid sequence may comprise amino acids corresponding to positions 1681 to 1689 of SEQ ID NO: 1.

The Factor VIII amino acid sequence may comprise at least one of the amino acid(s) corresponding to positions 1690 to 1696 of SEQ ID NO: 1. The Factor VIII amino acid sequence may comprise at least 2, at least 3, at least 4, at least 5, at least 6, or 7 amino acids corresponding to positions 1690 to 1696 of SEQ ID NO: 1. The Factor VIII amino acid sequence may comprise 7 or fewer, 6 or fewer, 5 or fewer, 4 or fewer, 3 or fewer, or 2 or fewer amino acids corresponding to positions 1690 to 1696 of SEQ ID NO: 1. The Factor VIII amino acid sequence may comprise between 2 and 8, or between 5 and 8 amino acids corresponding to positions 1690 to 1696 of SEQ ID NO: 1. The Factor VIII amino acid sequence may comprise at least 2 contiguous amino acids corresponding to positions 1690 to 1696 of SEQ ID NO: 1. The Factor VIII amino acid sequence may comprise at least 3, at least 4, at least 5, at least 6, or 7 contiguous amino acids corresponding to positions 1690 to 1696 of SEQ ID NO: 1. The Factor VIII amino acid sequence may comprise amino acids corresponding to positions 1695 to 1696, 1694 to 1696, 1693 to 1696, 1692 to 1696, 1691 to 1696, or 1690 to 1696 of SEQ ID NO: 1. The Factor VIII amino acid sequence may comprise amino acids corresponding to positions 1690 to 1696 of SEQ ID NO: 1.

Optionally, the Factor VIII amino acid sequence does not comprise amino acids corresponding to positions 741 to 1669 of SEQ ID NO: 1. Optionally, the Factor VIII amino acid sequence does not comprise amino acids corresponding to positions 741 to 1669 of SEQ ID NO: 1, and the Factor VIII amino acid sequence comprises amino acids corresponding to positions 724 to 740 of SEQ ID NO: 1. Optionally, the Factor VIII amino acid sequence does not comprise amino acids corresponding to positions 741 to 1669 of SEQ ID NO: 1, and the Factor VIII amino acid sequence comprises amino acids corresponding to positions 714 to 740 of SEQ ID NO: 1. Optionally, the Factor VIII amino acid sequence does not comprise amino acids corresponding to positions 741 to 1669 of SEQ ID NO: 1, and the Factor VIII amino acid sequence comprises amino acids corresponding to positions 1670 to 1689 of SEQ ID NO: 1. Optionally, the Factor VIII amino acid sequence does not comprise amino acids corresponding to positions 741 to 1669 of SEQ ID NO: 1, and the Factor VIII amino acid sequence comprises amino acids corresponding to positions 1670 to 1696 of SEQ ID NO: 1. Optionally, the Factor VIII amino acid sequence does not comprise amino acids corresponding to positions 741 to 1669 of SEQ ID NO: 1, and the Factor VIII amino acid sequence comprises amino acids corresponding to positions 724 to 740 and 1670 to 1689 of SEQ ID NO: 1. Optionally, the Factor VIII amino acid sequence does not comprise amino acids corresponding to positions 741 to 1669 of SEQ ID NO: 1, and the Factor VIII amino acid sequence comprises amino acids corresponding to positions 724 to 740 and 1670 to 1689 of SEQ ID NO: 1 and the tyrosine amino acids corresponding to positions 718, 719 and 723 of SEQ ID NO: 1. Optionally, the Factor VIII amino acid sequence does not comprise amino acids corresponding to positions 741 to 1669 of SEQ ID NO: 1, and the Factor VIII amino acid sequence comprises amino acids corresponding to positions 714 to 740 and 1670 to 1689 of SEQ ID NO: 1. Optionally, the Factor VIII amino acid sequence does not comprise amino acids corresponding to positions 741 to 1669 of SEQ ID NO: 1, and the Factor VIII amino acid sequence comprises amino acids corresponding to positions 714 to 740 and 1670 to 1696 of SEQ ID NO: 1. Optionally, the Factor VIII amino acid sequence does not comprise amino acids corresponding to positions 741 to 1669 of SEQ ID NO: 1, and the Factor VIII amino acid sequence comprises amino acids corresponding to positions 1 to 740 and 1670 to 2332 of SEQ ID NO: 1. The Factor VIII amino acid sequence may comprise SEQ ID NO: 9. The Factor VIII amino acid sequence may be SEQ ID NO: 9.

Optionally, the Factor VIII amino acid sequence does not comprise amino acids corresponding to positions 732 to 1669 of SEQ ID NO: 1. Optionally, the Factor VIII amino acid sequence does not comprise amino acids corresponding to positions 732 to 1669 of SEQ ID NO: 1, and the Factor VIII amino acid sequence comprises amino acids corresponding to positions 724 to 731 of SEQ ID NO: 1. Optionally, the Factor VIII amino acid sequence does not comprise amino acids corresponding to positions 732 to 1669 of SEQ ID NO: 1, and the Factor VIII amino acid sequence comprises amino acids corresponding to positions 714 to 731 of SEQ ID NO: 1. Optionally, the Factor VIII amino acid sequence does not comprise amino acids corresponding to positions 732 to 1669 of SEQ ID NO: 1, and the Factor VIII amino acid sequence comprises amino acids corresponding to positions 1670 to 1689 of SEQ ID NO: 1. Optionally, the Factor VIII amino acid sequence does not comprise amino acids corresponding to positions 732 to 1669 of SEQ ID NO: 1, and the Factor VIII amino acid sequence comprises amino acids corresponding to positions 1670 to 1696 of SEQ ID NO: 1. Optionally, the Factor VIII amino acid sequence does not comprise amino acids corresponding to positions 732 to 1669 of SEQ ID NO: 1, and the Factor VIII amino acid sequence comprises amino acids corresponding to positions 724 to 731 and 1670 to 1689 of SEQ ID NO: 1. Optionally, the Factor VIII amino acid sequence does not comprise amino acids corresponding to positions 732 to 1669 of SEQ ID NO: 1, and the Factor VIII amino acid sequence comprises amino acids corresponding to positions 724 to 731 and 1670 to 1689 of SEQ ID NO: 1 and the tyrosine amino acids corresponding to positions 718, 719 and 723 of SEQ ID NO: 1. Optionally, the Factor VIII amino acid sequence does not comprise amino acids corresponding to positions 732 to 1669 of SEQ ID NO: 1, and the Factor VIII amino acid sequence comprises amino acids corresponding to positions 714 to 731 and 1670 to 1689 of SEQ ID NO: 1. Optionally, the Factor VIII amino acid sequence does not comprise amino acids corresponding to positions 732 to 1669 of SEQ ID NO: 1, and the Factor VIII amino acid sequence comprises amino acids corresponding to positions 714 to 731 and 1670 to 1696 of SEQ ID NO: 1. Optionally, the Factor VIII amino acid sequence does not comprise amino acids corresponding to positions 732 to 1669 of SEQ ID NO: 1, and the Factor VIII amino acid sequence comprises amino acids corresponding to positions 1 to 731 and 1670 to 2332 of SEQ ID NO: 1.

Optionally, the Factor VIII amino acid sequence does not comprise amino acids corresponding to positions 729 to 1666 of SEQ ID NO: 1. Optionally, the Factor VIII amino acid sequence does not comprise amino acids corresponding to positions 729 to 1666 of SEQ ID NO: 1, and the Factor VIII amino acid sequence comprises amino acids corresponding to positions 724 to 728 of SEQ ID NO: 1. Optionally, the Factor VIII amino acid sequence does not comprise amino acids corresponding to positions 729 to 1666 of SEQ ID NO: 1, and the Factor VIII amino acid sequence comprises amino acids corresponding to positions 714 to 728 of SEQ ID NO: 1. Optionally, the Factor VIII amino acid sequence does not comprise amino acids corresponding to positions 729 to 1666 of SEQ ID NO: 1, and the Factor VIII amino acid sequence comprises amino acids corresponding to positions 1667 to 1689 of SEQ ID NO: 1. Optionally, the Factor VIII amino acid sequence does not comprise amino acids corresponding to positions 729 to 1666 of SEQ ID NO: 1, and the Factor VIII amino acid sequence comprises amino acids corresponding to positions 1667 to 1696 of SEQ ID NO: 1. Optionally, the Factor VIII amino acid sequence does not comprise amino acids corresponding to positions 729 to 1666 of SEQ ID NO: 1, and the Factor VIII amino acid sequence comprises amino acids corresponding to positions 724 to 728 and 1667 to 1689 of SEQ ID NO: 1. Optionally, the Factor VIII amino acid sequence does not comprise amino acids corresponding to positions 729 to 1666 of SEQ ID NO: 1, and the Factor VIII amino acid sequence comprises amino acids corresponding to positions 724 to 728 and 1667 to 1689 of SEQ ID NO: 1 and the tyrosine amino acids corresponding to positions 718, 719 and 723 of SEQ ID NO: 1. Optionally, the Factor VIII amino acid sequence does not comprise amino acids corresponding to positions 729 to 1666 of SEQ ID NO: 1, and the Factor VIII amino acid sequence comprises amino acids corresponding to positions 714 to 728 and 1667 to 1689 of SEQ ID NO: 1. Optionally, the Factor VIII amino acid sequence does not comprise amino acids corresponding to positions 729 to 1666 of SEQ ID NO: 1, and the Factor VIII amino acid sequence comprises amino acids corresponding to positions 714 to 728 and 1667 to 1696 of SEQ ID NO: 1. Optionally, the Factor VIII amino acid sequence does not comprise amino acids corresponding to positions 729 to 1666 of SEQ ID NO: 1, and the Factor VIII amino acid sequence comprises amino acids corresponding to positions 1 to 728 and 1667 to 2332 of SEQ ID NO: 1.

Optionally, the Factor VIII amino acid sequence does not comprise amino acids corresponding to positions 723 to 740 of SEQ ID NO: 1.

Optionally, the Factor VIII amino acid sequence may comprise any one of the amino acids corresponding to positions 713 to 745 of SEQ ID NO:1 directly linked to any one of the amino acids corresponding to positions 1640 to 1697 of SEQ ID NO:1. In this context the term “directly linked” indicates that two amino acids are joined by a peptide bond. Put another way, optionally, the Factor VIII amino acid sequence may comprise any one of the amino acids corresponding to positions 713 to 745 of SEQ ID NO:1 joined by a peptide bond to any one of the amino acids corresponding to positions 1640 to 1697 of SEQ ID NO:1.

Optionally, the Factor VIII amino acid sequence may comprise any one of the amino acids corresponding to positions 713 to 745 of SEQ ID NO:1 directly linked to any one of the amino acids corresponding to positions 1640 to 1690 of SEQ ID NO:1. Optionally, the Factor VIII amino acid sequence may comprise any one of the amino acids corresponding to positions 713 to 740 of SEQ ID NO:1 directly linked to any one of the amino acids corresponding to positions 1640 to 1697 of SEQ ID NO:1. Optionally, the Factor VIII amino acid sequence may comprise any one of the amino acids corresponding to positions 713 to 740 of SEQ ID NO:1 directly linked to any one of the amino acids corresponding to positions 1649 to 1690 of SEQ ID NO:1. Optionally, the Factor VIII amino acid sequence may comprise any one of the amino acids corresponding to positions 713 to 740 of SEQ ID NO:1 directly linked to any one of the amino acids corresponding to positions 1670 to 1689 of SEQ ID NO:1. Optionally, the Factor VIII amino acid sequence may comprise any one of the amino acids corresponding to positions 713 to 740 of SEQ ID NO:1 directly linked to any one of the amino acids corresponding to positions 1649 to 1670 of SEQ ID NO:1.

Optionally, the Factor VIII amino acid sequence may comprise any one of the amino acids corresponding to positions 721 to 740 of SEQ ID NO: 1 directly linked to any one of the amino acids corresponding to positions 1640 to 1697 of SEQ ID NO: 1. Optionally, the Factor VIII amino acid sequence may comprise any one of the amino acids corresponding to positions 721 to 740 of SEQ ID NO: 1 directly linked to any one of the amino acids corresponding to positions 1640 to 1690 of SEQ ID NO: 1. Optionally, the Factor VIII amino acid sequence may comprise any one of the amino acids corresponding to positions 723 to 740 of SEQ ID NO: linked to any one of the amino acids corresponding to positions 1640 to 1697 of SEQ ID NO:1. Optionally, the Factor VIII amino acid sequence may comprise any one of the amino acids corresponding to positions 723 to 740 of SEQ ID NO: linked to any one of the amino acids corresponding to positions 1640 to 1690 of SEQ ID NO:1. Optionally, the Factor VIII amino acid sequence may comprise any one of the amino acids corresponding to positions 721 to 731 of SEQ ID NO: 1 directly linked to any one of the amino acids corresponding to positions 1640 to 1697 of SEQ ID NO: 1. Optionally, the Factor VIII amino acid sequence may comprise any one of the amino acids corresponding to positions 721 to 731 of SEQ ID NO: 1 directly linked to any one of the amino acids corresponding to positions 1640 to 1690 of SEQ ID NO: 1. Optionally, the Factor VIII amino acid sequence may comprise any one of the amino acids corresponding to positions 731 to 741 of SEQ ID NO: linked to any one of the amino acids corresponding to positions 1640 to 1697 of SEQ ID NO:1. Optionally, the Factor VIII amino acid sequence may comprise any one of the amino acids corresponding to positions 731 to 741 of SEQ ID NO: linked to any one of the amino acids corresponding to positions 1640 to 1690 of SEQ ID NO:1. Optionally, the Factor VIII amino acid sequence may comprise any one of the amino acids corresponding to positions 721 to 740 of SEQ ID NO: 1 directly linked to any one of the amino acids corresponding to positions 1649 to 1690 of SEQ ID NO: 1. Optionally, the Factor VIII amino acid sequence may comprise any one of the amino acids corresponding to positions 723 to 740 of SEQ ID NO: 1 directly linked to any one of the amino acids corresponding to positions 1649 to 1690 of SEQ ID NO: 1. Optionally, the Factor VIII amino acid sequence may comprise any one of the amino acids corresponding to positions 721 to 731 of SEQ ID NO: 1 directly linked to any one of the amino acids corresponding to positions 1649 to 1690 of SEQ ID NO: 1. Optionally, the Factor VIII amino acid sequence may comprise any one of the amino acids corresponding to positions 731 to 741 of SEQ ID NO: linked to any one of the amino acids corresponding to positions 1649 to 1690 of SEQ ID NO:1. Optionally, the Factor VIII amino acid sequence may comprise any one of the amino acids corresponding to positions 721 to 740 of SEQ ID NO: 1 directly linked to any one of the amino acids corresponding to positions 1670 to 1689 of SEQ ID NO: 1. Optionally, the Factor VIII amino acid sequence may comprise any one of the amino acids corresponding to positions 723 to 740 of SEQ ID NO: 1 directly linked to any one of the amino acids corresponding to positions 1670 to 1689 of SEQ ID NO: 1. Optionally, the Factor VIII amino acid sequence may comprise any one of the amino acids corresponding to positions 721 to 731 of SEQ ID NO: 1 directly linked to any one of the amino acids corresponding to positions 1670 to 1689 of SEQ ID NO: 1. Optionally, the Factor VIII amino acid sequence may comprise any one of the amino acids corresponding to positions 731 to 741 of SEQ ID NO: linked to any one of the amino acids corresponding to positions 1670 to 1689 of SEQ ID NO:1. Optionally, the Factor VIII amino acid sequence may comprise any one of the amino acids corresponding to positions 721 to 740 of SEQ ID NO: 1 directly linked to any one of the amino acids corresponding to positions 1649 to 1670 of SEQ ID NO: 1. Optionally, the Factor VIII amino acid sequence may comprise any one of the amino acids corresponding to positions 723 to 740 of SEQ ID NO: 1 directly linked to any one of the amino acids corresponding to positions 1649 to 1670 of SEQ ID NO: 1. Optionally, the Factor VIII amino acid sequence may comprise any one of the amino acids corresponding to positions 721 to 731 of SEQ ID NO: 1 directly linked to any one of the amino acids corresponding to positions 1649 to 1670 of SEQ ID NO: 1. Optionally, the Factor VIII amino acid sequence may comprise any one of the amino acids corresponding to positions 731 to 741 of SEQ ID NO: linked to any one of the amino acids corresponding to positions 1649 to 1670 of SEQ ID NO:1.

Optionally, the Factor VIII amino acid sequence may comprise any one of the amino acids corresponding to positions 723 to 737 of SEQ ID NO:1 directly linked to any one of the amino acids corresponding to positions 1649 to 1690 of SEQ ID NO:1. Optionally, the Factor VIII amino acid sequence may comprise any one of the amino acids corresponding to positions 723 to 737 of SEQ ID NO:1 directly linked to any one of the amino acids corresponding to positions 1649 to 1670 of SEQ ID NO:1. Optionally, the Factor VIII amino acid sequence may comprise any one of the amino acids corresponding to positions 722 to 737 of SEQ ID NO:1 directly linked to any one of the amino acids corresponding to positions 1649 to 1689 of SEQ ID NO:1. Optionally, the Factor VIII amino acid sequence may comprise any one of the amino acids corresponding to positions 722 to 737 of SEQ ID NO:1 directly linked to any one of the amino acids corresponding to positions 1649 to 1670 of SEQ ID NO:1.

Optionally, the Factor VIII amino acid sequence may comprise the amino acid corresponding to position 722 of SEQ ID NO:1 directly linked to any one of the amino acids corresponding to positions 1640 to 1690 of SEQ ID NO:1. Optionally, the Factor VIII amino acid sequence may comprise the amino acid corresponding to position 722 of SEQ ID NO:1 directly linked to any one of the amino acids corresponding to positions 1649 to 1690 of SEQ ID NO:1. Optionally, the Factor VIII amino acid sequence may comprise the amino acid corresponding to position 722 of SEQ ID NO:1 directly linked to any one of the amino acids corresponding to positions 1670 to 1689 of SEQ ID NO:1. Optionally, the Factor VIII amino acid sequence may comprise the amino acid corresponding to position 722 of SEQ ID NO:1 directly linked to any one of the amino acids corresponding to positions 1649 to 1670 of SEQ ID NO:1. Optionally, the Factor VIII amino acid sequence may comprise the amino acid corresponding to position 722 of SEQ ID NO:1 directly linked to the amino acid corresponding to position 1667 of SEQ ID NO:1. Optionally, the Factor VIII amino acid sequence may comprise the amino acid corresponding to position 722 of SEQ ID NO:1 directly linked to the amino acid corresponding to position 1670 of SEQ ID NO:1.

Optionally, the Factor VIII amino acid sequence may comprise the amino acid corresponding to position 723 of SEQ ID NO:1 directly linked to any one of the amino acids corresponding to positions 1640 to 1690 of SEQ ID NO:1. Optionally, the Factor VIII amino acid sequence may comprise the amino acid corresponding to position 723 of SEQ ID NO:1 directly linked to any one of the amino acids corresponding to positions 1649 to 1690 of SEQ ID NO:1. Optionally, the Factor VIII amino acid sequence may comprise the amino acid corresponding to position 723 of SEQ ID NO:1 directly linked to any one of the amino acids corresponding to positions 1670 to 1689 of SEQ ID NO:1. Optionally, the Factor VIII amino acid sequence may comprise the amino acid corresponding to position 723 of SEQ ID NO:1 directly linked to any one of the amino acids corresponding to positions 1649 to 1670 of SEQ ID NO:1. Optionally, the Factor VIII amino acid sequence may comprise the amino acid corresponding to position 723 of SEQ ID NO:1 directly linked to the amino acid corresponding to position 1667 of SEQ ID NO:1. Optionally, the Factor VIII amino acid sequence may comprise the amino acid corresponding to position 723 of SEQ ID NO:1 directly linked to the amino acid corresponding to position 1670 of SEQ ID NO:1.

Optionally, the Factor VIII amino acid sequence may comprise the amino acid corresponding to position 725 of SEQ ID NO:1 directly linked to any one of the amino acids corresponding to positions 1640 to 1690 of SEQ ID NO:1. Optionally, the Factor VIII amino acid sequence may comprise the amino acid corresponding to position 725 of SEQ ID NO:1 directly linked to any one of the amino acids corresponding to positions 1649 to 1690 of SEQ ID NO:1. Optionally, the Factor VIII amino acid sequence may comprise the amino acid corresponding to position 725 of SEQ ID NO:1 directly linked to any one of the amino acids corresponding to positions 1670 to 1689 of SEQ ID NO:1. Optionally, the Factor VIII amino acid sequence may comprise the amino acid corresponding to position 725 of SEQ ID NO:1 directly linked to any one of the amino acids corresponding to positions 1649 to 1670 of SEQ ID NO:1.

Optionally, the Factor VIII amino acid sequence may comprise the amino acid corresponding to position 725 of SEQ ID NO:1 directly linked to the amino acid corresponding to position 1667 of SEQ ID NO:1. Optionally, the Factor VIII amino acid sequence may comprise the amino acid corresponding to position 725 of SEQ ID NO:1 directly linked to the amino acid corresponding to position 1670 of SEQ ID NO:1.

Optionally, the Factor VIII amino acid sequence may comprise the amino acid corresponding to position 728 of SEQ ID NO:1 directly linked to any one of the amino acids corresponding to positions 1640 to 1690 of SEQ ID NO:1. Optionally, the Factor VIII amino acid sequence may comprise the amino acid corresponding to position 728 of SEQ ID NO:1 directly linked to any one of the amino acids corresponding to positions 1649 to 1690 of SEQ ID NO:1. Optionally, the Factor VIII amino acid sequence may comprise the amino acid corresponding to position 728 of SEQ ID NO:1 directly linked to any one of the amino acids corresponding to positions 1670 to 1689 of SEQ ID NO:1. Optionally, the Factor VIII amino acid sequence may comprise the amino acid corresponding to position 728 of SEQ ID NO:1 directly linked to any one of the amino acids corresponding to positions 1649 to 1670 of SEQ ID NO:1. Optionally, the Factor VIII amino acid sequence may comprise the amino acid corresponding to position 728 of SEQ ID NO:1 directly linked to the amino acid corresponding to position 1667 of SEQ ID NO:1. Optionally, the Factor VIII amino acid sequence may comprise the amino acid corresponding to position 728 of SEQ ID NO:1 directly linked to the amino acid corresponding to position 1670 of SEQ ID NO:1.

Optionally, the Factor VIII amino acid sequence may comprise the amino acid corresponding to position 731 of SEQ ID NO:1 directly linked to any one of the amino acids corresponding to positions 1640 to 1690 of SEQ ID NO:1. Optionally, the Factor VIII amino acid sequence may comprise the amino acid corresponding to position 731 of SEQ ID NO:1 directly linked to any one of the amino acids corresponding to positions 1649 to 1690 of SEQ ID NO:1. Optionally, the Factor VIII amino acid sequence may comprise the amino acid corresponding to position 731 of SEQ ID NO:1 directly linked to any one of the amino acids corresponding to positions 1670 to 1689 of SEQ ID NO:1. Optionally, the Factor VIII amino acid sequence may comprise the amino acid corresponding to position 731 of SEQ ID NO:1 directly linked to any one of the amino acids corresponding to positions 1649 to 1670 of SEQ ID NO:1. Optionally, the Factor VIII amino acid sequence may comprise the amino acid corresponding to position 731 of SEQ ID NO:1 directly linked to the amino acid corresponding to position 1667 of SEQ ID NO:1. Optionally, the Factor VIII amino acid sequence may comprise the amino acid corresponding to position 731 of SEQ ID NO:1 directly linked to the amino acid corresponding to position 1670 of SEQ ID NO:1.

Optionally, the Factor VIII amino acid sequence may comprise the amino acid corresponding to position 737 of SEQ ID NO:1 directly linked to any one of the amino acids corresponding to positions 1640 to 1690 of SEQ ID NO:1. Optionally, the Factor VIII amino acid sequence may comprise the amino acid corresponding to position 737 of SEQ ID NO:1 directly linked to any one of the amino acids corresponding to positions 1649 to 1690 of SEQ ID NO:1. Optionally, the Factor VIII amino acid sequence may comprise the amino acid corresponding to position 737 of SEQ ID NO:1 directly linked to any one of the amino acids corresponding to positions 1670 to 1689 of SEQ ID NO:1. Optionally, the Factor VIII amino acid sequence may comprise the amino acid corresponding to position 737 of SEQ ID NO:1 directly linked to any one of the amino acids corresponding to positions 1649 to 1670 of SEQ ID NO:1. Optionally, the Factor VIII amino acid sequence may comprise the amino acid corresponding to position 737 of SEQ ID NO:1 directly linked to the amino acid corresponding to position 1667 of SEQ ID NO:1. Optionally, the Factor VIII amino acid sequence may comprise the amino acid corresponding to position 737 of SEQ ID NO:1 directly linked to the amino acid corresponding to position 1670 of SEQ ID NO:1.

Optionally, the Factor VIII amino acid sequence may comprise the amino acid corresponding to position 740 of SEQ ID NO:1 directly linked to any one of the amino acids corresponding to positions 1640 to 1690 of SEQ ID NO:1. Optionally, the Factor VIII amino acid sequence may comprise the amino acid corresponding to position 740 of SEQ ID NO:1 directly linked to any one of the amino acids corresponding to positions 1649 to 1690 of SEQ ID NO:1. Optionally, the Factor VIII amino acid sequence may comprise the amino acid corresponding to position 740 of SEQ ID NO:1 directly linked to any one of the amino acids corresponding to positions 1670 to 1689 of SEQ ID NO:1. Optionally, the Factor VIII amino acid sequence may comprise the amino acid corresponding to position 740 of SEQ ID NO:1 directly linked to any one of the amino acids corresponding to positions 1649 to 1670 of SEQ ID NO:1. Optionally, the Factor VIII amino acid sequence may comprise the amino acid corresponding to position 740 of SEQ ID NO:1 directly linked to the amino acid corresponding to position 1667 of SEQ ID NO:1. Optionally, the Factor VIII amino acid sequence may comprise the amino acid corresponding to position 740 of SEQ ID NO:1 directly linked to the amino acid corresponding to position 1670 of SEQ ID NO:1.

Optionally, the Factor VIII amino acid sequence may comprise any one of the amino acids corresponding to positions 722 to 740 of SEQ ID NO:1 directly linked to the amino acid corresponding to position 1649 of SEQ ID NO:1. Optionally, the Factor VIII amino acid sequence may comprise any one of the amino acids corresponding to positions 723 to 737 of SEQ ID NO:1 directly linked to the amino acid corresponding to position 1649 of SEQ ID NO:1. Optionally, the Factor VIII amino acid sequence may comprise any one of the amino acids corresponding to positions 725 to 731 of SEQ ID NO:1 directly linked to the amino acid corresponding to position 1649 of SEQ ID NO:1. Optionally, the Factor VIII amino acid sequence may comprise any one of the amino acids corresponding to positions 728 to 731 of SEQ ID NO:1 directly linked to the amino acid corresponding to position 1649 of SEQ ID NO:1.

ionally, the Factor VIII amino acid sequence may comprise any one of the amino acids corresponding to positions 722 to 740 of SEQ ID NO:1 directly linked to the amino acid corresponding to position 1667 of SEQ ID NO:1. Optionally, the Factor VIII amino acid sequence may comprise any one of the amino acids corresponding to positions 723 to 737 of SEQ ID NO:1 directly linked to the amino acid corresponding to position 1667 of SEQ ID NO:1. Optionally, the Factor VIII amino acid sequence may comprise any one of the amino acids corresponding to positions 725 to 731 of SEQ ID NO:1 directly linked to the amino acid corresponding to position 1667 of SEQ ID NO:1. Optionally, the Factor VIII amino acid sequence may comprise any one of the amino acids corresponding to positions 728 to 731 of SEQ ID NO:1 directly linked to the amino acid corresponding to position 1667 of SEQ ID NO:1.

Optionally, the Factor VIII amino acid sequence may comprise any one of the amino acids corresponding to positions 722 to 740 of SEQ ID NO:1 directly linked to the amino acid corresponding to position 1670 of SEQ ID NO:1. Optionally, the Factor VIII amino acid sequence may comprise any one of the amino acids corresponding to positions 723 to 737 of SEQ ID NO:1 directly linked to the amino acid corresponding to position 1670 of SEQ ID NO:1. Optionally, the Factor VIII amino acid sequence may comprise any one of the amino acids corresponding to positions 725 to 731 of SEQ ID NO:1 directly linked to the amino acid corresponding to position 1670 of SEQ ID NO:1. Optionally, the Factor VIII amino acid sequence may comprise any one of the amino acids corresponding to positions 728 to 731 of SEQ ID NO:1 directly linked to the amino acid corresponding to position 1670 of SEQ ID NO:1.

Optionally, the Factor VIII amino acid sequence may comprise any one of the amino acids corresponding to positions 722 to 740 of SEQ ID NO:1 directly linked to the amino acid corresponding to position 1689 of SEQ ID NO:1. Optionally, the Factor VIII amino acid sequence may comprise any one of the amino acids corresponding to positions 723 to 737 of SEQ ID NO:1 directly linked to the amino acid corresponding to position 1689 of SEQ ID NO:1. Optionally, the Factor VIII amino acid sequence may comprise any one of the amino acids corresponding to positions 725 to 731 of SEQ ID NO:1 directly linked to the amino acid corresponding to position 1689 of SEQ ID NO:1. Optionally, the Factor VIII amino acid sequence may comprise any one of the amino acids corresponding to positions 728 to 731 of SEQ ID NO:1 directly linked to the amino acid corresponding to position 1689 of SEQ ID NO:1.

Reference to “any one of the amino acids” corresponding to a first range of positions of SEQ ID NO: 1 (e.g. 723 to 737) directly linked to “any one of the amino acids” corresponding to a second range of positions of SEQ ID NO: 1 (e.g. 1649 to 1670) is intended to be inclusive, i.e. the higher and lower amino acid positions recited in the respective ranges are included.

Optionally, the Factor VIII polypeptide may comprise the amino acid sequence of any one of SEQ ID NOs: 95 to 122. Optionally, the Factor VIII polypeptide may comprise the amino acid sequence of any one of SEQ ID NOs: 95 to 98, 103 to 108, and 111 to 122. Optionally, the Factor VIII polypeptide may comprise the amino acid sequence of any one of SEQ ID NOs: 95 to 98, 103 to 107, and 111 to 122. Optionally, the Factor VIII polypeptide may comprise the amino acid sequence of SEQ ID NO: 117.

Optionally, the Factor VIII polypeptide may comprise a Factor VIII amino acid sequence which has at least 90% sequence identity to any one of SEQ ID NOs: 9-36. The Factor VIII polypeptide may comprise an amino acid sequence which is at least 95%, at least 96%, at least 98%, at least 99% or 100% identical to any one of SEQ ID NOs: 9-36. Optionally, the

Factor VIII polypeptide may be a Factor VIII amino acid sequence which has at least 90% sequence identity to any one of SEQ ID NOs:9-36. The Factor VIII polypeptide may be a Factor VIII amino acid sequence which has at least 95%, at least 96%, at least 97%, at least 98% or at least 99% sequence identity to any one of SEQ ID NOs: 9-36. The Factor VIII amino acid sequence may comprise any one of SEQ ID NOs: 9-36. The Factor VIII amino acid sequence may be any one of SEQ ID NOs: 9-36.

Optionally, the Factor VIII polypeptide may comprise a Factor VIII amino acid sequence which has at least 90% sequence identity to any one of SEQ ID NOs: 9 to 12, 17 to 22 and 25 to 36. The Factor VIII polypeptide may comprise an amino acid sequence which is at least 95%, at least 96%, at least 98%, at least 99% or 100% identical to any one of SEQ ID NOs: 9 to 12, 17 to 22 and 25 to 36. Optionally, the Factor VIII polypeptide may be a Factor VIII amino acid sequence which has at least 90% sequence identity to any one of SEQ ID NOs: 9 to 12, 17 to 22 and 25 to 36. The Factor VIII polypeptide may be a Factor VIII amino acid sequence which has at least 95%, at least 96%, at least 97%, at least 98% or at least 99% sequence identity to any one of SEQ ID NOs: 9 to 12, 17 to 22 and 25 to 36. The Factor VIII amino acid sequence may comprise any one of SEQ ID NOs: 9 to 12, 17 to 22 and 25 to 36. The Factor VIII amino acid sequence may be any one of SEQ ID NOs: 9 to 12, 17 to 22 and 25 to 36.

Optionally, the Factor VIII polypeptide may comprise a Factor VIII amino acid sequence which has at least 90% sequence identity to any one of SEQ ID NOs: 9 to 12, 17 to 21 and 25 to 36. The Factor VIII polypeptide may comprise an amino acid sequence which is at least 95%, at least 96%, at least 98%, at least 99% or 100% identical to any one of SEQ ID NOs: 9 to 12, 17 to 21 and 25 to 36. Optionally, the Factor VIII polypeptide may be a Factor VIII amino acid sequence which has at least 90% sequence identity to any one of SEQ ID NOs: 9 to 12, 17 to 21 and 25 to 36. The Factor VIII polypeptide may be a Factor VIII amino acid sequence which has at least 95%, at least 96%, at least 97%, at least 98% or at least 99% sequence identity to any one of SEQ ID NOs: 9 to 12, 17 to 21 and 25 to 36. The Factor VIII amino acid sequence may comprise any one of SEQ ID NOs: 9 to 12, 17 to 21 and 25 to 36. The Factor VIII amino acid sequence may be any one of SEQ ID NOs: 9 to 12, 17 to 21 and 25 to 36.

Optionally, the Factor VIII polypeptide may comprise a Factor VIII amino acid sequence which has at least 90% sequence identity to SEQ ID NO: 31. The Factor VIII polypeptide may comprise an amino acid sequence which is at least 95%, at least 96%, at least 98%, at least 99% or 100% identical to SEQ ID NO: 31. Optionally, the Factor VIII polypeptide may be a Factor VIII amino acid sequence which has at least 90% sequence identity to SEQ ID NO: 31. The Factor VIII polypeptide may be a Factor VIII amino acid sequence which has at least 95%, at least 96%, at least 97%, at least 98% or at least 99% sequence identity to SEQ ID NO: 31. The Factor VIII amino acid sequence may comprise SEQ ID NO: 31. The Factor VIII amino acid sequence may be SEQ ID NO: 31.

Amino acids “corresponding to” specified positions of a specified SEQ ID NO may be amino acids at the specified positions of the particular SEQ ID NO recited. For example, the amino acids “corresponding to positions 724 to 740 of SEQ ID NO: 1” may be the amino acids at positions 724 to 740 of SEQ ID NO: 1. Alternatively, amino acids “corresponding to” specified positions of a specified SEQ ID NO may be amino acids from an alternative amino acid sequence which correspond to the specified positions of the specified SEQ ID NO. For example, the amino acids “corresponding to positions 724 to 740 of SEQ ID NO: 1” may be the amino acids from an alternative amino acid sequence which correspond to positions 724 to 740 of SEQ ID NO: 1. It is within the capabilities of the person skilled in the art to determine which amino acids in an alternative amino acid sequence “correspond to” the specified positions in the specified SEQ ID NO. For example, the person skilled in the art merely needs to perform a sequence alignment of the alternative amino acid sequence with the specified SEQ ID NO using a suitable alignment algorithm such as that of Needleman and Wunsch described above, and determine which region of the alternative amino acid sequence aligns to the specified positions in the specified SEQ ID NO. For example, the skilled person is able to align the alternative amino acid sequence with SEQ ID NO: 1 and determine which amino acids align, and therefore correspond to, positions 724 to 740 of SEQ ID NO. 1.

A Polynucleotide Comprising a Factor VIII Nucleotide Sequence

The invention provides a polynucleotide comprising a Factor VIII nucleotide sequence, wherein the Factor VIII nucleotide sequence encodes a Factor VIII polypeptide and wherein the Factor VIII nucleotide sequence is not a wild-type Factor VIII nucleotide sequence. The Factor VIII nucleotide sequence may encode any of the Factor VIII amino acid sequences or Factor VIII polypeptides of the invention, or another Factor VIII amino acid sequence such as that of a wild-type Factor VIII polypeptide or a wild-type Factor VIII polypeptide lacking the B domain.

The term “polynucleotide” refers to a polymeric chain of nucleotides of any length, e.g. deoxyribonucleotides, ribonucleotides, or analogs thereof. For example, the polynucleotide may comprise DNA (deoxyribonucleotides) or RNA (ribonucleotides). The polynucleotide may consist of DNA. The polynucleotide may be mRNA. Since the polynucleotide may comprise RNA or DNA, all references to T (thymine) nucleotides may be replaced with U (uracil).

The Factor VIII nucleotide sequence encodes a Factor VIII polypeptide. A “sequence” that “encodes” refers to a nucleotide sequence comprising codons that encode the encoded amino acid sequence. For example, a nucleotide sequence that encodes a Factor VIII polypeptide comprises codons that encode the Factor VIII polypeptide. A suitable nucleotide sequence is provided in SEQ ID NO:3. The Factor VIII nucleotide sequence may comprise one or more regions of one or more non-coding nucleotides, such as introns. The regions of non-coding nucleotides may interrupt the sequence of codons that encode the encoded amino acid sequence. Thus the sequence of codons that encode the encoded amino acid sequence may be contiguous in sequence or separated by one or more regions of non-coding nucleotides. Preferably the Factor VIII nucleotide sequence does not comprise any non-coding nucleotides. Herein, the stop codon will not be considered non-coding nucleotides.

The following Table (Table 1) describes codons that encode each amino acid:

| Amino Acid | Codon |
|---|---|
| Phenylalanine | TTC |
| | TTT |
| Leucine | TTA |
| | TTG |
| | CTT |
| | CTC |
| | CTA |
| | CTG |
| Isoleucine | ATT |
| | ATC |
| | ATA |
| Methionine | ATG |
| Valine | GTT |
| | GTC |
| | GTA |
| | GTG |
| Serine | TCT |
| | TCC |
| | TCA |
| | TCG |
| | AGT |
| | AGC |
| Arginine | CGT |
| | CGC |
| | CGA |
| | CGG |
| | AGA |
| | AGG |
| Proline | CCT |
| | CCC |
| | CCA |
| | CCG |
| Threonine | ACT |
| | ACC |
| | ACA |
| | ACG |
| Alanine | GCT |
| | GCC |
| | GCA |
| | GCG |
| Tyrosine | TAT |
| | TAC |
| Histidine | CAT |
| | CAC |
| Glutamine | CAA |
| | CAG |
| Glycine | GGT |
| | GGC |
| | GGA |
| | GGG |
| Asparagine | AAT |
| | AAC |
| Lysine | AAA |
| | AAG |
| Aspartic Acid | GAT |
| | GAC |
| Glutamic Acid | GAA |
| | GAG |
| Cysteine | TGT |
| | TGC |
| Tryptophan | TGG |

The corresponding RNA codons will contain Us in place of the Ts in the Table (Table 1) above.

The Factor VIII nucleotide sequence may encode a mature Factor VIII polypeptide. Optionally, the Factor VIII nucleotide sequence does not encode all or a portion of a signal peptide. The Factor VIII nucleotide sequence may encode all or part of a signal peptide, such as any or part of any of the signal peptides described herein.

The Factor VIII nucleotide sequence of the invention comprises a sequence of nucleotides which is not found in the corresponding portion of a wild-type Factor VIII nucleotide sequence. The Factor VIII nucleotide may comprise one or more portions which is not wild-type sequence (i.e. a sequence found in the corresponding portion of a wild-type Factor VIII nucleotide sequence). Put another way, at least a portion of the Factor VIII nucleotide sequence is not a sequence corresponding to a wild-type nucleotide sequence encoding Factor VIII. For example, one or more of the wild-type nucleotides may be absent (e.g. deleted) in the Factor VIII nucleotide sequence of the invention in comparison to a corresponding portion of the wild-type Factor VIII nucleotide sequence. Alternatively or additionally, the Factor VIII nucleotide sequence of the invention may comprise one or more additional nucleotides in comparison to a corresponding portion of the wild-type Factor VIII nucleotide sequence. Alternatively or additionally, the Factor VIII nucleotide sequence of the invention may comprise one or more nucleotide(s) in place of wild-type nucleotide(s) (e.g. substitutions) in comparison to a corresponding portion of the wild-type Factor VIII nucleotide sequence. For example, the Factor VIII nucleotide sequence of the invention may comprise one or more different codons in place of the wild-type codon(s) (i.e. the codon found in the corresponding position in a wild-type Factor VIII nucleotide sequence), wherein a “different” codon encodes a different amino acid to the amino acid encoded by the wild-type codon. As a further example, the Factor VIII nucleotide sequence of the invention may comprise one or more alternative codon(s) in place of the wild-type codon(s), wherein an “alternative” codon is a codon which has a different sequence to the wild-type codon but which encodes the same amino acid as the wild-type codon (i.e. a degenerate codon). Table 1 describes the codons which encode each amino acid.

The Factor VIII nucleotide sequence of the invention may comprise one or more (e.g. two, three, four, five, six or more) portions which are codon-optimised. The Factor VIII nucleotide sequence of the invention may be codon-optimised. As mentioned above, the genetic code is degenerate, and many amino acids may be encoded by more than one alternative codon. However, the genetic code of different organisms, tissues or cells may be biased towards using one particular codon for encoding a particular amino acid. A nucleotide sequence that is “codon-optimised” may express at higher levels in a particular cell type such as Huh7 cells. Preferably, a codon-optimised nucleic acid sequence is modified relative to a wild-type sequence whilst the amino acid sequence encoded by the nucleotide sequence is not modified.

By describing a nucleotide sequence as “codon-optimised”, all the codons do not need to be optimised. Thus the portion of the Factor VIII nucleotide sequence that is codon-optimised may comprise one or more alternative codon(s) in place of the wild-type codon(s) when compared to the corresponding portion of a wild-type Factor VIII nucleotide sequence. If the Factor VIII nucleotide sequence comprises a portion that is codon-optimised, the polypeptide encoded by the Factor VIII nucleotide sequence may be expressed at a higher level than a polypeptide encoded by a wild-type Factor VIII nucleotide sequence. The portion of the Factor VIII nucleotide sequence that is codon-optimised may be codon-optimised for expression in human liver cells. Thus the polypeptide encoded by the Factor VIII nucleotide sequence may be expressed at a higher level than the polypeptide encoded by an equivalent non-codon-optimised (e.g. a wild-type) Factor VIII nucleotide sequence when the sequences are expressed in human liver cells. An “equivalent” non-codon optimised Factor VIII nucleotide sequence is identical (i.e. encodes the same Factor VIII polypeptide and comprises the same transcriptional regulatory elements etc.) except the codons used to encode the Factor VIII polypeptide will correspond to the corresponding codons of a wild-type Factor VIII sequence such as SEQ ID NO: 2. Typically, the codon-optimised portion of the Factor VIII nucleotide sequence does not comprise the stop codon.

A portion of the Factor VIII nucleotide sequence that is codon-optimised may encode a contiguous portion of a Factor VIII polypeptide. Optionally, the portion of the Factor VIII nucleotide sequence that is codon-optimised (or the Factor VIII nucleotide sequence) does not encode at least one amino acid(s) corresponding to positions 746 to 1639 of SEQ ID NO: 1. Optionally, the portion of the Factor VIII nucleotide sequence that is codon-optimised (or the Factor VIII nucleotide sequence) does not encode amino acids corresponding to positions 746 to 1639 of SEQ ID NO: 1. Optionally, the portion of the Factor VIII nucleotide sequence that is codon-optimised (or the Factor VIII nucleotide sequence) does not encode one or more amino acid(s) of the amino acid sequence of the modified BDR region. Optionally, the portion of the Factor VIII nucleotide sequence that is codon-optimised (or the Factor VIII nucleotide sequence) does not encode the amino acid sequence of the modified BDR region or the wild-type BDR region.

Optionally the entire Factor VIII nucleotide sequence may be codon-optimised.

The Factor VIII nucleotide sequence may comprise a sequence of nucleotides immediately juxtaposed to a further sequence of nucleotides or connected by a linker (e.g. a short linker), where the two sequences of nucleotides are found in a wild-type Factor VIII nucleotide sequence, but the two sequences of nucleotides are not immediately juxtaposed in a wild-type Factor VIII nucleotide sequence. Put another way, the nucleotides between the two sequences of nucleotides may be absent (e.g. deleted) in the Factor VIII nucleotide sequence in comparison to the corresponding portion of the wild-type Factor VIII nucleotide sequence. The Factor VIII nucleotide sequence may thus comprise a portion which is not wild type. For example, the Factor VIII nucleotide sequence may comprise nucleotides which encode the amino acids which correspond to positions 724 to 731 of SEQ ID NO: 1 immediately juxtaposed to nucleotides which encode the amino acids which correspond to positions 1670 to 1689 of SEQ ID NO: 1. Optionally, the two nucleotides sequences which are immediately juxtaposed are not the same as the corresponding wild-type sequences. The two nucleotides sequences which are immediately juxtaposed may vary in comparison to the corresponding portion of the wild-type sequence in any of the ways described herein, such as by being codon-optimised. As an example, the Factor VIII nucleotide sequence may comprise nucleotides 1 to 2193 of SEQ ID NO:3 immediately juxtaposed to nucleotides 2326 to 4314 of SEQ ID NO: 3 (or in other words, the Factor VIII nucleotide sequence may not comprise nucleotides 2194 to 2325 of SEQ ID NO:3, i.e. these nucleotides may be absent). As a further example, the Factor VIII nucleotide sequence may consist of nucleotides 1 to 2193 and 2326 to 4314 of SEQ ID NO:3.

As yet further examples, the Factor VIII nucleotide sequence may comprise or consist of nucleotides which encode the amino acids which correspond to the positions of SEQ ID NO:1 shown in Table 2 for the FVIII polypeptides having an amino acid sequence set forth in any one of SEQ ID NOs:9-36. These FVIII polypeptides each comprise at least a first sequence of amino acids and a second sequence of amino acids. A number of the FVIII polypeptides further comprise a third sequence of amino acids. The FVIII polypeptides are encoded by Factor VIII nucleotide sequences which comprise first and second (and optionally third) sequences of nucleotides which are not immediately juxtaposed in a wild-type Factor VIII nucleotide sequence. The Factor VIII nucleotide sequences may comprise or consist of such sequences of nucleotides immediately juxtaposed to one-another, such that the Factor VIII polypeptide encoded by the Factor VIII nucleotide comprises or consists of the amino acids of SEQ ID NO:1 indicated in Table 2.

TABLE 2
exemplary FVIII polypeptide sequences

| SEQ ID NO: | Amino acids corresponding to SEQ ID NO: 1 (deletion) |
|---|---|
| 9 | 1-740, 1670-2332 (741-1669) |
| 10 | 1-740, 1690-2332 (741-1689) |
| 11 | 1-740, 1689-2332 (741-1688) |
| 12 | 1-740, 1670-1680, 1689-2332 (741-1669, 1681-1688) |
| 13 | 1-713, 1670-2332 (714-1669) |
| 14 | 1-713, 1670-1680, 1689-2332 (714-1669, 1681-1688) |
| 15 | 1-713, 1689-2332 (714-1688) |
| 16 | 1-713, 1649-2332 (714-1648) |
| 17 | 1-737, 1649-2332 (738-1648) |
| 18 | 1-734, 1649-2332 (735-1648) |
| 19 | 1-731, 1649-2332 (732-1648) |
| 20 | 1-728, 1649-2332 (729-1648) |
| 21 | 1-725, 1649-2332 (726-1648) |
| 22 | 1-722, 1649-2332 (723-1648) |
| 23 | 1-719, 1649-2332 (720-1648) |
| 24 | 1-716, 1649-2332 (717-1648) |
| 25 | 1-737, 1652-2332 (738-1651) |
| 26 | 1-737, 1655-2332 (738-1654) |
| 27 | 1-737, 1658-2332 (738-1665) |
| 28 | 1-737, 1661-2332 (738-1660) |
| 29 | 1-737, 1664-2332 (738-1663) |
| 30 | 1-737, 1667-2332 (738-1666) |
| 31 | 1-731, 1670-2332 (732-1669) |
| 32 | 1-731, 1670-1680, 1689-2332 (732-1669, 1681-1688) |
| 33 | 1-722, 1670-2332 (723-1669) |
| 34 | 1-722, 1670-1680, 1689-2332 (723-1669, 1681-1688) |
| 35 | 1-728, 1670-2332 (729-1669) |
| 36 | 1-728, 1667-2332 (729-2332) |

The “corresponding portion of the wild-type Factor VIII nucleotide sequence” may comprise more than one region from the wild-type Factor VIII nucleotide sequence. For example, as described herein, the Factor VIII nucleotide sequence may comprise a sequence of nucleotides immediately juxtaposed to a further sequence nucleotides, where the two sequences of nucleotides are found in a wild-type Factor VIII nucleotide sequence, but the two sequences of nucleotides are not immediately juxtaposed in a wild-type Factor VIII nucleotide sequence. Thus, the corresponding portion of the wild-type Factor VIII nucleotide sequence comprises more than one region from the wild-type Factor VIII nucleotide sequence. It is within the capabilities of the person skilled in the art to determine which nucleotides in wild-type Factor VIII nucleotide sequence are the “corresponding portion” to the Factor VIII nucleotide sequence of the invention. For example, the person skilled in the art merely needs to perform a sequence alignment of the two nucleotide sequences using a suitable alignment algorithm such as that of Needleman and Wunsch described above, and determine which region or regions of the two nucleotide sequences align. For example, the skilled person is able to align a nucleotide sequence (such as SEQ ID NO: 3) with a wild-type nucleotide sequence (such as SEQ ID NO: 2) and determine which nucleotides align, and therefore determine which nucleotides in wild-type Factor VIII nucleotide sequence are the “corresponding portion” to the portion of the Factor VIII nucleotide sequence which is not wild-type. For example, positions 1 to 2193 and 2326 to 4314 of SEQ ID NO: 3 correspond to positions 1 to 2193 and 5008 to 6996 of SEQ ID NO: 2. Preferably, the skilled person aligns the amino acid sequences encoded by the nucleotide sequences to determine which nucleotides align. For example, the skilled person is able to align the amino acid sequence encoded by a nucleotide sequence (such as SEQ ID NO: 3) with the amino acid sequence encoded by a wild-type nucleotide sequence (such as SEQ ID NO: 2) and determine which amino acids align and therefore, in turn, determine which nucleotides align. Therefore a skilled person can determine which nucleotides in wild-type Factor VIII nucleotide sequence are the “corresponding portion” to the Factor VIII nucleotide sequence of the invention.

Optionally, the Factor VIII nucleotide sequence encodes at least one amino acid corresponding to positions 746 to 1639 of SEQ ID NO: 1. Optionally, the Factor VIII nucleotide sequence encodes at least 2, at least 5, at least 10 or at least 25 amino acids corresponding to positions 746 to 1639 of SEQ ID NO: 1. Optionally, the Factor VIII nucleotide sequence encodes 88 or fewer, 74 or fewer, 50 or fewer, or 25 or fewer amino acids corresponding to positions 746 to 1639 of SEQ ID NO: 1. Optionally, the Factor VIII nucleotide sequence encodes a maximum of 88 amino acids corresponding to positions 746 to 1639 of SEQ ID NO: 1. Optionally, the Factor VIII nucleotide sequence encodes a maximum of 74 amino acids corresponding to positions 746 to 1639 of SEQ ID NO: 1. Optionally, the Factor VIII nucleotide sequence encodes between one and 89, between one and 75, between one and 50, between one and 25, or between one and 10 amino acids corresponding to positions 746 to 1639 of SEQ ID NO: 1.

Optionally, the Factor VIII nucleotide sequence encodes a region of the Factor VIII amino acid sequence which does not comprise amino acids corresponding to positions 746 to 1639 of SEQ ID NO: 1. The “Factor VIII nucleotide sequence” which “encode(s) a region of the Factor VIII amino acid sequence which does not comprise amino acids corresponding to positions 746 to 1639 of SEQ ID NO: 1” may comprise any of the nucleotides from the Factor VIII nucleotide sequence which encode any amino acids of the Factor VIII amino acid sequence except for nucleotides which encode the amino acids corresponding to positions 746 to 1639 of SEQ ID NO: 1. Correspondingly, the “region of the Factor VIII amino acid sequence which does not comprise amino acids corresponding to positions 746 to 1639 of SEQ ID NO: 1” may comprise any amino acids of the Factor VIII amino acid sequence except for the amino acids corresponding to positions 746 to 1639 of SEQ ID NO: 1.

For example, the “Factor VIII nucleotide sequence” may comprise nucleotides which encode one or more amino acids corresponding to positions 1 to 745 of SEQ ID NO: 1. As a further example, the “Factor VIII nucleotide sequence” may comprise nucleotides which encode one or more amino acids corresponding to positions 1640 to 2332 of SEQ ID NO: 1. As yet a further example, the “Factor VIII nucleotide sequence” may comprise nucleotides which encode one or more amino acids corresponding to positions 1 to 745 of SEQ ID NO: 1 and nucleotides which encode one or more amino acids corresponding to 1640 to 2332 of SEQ ID NO: 1.

As yet a further example, the “Factor VIII nucleotide sequence” may comprise nucleotides which encode one or more amino acids corresponding to positions 1 to 713 of SEQ ID NO: 1. As yet a further example, the “Factor VIII nucleotide sequence” may comprise nucleotides which encode one or more amino acids corresponding to positions 1696 to 2332 of SEQ ID NO: 1. As yet a further example, the “Factor VIII nucleotide sequence” may comprise nucleotides which encode one or more amino acids corresponding to positions 1 to 713 of SEQ ID NO: 1 and nucleotides which encode one or more amino acids corresponding to 1696 to 2332 of SEQ ID NO: 1.

Optionally, the amino acids of the “region of the Factor VIII amino acid sequence which does not comprise amino acids corresponding to positions 746 to 1639 of SEQ ID NO: 1” are contiguous. Optionally, the amino acids of the “region of the Factor VIII amino acid sequence which does not comprise amino acids corresponding to positions 746 to 1639 of SEQ ID NO: 1” are not contiguous.

Optionally, the “Factor VIII nucleotide sequence which encodes a region of the Factor VIII amino acid sequence” is codon-optimised as described herein.

Optionally, the Factor VIII nucleotide sequence may be at least 85.0%, at least 85.5%, at least 86.0%, at least 86.5%, at least 87%, at least 87.5%, at least 88%, at least 90%, at least 92%, at least 95%, at least 98%, at least 99%, or 100% identical to the nucleotides (i.e. the portions) of SEQ ID NO:3-6 (in particular SEQ ID NO:3) which encode a Factor VIII polypeptide of the invention. Optionally the Factor VIII nucleotide sequence may not comprise nucleotides of SEQ ID NO:3-6 (in particular SEQ ID NO:3) which do not encode a Factor VIII polypeptide of the invention. Optionally, the Factor VIII nucleotide sequence may comprise the nucleotides of any one of SEQ ID NOs:3-6 (in particular SEQ ID NO:3) which encode a Factor VIII polypeptide set forth in any one of SEQ ID NOs:9-36. Optionally, the Factor VIII nucleotide sequence may not comprise nucleotides of SEQ ID NO:3-6 (in particular SEQ ID NO:3) which do not encode a Factor VIII polypeptide set forth in any one of SEQ ID NOs:9-36. Optionally, the Factor VIII nucleotide sequence may consist of the nucleotides of SEQ ID NO:3-6 (in particular SEQ ID NO:3) which encode a Factor VIII polypeptide of the invention.

Optionally, the Factor VIII nucleotide sequence may be at least 88% identical to the nucleotides (i.e. the portions) of SEQ ID NO:3. Optionally, the Factor VIII nucleotide sequence may be at least 89% identical to the nucleotides (i.e. the portions) of SEQ ID NO:3. Optionally, the Factor VIII nucleotide sequence may be at least 88% identical to the nucleotides (i.e. the portions) of SEQ ID NO:3. Optionally, the Factor VIII nucleotide sequence may be at least 90% identical to the nucleotides (i.e. the portions) of SEQ ID NO:3.

The “Factor VIII nucleotide sequence which encodes a region of the Factor VIII amino acid sequence” may be at least 85.5%, at least 86.0%, at least 88%, at least 90%, at least 92%, at least 95%, at least 98%, at least 99%, or 100% identical to the portion of SEQ ID NO: 3 which encodes the “region of the Factor VIII amino acid sequence”. The “Factor VIII nucleotide sequence which encodes a region of the Factor VIII amino acid sequence” may be at least 85.0%, at least 85.5%, at least 86.0%, at least 88%, at least 90%, at least 92%, at least 95%, at least 98%, at least 99%, or 100% identical to the portion of SEQ ID NO: 4 which encodes the “region of the Factor VIII amino acid sequence”. The “Factor VIII nucleotide sequence which encodes a region of the Factor VIII amino acid sequence” may be at least 85.0%, at least 85.5%, at least 86.0%, at least 88%, at least 90%, at least 92%, at least 95%, at least 98%, at least 99%, or 100% identical to the portion of SEQ ID NO: 5 which encodes the “region of the Factor VIII amino acid sequence”. The “Factor VIII nucleotide sequence which encodes a region of the Factor VIII amino acid sequence” may be at least 86.5%, at least 87.0%, at least 87.5%, at least 88%, at least 90%, at least 92%, at least 95%, at least 98%, at least 99%, or 100% identical to the portion of SEQ ID NO: 6 which encodes the “region of the Factor VIII amino acid sequence”. The “region of the Factor VIII amino acid sequence” may comprise at least 650, at least 700, at least 720, or 745 amino acids corresponding to positions 1 to 745 of SEQ ID NO: 1. The “region of the Factor VIII amino acid sequence” may comprise at least 550, at least 600, at least 650, at least 700, or 712 amino acids corresponding to positions 1640 to 2332 of SEQ ID NO: 1. The “region of the Factor VIII amino acid sequence” may comprises at least 1200, at least 1300, at least 1400, at least 1450, or 1457 amino acids corresponding to positions 1 to 745 and 1640 to 2332 of SEQ ID NO: 1.

The “region of the Factor VIII amino acid sequence” may comprise amino acids corresponding to positions 714 to 745 of SEQ ID NO: 1 or a fragment thereof. The “region of the Factor VIII amino acid sequence” may comprise at least 10, at least 15, at least 20, at least 25, at least 30, or 32 amino acids corresponding to positions 714 to 745 of SEQ ID NO: 1. The “region of the Factor VIII amino acid sequence” may comprise amino acids corresponding to positions 1640 to 1696 of SEQ ID NO: 1 or a fragment thereof. The “region of the Factor VIII amino acid sequence” may comprise at least 20, at least 25, at least 30, at least 35, at least 40, at least 45, at least 50, at least 55, or 57 amino acids corresponding to positions 1640 to 1696 of SEQ ID NO: 1. The “region of the Factor VIII amino acid sequence” may comprise amino acids corresponding to positions 714 to 745 and 1640 to 1696 of SEQ ID NO: 1 or a fragment thereof. The “region of the Factor VIII amino acid sequence” may comprise at least 30, at least 35, at least 40, at least 45, at least 50, at least 55, at least 60, at least 65, at least 70, at least 75, at least 80, at least 85, or 89 amino acids corresponding to positions 714 to 745 and 1640 to 1696 of SEQ ID NO: 1.

Optionally, the “region of the Factor VIII amino acid sequence” does not comprise the modified BDR region. The “region of the Factor VIII amino acid sequence” may comprise at least 650, at least 700, at least 710, or 713 amino acids corresponding to positions 1 to 713 of SEQ ID NO: 1. The “region of the Factor VIII amino acid sequence” may comprise at least 550, at least 600, at least 650, or 655 amino acids corresponding to positions 1697 to 2332 of SEQ ID NO: 1. The “region of the Factor VIII amino acid sequence” may comprise at least 1200, at least 1300, at least 1350, or 1368 amino acids corresponding to positions 1 to 713 and 1697 to 2332 of SEQ ID NO: 1.

The portion of the Factor VIII nucleotide sequence that is codon-optimised may be at least 1500, at least 2000, at least 2500, at least 3000, at least 3500, at least 4000, at least 4100, at least 4200, at least 4300, or at least 4350 nucleotides in length. The portion of the Factor VIII nucleotide sequence that is codon-optimised may be 4371 or fewer nucleotides in length. The portion of the Factor VIII nucleotide sequence that is codon-optimised may be between 1500 and 4372, between 2500 and 4372, between 3500 and 4372, between 4000 and 4372, or around 4371 nucleotides in length.

The portion of the Factor VIII nucleotide sequence that is codon-optimised may be at least 1500, at least 2000, at least 2500, at least 3000, at least 3500, at least 4000, or at least 4100 nucleotides in length. The portion of the Factor VIII nucleotide sequence that is codon-optimised may be 4104 or fewer nucleotides in length. The portion of the Factor VIII nucleotide sequence that is codon-optimised may be between 1500 and 4105, between 2500 and 4105, between 3500 and 4105, between 4000 and 4105, or around 4104 nucleotides in length.

The portion of the Factor VIII nucleotide sequence that is codon-optimised may encode a mature Factor VIII polypeptide. Optionally, the portion of the Factor VIII nucleotide sequence that is codon-optimised does not encode all or a portion of a signal peptide. The portion of the Factor VIII nucleotide sequence that is codon-optimised may encode all or a portion of a signal peptide.

A codon-optimised nucleotide sequence may comprise at least one more “preferred” codons than a corresponding nucleotide sequence which is not codon-optimised. A codon-optimised nucleotide sequence may comprise a higher percentage of “preferred” codons than a corresponding nucleotide sequence which is not codon-optimised. A codon-optimised nucleotide sequence may comprise at least one fewer “non-preferred” codons than a corresponding nucleotide sequence which is not codon-optimised. A codon-optimised nucleotide sequence may comprise a lower percentage of “non-preferred” codons than a corresponding nucleotide sequence which is not codon-optimised. Preferred codons for expression of Factor VIII in human liver cells are underlined in Table 1.

In the portion of the Factor VIII amino acid sequence that is codon-optimised, at least 50%, at least 55%, at least 60%, at least 61%, at least 62%, at least 63%, at least 64%, at least 65%, at least 69%, at least 70%, at least 75%, or at least 78% of the codons may be selected from the group consisting of: TTC, CTG, ATC, GTG, TCC, AGT, AGC, CCT, CCC, ACC, ACA, GCC, TAC, CAC, CAG, AAC, AAG, GAC, GAG, TGT, AGA, and GGC. Preferably, at least 60% of the codons are selected from the above group.

Optionally, the Factor VIII nucleotide sequence may comprise more codons encoding phenylalanine than a non-codon-optimised nucleotide sequence which are TTC. Optionally the Factor VIII nucleotide sequence may comprise fewer codons encoding phenylalanine than a non-codon-optimised sequence which are TTT.

Optionally, the Factor VIII nucleotide sequence may comprise more codons encoding leucine than a non-codon-optimised nucleotide sequence which are CTG. Optionally, the Factor VIII nucleotide sequence may comprise fewer codons encoding leucine than a non-codon-optimised sequence which are TTA, TTG, CTT, CTC or CTA. Optionally, the Factor VIII nucleotide sequence may comprise no codons encoding leucine which are TTA. Optionally, the Factor VIII nucleotide sequence may comprise no codons encoding leucine which are TTG. Optionally, the Factor VIII nucleotide sequence may comprise no codons encoding leucine which are CTC. Optionally, the Factor VIII nucleotide sequence may comprise no codons encoding leucine which are CTA.

Optionally, the Factor VIII nucleotide sequence may comprise more codons encoding isoleucine than a non-codon-optimised nucleotide sequence which are ATC. Optionally, the Factor VIII nucleotide sequence may comprise fewer codons encoding isoleucine than a non-codon-optimised sequence which are ATT or ATA. Optionally, the Factor VIII nucleotide sequence may comprise no codons encoding isoleucine which are ATA.

Optionally, the Factor VIII nucleotide sequence may comprise more codons encoding valine than a non-codon-optimised nucleotide sequence which are GTG. Optionally, the Factor VIII nucleotide sequence may comprise fewer codons encoding valine than a non-codon-optimised sequence which are GTT, GTC or GTA. Optionally, the Factor VIII nucleotide sequence may comprise no codons encoding valine which are GTC. Optionally, the Factor VIII nucleotide sequence may comprise no codons encoding valine which are GTA.

Optionally, the Factor VIII nucleotide sequence may comprise more codons encoding serine than a non-codon-optimised nucleotide sequence which are TCC, AGT or AGC. Optionally, the Factor VIII nucleotide sequence may comprise fewer codons encoding serine than a non-codon-optimised sequence which are TCT, TCA or TCG. Optionally, the Factor VIII nucleotide sequence may comprise no codons encoding serine which are TCA. Optionally, the Factor VIII nucleotide sequence may comprise no codons encoding serine which are TCG.

Optionally, the Factor VIII nucleotide sequence may comprise more codons encoding proline than a non-codon-optimised nucleotide sequence which are CCT or CCC. Optionally, the Factor VIII nucleotide sequence may comprise fewer codons encoding proline than a non-codon-optimised sequence which are CCA or CCG. Optionally, the Factor VIII nucleotide sequence may comprise no codons encoding proline which are CCG.

Optionally, the Factor VIII nucleotide sequence may comprise more codons encoding threonine than a non-codon-optimised nucleotide sequence which are ACC or ACA. Optionally, the Factor VIII nucleotide sequence may comprise fewer codons encoding threonine than a non-codon-optimised sequence which are ACT or ACG. Optionally, the Factor VIII nucleotide sequence may comprise no codons encoding threonine which are ACT.

Optionally, the Factor VIII nucleotide sequence may comprise more codons encoding alanine than a non-codon-optimised nucleotide sequence which are GCC. Optionally, the Factor VIII nucleotide sequence may comprise fewer codons encoding alanine than a non-codon-optimised sequence which are GCT, GCA or GCG. Optionally, the Factor VIII nucleotide sequence may comprise no codons encoding alanine which are GCG.

Optionally, the Factor VIII nucleotide sequence may comprise more codons encoding tyrosine than a non-codon-optimised nucleotide sequence which are TAC. Optionally, the Factor VIII nucleotide sequence may comprise fewer codons encoding phenylalanine than a non-codon-optimised sequence which are TAT.

Optionally, the Factor VIII nucleotide sequence may comprise more codons encoding histidine than a non-codon-optimised nucleotide sequence which are CAC. Optionally, the Factor VIII nucleotide sequence may comprise fewer codons encoding histidine than a non-codon-optimised sequence which are CAT.

Optionally, the Factor VIII nucleotide sequence may comprise more codons encoding glutamine than a non-codon-optimised nucleotide sequence which are CAG. Optionally, the Factor VIII nucleotide sequence may comprise fewer codons encoding glutamine than a non-codon-optimised sequence which are CAA.

Optionally, the Factor VIII nucleotide sequence may comprise more codons encoding asparagine than a non-codon-optimised nucleotide sequence which are AAC. Optionally, the Factor VIII nucleotide sequence may comprise fewer codons encoding asparagine than a non-codon-optimised sequence which are AAT.

Optionally, the Factor VIII nucleotide sequence may comprise more codons encoding lysine than a non-codon-optimised nucleotide sequence which are AAG. Optionally, the Factor VIII nucleotide sequence may comprise fewer codons encoding lysine than a non-codon-optimised sequence which are AAA.

Optionally, the Factor VIII nucleotide sequence may comprise more codons encoding aspartic acid than a non-codon-optimised nucleotide sequence which are GAT. Optionally, the Factor VIII nucleotide sequence may comprise fewer codons encoding aspartic acid than a non-codon-optimised sequence which are GAC.

Optionally, the Factor VIII nucleotide sequence may comprise more codons encoding aspartic acid than a non-codon-optimised nucleotide sequence which are GAC. Optionally, the Factor VIII nucleotide sequence may comprise fewer codons encoding aspartic acid than a non-codon-optimised sequence which are GAT.

Optionally, the Factor VIII nucleotide sequence may comprise more codons encoding glutamic acid than a non-codon-optimised nucleotide sequence which are GAG. Optionally, the Factor VIII nucleotide sequence may comprise fewer codons encoding glutamic acid than a non-codon-optimised sequence which are GAA.

Optionally, the Factor VIII nucleotide sequence may comprise more codons encoding cysteine than a non-codon-optimised nucleotide sequence which are TGT. Optionally, the Factor VIII nucleotide sequence may comprise fewer codons encoding cysteine than a non-codon-optimised sequence which are TGC.

Optionally, the Factor VIII nucleotide sequence may comprise more codons encoding arginine than a non-codon-optimised nucleotide sequence which are AGA. Optionally, the Factor VIII nucleotide sequence may comprise fewer codons encoding arginine than a non-codon-optimised sequence which are CGT, CGC, CGA, CGG or AGG. Optionally, the Factor VIII nucleotide sequence may comprise no codons encoding arginine which are CGC. Optionally, the Factor VIII nucleotide sequence may comprise no codons encoding arginine which are CGG.

Optionally, the Factor VIII nucleotide sequence may comprise more codons encoding glycine than a non-codon-optimised nucleotide sequence which are GGC. Optionally, the Factor VIII nucleotide sequence may comprise fewer codons encoding glycine than a non-codon-optimised sequence which are GGT, GGA or GGG. Optionally, the Factor VIII nucleotide sequence may comprise no codons encoding glycine which are GGT. Optionally, the Factor VIII nucleotide sequence may comprise no codons encoding glycine which are GGG.

A polypeptide encoded by the Factor VIII nucleotide sequence may be expressed in human liver cells at higher levels compared to a reference wild-type Factor VIII nucleotide sequence.

Thus the Factor VIII polypeptide encoded by the Factor VIII nucleotide sequence may be expressed at a higher level than a polypeptide encoded by a wild-type Factor VIII nucleotide sequence when the sequences are expressed in human liver cells. For example, a polypeptide encoded by the Factor VIII nucleotide sequence may be expressed in human liver cells at least 1.1 fold, at least 1.2 fold, at least 1.5 fold, at least 1.8 fold, at least 2 fold, at least 5 fold, at least 10 fold, at least 20 fold, at least 30 fold, at least 40 fold, or at least 50 fold higher compared to a reference wild-type Factor VIII nucleotide sequence. The “reference wild-type Factor VIII nucleotide sequence” may be any Factor VIII nucleotide sequence that uses wild type codons to encode a Factor VIII polypeptide (which may itself be a wild-type Factor VIII polypeptide or may be a modified Factor VIII polypeptide such as a Factor VIII polypeptide of the invention comprising a modified BDR), such as the Factor VIII nucleotide sequence of SEQ ID NO: 2. The reference wild-type Factor VIII nucleotide is an “equivalent” factor VIII nucleotide sequence i.e. the reference wild-type Factor VIII nucleotide encodes the same Factor VIII polypeptide as the Factor VIII polynucleotide to which it is being compared.

The Factor VIII polypeptide encoded by the Factor VIII nucleotide sequence may be expressed in human liver cells at at least 50%, at least 60%, at least 70%, at least 75%, at least 80%, at least 85%, at least 90%, at least 95%, at least 98%, at least 99%, or at least 100% of the level expressed by a the Factor VIII encoded by the polynucleotide of SEQ ID NO: 3. The Factor VIII polypeptide encoded by the Factor VIII nucleotide sequence may be expressed in human liver cells at at least 50%, at least 60%, at least 70%, at least 75%, at least 80%, at least 85%, at least 90%, at least 95%, at least 98%, at least 99%, or at least 100% of the level expressed by a the Factor VIII polypeptide encoded by the polynucleotide of SEQ ID NO: 4. The Factor VIII polypeptide encoded by the Factor VIII nucleotide sequence may be expressed in human liver cells at at least 50%, at least 60%, at least 70%, at least 75%, at least 80%, at least 85%, at least 90%, at least 95%, at least 98%, at least 99%, or at least 100% of the level expressed by the Factor VIII polypeptide encoded by the polynucleotide of SEQ ID NO: 5. The Factor VIII polypeptide encoded by the Factor VIII nucleotide sequence may be expressed in human liver cells at at least 50%, at least 60%, at least 70%, at least 75%, at least 80%, at least 85%, at least 90%, at least 95%, at least 98%, at least 99%, or at least 100% of the level expressed by the Factor VIII polypeptide encoded by the polynucleotide of SEQ ID NO: 6.

The Factor VIII nucleotide sequence encoding the Factor VIII amino acid sequence may comprise or consist of a sequence that is at least 85.5%, at least 86.0%, at least 88%, at least 90%, at least 92%, at least 95%, at least 98%, at least 99%, or 100% identical to the portion of SEQ ID NO: 3 which encodes the Factor VIII amino acid sequence. The Factor VIII nucleotide sequence encoding the Factor VIII amino acid sequence may comprise or consist of a sequence that is at least 85.5%, at least 86.0%, at least 88%, at least 90%, at least 92%, at least 95%, at least 98%, at least 99%, or 100% identical to SEQ ID NO: 3. The Factor VIII nucleotide sequence encoding the Factor VIII amino acid sequence may comprise or consist of the sequence of SEQ ID NO: 3.

The Factor VIII nucleotide sequence encoding the Factor VIII amino acid sequence may comprise or consist of a sequence that is at least 85.0%, at least 85.5%, at least 86.0%, at least 88%, at least 90%, at least 92%, at least 95%, at least 98%, at least 99%, or 100% identical to the portion of SEQ ID NO: 4 which encodes the Factor VIII amino acid sequence. The Factor VIII nucleotide sequence encoding the Factor VIII amino acid sequence may comprise or consist of a sequence that is at least 85.0%, at least 85.5%, at least 86.0%, at least 88%, at least 90%, at least 92%, at least 95%, at least 98%, at least 99%, or 100% identical to SEQ ID NO: 4. The Factor VIII nucleotide sequence encoding the Factor VIII amino acid sequence may comprise or consist of the sequence of SEQ ID NO: 4.

The Factor VIII nucleotide sequence encoding the Factor VIII amino acid sequence may comprise or consist of a sequence that is at least 85.0%, at least 85.5%, at least 86.0%, at least 88%, at least 90%, at least 92%, at least 95%, at least 98%, at least 99%, or 100% identical to the portion of SEQ ID NO: 5 which encodes the Factor VIII amino acid sequence. The Factor VIII nucleotide sequence encoding the Factor VIII amino acid sequence may comprise or consist of a sequence that is at least 85.0%, at least 85.5%, at least 86.0%, at least 88%, at least 90%, at least 92%, at least 95%, at least 98%, at least 99%, or 100% identical to SEQ ID NO: 5. The Factor VIII nucleotide sequence encoding the Factor VIII amino acid sequence may comprise or consist of the sequence of SEQ ID NO: 5.

The Factor VIII nucleotide sequence encoding the Factor VIII amino acid sequence may comprise or consist of a sequence that is at least 86.5%, at least 87.0%, at least 87.5%, at least 88.0%, at least 90%, at least 92%, at least 95%, at least 98%, at least 99%, or 100% identical to the portion of SEQ ID NO: 6 which encodes the Factor VIII amino acid sequence. The Factor VIII nucleotide sequence encoding the Factor VIII amino acid sequence may comprise or consist of a sequence that is at least 86.5%, at least 87.0%, at least 87.5%, at least 88.0%, at least 90%, at least 92%, at least 95%, at least 98%, at least 99%, or 100% identical to SEQ ID NO: 6. The Factor VIII nucleotide sequence encoding the Factor VIII amino acid sequence may comprise or consist of the sequence of SEQ ID NO: 6.

The Factor VIII nucleotide sequence encoding the Factor VIII amino acid sequence may comprise or consist of the sequence of SEQ ID NO: 39, SEQ ID NO: 40, SEQ ID NO: 41 or SEQ ID NO: 42. Preferably, the Factor VIII nucleotide sequence encoding the Factor VIII amino acid sequence comprises or consists of the sequence of SEQ ID NO: 39.

A gain of function mutation —embodiments relating to “one or more substitution mutations”

It is believed that the dissociation of the respective components of the activated Factor VIII heterotrimeric complex (and in particular, dissociation of the A2 domain from the A1 domain and A3-C1-C2 domains) is one of the principal mechanisms for the loss of Factor VIII activity following activation of the clotting cascade. Without wishing to be bound by theory, it is believed that amino acid substitution mutations disclosed herein may serve to prevent or delay dissociation of the respective components of the Factor VIII heterotrimeric complex following activation of the Factor VIII polypeptide, thereby extending the time for which the active FVIIIa is intact, and thus active.

Optionally, the Factor VIII polypeptides of the invention may comprise one or more amino acid substitutions (substitution mutations), compared to a corresponding wild-type Factor VIII amino acid sequence, which increase the stability/expression and/or specific activity of the Factor VIII polypeptide relative to a reference Factor VIII polypeptide (e.g. a reference wild-type Factor VIII polypeptide).

More particularly, the one or more substitution mutation may be located at an inter-domain interface between two domains of the Factor VIII polypeptide. In particular, the one or more substitution mutations may be located at the inter-domain interface selected from the A1/A3 domain interface, the A2/A3 domain interface, or the A1/C2 domain interface. The Factor VIII amino acid sequence therefore may comprise a substitution mutation in the A1, A2, A3 or C2 domain. Three-dimensional crystal structures of Factor VIII are available (for example, the PDB accession numbers 2RZE or 4BDV); and it would therefore be routine to identify amino acids within these domains, and more particularly amino acids which are situated at specified inter-domain interfaces, which may be substituted. With regard to wild-type Factor VIII, the A1 domain consists of amino acids corresponding to positions 1-329 of SEQ ID NO:1; the A2 domain consists of amino acids corresponding to positions 380-711 of SEQ ID NO:1; the A3 domain consists of amino acids corresponding to positions 1694-2021 of SEQ ID NO:1; the C1 domain consists of amino acids corresponding to positions 2021-2169 of SEQ ID NO:1; and the C2 domain consists of amino acids corresponding to positions 2174-2332 of SEQ ID NO:1. The last six amino acids (corresponding to positions 2327 to 2332 of SEQ ID NO: 1) may not be considered part of the C2 domain. Thus, optionally, the C2 domain consists of the amino acids corresponding to positions 2174-2326 of SEQ ID NO: 1.

Optionally, a Factor VIII polypeptide may comprise a Factor VIII amino acid sequence which comprises one or more substitution mutations at an inter-domain interface selected from the group consisting of:
a. the A1/A3 domain interface;
b. the A2/A3 domain interface; or
c. the A1/C2 domain interface
   wherein:
(i) the one or more substitution mutations comprises substitution of an amino acid with a more hydrophobic amino acid; or
(ii) the one or more substitution mutations comprises substitution of a pair of amino acids in the respective domains with cysteine residues;
   and wherein the Factor VIII polypeptide has higher specific activity than a reference wild-type Factor VIII polypeptide.

Optionally, a Factor VIII polypeptide may comprise a Factor VIII amino acid sequence which comprises one or more substitution mutations at an inter-domain interface selected from the group consisting of:
a. the A1/A3 domain interface;
b. the A2/A3 domain interface; or
c. the A1/C2 domain interface
   wherein:
(i) the one or more substitution mutations comprises substitution of an amino acid with a more hydrophobic amino acid; or
(ii) the one or more substitution mutations comprises substitution of a pair of amino acids in respective domains with cysteine residues;
   and wherein the Factor VIII polypeptide has higher stability than a reference wild-type Factor VIII polypeptide.

Optionally, a Factor VIII polypeptide may comprise a Factor VIII amino acid sequence which comprises one or more substitution mutations at an inter-domain interface selected from the group consisting of:
a. the A1/A3 domain interface;
b. the A2/A3 domain interface; or
c. the A1/C2 domain interface
   wherein:
(i) the one or more substitution mutations comprises substitution of an amino acid with a more hydrophobic amino acid; or
(ii) the one or more substitution mutations comprises substitution of a pair of amino acids in respective domains with cysteine residues;
   and wherein the Factor VIII polypeptide is expressed at a higher level in a host cell than a reference wild-type Factor VIII polypeptide.

Optionally, a Factor VIII polypeptide may comprise a Factor VIII amino acid sequence which comprises one or more substitution mutations selected from the group consisting of:
a. a substitution of an amino acid corresponding to M662 or H693 of SEQ ID NO: 1; or
b. a substitution of a pair of amino acids comprising a first amino acid and a second amino acid with cysteine residues, wherein:
   1. the first amino acid corresponds to M147, S149 or S289 of SEQ ID NO: 1 and the second amino acid corresponds to E1969, E1970 or N1977 of SEQ ID NO: 1;
   2. the first amino acid corresponds to T667, T669, N684, L687, 1689, S695 or F697 of SEQ ID NO: 1 and the second amino acid corresponds to S1791, G1799, A1800, R1803, E1844, S1949, G1981, V1982, or Y1979 of SEQ ID NO: 1; or
   3. the first amino acid corresponds to A108, T118 or V137 of SEQ ID NO: 1 and the second amino acid corresponds to N2172, Q2329 or Y2332 of SEQ ID NO: 1.

In particular embodiments, a Factor VIII polypeptide comprising a Factor VIII amino acid sequence comprising one or more substitution mutations at the A1/A3 domain interface, the A2/A3 domain interface or the A1/C2 domain interface as disclosed herein may have higher specific activity than a reference wild-type Factor VIII polypeptide. Exemplary substitution mutations which increase the specific activity of a Factor VIII polypeptide are disclosed herein.

Factor VIII is a cofactor for Factor X in the clotting cascade, and once activated acts in combination with activated Factor IX to activate Factor X. Factor VIII does not independently possess enzymatic activity as such. Thus, reference to the activity or specific activity of a Factor VIII polypeptide comprising a Factor VIII amino acid sequence which comprises one or more substitution mutations refers to the observed activity or specific activity in a functional assay for determining the activity of Factor X, in which Factor VIII is able to act as a cofactor for Factor X in combination with Factor IX (i.e. Factor VIII cofactor activity (FVIII:C)). Similarly, reference to a Factor VIII polypeptide comprising a Factor VIII amino acid sequence which comprises one or more substitution mutations having higher activity or specific activity than a reference Factor VIII polypeptide (such as a wild-type Factor VIII polypeptide) refers to the observed activity or specific activity of Factor X in a functional assay being increased for said Factor VIII polypeptide, relative to the observed activity or specific activity of Factor X in said assay for a reference Factor VIII polypeptide.

For the purposes of determining whether a Factor VIII polypeptide comprising a Factor VIII amino acid sequence comprising one or more substitution mutations has increased specific activity relative to a reference Factor VIII polypeptide such as a reference wild-type Factor VIII polypeptide, the activity of the Factor VIII polypeptide may be measured by a two stage chromogenic Factor Xa assay. For example, a suitable chromogenic assay is as follows. The Factor VIII polypeptide is mixed with human Factor X polypeptide and Factor IXa polypeptide, thrombin, phospholipids and calcium. The thrombin activates the Factor VIII polypeptide to form Factor VIIIa polypeptide. The thrombin-activated Factor VIII polypeptide forms an enzymatic complex with Factor IXa polypeptide, phospholipids and calcium, which enzymatic complex can catalyse the conversion of Factor X polypeptide to Factor Xa polypeptide. The activity of the Factor Xa polypeptide can catalyse cleavage of a chromogenic substrate (e.g. SXa-11) to produce pNA. The level of pNA generated can be measured by determining colour development at 405 nm (e.g. measured by absorbance). Factor X polypeptide, and therefore Factor Xa polypeptide, is provided in excess. Therefore the limiting factor is Factor VIIIa polypeptide. Thus, the level of pNA generated is proportional to the amount of the Factor Xa polypeptide generated by Factor FVIIIa polypeptide in the sample, which is proportional to the activity of Factor FVIIIa polypeptide in the sample. The activity of Factor FVIIIa polypeptide in the sample is a measure of the cofactor activity of the Factor FVIII polypeptide in the sample.

For example, a suitable chromogenic assay is the BIOPHEN FVIII:C assay (Ref: 221406) manufactured by HYPHEN BioMed as used in the Examples. The activity of the Factor VIII polypeptide may be measured using the BIOPHEN FVIII:C assay. More particularly, the activity of the Factor VIII polypeptide may be measured using the BIOPHEN FVIII:C assay according to the protocol described below in Example 1.

The Factor VIII polypeptide comprising a Factor VIII amino acid sequence comprising one or more substitution mutations may have a specific activity which is higher than the specific activity of a reference Factor VIII polypeptide (such as a reference wild-type Factor VIII polypeptide). The Factor VIII polypeptide may have a specific activity which is at least 1.1 fold, at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of a reference Factor VIII polypeptide. The Factor VIII polypeptide may have a specific activity which is between 1.2 fold and 5.5 fold, or between 1.5 fold and 5 fold, higher than the specific activity of a reference Factor VIII polypeptide. When referring to fold changes of activity, the term “between” includes the specified values. Thus, for example, “between 1.2 fold and 5.5 fold” includes the values 1.2 and 5.5.

In further embodiments, a Factor VIII polypeptide comprising a Factor VIII amino acid sequence comprising one or more substitution mutations at the A1/A3 domain interface, the A2/A3 domain interface or the A1/C2 domain interface as disclosed herein may have higher stability than a reference wild-type Factor VIII polypeptide.

A Factor VIII polypeptide having higher stability than a reference Factor VIII polypeptide retains a higher proportion of its Factor VIII activity over time than a reference Factor VIII polypeptide. The Factor VIII polypeptide of the invention may have a higher stability than a reference Factor VIII polypeptide prior to being activated, i.e. the Factor VIII polypeptide may have higher stability in its inactive form than a reference Factor VIII polypeptide in its inactive form. Put another way, an inactive Factor VIII polypeptide of the invention may have a higher stability than an inactive reference Factor VIII polypeptide. Alternatively or additionally, the Factor VIII polypeptide of the invention may have a higher stability than a reference Factor VIII polypeptide when activated. Put another way, an active Factor VIII polypeptide of the invention may have a higher stability than an active reference Factor VIII polypeptide.

A Factor VIII polypeptide comprising a Factor VIII amino acid sequence comprising one or more substitution mutations may have higher stability than a reference Factor VIII polypeptide prior to activation. A higher proportion of a Factor VIII polypeptide having a higher stability prior to activation than a reference Factor VIII polypeptide may remain capable of being activated over time than a reference Factor VIII polypeptide. Stability of a Factor VIII polypeptide prior to activation may be determined by measuring residual Factor VIII activity in a sample over time. Aliquots of a sample containing inactivated Factor VIII polypeptide may be removed at suitable time points, and Factor VIII polypeptide activity may be determined by activating the Factor VIII polypeptide in an aliquot and performing a two stage Factor X chromogenic assay as described herein. Activity at a given time point may then be compared to the initial Factor VIII activity of the sample (i.e. by determining Factor VIII activity at an initial time point), and the residual Factor VIII activity may be calculated as a percentage of the initial Factor VIII activity. Thus, when activated, the Factor VIII polypeptide having higher stability prior to activation than a reference Factor VIII polypeptide will have a higher residual activity (i.e. as a percentage of the initial Factor VIII polypeptide activity) than the reference Factor VIII polypeptide.

Optionally, residual activity of an inactive Factor VIII polypeptide in a sample may be measured over the course of five, ten, fifteen, twenty, twenty-five, thirty, forty-five or sixty minutes, or over the course of 2 hours, 3 hours, 4 hours, 5 hours, 6 hours, 7 hours, 8 hours, 9 hours, 10 hours, 11 hours, 12 hours, 18 hours or 24 hours, or over the course of 2 days, 3 days, 4 days, 5 days, 6 days or 7 days. Aliquots of a sample may be taken at a series of time points, e.g. two or more, three or more, four or more, five or more or six or more time points, in addition to taking an aliquot of the sample at an initial time point and Factor VIII activity in each aliquot may be determined. Comparing Factor VIII activity at each time point with Factor VIII activity at the initial time point allows residual Factor VIII activity to be determined. Optionally, specific activity may be determined at each time point.

A Factor VIII polypeptide comprising a Factor VIII amino acid sequence comprising one or more substitution mutations may have higher stability than a reference Factor VIII polypeptide when activated. A Factor VIII polypeptide having a higher stability when activated than a reference FVIII polypeptide may retain a higher proportion of its Factor VIII activity over time than a reference Factor VIII polypeptide following activation. Stability of a Factor VIII polypeptide following activation may be determined by measuring Factor VIII polypeptide activity in a sample over time, i.e. in a FVIIIa activity decay assay. A Factor VIII polypeptide may be activated using thrombin for 1 minute at 23° C. and immediately quenched using hirudin to inactivate thrombin. Aliquots may be removed at suitable time points and the activity may be determined using a Factor X chromogenic assay.

Optionally, activity of an active Factor VIII polypeptide in a sample may be measured over the course of five, ten, fifteen, twenty, twenty-five or thirty minutes following activation. Activity may be determined at a series of time points, e.g. two or more, three or more, four or more, five or more or six or more time points following activation, in addition to determining the activity of Factor VIII in a sample following activation (e.g. immediately following activation). By way of representative example, the activity of Factor VIII in a sample may be determined immediately following activation, and further measurements may be taken at a series of up to six time points within twenty minutes of activation. Optionally, specific activity may be determined at each time point.

A Factor VIII polypeptide comprising a Factor VIII amino acid sequence comprising one or more substitution mutations having higher stability than a reference Factor VIII polypeptide may have a longer half-life relative to a reference Factor VIII polypeptide. Optionally, the Factor VIII polypeptide has a longer half-life than a reference Factor VIII polypeptide prior to activation. Optionally, the Factor VIII polypeptide has a longer half-life than a reference Factor VIII when activated. Optionally, the Factor VIII polypeptide has a half-life which is at least 1.1, at least 1.2, at least 1.5, at least 1.7, at least 1.8, at least 2, at least 2.2, at least 2.5, at least 2.8 or at least 3 times the half-life of a reference wild-type Factor VIII polypeptide. Optionally, the Factor VIII polypeptide has a half-life when activated which is at least 1.1, at least 1.2, at least 1.5, at least 1.7, at least 1.8, at least 2, at least 2.2, at least 2.5, at least 2.8 or at least 3 times the half-life of a reference Factor VIII polypeptide when activated.

Optionally, the Factor VIII polypeptide has a half-life when activated with is between 1.1 and 3, between 1.2 and 3, between 1.5 and 3, between 1.7 and 3, between 1.8 and 3, between 2 and 3, between 2.2 and 3, between 2.5 and 3, or between 2.8 and 3 times the half-life of a reference Factor VIII polypeptide when activated. Optionally, the Factor VIII polypeptide comprising a Factor VIII amino acid sequence comprising one or more substitution mutations may have higher stability in a liquid (i.e. when in solution). Optionally, the Factor VIII polypeptide has a longer half-life in a liquid. Optionally, the liquid is conditioned medium, such as conditioned medium in which a host cell expressing a Factor VIII polypeptide is cultured. Optionally, the liquid is a biological sample. A biological sample may be blood, serum or plasma. Optionally, the Factor VIII polypeptide may have higher stability in plasma. Optionally, the Factor VIII polypeptide may have a longer half-life in plasma.

In yet further embodiments, a Factor VIII polypeptide comprising a Factor VIII amino acid sequence comprising a substitution of one or more amino acids at the A1/A3 domain interface, the A2/A3 domain interface or the A1/C2 domain interface as disclosed herein may be expressed at a higher level in a host cell than a reference Factor VIII polypeptide. For example, a Factor VIII polypeptide comprising the substitution may be expressed at least 1.1 fold, at least 1.2 fold, at least 1.5 fold, at least 1.8 fold, at least 2 fold, at least 2.5 fold, at least 3 fold, at least 3.5 fold, at least 4 fold, at least 4.5 fold or at least 5 fold higher compared to a reference Factor VIII polypeptide. Optionally, the Factor VIII polypeptide comprising the substitution may be expressed between 1.1 fold and 5 fold, between 1.2 fold and 5 fold, between 1.5 fold and 5 fold, between 1.8 fold and 5 fold, between 2 fold and 5 fold, between 2.5 fold and 5 fold, between 3 fold and 5 fold, between 3.5 fold and 5 fold, between 4 fold and 5 fold or between 4.5 fold and 5 fold higher compared to a reference Factor VIII polypeptide. Optionally, the Factor VIII polypeptide is secreted by a host cell at a higher level than a reference Factor VIII polypeptide.

The level of expression of a Factor VIII polypeptide (and therefore whether a Factor VIII polypeptide comprising a Factor VIII amino acid sequence comprising one or more substitution mutations is expressed at a higher level in a host cell than a referenced wild-type Factor VIII polypeptide) may typically be determined by measuring the level of Factor VIII polypeptide in a sample. The level of expression of a Factor VIII polypeptide of the invention in a host cell may be compared with the level of expression of a reference Factor VIII polypeptide in a host cell. This may be determined quantitatively. For example, the level of expression of a Factor VIII polypeptide may be determined by an ELISA assay as described above. Alternatively, the level of expression of a Factor VIII polypeptide may be determined semi-quantitatively, for example, by SDS-PAGE electrophoresis or by Western blot. In certain embodiments, expression in plasma can be determined by the Asserachrom assay as described in the Examples.

The level of expression of a Factor VIII polypeptide that is secreted by a host cell is typically determined, i.e. in contrast to the level of the Factor VIII polypeptide that is retained intracellularly by a host cell. This may be determined, for example, by separating cells (e.g. host cells) from a liquid containing the Factor VIII polypeptide (e.g. a biological sample, such as blood, serum or plasma, or culture medium such as conditioned medium in which a host cell expressing a Factor VIII polypeptide is cultured) and determining the level of expression of a Factor VIII polypeptide in the liquid. Cells may be separated, for example, by centrifugation or filtration, and/or the liquid may be decanted (e.g. by pipetting) from the cells. The level of expression of a Factor VIII polypeptide may be determined in a biological sample, such as blood, serum or plasma, or may be determined in a culture medium, such as conditioned medium in which a host cell expressing a Factor VIII polypeptide is cultured.

A host cell may be any eukaryotic host cell expressing a Factor VIII polypeptide comprising a Factor VIII amino acid sequence comprising one or more substitution mutations (or a reference Factor VIII polypeptide). Optionally, the host cell may be an insect cell expressing a Factor VIII polypeptide. Typically, the host cell may be a mammalian host cell expressing a Factor VIII polypeptide. Mammalian host cells include human, dog, pig, mouse, hamster, or guinea pig cells. More particularly, the host cell may be a mammalian liver cell, and more particularly may be a human liver cell. Optionally, the host cell may be an Huh7 cell. Optionally, the host cell may be a host cell within an organism. Optionally, expression may be in vivo expression. Optionally, expression may be in vivo expression and expression in plasma may be determined.

The Factor VIII polypeptides comprising a Factor VIII amino acid sequence comprising one or more substitution mutations may have higher specific activity and/or stability and/or may be expressed at a higher level in a host cell than a reference Factor VIII polypeptide. Optionally, the reference Factor VIII polypeptide may be a wild-type Factor VIII polypeptide, i.e. the reference Factor VIII polypeptide may be a reference wild-type Factor VIII polypeptide. The “reference wild-type Factor VIII polypeptide” may be any wild-type Factor FVIII polypeptide, such as the Factor VIII polypeptide of SEQ ID NO: 1. Optionally, the reference Factor VIII polypeptide is a beta domain deleted Factor VIII polypeptide. Optionally, the reference Factor VIII polypeptide has an amino acid sequence set forth in SEQ ID NOs: 7 or 8. Optionally, the reference Factor VIII polypeptide comprises the Factor VIII amino acid sequence of the Factor VIII polypeptide comprising a Factor VIII amino acid sequence comprising one or more substitution mutations, but does not comprise the one or more substitution mutations.

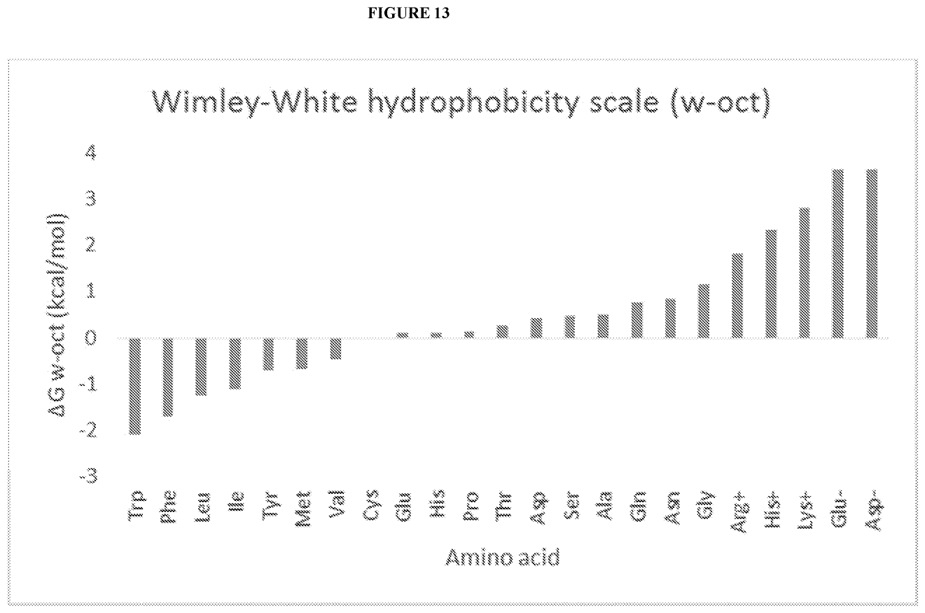

The one or more substitution mutations may comprise substitution of an amino acid at an inter-domain interface with a more hydrophobic amino acid. Biophysical studies have been performed to establish the hydrophobicity of the twenty naturally occurring amino acids, or more particularly, the relative hydrophobicity of the amino acids, and hydrophobicity scales list the hydropathy of each of the amino acids. One such example is the Wimley-White whole-residue hydrophobicity scale, which calculates the free energy of transfer of an amino acid from an aqueous phase to a non-aqueous phase (octanol). The term “a more hydrophobic amino acid” refers to an amino acid which has a more favourable (more negative AG) free energy value for the transition from an aqueous phase to octanol according to the Wimley-White hydrophobicity scale. The free energy for the aqueous phase to octanol transition is shown in Table 3 below, and is shown graphically in FIG. 13. Starting with the most hydrophobic amino acid, the Wimley-White hydrophobicity scale lists the hydrophobicity of amino acids in the following order: tryptophan, phenylalanine, leucine, isoleucine, tyrosine, methionine, valine, cysteine, glutamic acid (uncharged), histidine (uncharged), proline, threonine, aspartic acid (uncharged), serine, alanine, glutamine, asparagine, glycine, arginine (positively charged), histidine (positively charged), lysine (positively charged), glutamic acid (negatively charged), aspartic acid (negatively charged).

TABLE 3
Wimley-White whole residue hydrophobicity
scale for water to octanol transition.

| Amino | Octanol Scale |
|---|---|
| acid | ΔG w-oct (kcal/mol) |
| Trp | −2.09 |
| Phe | −1.71 |
| Leu | −1.25 |
| Ile | −1.12 |
| Tyr | −0.71 |
| Met | −0.67 |
| Val | −0.46 |
| Cys | −0.02 |
| Glu | 0.11 |
| His | 0.11 |
| Pro | 0.14 |
| Thr | 0.25 |
| Asp | 0.43 |
| Ser | 0.46 |
| Ala | 0.5 |
| Gln | 0.77 |
| Asn | 0.85 |
| Gly | 1.15 |
| Arg+ | 1.81 |
| His+ | 2.33 |
| Lys+ | 2.8 |
| Glu− | 3.63 |
| Asp− | 3.64 |

The one or more substitution mutations may stabilise the interaction between two or more of the domains of a Factor VIII polypeptide. For example, the one or more substitution mutations at an inter-domain interface may stabilise the interaction between the respective domains of the Factor VIII polypeptide. In particular, one or more substitution mutations at the A1/A3 inter-domain interface may stabilise the interaction of the A1 and A3 domains, one or more substitution mutations at the A2/A3 inter-domain interface may stabilise the interaction of the A2 and A3 domains and one or more substitution mutations at the A1/C2 inter-domain interface may stabilise the interaction of the A1 and C2 domains. Optionally, the one or more substitution mutations of the invention may stabilise the interaction of the respective domains of a Factor VIII polypeptide when activated. In particular, the one or more substitution mutations of the invention may stabilise the interaction of the A1 and A3 domains, the A2 and A3 domains, and/or the A1 and C2 domains (i.e. of the respective domains) of the Factor VIII polypeptide when activated.

Optionally, the one or more substitution mutations may stabilise the interaction of the A1 domain, the A2 domain, and the A3-C1-C2 domains of the activated Factor VIII heterotrimeric complex. Optionally, the one or more substitution mutations may stabilise the interaction of the A2 domain with the A1 domain and A3-C1-C2 domains of the activated Factor VIII heterotrimeric complex. Optionally, the one or more substitution mutations may prevent or delay dissociation of the A2 domain from the A1 domain and A3-C1-C2 domains of the activated Factor VIII heterotrimeric complex.

The interaction of the A1 domain, A2 domain, and A3-C1-C2 domains of Factor VIII may be determined by surface plasmon resonance (SPR, or Biacore). Different components may be isolated separately, and one of the domains may be immobilised on the surface of an SPR chip; following immobilisation, the other domains may be injected into a flow cell and the binding and dissociation kinetics (kₒₙ and k_{off}) for the interaction of the domains may be monitored over time. Alternatively, inactive Factor VIII may be immobilised on an SPR chip and thrombin may be injected into a flow cell to activate Factor VIII; a drop in signal may be monitored over time, which represents the dissociation (loss of mass) of the respective components of the Factor VIII heterotrimeric complex (k_{off}). By either means, comparison with a reference Factor VIII polypeptide (e.g. a reference wild-type Factor VIII polypeptide) allows the stability of the interaction of the domains to be determined. The use of surface plasmon resonance to determine the stability of FVIIIa is described in Gale et al. 2006. Journal of Thrombosis and Haemostasis 4, 1315-1322.

Inter-domain interactions within a polypeptide are typically mediated by interactions between amino acid side chains in each of the respective domains. Interactions between the amino acid side-chains in respective domains may be non-covalent interactions. Alternatively, the amino acid side-chains in respective domains may form a covalent bond (e.g. a disulphide bond). The interaction between the domains of the Factor VIII polypeptide may therefore be stabilised (i.e. relative to a reference Factor VIII polypeptide) by amino acid substitution(s) (substitution mutations) which stabilise the interaction of the respective domains (e.g. the A1 and A3, A2 and A3 or A1 and C2 domains) of the Factor VIII polypeptide.

The side chains of aromatic amino acids phenylalanine, tyrosine, histidine and tryptophan may interact with one-another by pi-stacking interactions. In pi-stacking interactions, pairs of aromatic side-chains may typically align their respective aromatic rings in an off-centred parallel orientation. Alternatively, pairs of aromatic side-chains may align their respective aromatic rings in a T-shaped, perpendicular orientation to one-another. Optionally, the Factor VIII polypeptide may comprise a Factor VIII amino acid sequence which comprises one or more substitution mutations which increases pi-stacking interactions between amino acid side-chains of the respective domains.

The side chains of hydrophobic amino acids glycine, alanine, valine, isoleucine, leucine, phenylalanine, tryptophan, tyrosine and methionine typically cluster together within the hydrophobic core of a protein. Minimising the number of hydrophobic side chains exposed to water is a principal driving force behind protein folding, and hydrophobic packing contributes to stabilising protein structure. Conversely, the side chains of charged and polar amino acids are situated on the water-exposed surface of the protein where they interact with the surrounding water molecules. Optionally, the Factor VIII polypeptide may comprise a Factor VIII amino acid sequence which comprises one or more substitution mutations which increases hydrophobic packing between the amino acid side-chains of the respective domains (i.e. providing a more hydrophobic environment for the side chains of aliphatic/hydrophobic amino acids to interact with one another and more efficiently exclude water.

Optionally, the one or more substitution mutations comprises substitution of a charged or polar amino acid at an inter-domain interface with a hydrophobic amino acid. Optionally, the one or more substitution mutations comprises substitution of a charged or polar amino acid at an inter-domain interface with glycine, alanine, valine, isoleucine, leucine, phenylalanine, tryptophan, tyrosine or methionine. Optionally, the one or more substitution mutations comprises substitution of a hydrophobic amino acid at an inter-domain interface with a more hydrophobic amino acid.

Amino acid side chains in the respective domains may interact with one another in an unfavourable manner, due to the orientation of the respective domains relative to one-another. For example, amino acid side chains may be forced to adopt an energetically unfavourable conformation due to the orientation of the respective domains relative to one-another (steric clashing). Alternatively, an amino acid side chain may be positioned in proximity to a similarly charged amino acid side chain in another domain (an unfavourable electrostatic interaction).

Optionally, the one or more substitution mutations may eliminate unfavourable interactions between the amino acid side-chains of the respective domains. Optionally, the one or more substitution mutations may reduce steric clashing between amino acid side chains of the respective domains. Optionally, the one or more substitution mutations to reduce steric clashing may comprise substitution of a large hydrophobic amino acid with a smaller amino acid. Optionally, the one or more substitution mutations may comprise substitution of a large hydrophobic amino acid with isoleucine, leucine, valine, alanine or glycine. Optionally, the large hydrophobic amino acid is an aromatic amino acid. Optionally, the one or more substitution mutations may reduce unfavourable electrostatic interactions between amino acid side chains of the respective domains. Optionally, the one or more substitution mutations may comprise substitution of a positively charged amino acid. Optionally, the one or more substitution mutations may comprise substitution of a positively charged amino acid with a negatively charged amino acid. Optionally, the negatively charged amino acid is aspartic acid or glutamic acid. Optionally, the one or more substitution mutations may comprise substitution of a negatively charged amino acid. Optionally, the one or more substitution mutations may comprise substitution of a negatively charged amino acid with a positively charged amino acid. Optionally, the positively charged amino acid is arginine or lysine. Optionally, the one or more substitution mutations may comprise substitution of a charged amino acid with an uncharged amino acid. Optionally, the uncharged amino acid may be a polar amino acid. Optionally, the uncharged amino acid may be asparagine or glutamine. Optionally, the uncharged amino acid may be serine or threonine. Optionally, the uncharged amino acid may be valine, isoleucine, leucine, alanine or glycine.

Optionally, the one or more substitution mutation may comprise a substitution of one or more surface-inaccessible amino acids at an inter-domain interface. Three-dimensional crystal structures of Factor VIII are available (for example, the PDB accession numbers 2RZE or 4BDV); and it would therefore be routine to identify amino acids at an inter-domain interface which are surface-inaccessible. Optionally, the surface-inaccessible amino acid is surface-inaccessible in a Factor VIII polypeptide when activated.

The Factor VIII polypeptide may comprise a Factor VIII amino acid sequence which comprises one or more substitution mutations at an inter-domain interface selected from the A1/A3 interface, the A2/A3 interface, or the A1/C2 interface, wherein the amino acid which is substituted is methionine or histidine. Methionine or histidine residues at these inter-domain interfaces may be identified, for example, from a three-dimensional crystal structure of Factor VIII. Optionally, the methionine or histidine may be substituted with a more hydrophobic amino acid. Optionally, a methionine residue may be substituted with tyrosine, isoleucine, leucine, phenylalanine or tryptophan. Optionally, a histidine residue may be substituted with glutamic acid, cysteine, valine, methionine, tyrosine, isoleucine, leucine, phenylalanine or tryptophan. Optionally, the Factor VIII amino acid sequence may comprise one or more substitution mutations, wherein the amino acid which is substituted is the methionine residue corresponding to the amino acid at position 662 of SEQ ID NO: 1 (M662), or the histidine residue corresponding to the amino acid at position 693 of SEQ ID NO:1 (11693). Optionally, M662 may be substituted with alanine, cysteine, glutamic acid, glycine or serine. Optionally, H693 may be substituted with arginine. Optionally, M662 or H693 may be substituted with a more hydrophobic amino acid. Optionally, M662 may be substituted with tyrosine, isoleucine, leucine, phenylalanine or tryptophan. Optionally, H693 may be substituted with glutamic acid, cysteine, valine, methionine, tyrosine, isoleucine, leucine, phenylalanine or tryptophan.

It is within the capabilities of the person skilled in the art to determine the residue which “corresponds to” a particular amino acid of SEQ ID NO: 1. For example, the person skilled in the art merely needs to perform a sequence alignment of the wild-type Factor VIII amino acid sequence with SEQ ID NO: 1 using a suitable alignment algorithm such as that of Needleman and Wunsch described above, and determine which residue aligns with the amino acid of SEQ ID NO: 1.

Optionally, the one or more substitution mutations comprises substitution of an amino acid at an inter-domain interface with an aromatic amino acid. Optionally, the one or more substitution mutations comprises substitution of an amino acid at an inter-domain interface with phenylalanine, tyrosine, histidine or tryptophan. Optionally, the one or more substitution mutations comprises substitution of a methionine residue at an inter-domain interface with phenylalanine, tyrosine or tryptophan. Optionally, the one or more substitution mutations comprises substitution of a histidine residue at an inter-domain interface with phenylalanine, tyrosine or tryptophan. Optionally, the one or more substitution mutations comprises the M662F, M662W or M662Y substitution. Optionally, the one or more substitution mutations comprises the H693F, H693W or H693Y substitution. Optionally, the one or more substitution mutations comprises the M662W substitution. Optionally, the one or more substitution mutations comprises the H693W or H693Y substitution. Optionally, the one or more substitution mutations comprises the H693W substitution. Optionally, the one or more substitution mutations comprises M662W and H693W substitutions.

Thus, in a particular embodiment, the Factor VIII polypeptide comprises a Factor VIII amino acid sequence which comprises one or more substitution mutations, wherein the one or more substitution mutations comprises the M662W substitution. In a further embodiment, the Factor VIII polypeptide comprises a Factor VIII amino acid sequence which comprises one or more substitution mutations, wherein the one or more substitution mutations comprises the H693W substitution. In yet a further embodiment, the Factor VIII polypeptide comprises a Factor VIII amino acid sequence which comprises one or more substitution mutations, wherein the one or more substitution mutations comprise the M662W and H693W substitutions.

Optionally, the Factor VIII polypeptide may comprise a Factor VIII amino acid sequence as set forth in any one of SEQ ID NOs: 77, 78, 79, 80, 84 or 85, or an amino acid sequence which is at least 90% at least 95%, at least 96%, at least 97%, at least 98% or at least 99% identical to an amino acid sequence set forth in any one of SEQ ID NOs: 77, 78, 79, 80, 84 or 85.

Optionally, the one or more substitution mutations does not comprise substitution of M662 with a less hydrophobic amino acid. Optionally, the one or more substitution mutations does not comprise M662I. Optionally, the one or more substitution mutations does not comprise M662C.

Optionally, the one or more substitution mutations does not comprise M662K. Optionally, the one or more substitution mutations does not comprise M662K if the Factor VIII amino acid sequence comprises an aspartic acid residue at the position corresponding to position 1828 of SEQ ID NO:1 (D1828). Optionally, the one or more substitution mutations does not comprise M662K if the Factor VIII amino acid sequence comprises the sequence MAPTKDEFDCKA at the positions corresponding to positions 1823-1834 of SEQ ID NO:1.

Optionally, the one or more substitution mutations does not comprise substitution of A108. Optionally, the one or more substitution mutations does not comprise substitution of A108 with a more hydrophobic amino acid. Optionally, the one or more substitutions does not comprise A1081.

Optionally, the one or more substitution mutations does not comprise substitution of a negatively-charged amino acid. Optionally, the one or more substitution mutations does not comprise substitution of a negatively-charged amino acid with an uncharged amino acid. Optionally, the one or more substitution mutations does not comprise substitution of D27, E272, E287, D302, D519, E540, E665, D666, E683, D696, D1795, E1829, E1984. Optionally, the one or more substitution mutations does not comprise substitution of D27, E272, E287, D302, D519, E540, E665, D666, E683, D696, D1795, E1829, E1984 with an uncharged amino acid. Optionally, the one or more substitution mutations does not comprise substitution of D27, E272, E287, D302, D519, E540, E665, D666, E683, D696, D1795, E1829, E1984 with alanine or valine. Optionally, the one or more substitution mutations does not comprise D519L, D519Q, D519T or D519V. Optionally, the one or more substitution mutations does not comprise substitution of two or more of D519, E665 and E1984. Optionally, the one or more substitution mutations does not comprise substitution of two or more of D519, E665 and E1984 with alanine or valine.

Optionally, the one or more substitution mutations does not comprise substitution of a positively-charged amino acid. Optionally, the one or more substitution mutations does not comprise substitution of a positively-charged amino acid with an uncharged amino acid. Optionally, the one or more substitution mutations does not comprise substitution of K380, R490, K512, K523, R527, K556, R562, K570 or R571. Optionally, the one or more substitution mutations does not comprise substitution of K380, R490, K512, K523, R527, K556, R562, K570 or R571 with an uncharged amino acid.

Optionally, the one or more substitution mutations does not comprise substitution of S313,

H317, T522, S524, R531, N538, S650, S654, N684, S695, S1791, Q1820, S1949, N1950 or R1966. Optionally, the one or more substitution mutations does not comprise substitution of S313, H317, T522, S524, R531, N538, S650, S654, N684, S695, S1791, Q1820, S1949, N1950 or R1966 with alanine. Optionally, the one or more substitution mutations does not comprise substitution of Y476, Y664, Y1786 or Y1792. Optionally, the one or more substitution mutations does not comprise substitution of Y476, Y664, Y1786 or Y1792 with phenylalanine.

Optionally, the one or more substitution mutations does not comprise substitution of K659. Optionally, the one or more substitution mutations does not comprise a non-conservative substitution of K659. Optionally, the one or more substitution mutations does not comprise K659D, K659E, K659Y, K659N, K659Q, K659T, K659S, K659C, K659W, K659F, K659P, K659M, K659V, K659L, K6591, K659Y, K659G or K659A. Optionally, the one or more substitution mutations does not comprise substitution of E665. Optionally, the one or more substitution mutations does not comprise a non-conservative substitution of E665. Optionally, the one or more substitution mutations does not comprise E665V, E6651, E665M, E665N or E665Y.

Optionally, the one or more substitution mutations comprises substitution of K659. Optionally, the one or more substitution mutations comprises K659Q, K659G, K6591 or K659F. Optionally, the one or more substitution mutations comprises substitution of E665. Optionally, the one or more substitution mutations comprises E665R, E665Q, E665H, E665K, E665M, E665F, E665W or E665Y.

In certain embodiments, the Factor VIII polypeptide may comprise a Factor VIII amino acid sequence which comprises one or more substitution mutations at an inter-domain interface selected from the A1/A3 domain interface, the A2/A3 domain interface or the A1/C2 domain interface, wherein at least one of the one or more substitution mutations comprises substitution of a pair of amino acids in the respective domains with cysteine residues. Put another way, the at least one or more substitution mutations may comprise substitution of a cysteine residue in a first domain (e.g. the A1 or A2 domain), and substitution of a cysteine residue in a second domain (e.g. the A3 or C2 domain). The pair of amino acids may therefore comprise a first amino (in a first domain) and a second amino acid (in a second domain). Put another way, the at least one or more substitution mutations may comprise substitution of a first amino acid (in a first domain), and a second amino acid (in a second domain) with cysteine residues. Thus, the Factor VIII polypeptide may comprise a Factor VIII amino acid sequence which comprises one or more substitution mutations which comprise substitution of a pair of cognate amino acids in two respective domains of the Factor VIII polypeptide with cysteine residues.

Optionally, the substitution of a pair of amino acids in the respective domains with cysteine residues allows the cysteine residues to form a disulphide bond between the respective domains. The formation of a disulphide bond may be determined by mass spectroscopy, and in particular by the analysing the fragmentation pattern of a polypeptide suspected of containing a disulphide bond, optionally following limited proteolysis, for example as outlined in Gorman et al. 2002. Mass Spectrometry Reviews 21, 183-216. Alternatively, the formation of a disulphide bond may be determined by performing limited proteolysis on a polypeptide and analysing the resulting protein fragments by SDS-PAGE under both reducing and non-reducing conditions, optionally in combination with N-terminal sequencing.

Optionally, the pair of amino acids which are substituted with cysteine residues may be in the A1 and A3 domains. Optionally, the first amino acid may correspond to M147, S149 or S289 of SEQ ID NO:1 and the second amino acid may correspond to E1969, E1970 or N1977 of SEQ ID NO:1. Optionally, the one or more substitution mutations comprises a pair of substitution mutations selected from the list consisting of (i) S289C and N1977C, (ii) M147C and E1970C, and (iii) S149C and E1969C.

Optionally, the pair of amino acids which are substituted with cysteine residues may be in the A2 and A3 domains. Optionally, the first amino acid corresponds to T667, T669, N684, L687, 1689, S695 or F697 of SEQ ID NO: 1 and the second amino acid corresponds to S1791, G1799, A1800, R1803, E1844, S1949, G1981, V1982, or Y1979 of SEQ ID NO: 1. Optionally, the one or more substitution mutations comprises a pair of substitution mutations selected from the list consisting of (i) T669C and V1982C, (ii) L687C and A1800C, (iii) I689C and G1799C, (iv) F697C and S1949C, (v) T667C and G1981C, (vi) T669C and Y1979C, (vii) N684C and S1791C, (viii) L687C and R1803C, and (ix) S695C and E1844C.

Optionally, the pair of amino acids which are substituted with cysteine residues may be in the A1 and C2 domains. Optionally, the first amino acid corresponds to A108, T118 or V137 of SEQ ID NO: 1 and the second amino acid corresponds to N2172, Q2329 or Y2332 of SEQ ID NO: 1. Optionally, the one or more substitution mutations comprises a pair of substitution mutations selected from the list consisting of (i) A108C and Q2329C, (ii) T118C and N2172C, and (iii) V137C and Y2332C.

Optionally, the one or more substitution mutations does not comprise a substitution of any one of the amino acids corresponding to positions 656-667 of SEQ ID NO:1 (YTFKHKMVYEDT) with a cysteine residue. Optionally, the one or more substitution mutations does not comprise a substitution of any one of the amino acids corresponding to positions 1823-1834 of SEQ ID NO:1 (MAPTKDEFDCKA) with a cysteine residue. Optionally, the one or more substitution mutations does not comprise a first substitution mutation comprising a substitution of any one of the amino acids corresponding to positions 656-667 of SEQ ID NO:1 (YTFKHKMVYEDT) with a cysteine residue and a second substitution mutation comprising a substitution of any one of the amino acids corresponding to positions 1823-1834 of SEQ ID NO:1 (MAPTKDEFDCKA) with a cysteine residue.

Optionally, the one or more substitution mutations does not comprise a pair of substitution mutations selected from the list consisting of Y656C and A1834C, T657C and K1833C, K659C and D1831C, H660C and F1830C, K662C and E1829C, M662C and D1828C, V663C and K1827C, Y664C and T1826C, E665C and P1825C, D666C and A1824C, and T667C and M1823C. Optionally, the one or more substitution mutations does not comprise a pair of substitution mutations selected from the list consisting of M662C and D1828C, and Y664C and T1826C. Optionally, the one or more substitution mutations does not comprise R121C and L2302C substitutions. Optionally, the one or more substitution mutations does not comprise a pair of substitution mutations selected from the list consisting of M662C and D1828C, S268C and F673C, 1312C and P672C, S313C and A644C, M662C and K1827C, Y664C and T1826C, P264C and Q645C, R282C and T522C, S285C and F673C, H311C and F673C, S314C and A644C, S314C and Q645C, V663C and E1829C, N694C and P1980C, and S695C and E1844C.

In particular embodiments, the one or more substitutions may comprise a pair of substitution mutations selected from the list consisting of (i) L687C and A1800C; (ii) N684C and S1791C; and (iii) S695C and E1844C. Optionally, the Factor VIII polypeptide may comprise a Factor VIII amino acid sequence as set forth in any one of SEQ ID NOs: 81, 82, 83, 86, 87 or 88, or an amino acid sequence which is at least 90% at least 95%, at least 96%, at least 97%, at least 98% or at least 99% identical to an amino acid as sequence set forth in any one of SEQ ID NOs: 81, 82, 83, 86, 87 or 88.

As outlined above, activation of Factor VIII requires proteolytic cleavage by thrombin. Following thrombin cleavage, Factor VIII forms a heterotrimeric complex consisting of the A1 domain, A2 domain, and A3-C1-C2 domains. In the course of activation, Factor VIII undergoes a conformational change. More specifically, in the course of activation, the orientation of the A2 domain relative to the A1 and A3-C1-C2 domains is altered. The A2 domain of activated Factor VIII thus is in a different orientation with respect to the A1, A3, C1 and C2 domains, to inactive Factor VIII. The A2 domain may therefore be seen to pivot with respect to the other domains of Factor VIII during the course of activation. The maximum activity of wild-type Factor VIII is typically seen approximately two minutes following activation.

Without wishing to be bound by theory, it is believed that the conformation change undertaken by the A2 domain may be required for Factor VIII activation. A substitution mutation which stabilises the conformation A2 domain in the orientation of the inactive Factor VIII may therefore inhibit activation of the Factor VIII polypeptide. In certain embodiments, the one or more substitution mutations do not inhibit activation of the Factor VIII polypeptide. Optionally, the one or more substitution mutations do not inhibit activation of the Factor VIII polypeptide relative to the activation of a reference wild-type Factor VIII polypeptide. Optionally, a Factor VIII polypeptide comprising a Factor VIII amino acid sequence which comprises one or more substitution mutations at the A1/A3, A2/A3 or A1/C2 domain interface is not activated more slowly than a reference wild-type Factor VIII polypeptide.

Inhibition of Factor VIII activation may be determined by comparing the time taken to achieve maximum Factor VIII activity following activation with that of a reference wild-type Factor VIII polypeptide. Thrombin may be added to a sample containing a Factor VIII polypeptide and Factor VIII activation may be monitored by taking aliquots of the sample at suitable time points and determining FVIII activity for each aliquot. Optionally FVIII activity may be measured over the course of five, ten, fifteen or twenty minutes following activation. Activity may be determined at a series of time points, e.g. two or more, three or more, four or more, five or more or six or more time points following activation. Optionally Factor VIII activity may be determined immediately prior to activation. By way of representative example, Factor VIII activity may be determined immediately following activation, and further measurements may be taken every thirty seconds following activation. Optionally, relative Factor VIII activity may be determined by calculating the fold-increase in Factor VIII activity at a given time point, compared to Factor VIII activity immediately following activation. Optionally, specific activity may be determined at each time point.

Optionally, the Factor VIII polypeptide may comprise a Factor VIII amino acid sequence which comprises an amino acid sequence that is at least 90%, at least 95%, at least 96%, at least 97%, at least 98%, at least 99% or 100% identical to the amino acid sequence set forth in SEQ ID NO: 77 or 78. Optionally, the Factor VIII polypeptide may comprise a Factor VIII amino acid sequence which consists of an amino acid sequence that is at least 90%, at least 95%, at least 96%, at least 97%, at least 98%, at least 99% or 100% identical to the amino acid sequence set forth in SEQ ID NO: 77 or 78. Optionally, the Factor VIII polypeptide may comprise a Factor VIII amino acid sequence which comprises the amino acid sequence set forth in SEQ ID NO: 77 or 78. Optionally, the Factor VIII polypeptide may comprise a Factor VIII amino acid sequence which consists of the amino acid sequence set forth in SEQ ID NO: 77 or 78.

Optionally, the Factor VIII nucleotide sequence encoding the Factor VIII amino acid sequence may be at least 85%, at least 90%, at least 95%, at least 96%, at least 97%, at least 98%, at least 99% or 100% identical to a nucleotide sequence comprising at least 4047, at least 4071, at least 4104, at least 4128, at least 4131, at least 4155, at least 4158, at least 4173, at least 4182, at least 4254 or at least 4263 nucleotides (optionally at least 4047, at least 4071, at least 4104, at least 4128, at least 4131, at least 4155, at least 4158, at least 4173, at least 4182, at least 4254 or at least 4263 contiguous nucleotides, or at least 4047, at least 4071, at least 4104, at least 4128, at least 4131, at least 4155, at least 4158, at least 4173, at least 4182, at least 4254 or at least 4263 nucleotides from up to 5, up to 4, up to 3 or up to 2 regions of SEQ ID NO:3, 4, 5 or 6) of SEQ ID NO:3, 4, 5 or 6. Optionally, the Factor VIII nucleotide sequence may comprise a nucleotide sequence that differs from the nucleotide sequence comprising at least 4047, at least 4071, at least 4104, at least 4128, at least 4131, at least 4155, at least 4158, at least 4173, at least 4182, at least 4254 or at least 4263 nucleotides of SEQ ID NO:3, 4, 5 or 6 by 40 or fewer, 35 or fewer, 30 or fewer, 25 or fewer, 20 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 nucleotides. Optionally, the Factor VIII nucleotide sequence may comprise a nucleotide sequence that differs from the nucleotide sequence comprising at least 4047, at least 4071, at least 4104, at least 4128, at least 4131, at least 4155, at least 4158, at least 4173, at least 4182, at least 4254 or at least 4263 nucleotides of SEQ ID NO:3, 4, 5 or 6 only by alternative codons encoding the one or more substitution mutations.

Optionally, the Factor VIII nucleotide sequence encoding the Factor VIII amino acid sequence may be at least 85%, at least 90%, at least 95%, at least 96%, at least 97%, at least 98%, or at least 99% identical to the nucleotide sequence set forth in SEQ ID NO:39, 40, 41 or 42. Optionally, the Factor VIII nucleotide sequence may comprise a nucleotide sequence that differs from the nucleotide set forth in SEQ ID NO:39, 40, 41 or 42 by 43 or fewer, 40 or fewer, 35 or fewer, 30 or fewer, 25 or fewer, 20 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 nucleotides. Optionally, the Factor VIII nucleotide sequence may comprise a nucleotide sequence that differs from the nucleotide sequence set forth in SEQ ID NO:39, 40, 41 or 42 only by alternative codons encoding the one or more substitution mutations.

Optionally, the Factor VIII nucleotide sequence encoding the Factor VIII amino acid sequence may comprise a nucleotide sequence that is at least 85%, at least 90%, at least 95%, at least 96%, at least 97%, at least 98%, at least 99% or 100% identical to the nucleotide set forth in SEQ ID NO:75. Optionally, the Factor VIII nucleotide sequence may consist of a nucleotide sequence that is at least 85%, at least 90%, at least 95%, at least 96%, at least 97%, at least 98%, at least 99% or 100% identical to the nucleotide sequence set forth in SEQ ID NO:75. Optionally, the Factor VIII nucleotide sequence may comprise the nucleotide sequence set forth in SEQ ID NO:75. Optionally, the Factor VIII nucleotide sequence may consist of the nucleotide sequence set forth in SEQ ID NO:75.

Optionally, the Factor VIII nucleotide sequence encoding the Factor VIII amino acid sequence may comprise a nucleotide sequence that is at least 85%, at least 90%, at least 95%, at least 96%, at least 97%, at least 98%, at least 99% or 100% identical to the nucleotide set forth in SEQ ID NO:76. Optionally, the Factor VIII nucleotide sequence encoding the Factor VIII amino acid sequence may comprise a nucleotide sequence that is at least 85%, at least 90%, at least 95%, at least 96%, at least 97%, at least 98%, at least 99% or 100% identical to a nucleotide sequence comprising at least 4047 nucleotides of SEQ ID NO: 76.

Optionally, the Factor VIII nucleotide sequence may consist of a nucleotide sequence that is at least 85%, at least 90%, at least 95%, at least 96%, at least 97%, at least 98%, at least 99% or 100% identical to the nucleotide sequence set forth in SEQ ID NO:76. Optionally, the Factor VIII nucleotide sequence may comprise the nucleotide sequence set forth in SEQ ID NO:76. Optionally, the Factor VIII nucleotide sequence may consist of the nucleotide sequence set forth in SEQ ID NO:76.

A recombinant AAV construct comprising a polynucleotide

The invention provides a recombinant AAV construct which comprises a polynucleotide comprising a Factor VIII nucleotide sequence, wherein the Factor VIII nucleotide sequence encodes a Factor VIII polypeptide comprising a Factor VIII amino acid sequence. The polynucleotide may be any polynucleotide of the invention. The Factor VIII nucleotide sequence may be any Factor VIII nucleotide sequence of the invention. The Factor VIII polypeptide may be any Factor VIII polypeptide of the invention.

The recombinant AAV construct is preferably less than 4900 nucleotides in length. The recombinant AAV construct may be between 4700 and 4900, between 4800 and 4900, between 4850 and 4900, between 4825 and 4875 or around 4845 nucleotides in length. The recombinant AAV construct may be around 4845 nucleotides in length. Optionally the recombinant AAV construct may comprise a polynucleotide comprising a Factor VIII nucleotide sequence of less than 4318 nucleotides in length. Optionally, the Factor VIII nucleotide sequence may be between 4318 and 4046, between 4318 and 4070, between 4264 and 4127, between 4210 and 4151 or around 4182 nucleotides in length. Optionally, the Factor VIII nucleotide sequence may be between 4318 and 4151, between 4318 and 4210, between 4318 and 4208, between 4318 and 4281, between 4318 and 4280, or around 4314 nucleotides in length. Optionally, the Factor VIII nucleotide sequence may be between 4318 and 4151, between 4318 and 4210, between 4318 and 4281, or around 4314 nucleotides in length. Optionally, the Factor VIII nucleotide sequence may be around 4314 nucleotides in length. Optionally, the Factor VIII nucleotide sequence may be around 4182 nucleotides in length.

The recombinant AAV construct may be between 4700 and 4900, between 4850 and 4900, or around 4883 nucleotides in length. The recombinant AAV construct may be less than 4850, less than 4800, or less than 4750 nucleotides in length. The recombinant AAV construct may be between 4700 and 4900, between 4700 and 4850, between 4700 and 4800, between 4700 and 4750 or around 4712 nucleotides in length.

The recombinant AAV construct may be less than 4850, less than 4800, or less than 4750 nucleotides in length. The recombinant AAV construct may be between 4700 and 4900, between 4700 and 4850, between 4700 and 4800, between 4700 and 4750 or around 4713 nucleotides in length. The recombinant AAV construct may be around 4713 nucleotides in length. Optionally, the recombinant AAV construct may comprise a polynucleotide comprising a Factor VIII nucleotide sequence of less than 4318 nucleotides in length. Optionally, the Factor VIII nucleotide sequence may be between 4318 and 4046, between 4264 and 4070, between 4264 and 4103, between 4255 and 4127, between 4246 and 4130, between 4237 and 4154, between 4228 and 4157, between 4219 and 4166, between 4210 and 4169, between 4201 and 4172, between 4192 and 4175 or around 4182 nucleotides in length.

Optionally, the recombinant AAV construct is between 4500 and 4900, between 4500 and 4850, between 4500 and 4750, around 4715 or around 4713 nucleotides in length. Optionally, the recombinant AAV construct is 4713 nucleotides in length.

The recombinant AAV construct may be between 4700 and 4850, between 4700 and 4800, between 4700 and 4750 or around 4713 nucleotides in length, and may optionally comprise a polynucleotide comprising a Factor VIII nucleotide sequence of less than 4318 nucleotides in length. Optionally, the Factor VIII nucleotide sequence may be between 4318 and 4046, between 4264 and 4070, between 4264 and 4103, between 4255 and 4127, between 4246 and 4130, between 4237 and 4154, between 4228 and 4157, between 4219 and 4166, between 4210 and 4169, between 4201 and 4172, between 4192 and 4175 or around 4182 nucleotides in length.

Optionally, the recombinant AAV construct may be around 4713 nucleotides in length and the Factor VIII nucleotide sequence may be between 4228 and 4157, between 4219 and 4166, between 4210 and 4169, between 4201 and 4172, between 4192 and 4175 or around 4131, around 4134, around 4155, around 4158, around 4161, around 4167, around 4173 or around 4182 nucleotides in length. Optionally, the recombinant AAV construct may be around 4713 nucleotides in length and the Factor VIII nucleotide sequence may be 4131, 4134, 4155, 4158 , 4161, 4167, 4173 or 4182 nucleotides in length. Optionally the recombinant AAV construct may be 4713 nucleotides in length and the Factor VIII nucleotide sequence may be SEQ ID NO:39.

In some cases, reference to the lengths of the Factor VIII nucleotide sequences in the above passages in the context of the recombinant AAV construct does not include a ‘stop’ codon —Factor VIII nucleotide sequences which include a stop codon would be three nucleotides longer than the nucleotide sequences recited above. In some embodiments, the Factor VIII nucleotide sequences may comprise a stop codon.

Optionally, the Factor VIII amino acid sequence does not comprise amino acids corresponding to positions 746 to 1639 of SEQ ID NO: 1. The Factor VIII amino acid sequence may comprise SEQ ID NO: 7. The Factor VIII amino acid sequence may be SEQ ID NO: 7. The Factor VIII amino acid sequence may comprise SEQ ID NO: 8. The Factor VIII amino acid sequence may be SEQ ID NO: 8.

The recombinant AAV construct may be single-stranded. The recombinant AAV construct may be an AAV genome.

The recombinant AAV construct may further comprise a transcriptional regulatory element (TRE). The transcriptional regulatory element may comprise a liver-specific promoter. The transcription regulatory element may be fewer than 270 nucleotides in length.

The transcription regulatory element of the invention may comprise, or may be, a promoter. The promoter may be a liver-specific promoter and/or may further comprise an enhancer. Optionally, the transcription regulatory element is liver-specific. In some embodiments, the transcription regulatory element or the promoter is liver-specific if it promotes protein expression at higher levels in liver cells compared to cells from at least one other organ or tissue.

Optionally, the transcription regulatory element or the promoter is liver-specific if it promotes protein expression at higher levels in liver cells compared to cells from at least one other organ or tissue and the transcription regulatory element or the promoter promotes protein expression in the cells from at least one other organ or tissue at a level less than 40%, less than 30%, less than 25%, less than 15%, less than 10%, or less than 5% of the level that the transcription regulatory element or the promoter promotes protein expression in liver cells.

Optionally, the cells from at least one other organ or tissue are at least one of kidney cells, pancreatic cells, breast cells, neuroblastoma cells, lung cells, and early B cells. Optionally, the cells from at least one other organ or tissue are kidney cells, pancreatic cells, breast cells, neuroblastoma cells, lung cells, and early B cells. Optionally, the cells from at least one other organ or tissue are at least one of HEK293T cells, PANC1 cells, BxPC-3 cells, MCF7 cells, 1643 cells, MRC-9 cells, and 697 cells. Optionally, the cells from at least one other organ or tissue are HEK293T cells, PANC1 cells, BxPC-3 cells, MCF7 cells, 1643 cells, MRC-9 cells, and 697 cells.

Optionally, whether or not the transcription regulatory element or the promoter is liver-specific may be determined by transducing Huh7 cells and comparator cells with a vector comprising the transcription regulatory element or promoter operably linked to a transgene and comparing the number of Huh7 cells that express the transgene with the number of comparator cells that express the transgene. The comparator cells can be non-liver cells. For example, if the user wishes to determine whether the transcription regulatory element or promoter promotes expression at higher levels in liver cells compared to breast cells, the user may transduce Huh7 cells and comparator cells that are breast cells such as MCF7 cells. If the number of Huh7 cells that express the transgene is significantly higher than the number of comparator cells that express the transgene then the promoter or transcription regulatory element is liver-specific.

The transgene may be GFP, in which case the user may determine the number of cells (e.g. the number of Huh7 cells or the number of comparator cells) that express the transgene using fluorescence microscopy and counting the number of cells that fluoresce green.

Optionally, the transcription regulatory element comprises a core nucleotide sequence which comprises or consists of a nucleotide sequence having at least 95% identity to SEQ ID NO: 43, or a sequence which differs from SEQ ID NO: 43 by a single nucleotide, and wherein the transcription regulatory element is between 80 and 280 nucleotides in length; optionally wherein the transcription regulatory element is between 80 and 225 nucleotides in length. Optionally, the transcription regulatory element comprises a core nucleotide sequence which comprises or consists of a sequence having at least 95% identity to SEQ ID NO: 43, or a sequence which differs from SEQ ID NO: 43 by a single nucleotide, wherein the transcription regulatory element:
(a) does not comprise at least 20, at least 30 or at least 40 consecutive nucleotides of SEQ ID NO: 48 and/or
(b) does not comprise at least 20, at least 30 or at least 40 consecutive nucleotides of SEQ ID NO: 49;
and wherein the transcription regulatory element is between 80 and 280 nucleotides in length. Such transcription regulatory elements are shortened versions of the known HLP2 transcription regulatory element (TRE).

The present inventors have surprisingly determined that a substantial number of nucleotides from the known HLP2 TRE can be deleted or modified without significantly impacting the efficacy of the TRE. In particular, the present inventors have surprisingly found that the short transcription regulatory element FRE 72 is of comparable efficacy (i.e. at least 50% or better activity by comparison) with the HLP2 TRE despite being of considerably shorter length. Indeed, the “core nucleotide sequence” (i.e. SEQ ID NO: 43 or a variant thereof) which is present in the HLP2 TRE can have some minimal efficacy as a TRE, but when it is lengthened to form the FRE 72 TRE (SEQ ID NO: 51) it provides even greater efficacy. As set out above, the present invention relates to recombinant AAV constructs that are shorter than 4900 nucleotides in length, and in that context short TREs are advantageous.

The present inventors have also discovered that the efficacy of the TREs can be increased by inclusion of additional nucleotide sequence(s), including nucleotide sequences 3′ to the core nucleotide sequence. Accordingly, the TRE may further comprise a nucleotide sequence located 3′ to the core nucleotide sequence.

Optionally, the transcription regulatory element further comprises a nucleotide sequence located 3′ to the core nucleotide sequence, and the nucleotide sequence located 3′ to the core nucleotide sequence comprises or consists of a nucleotide sequence:
a. of SEQ ID NO: 44, or a sequence which differs from SEQ ID NO: 44 by a single nucleotide;
b. of SEQ ID NO: 45, or a sequence which differs from SEQ ID NO: 45 by a single nucleotide;
c. of SEQ ID NO: 46, or a sequence which differs from SEQ ID NO: 46 by a single nucleotide; or
d. having at least 95% identity to SEQ ID NO: 47, or a sequence which differs from SEQ ID NO: 47 by a single nucleotide.

Optionally, the nucleotide sequence located 3′ to the core nucleotide sequence is shorter than 50 nucleotides; optionally is shorter than 40 nucleotides; and optionally is shorter than 30 nucleotides.

Optionally, the transcription regulatory element:
a. does not comprise a nucleotide sequence according to SEQ ID NO: 48, or does not comprise at least 20, at least 30 or at least 40 consecutive nucleotides of SEQ ID NO: 48; and/or
b. does not comprise a nucleotide sequence according to SEQ ID NO: 49 or does not comprise at least 20, at least 30 or at least 40 consecutive nucleotides of SEQ ID NO: 49.

Optionally, the transcription regulatory element:
a. does not comprise a sequence having at least 90% or at least 95% identity to SEQ ID NO: 48; and/or
b. does not comprise a sequence having at least 90% or at least 95% identity to SEQ ID NO: 49.

Optionally, the transcription regulatory element is shorter than 200 nucleotides, optionally shorter than 150 nucleotides, optionally shorter than 125 nucleotides. Optionally, the transcription regulatory element is at least 85 nucleotides in length, optionally at least 100 nucleotides in length, optionally at least 110 nucleotides in length.

Optionally, the transcription regulatory element terminates in a ten-nucleotide sequence selected from:
a. acagtgaatc (SEQ ID NO: 123); or
b. ctcctcagct (SEQ D NO: 124).

Optionally, the core nucleotide sequence is 73-80 nucleotides in length. Optionally, the core nucleotide sequence has at least 95% identity, and optionally at least 98% identity, to SEQ ID NO: 50. FRE72 comprises an extended core nucleotide sequence (corresponding to SEQ ID NO: 50 or a variant thereof). The extended core nucleotide sequence may be identical to SEQ ID NO: 50.

SEQ ID NO: 51 defines a transcription regulatory element of the invention (allocated the internal designation “FRE72”) having properties which are detailed in the examples below. It comprises the “extended core nucleotide sequence” of nucleotides 163-242 of HLP2, while the 5′ section lacks nucleotides 1-162 of HLP2 (and therefore completely lacks nucleotides 118-162 of HLP2). FRE72 can therefore be defined as not having a 5′ section (i.e. as not having a nucleotide sequence that is 5′ to the “extended core nucleotide sequence”). Alternatively, if FRE72 is instead considered to have the core nucleotide sequence of nucleotides 170-242 of HLP2, then it can be considered to have a 5′ section consisting of nucleotides 163-169 of HLP2. The 3′ section lacks nucleotides 243-296 of HLP2 (and therefore completely lacks nucleotides 243-283 of HLP2) and includes nucleotides 297-335 of HLP2. The overall length of FRE72 is 119 nucleotides.

FRE72 therefore provides a transcription regulatory element of the invention which (inter alia):
comprises the extended core nucleotide sequence as defined by SEQ ID NO: 50;
does not comprise a nucleotide sequence according to SEQ ID NO: 48;
does not comprise a nucleotide sequence according to SEQ ID NO: 49;
comprises a nucleotide sequence which is located 5′ to the core nucleotide sequence and which has less than 60% identity to SEQ ID NO: 48; and
comprises a nucleotide sequence located 3′ to the core nucleotide and which includes 3 TSS (transcriptional start site) defined by SEQ ID NOs: 44, 45 and 46.

Optionally, the transcription regulatory element comprises or consists of a sequence which has at least 90% identity, optionally at least 95% identity, or optionally at least 98% identity to SEQ ID NO: 51. Optionally, the transcription regulatory element comprises or consists of a sequence of SEQ ID NO: 51.

Optionally, the transcription regulatory element is operably linked to the Factor VIII nucleotide sequence.

Optionally, the transcription regulatory element may express a Factor VIII nucleotide sequence to which it is operably linked at 50% or better, 80% or better or 100% or better compared to the HLP2 TRE (defined by SEQ ID NO: 52).

The skilled person may compare the expression of a Factor VIII nucleotide sequence using a transcription regulatory element to the expression using the HLP2 TRE (SEQ ID NO: 52) by comparing the level of a Factor VIII polypeptide expressed under the control of a transcription regulatory element with the level of Factor VIII polypeptide expressed under the control of the HLP2 TRE in an in vitro or an in vivo system.

For example, to compare the level of Factor VIII polypeptide expressed in an in vitro system, the skilled person could transduce host cells with a vector comprising the TRE of interest operably linked to the Factor VIII nucleotide sequence (test cells), and some cells with a vector comprising HLP2 operably linked to the Factor VIII nucleotide sequence (reference cells). The cells may be cultured under conditions suitable for expressing the Factor VIII nucleotide sequence, and the level of Factor VIII polypeptide expressed in the test cells and reference cells can be compared. Suitable host cells include cultured human liver cells, such as Huh7 cells. The level of Factor VIII polypeptide should be normalised to reflect the number of cells that have been transfected using a luciferase assay. In the luciferase assays the test cells and the reference cells are also transfected using an equivalent vector (identical except for the promoter and the transgene) comprising a luciferase transgene, and the proportion of cells that are transfected by the vector will be proportionate to the fluorescent signal produced by the luciferase expressed from the vector comprising the luciferase transgene.

Similarly, to compare the level of Factor VIII nucleotide sequence expressed in an in vivo system, the skilled person could inject some mice (such as C57BL/6 mice) with a viral particle comprising the TRE of the invention operably linked to the Factor VIII nucleotide sequence (test mice), and some equivalent mice with a viral particle comprising HLP2 operably linked to the Factor VIII nucleotide sequence (reference mice). The mice may be culled and the level of Factor VIII polypeptide in the blood of the test mice may be compared to the level of Factor VIII polypeptide in the blood of the reference mice. The level of Factor VIII polypeptide may be normalised to the number of vector genomes per liver cell.

The level of Factor VIII polypeptide can be assessed using an ELISA. In an example of an ELISA assay, an antibody that binds to the Factor VIII polypeptide encoded by the transgene could be bound to a plate. The sample, comprising the Factor VIII polypeptide at unknown concentration, could be passed over the plate. A second detection antibody that binds to the polypeptide encoded by the transgene could be applied to the plate, and any excess washed off. The detection antibody that remains (i.e. is not washed off) will be bound to the polypeptide encoded by the transgene. The detection antibody could be linked to an enzyme such as horse radish peroxidase. The level of detection antibody that binds to the Factor VIII polypeptide on the plate could be measured by measuring the amount of the detection antibody. For example, if the detection antibody is linked to horse radish peroxidase, the horse radish peroxidase can catalyse the production of a blue reaction product from a substrate such as TMB (3,3′,5,5′-tetramethylbenzidine), and the level of the blue product can be detected by absorbance at 450 nm. The level of the blue product is proportional to the amount of detection antibody that remained after the washing step, which is proportional to the amount of the polypeptide encoded by the transgene in the sample. Alternatively, for example when using purified protein, the level of Factor VIII polypeptide may be determined spectrophotometrically.

For example, a suitable ELISA assay kit is the Asserachrom assay as described in the Examples. The level of the Factor VIII polypeptide may be measured using the Asserachrom assay.

Alternatively, the skilled person may assess the level of Factor VIII polypeptide by determining the activity of the Factor VIII polypeptide.

For example, the level of Factor VIII polypeptide may be determined using a chromogenic assay, such as a chromogenic assay that measures cofactor activity. For example, a suitable chromogenic assay is as follows. The Factor VIII polypeptide is mixed with human Factor X polypeptide and Factor IXa polypeptide, thrombin, phospholipids and calcium. The thrombin activates the Factor VIII polypeptide (having Factor VIII activity such as a Factor VIII polypeptide) to form Factor VIIIa polypeptide. The thrombin-activated polypeptide forms an enzymatic complex with Factor IXa polypeptide, phospholipids and calcium, which enzymatic complex can catalyse the conversion of Factor X polypeptide to Factor Xa polypeptide. The activity of the Factor Xa polypeptide can catalyse cleavage of a chromogenic substrate (e.g. SXa-11) to produce pNA. The level of pNA generated can be measured by determining colour development at 405 nm (e.g. measured by absorbance). Factor X polypeptide, and therefore Factor Xa polypeptide, is provided in excess. Therefore the limiting factor is Factor VIIIa polypeptide. Thus, the level of pNA generated is proportional to the amount of the Factor Xa polypeptide generated by the Factor VIII polypeptide in the sample, which is proportional to the activity of Factor VIII polypeptide in the sample. The activity of Factor VIII polypeptide in the sample is a measure of the cofactor activity of Factor VIII polypeptide in the sample.

For example, a suitable chromogenic assay is the BIOPHEN FVIII:C assay (Ref: 221406) manufactured by HYPHEN BioMed as used in the Examples. The activity of Factor VIII polypeptide may be measured using the BIOPHEN FVIII:C assay.

The recombinant AAV construct may further comprise a nucleotide sequence encoding a signal peptide. The signal peptide may be a wild-type Factor VIII signal peptide. The signal peptide may comprise the wild type FVIII signal peptide set forth in SEQ ID NO:53. Optionally, the signal peptide is not a wild-type Factor VIII signal peptide.

Optionally, the Factor VIII polypeptide encoded by the Factor VIII nucleotide sequence is expressed in plasma at a higher level following administration of the recombinant AAV construct compared to the administration of an equivalent dose of an equivalent recombinant AAV construct which comprises a wild-type Factor VIII signal peptide. The “equivalent recombinant AAV construct which comprises a wild-type Factor VIII signal peptide” is the same as the recombinant AAV construct of the invention except that the signal peptide is a wild-type Factor VIII signal peptide. Expression in plasma can be determined by the Asserachrom assay as described in the Examples.

Optionally, the Factor VIII polypeptide thereof encoded by the Factor VIII nucleotide sequence is expressed in plasma at least 1.1 fold, at least 1.2 fold, at least 1.5 fold, at least 1.8 fold, at least 2 fold, at least 5 fold higher compared to the equivalent recombinant AAV construct which comprises a wild-type Factor VIII signal peptide.

The signal peptide may comprise a sequence that is at least 90%, at least 92%, at least 95%, at least 98%, at least 99% or 100% identical to the sequence of any one of SEQ ID NOs: 56, 58 or 60. The signal peptide may comprise a sequence that is at least 90%, at least 92%, at least 95%, at least 98%, at least 99% or 100% identical to SEQ ID NO: 60. The signal peptide may comprise a sequence that is at least 90%, at least 92%, at least 95%, at least 98%, at least 99% or 100% identical to SEQ ID NO: 58. The signal peptide may comprise a sequence that is at least 90%, at least 92%, at least 95%, at least 98%, at least 99% or 100% identical to SEQ ID NO: 56. The signal peptide may comprise SEQ ID NO: 60. The signal peptide may comprise SEQ ID NO: 58. Preferably, the signal peptide may comprise SEQ ID NO: 56.

Preferably, the nucleotide sequence encoding the signal peptide is fewer than 57 nucleotides in length. More preferably, the nucleotide sequence encoding the signal peptide is around 54 nucleotides in length. The nucleotide sequence encoding the signal peptide may be around 54 nucleotides in length and encode the sequence of SEQ ID NO: 56. The nucleotide sequence encoding the signal peptide may be around 54 nucleotides in length and encode the sequence of SEQ ID NO:58. Optionally, the nucleotide sequence encoding the signal peptide may be codon optimised. Optionally, the nucleotide sequence encoding the signal peptide is not codon optimised. The nucleotide sequence encoding the signal peptide set forth in SEQ ID NO:56 may have the sequence of SEQ ID NO:57. The nucleotide sequence encoding the signal peptide set forth in SEQ ID NO:58 may have the sequence of SEQ ID NO:59.

The recombinant AAV construct may further comprise a polyA nucleotide sequence. The polyA nucleotide sequence may comprises the nucleotide sequence of any one of SEQ ID NOs: 63 to 66. The polyA nucleotide sequence may be synthetic. The polyA nucleotide sequence may be fewer than 50 nucleotides in length. The polyA nucleotide sequence may be between 16 and 50 nucleotides in length. The polyA nucleotide sequence may be around 49 nucleotides in length. The polyA nucleotide sequence may comprise the nucleotide sequence of SEQ ID NO: 65.

The recombinant AAV construct may further comprise one or two ITR(s). The or each ITR may be a wild-type ITR. The or each ITR may be an AAV2 ITR. The nucleotide sequence of the or each ITR may be fewer than 157, or fewer than 154 nucleotides in length. The nucleotide sequence of the or each ITR may be around 145 nucleotides in length. The nucleotide sequence of a 5′ ITR may comprise the nucleotide sequence of SEQ ID NO: 67. The nucleotide sequence of a 3′ ITR may comprise the nucleotide sequence of SEQ ID NO:70. The nucleotide sequence of a 5′ ITR may comprise the nucleotide sequence of SEQ ID NO: 67 and the nucleotide sequence of a 3′ ITR may comprise the nucleotide sequence of SEQ ID NO:70.

The Factor VIII nucleotide sequence encoding the Factor VIII amino acid sequence may comprise the sequence of SEQ ID NO: 39 or 75. The nucleotide sequence encoding the signal peptide may comprise SEQ ID NO: 57. The Factor VIII nucleotide sequence encoding the Factor VIII amino acid sequence may comprise the sequence of SEQ ID NO: 39 or 75 and the nucleotide sequence encoding the signal peptide may comprise SEQ ID NO: 57.

The recombinant AAV construct may comprise a transcriptional regulatory element which is a liver-specific promoter, and the liver-specific promoter may comprise the nucleotide sequence of SEQ ID NO: 51. The recombinant AAV construct may comprise two ITRs and a polyA nucleotide sequence, wherein the nucleotide sequence of the 5′ ITR is SEQ ID NO: 67 and the sequence of the 3′ ITR is SEQ ID NO:70, and the polyA nucleotide sequence comprises the nucleotide sequence of SEQ ID NO: 65. The AAV construct may have the sequence SEQ ID NO:62 or 71.

The recombinant AAV construct may comprise or consist of SEQ ID NO: 71.

The activity of the polypeptide encoded by the Factor VIII nucleotide sequence achieved may be higher following administration of the recombinant AAV construct compared to the administration of an equivalent dose of an equivalent recombinant AAV construct which comprises the AAV construct of SEQ ID NO: 1 of WO 2017/053677 (SEQ ID NO: 72). The activity of the polypeptide encoded by the Factor VIII nucleotide sequence achieved may be at least 1.1 fold, at least 1.2 fold, at least 1.5 fold, at least 1.8 fold, at least 2 fold, or at least 5 fold higher compared to the equivalent recombinant AAV construct which comprises the AAV construct of SEQ ID NO: 1 of WO 2017/053677 (SEQ ID NO: 72). The activity of the polypeptide encoded by the Factor VIII nucleotide sequence achieved may be between 1.1 fold and 5 fold, or between 1.5 fold and 3 fold, higher compared to the equivalent recombinant AAV construct which comprises the AAV construct of SEQ ID NO: 1 of WO 2017/053677 (SEQ ID NO: 72).

When recombinant AAV constructs of the invention which are less than 4900 nucleotides in length are used to produce AAV viral particles, the vector genome yield may be increased when compared to the vector genome yield obtained when a comparator recombinant AAV construct which is more than 4900 nucleotides in length is used.

Optionally, the vector genome yield is at least 1.5, at least 2, at least 2.25, at least 2.5, at least 2.75, at least 3, at least 4, or at least 5 fold higher than the vector genome yield obtained when the comparator recombinant AAV construct (of more than 4900 nucleotides in length) is used. Optionally, the vector genome yield is between 1.25 and 3 fold higher, between 1.5 and 3 fold higher, or between 2 and 3 fold higher than the vector genome yield obtained when the comparator recombinant AAV construct (of more than 4900 nucleotides in length) is used.

When a recombinant AAV construct of the invention which is less than 4900 nucleotides in length is used to produce AAV viral particles, the vector genome to total particle ratio may be increased when compared to the vector genome to total particle ratio obtained when a comparator recombinant AAV construct which is more than 4900 nucleotides in length is used.

Optionally, the vector genome to total particle ratio is at least 1.25, at least 1.5, at least 1.75, at least 2, at least 2.25, at least 2.5, at least 2.75, at least 3, at least 3.25, at least 3.5, at least 4, or at least 5 fold higher than the vector genome to total particle ratio obtained when the comparator recombinant AAV construct (which is more than 4900 nucleotides in length) is used. Optionally, the vector genome to total particle ratio is between 1.25 and 4 fold higher, or between 1.5 and 3.5 fold higher than the vector genome to total particle ratio obtained when the comparator recombinant AAV construct (which is more than 4900 nucleotides in length) is used.

When a recombinant AAV construct of the invention which is less than 4900 nucleotides in length is used to produce AAV viral particles, the nucleic acid impurity level may be decreased when compared to the nucleic acid impurity level obtained when a comparator recombinant AAV construct which is more than 4900 nucleotides in length is used. Optionally, the nucleic acid impurity level may be a plasmid-derived impurity level. Optionally, the nucleic acid impurity is an AAV cap-derived nucleic acid.

Optionally, the nucleic acid impurity level is 85% or lower, 75% or lower, 60% or lower, 50% or lower, 40% or lower, 30% or lower, or 20% or lower than the nucleic acid impurity level obtained when the comparator recombinant AAV construct is used. Optionally, the nucleic acid impurity level is between 40% and 80%, or between 50% and 70%, or between 15% and 55% of the nucleic acid impurity level obtained when the comparator recombinant AAV construct is used.

Optionally, when the recombinant AAV construct of the invention of less than 4900 nucleotides in length and the comparator recombinant AAV construct are used to produce AAV viral particles, the same AAV production system is used in each case. Optionally said AAV production system is a mammalian production system. Optionally said AAV production system is a two-plasmid system consisting of a helper plasmid comprising sequences encoding AAV Rep functions and a vector plasmid comprising sequences encoding AAV Cap functions.

Optionally, when the AAV production system is a two-plasmid system, such as a two-plasmid system consisting of a helper plasmid comprising AAV Rep functions and a vector plasmid comprising AAV Cap functions, the molar ratio of helper plasmid to vector plasmid is between about 12:1 and 1:12, between about 1:10 and 10:1, between about 1:5 and 5:1, between about 1:3 and 4:3 or between about 1:2 and 1:3. Optionally the molar ratio of helper plasmid to vector plasmid is around 4:3 or 1:3.

Optionally, the comparator recombinant AAV construct is more than 4910, more than 4920, more than 4930, more than 4940, more than 4950, more than 4960, more than 4970, more than 4980, more than 4990 or more than 5000 nucleotides in length. Optionally, the comparator recombinant AAV construct comprises or consists of SEQ ID NO: 72.

Features relating to how the recombinant AAV construct of the invention may be used are discussed further in the sections entitled “Production of AAV viral particles, increasing vector genome yield, increasing vector genome to total particle ratio, and decreasing nucleic acid impurity levels”, “AAV production system, two-plasmid system, vector plasmid and helper plasmid”, and “Transfecting, culturing and harvesting”.

A viral particle comprising the construct

The invention also provides an AAV viral particle comprising the recombinant AAV construct of the invention.

The invention further provides a viral particle comprising a recombinant genome comprising polynucleotides of the invention. For the purposes of the present invention, the term “viral particle” refers to all or part of a virion. For example, the viral particle comprises a recombinant genome and may further comprise a capsid. The viral particle may be a gene therapy vector. Herein, the terms “viral particle” and “vector” are used interchangeably. For the purpose of the present application, a“gene therapy” vector is a viral particle that can be used in gene therapy, i.e. a viral particle that comprises all the required functional elements to express a transgene, such as a Factor IX nucleotide sequence, in a host cell after administration.

Suitable viral particles include a parvovirus, a retrovirus, a lentivirus or a herpes simplex virus. The parvovirus may be an adeno-associated virus (AAV). The viral particle is preferably a recombinant adeno-associated viral (AAV) vector or a lentiviral vector. More preferably, the viral particle is an AAV viral particle. The terms AAV and rAAV are used interchangeably herein.

The genomic organization of all known AAV serotypes is very similar. The genome of AAV is a linear, single-stranded DNA molecule that is less than about 5,000 nucleotides in length. Inverted terminal repeats (ITRs) flank the unique coding nucleotide sequences for the non-structural replication (Rep) proteins and the structural (VP) proteins. The VP proteins (VP1, -2 and -3) form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wild type (wt) AAV infection in mammalian cells the Rep genes (i.e. encoding Rep78 and Rep52 proteins) are expressed from the P5 promoter and the P19 promoter, respectively, and both Rep proteins have a function in the replication of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

AAV sequences that may be used in the present invention for the production of AAV vectors can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chiorini et al, 1997; Srivastava et al, 1983; Chiorini et al, 1999; Rutledge et al, 1998; and Wu et al, 2000. AAV serotype 1, 2, 3, 3B, 4, 5, 6, 7, 8, 9, 10, 11 or 12 may be used in the present invention. The sequences from the AAV serotypes may be mutated or engineered when being used in the production of gene therapy vectors.

Optionally, an AAV vector comprises ITR sequences which are derived from AAV1, AAV2, AAV4 and/or AAV6. Preferably the ITR sequences are AAV2 ITR sequences. Herein, the term AAVx/y refers to a viral particle that comprises some components from AAVx (wherein x is a AAV serotype number) and some components from AAVy (wherein y is the number of the same or different serotype). For example, an AAV2/8 vector may comprise a portion of a viral genome, including the ITRs, from an AAV2 strain, and a capsid derived from an AAV8 strain.

In an embodiment, the viral particle is an AAV viral particle comprising a capsid. AAV capsids are generally formed from three proteins, VP1, VP2 and VP3. The amino acid sequence of VP1 comprises the sequence of VP2. The portion of VP1 which does not form part of VP2 is referred to as VP lunique or VP1U. The amino acid sequence of VP2 comprises the sequence of VP3. The portion of VP2 which does not form part of VP3 is referred to as VP2unique or VP2U. The viral particle may comprise a capsid. The capsid may be selected from the group consisting of:
(i) a capsid comprising a sequence which is at least 96%, at least 98%, at least 99%, at least 99.5%, at least 99.8% or 100% identical to SEQ ID NO: 68;
(ii) a capsid comprising a sequence which is at least 96%, at least 98%, at least 99%, at least 99.5%, at least 99.8%, or 100% identical to SEQ ID NO: 69;
(iii) a liver-tropic capsid; and
(iv) an AAV5 capsid.

The capsid may be selected from the group consisting of:
(i) a capsid comprising a sequence which is at least 96%, at least 98%, at least 99%, at least 99.5%, at least 99.8% or 100% identical to SEQ ID NO: 91;
(ii) a capsid comprising a sequence which is at least 96%, at least 98%, at least 99%, at least 99.5%, at least 99.8% or 100% identical to SEQ ID NO: 92; and
(iii) an AAV6 capsid.

A viral particle of the invention may be a “hybrid” particle in which the viral ITRs and viral capsid are from different parvoviruses, such as different AAV serotypes. Preferably, the viral ITRs and capsid are from different serotypes of AAV, in which case such viral particles are known as transcapsidated or pseudotyped. Likewise, the parvovirus may have a “chimeric” capsid (e.g., containing sequences from different parvoviruses, preferably different AAV serotypes) or a “targeted” capsid (e. g., a directed tropism).

The activity of the polypeptide encoded by the Factor VIII nucleotide sequence achieved may be higher following administration of the AAV viral particle compared to the administration of an equivalent dose of an equivalent AAV viral particle which comprises the AAV construct of SEQ ID NO: 1 of WO 2017/053677 (SEQ ID NO: 72). The activity of the polypeptide encoded by the Factor VIII nucleotide sequence achieved may be at least 1.1 fold, at least 1.2 fold, at least 1.5 fold, at least 1.8 fold, at least 2 fold, or at least 5 fold higher compared to the equivalent AAV viral particle which comprises the AAV construct of SEQ ID NO: 1 of WO 2017/053677 (SEQ ID NO: 72). The activity of the polypeptide encoded by the Factor VIII nucleotide sequence achieved may be between 1.1 fold and 5 fold, or between 1.5 fold and 3 fold, higher compared to the equivalent AAV viral particle which comprises the AAV construct of SEQ ID NO: 1 of WO 2017/053677 (SEQ ID NO: 72).

Production of AAV viral particles, increasing vector genome yield, increasing vector genome to total particle ratio, and decreasing nucleic acid impurity levels

The recombinant AAV construct of the invention may be used to produce a population of AAV viral particles. The present invention provides use of the recombinant AAV construct of the invention for producing a population of AAV viral particles. Optionally, the use comprises transfecting a host cell with the recombinant AAV construct of the invention and culturing the host cell under conditions suitable for AAV viral particle production.

The present invention also provides a method for producing a population of AAV viral particles comprising:
a) obtaining the recombinant AAV construct of the invention;
b) transfecting a host cell with the said recombinant AAV construct; and
c) culturing the host cell under conditions suitable for AAV viral particle production.

Preferably the recombinant AAV construct used to produce the population of AAV viral particles is less than 4900 nucleotides in length. In other words, preferably a recombinant AAV construct of the invention (e.g. as described above) which is less than 4900 nucleotides in length may be used to produce the population of AAV viral particles. For example, the recombinant AAV construct may be less than 4800 nucleotides in length.

When using the recombinant AAV construct of the invention which is less than 4900 nucleotides in length, optionally, the population of AAV viral particles:
a) has an increased vector genome yield when compared to the vector genome yield obtained when a comparator recombinant AAV construct is used;
b) has an increased vector genome to total particle ratio when compared to the vector genome to total particle ratio obtained when a comparator recombinant AAV construct is used; and/or
c) has a decreased nucleic acid impurity level when compared to the nucleic acid impurity level obtained when a comparator recombinant AAV construct is used;
   wherein the comparator recombinant AAV construct is more than 4900 nucleotides in length.

The present invention also provides use of the recombinant AAV construct of the invention which is less than 4900 nucleotides in length for:
a) increasing the vector genome yield during AAV viral particle production when compared to the vector genome yield obtained when a comparator recombinant AAV construct is used;
b) increasing the vector genome to total capsid ratio during AAV viral particle production when compared to the vector genome to total capsid ratio obtained when a comparator recombinant AAV construct is used; and/or
c) decreasing the nucleic acid impurity level during AAV viral particle production when compared to the nucleic acid impurity level obtained when a comparator recombinant AAV construct is used;
   wherein the comparator recombinant AAV construct is more than 4900 nucleotides in length.

In some embodiments, the use comprises transfecting a host cell with the recombinant AAV construct of the invention which is less than 4900 nucleotides in length and culturing the host cell under conditions suitable for AAV viral particle production.

The present invention also provides a method for increasing the vector genome yield during the production of a population of AAV viral particles, comprising:
a) obtaining the recombinant AAV construct of the invention which is less than 4900 nucleotides in length;
b) transfecting a host cell with the said recombinant AAV construct; and
c) culturing the host cell under conditions suitable for AAV viral particle production;
   wherein the vector genome yield is increased when compared to the vector genome yield obtained when a comparator recombinant AAV construct of more than 4900 nucleotides in length is used.

Optionally, vector genome yield is increased when a recombinant AAV construct of the invention which is less than 4900 nucleotides in length is used to produce AAV viral particles when compared to the vector genome yield obtained when a comparator recombinant AAV construct which is more than 4900 nucleotides in length is used.

In some embodiments, the term “yield” refers to the amount of AAV particles that are produced. In some embodiments, the “yield” may be expressed as the “vector genome yield”. The “vector genome yield” relates to the number of vector genomes that are produced. The “vector genome yield” may be expressed as the number of vector genomes (vg) per ml of medium (as measured on bulk product prior to harvesting, purifying and/or concentrating the population of AAV viral particles). The number of vector genomes may be determined using qPCR. Optionally, the vector genome yield is at least 1.5, at least 2, at least 2.25, at least 2.5, at least 2.75, at least 3, at least 4, or at least 5 fold higher than the vector genome yield obtained when the comparator recombinant AAV construct (of more than 4900 nucleotides in length) is used. Optionally, the vector genome yield is between 1.25 and 3 fold higher, between 1.5 and 3 fold higher, or between 2 and 3 fold higher than the vector genome yield obtained when the comparator recombinant AAV construct (of more than 4900 nucleotides in length) is used. Optionally, the vector genome yield is between 1.5 and 2 fold higher than the vector genome yield obtained when the comparator recombinant AAV construct (of more than 4900 nucleotides in length) is used.

The present invention also provides a method for increasing the vector genome to total particle ratio during the production of a population of AAV viral particles, comprising:
a) obtaining the recombinant AAV construct of the invention which is less than 4900 nucleotides in length;
b) transfecting a host cell with the said recombinant AAV construct; and
c) culturing the host cell under conditions suitable for AAV viral particle production;
   wherein the vector genome to total particle ratio is increased when compared to the vector genome to total particle ratio obtained when a comparator recombinant AAV construct of more than 4900 nucleotides in length is used.

Optionally, vector genome to total particle ratio is increased when a recombinant AAV construct of the invention which is less than 4900 nucleotides in length is used to produce AAV viral particles when compared to the vector genome to total particle ratio obtained when a comparator recombinant AAV construct which is more than 4900 nucleotides in length is used.

The vector genome to total particle ratio may be expressed herein as the percentage of the total number of particles (capsids) that notionally comprise a vector genome or at least a partial such genome (assuming one (partial) genome per capsid) as determined using qPCR. The vector genome to total particle ratio may be determined using qPCR to determine the number of vector genomes (as discussed above), and using a capsid-specific ELISA to measure the total number of particles. For example, the capsid-specific ELISA may comprise exposing the AAV particles to an antibody that binds to the capsid protein. If, for example, the capsid protein is from an AAV2 serotype, the antibody may be an antibody that binds to the AAV2 capsid. For example, the user may coat a plate with an antibody that is specific for the capsid. The user may then pass the AAV particles over the surface of the plate. The particles will bind to the antibody and be immobilised on the plate. The plate may then be washed to remove contaminants. The amount of particle present can then be detected by addition of a detection antibody that can bind to the capsid and is conjugated to a detection agent such as streptavidin peroxidase. The amount of particle present will be proportional to the colour change obtained when the streptavidin peroxidase is exposed to the chromogenic substrate TMB (tetramethylbenzidine).

Optionally, the vector genome to total particle ratio is at least 1.25, at least 1.5, at least 1.75, at least 2, at least 2.25, at least 2.5, at least 2.75, at least 3, at least 3.25, at least 3.5, at least 4, or at least 5 fold higher than the vector genome to total particle ratio obtained when the comparator recombinant AAV construct (which is more than 4900 nucleotides in length) is used. Optionally, the vector genome to total particle ratio is between 1.25 and 4 fold higher, or between 1.5 and 3.5 fold higher than the vector genome to total particle ratio obtained when the comparator recombinant AAV construct (which is more than 4900 nucleotides in length) is used. Optionally, the vector genome to total particle ratio is between 1.4 and 1.6 fold higher than the vector genome to total particle ratio obtained when the comparator recombinant AAV construct (which is more than 4900 nucleotides in length) is used.

When producing AAV particles which are, for example, suitable for use therapeutically, such as in gene therapy, there is a desire to reduce the amount of nucleic acid impurities present. The inventors have found that using a recombinant AAV construct of the invention which is less than 4900 nucleotides in length resulted in lower nucleic acid impurity levels than when using a comparator recombinant AAV construct which is longer than 4900 nucleotides in length.

The present invention also provides a method for decreasing the nucleic acid impurity level during the production of a population of AAV viral particles, comprising:
a) obtaining the recombinant AAV construct of the invention which is less than 4900 nucleotides in length;
b) transfecting a host cell with the said recombinant AAV construct; and
c) culturing the host cell under conditions suitable for AAV viral particle production;
   wherein the nucleic acid impurity level is decreased when compared to the nucleic acid impurity level obtained when a comparator recombinant AAV construct of more than 4900 nucleotides in length is used.

Optionally, nucleic acid impurity level is decreased when a recombinant AAV construct of the invention which is less than 4900 nucleotides in length is used to produce AAV viral particles when compared to the nucleic acid impurity level obtained when a comparator recombinant AAV construct which is more than 4900 nucleotides in length is used.

The term “nucleic acid impurity” refers to the nucleic acid, present in the population of AAV viral particles, which was not intended to be packaged. In general, the intention is to package the recombinant AAV construct of the invention as a vector genome (AAV genome). AAV viral particles can be generated using plasmids which provide the AAV Rep and Cap functions and provide the vector genome. The plasmids can also provide the adenoviral helper functions or the helper functions can be provided through co-infection with adenovirus. Such plasmids are discussed further in the section entitled: “AAV production system, two-plasmid system, vector plasmid and helper plasmid”. Generally, any sequence which is present in the population of AAV viral particles produced, and which is not present in the vector genome, is considered a nucleic acid impurity. For example, any plasmid-derived sequence which does not form part of the vector genome is considered a nucleic acid impurity, i.e. a “plasmid-derived nucleic acid impurity”. As an example, full or partial sequences encoding Cap and/or Rep proteins are considered plasmid-derived nucleic acid impurities. Optionally, the plasmid-derived nucleic acid impurity is an AAV cap-derived nucleic acid, i.e. a nucleic acid comprising part or all of a sequence encoding a Cap protein. As a further example, prokaryotic DNA sequences, such as antibiotic resistance genes or sequences derived from the plasmid backbone, are considered plasmid-derived nucleic acid impurities. As an example, nucleic acid sequences of the kanamycin resistance gene (kanR) are considered plasmid-derived nucleic acid impurities. The plasmid backbone is the bacterial sequence needed to amplify the plasmids in bacterial host cells. The plasmid backbone may be the region of the plasmid that is not derived from adenovirus or AAV or is not situated between two AAV-derived ITRs. On the other hand, any sequences which are present in the vector genome intended to be packaged, such as the Factor VIII nucleotide sequence, the nucleic acid sequence encoding the signal peptide, the sequence of the ITR(s), the sequence of the transcriptional regulatory element, the sequence encoding the signal peptide, and/or the polyA sequence, are not considered nucleic acid impurities. Nucleic acid impurities may also derive from the genome of the host cell from which the AAV can be produced , and are referred to as “host cell-derived nucleic acid impurity”. The term “nucleic acid impurity level” refers to the amount of nucleic acid impurity present in the population of AAV viral particles.

Optionally, the nucleic acid impurity level is 85% or lower, 75% or lower, 60% or lower, 50% or lower, 40% or lower, 30% or lower, 20% or lower, or 15% or lower than the nucleic acid impurity level obtained when the comparator recombinant AAV construct is used. Optionally, the nucleic acid impurity level is 85% or lower, 75% or lower, 60% or lower, 50% or lower, 40% or lower, 30% or lower, or 20% or lower than the nucleic acid impurity level obtained when the comparator recombinant AAV construct is used. Optionally, the nucleic acid impurity level is between 40% and 80%, between 50% and 70%, or between 15% and 55% of the nucleic acid impurity level obtained when the comparator recombinant AAV construct is used. Optionally, the nucleic acid impurity level is between 40% and 60%, or between 45% and 55% of the nucleic acid impurity level obtained when the comparator recombinant AAV construct is used. The nucleic acid impurity level being, e.g., “70% or lower”, corresponds to a fold change value of 0.7 or lower relative to the nucleic acid impurity level when a comparator recombinant AAV construct (which is more than 4900 nucleotides in length) is used. The fold change value is calculated by dividing the nucleic acid impurity level when the recombinant AAV construct (which is less than 4900 nucleotides in length) is used by the nucleic acid impurity level when a comparator recombinant AAV construct (which is more than 4900 nucleotides in length) is used.

The nucleic acid impurity level may be measured by qPCR. Suitable qPCR is discussed in Example 15. Optionally, the qPCR may use primers which bind to a sequence encoding cap. Optionally, the qPCR may use primers which bind to nucleic acid sequences of the kanamycin resistance gene (kanR). The AAV viral particles may be purified prior to the qPCR analysis. The purification may be by any of the methods of purification described herein, such as affinity chromatography.

The “comparator recombinant AAV construct” is more than 4900 nucleotides in length. The comparator recombinant AAV construct comprises a polynucleotide comprising a Factor VIII nucleotide sequence, wherein the Factor VIII nucleotide sequence encodes a Factor VIII polypeptide comprising a Factor VIII amino acid sequence. Optionally, the comparator recombinant AAV construct is more than 4910, more than 4920, more than 4930, more than 4940, more than 4950, more than 4960, more than 4970, more than 4980, more than 4990 or more than 5000 nucleotides in length. Optionally, the comparator recombinant AAV construct comprises SEQ ID NO: 72 (AAV construct of SEQ ID NO: 1 of WO 2017/053677). Optionally, the comparator recombinant AAV construct consists of SEQ ID NO: 72 (AAV construct of SEQ ID NO: 1 of WO 2017/053677).

When referring to percent changes in nucleic acid impurity level, fold changes in vector genome to total particle ratio, or fold changes in vector genome yield, the term “between” includes the specified values.

AAV production system, two plasmid system, vector plasmid and helper plasmid

When AAV viral particles are produced according to the invention, any AAV production system may be used. When the recombinant AAV construct of the invention and the comparator recombinant AAV construct are used to produce AAV viral particles, the same AAV production system may be used in each case. Optionally, the AAV production system is an insect cell or baculovirus production system. Optionally, the AAV production system is a mammalian production system. Optionally, a producer cell line is used in which the recombinant AAV construct and/or the AAV rep and/or cap genes are incorporated into the host cell genome, wherein infection of the producer cell with helper virus facilitates AAV production. Alternatively, optionally, plasmids are used to produce AAV viral particles. Optionally, the plasmids are circular nucleic acid molecules. Optionally, the plasmids are nucleic acid molecules that are of bacterial origin. Optionally, the AAV production system is a two-plasmid system. The phrase “two plasmid system” refers to a system that comprises two plasmids (which may be referred to as a vector plasmid and a helper plasmid), and can be used without the need for additional plasmids to produce AAV viral particles. Optionally, the two-plasmid system can be used to produce AAV viral particles without the need for genetic material originating from a host cell, optionally with the exception of a gene encoding E1A/B. However, the two-plasmid system may comprise additional non-plasmid components. Optionally, the two-plasmid system does not comprise a helper virus. Optionally, the two-plasmid system comprises all the necessary genetic information for the production of AAV viral particles. For example, the two-plasmid system may comprise at least one rep gene, at least one cap gene and at least one helper gene. Optionally, the two-plasmid system comprises all the necessary genetic information required for the production of AAV viral particles suitable for use in gene therapy. Optionally, the two-plasmid system may be the trans-split two plasmid system as described in WO 2020/208379.

Optionally, the two-plasmid system comprises a helper plasmid and a vector plasmid. Optionally, the two-plasmid system consists of a helper plasmid and a vector plasmid. The term “helper” is not intended to be limiting. Accordingly, a “helper plasmid” is any plasmid that is suitable for use alongside a vector plasmid in a two-plasmid system. Optionally, the helper plasmid comprises at least one rep gene. Optionally, the helper plasmid comprises sequences encoding AAV Rep functions. Optionally, the helper plasmid comprises at least one helper gene. The term “vector plasmid” is not intended to be limiting. Accordingly, a “vector plasmid” is any plasmid that is suitable for use alongside a helper plasmid in a two-plasmid system. Optionally, the vector plasmid comprises at least one cap gene. Optionally, the vector plasmid comprises sequences encoding AAV Cap functions. Optionally, the vector plasmid comprises the recombinant AAV construct of the invention (or the comparator recombinant AAV construct).

Optionally, the vector plasmid backbone encompasses any nucleotides except the at least one cap gene, a promoter (region) operably linked to the at least one cap gene and the recombinant AAV construct of the invention (or the comparator recombinant AAV construct). Optionally, the helper plasmid backbone encompasses any nucleotides except the at least one helper gene and associated adenovirus-derived regulatory elements, and the at least one rep gene and associated AAV-derived regulatory elements.

Optionally, the molar ratio of helper plasmid to vector plasmid is between about 12:1 and 1:12, between about 1:10 and 10:1, between about 1:5 and 5:1, or between about 1:3 and 4:3. Optionally, the molar ratio of helper plasmid to vector plasmid is between 1:2 and 1:4, or around 1:3. Optionally, the molar ratio of helper plasmid to vector plasmid is between about 1:3 and 4:3. Optionally, the molar ratio of helper plasmid to vector plasmid is around 4:3. When referring to the molar ratio, the term “between” includes the specified values.

Transfecting, culturing and harvesting

In some embodiments, the methods of the invention may comprise steps of obtaining the recombinant AAV construct of the invention, optionally which is less than 4900 nucleotides in length as outlined above, transfecting a host cell with the said recombinant AAV construct, and culturing the host cell under conditions suitable for AAV viral particle production. The uses of the invention may comprise transfecting a host cell with the recombinant AAV construct of the invention, optionally which is less than 4900 nucleotides in length as outlined above, and culturing the host cell under conditions suitable for AAV viral particle production.

Transfecting a host cell with the said recombinant AAV construct may comprise exposing the host cell to the said recombinant AAV construct in conditions suitable for transfection. For example, the user of the method or use may add a transfection agent (addition of a transfection agent would be considered to be a condition suitable for transfection). Alternatively, calcium phosphate transfection, electroporation or cationic liposomes could be used. Optionally, the step of transfecting the host cell takes place when the host cell has grown to confluence.

Culturing the host cell under conditions suitable for AAV viral particle production refers to culturing the host cell under conditions at which it can grow and AAV can replicate. For example, the host cell may be cultured at a temperature between 32° C. and 40° C., between 34° C. and 38° C., between 35° C. and 38° C. or around 37° C. When referring to temperature, the term “between” includes the specified values. Optionally, the host cell may be cultured in the presence of a complete cell culture medium such as Dulbecco's Modified Eagle's Medium (DM EM). A completed cell culture medium is a medium that provides all the essential nutrients required for growth of the host cell. Optionally, the complete cell culture medium is supplemented with serum, such as fetal bovine serum or bovine serum albumin.

In some embodiments, the host cell is selected from the group consisting of a HEK293T cell, a HEK293 cell, a HEK293EBNA cell, a CAP cell, a CAP-T cell, an AGE1.CR cell, a PerC6 cell, a C139 cell, and an EB66 cell. Optionally, the host cell is a cell that expresses a functional E1A/B protein.

In some embodiments, the methods further comprise a step of harvesting the AAV viral particles to provide a population of AAV viral particles.

In some embodiments, the methods or uses further comprise a step of purifying the AAV viral particles. In general, a step of purifying the AAV viral particles will involve increasing the concentration of the AAV viral particles compared to other components. Optionally, the step of purifying the AAV viral particles results in isolated AAV viral particles.

Any suitable purification method may be used. Optionally, the step of purifying the AAV viral particles is carried out using a technique selected from the group consisting of gradient density centrifugation (such as CsCl or Iodixanol gradient density centrifugation), filtration, ion exchange chromatography, size exclusion chromatography, affinity chromatography and hydrophobic interaction chromatography.

In some embodiments, the methods or uses comprise further concentrating the AAV viral particles using ultracentrifugation, tangential flow filtration, or gel filtration.

In some embodiments, the methods or uses further comprise formulating the AAV viral particles with a pharmaceutically acceptable excipient. The pharmaceutically acceptable excipients may comprise carriers, diluents and/or other medicinal agents, pharmaceutical agents or adjuvants, etc. Optionally, the pharmaceutically acceptable excipients comprise saline solution. Optionally, the pharmaceutically acceptable excipients comprise human serum albumin.

In some embodiments, the recombinant AAV construct used in the methods or uses of the invention may be present in a plasmid, such as a vector plasmid. Methods for producing AAV viral particles using a recombinant AAV construct of the invention, or uses of the recombinant AAV construct of the invention for producing AAV viral particles, thus encompass the use of a recombinant AAV construct of the invention when present in such a plasmid. For example a plasmid, such as a vector plasmid, may comprise the recombinant AAV construct. Thus a plasmid, such as a vector plasmid, comprising the recombinant AAV construct may be used in the uses and methods of the invention described above. The plasmid may be part of a two-plasmid system. For example, a two-plasmid system comprising a plasmid (such as a vector plasmid) which comprises the recombinant AAV construct may be used in the uses and methods of the invention described above.

For the purposes of comparing properties of a population of AAV viral particles (e.g. the vector genome yield, the vector genome to total particle ratio and/or the nucleic acid impurity level) when a recombinant AAV construct of the invention is used, with a population of AAV viral particles when a comparator recombinant AAV construct is used, it is preferred that the same AAV production system is used in each case. For example, when the recombinant AAV construct used in the methods or uses of the invention is present in a plasmid, such as a vector plasmid, the comparator recombinant AAV construct is also present in an equivalent plasmid, such as an equivalent vector plasmid. Similarly. when the recombinant AAV construct used in the methods or uses of the invention is present in a plasmid which is part of a two-plasmid system, the comparator recombinant AAV construct is also present in an equivalent plasmid as part of an equivalent two-plasmid system.

Population of AAV Viral Particles

The present invention also provides a population of AAV viral particles obtained by any one of the above methods of the invention. The present invention also provides a population of AAV viral particles obtainable by any one of the above methods of the invention.

The population of AAV viral particles may be used in the methods and uses described below. The composition of the invention as described below may comprise the population of AAV viral particles.

Compositions, Methods and Uses

In a further aspect of the invention, there is provided a composition comprising the polynucleotide or construct/viral particle of the invention and a pharmaceutically acceptable excipient.

The pharmaceutically acceptable excipients may comprise carriers, diluents and/or other medicinal agents, pharmaceutical agents or adjuvants, etc. Optionally, the pharmaceutically acceptable excipients comprise saline solution. Optionally, the pharmaceutically acceptable excipients comprise human serum albumin.

The invention further provides a polynucleotide, construct/viral particle or composition of the invention for use in a method of treatment. Optionally the method of treatment comprises administering an effective amount of the polynucleotide or construct/viral particle of the invention to a patient. Optionally the method of treatment comprises administering an effective amount of the composition of the invention to a patient.

The invention further provides use of the polynucleotide, construct/viral particle or composition of the invention in the manufacture of a medicament for use in a method of treatment. Optionally the method of treatment comprises administering an effective amount of the polynucleotide or construct/viral particle of the invention to a patient. Optionally the method of treatment comprises administering an effective amount of the composition of the invention to a patient.

Optionally the method of treatment is a gene therapy. A “gene therapy” involves administering a construct/viral particle of the invention that is capable of expressing a transgene (such as a Factor VIII nucleotide sequence) in the host to which it is administered.

Optionally, the method of treatment is a method of treating a coagulopathy such as haemophilia (for example haemophilia A or B) or Von Willebrands' disease. Preferably, the coagulopathy is characterised by increased bleeding and/or reduced clotting. Optionally, the method of treatment is a method of treating haemophilia, for example haemophilia A. In some embodiments, the patient is a patient suffering from haemophilia A. Optionally the patient has antibodies or inhibitors to Factor VIII. Optionally, the polynucleotide and/or construct/viral particle is administered intravenously. Optionally, the polynucleotide and/or construct/viral particle is for administration only once (i.e. a single dose) to a patient.

Optionally, the method of the invention may further comprise a step of determining whether the patient has been partially or fully treated (e.g. determining that the patient has been partially or fully treated for the symptoms of haemophilia A) by administration of the gene therapy vector. Partially or fully treating haemophilia A may refer to improving the clotting of a patient suffering from haemophilia A, and reducing the risk of an uncontrolled bleeding event. Partially or fully treating haemophilia A may refer to reducing the number and/or frequency of uncontrolled bleeding events or internal bleeding e.g. in joints. A patient who is partially or fully treated may suffer fewer uncontrolled bleeding events per year. When haemophilia A is “treated” in the above method, this means that one or more symptoms of haemophilia are ameliorated. It does not mean that the symptoms of haemophilia are completely remedied so that they are no longer present in the patient, although in some methods, this may be the case. The method of treatment may result in one or more of the symptoms of haemophilia A being less severe than before treatment. Partially or fully treating haemophilia A may refer to increasing the amount or activity of FVIII present in the plasma of the patient. Optionally, relative to the situation pre-administration, the method of treatment results in an increase in the amount/concentration of circulating Factor VIII in the blood of the patient, and/or the overall level of Factor VIII activity detectable within a given volume of blood of the patient, and/or the specific activity (activity per amount of Factor VIII protein) of the Factor VIII in the blood of the patient. Partially treating haemophilia may refer to converting a patient with severe (<1% normal blood clotting factor activity) or moderately severe haemophilia (<2% normal blood clotting factor activity) to a patient with mild haemophilia (5-40% normal blood clotting factor activity). Fully treating haemophilia may refer to increasing the blood clotting factor activity of a patient with severe, moderately severe or mild haemophilia to within the normal range (50%-150% normal blood clotting factor activity).

A patient who is partially or fully treated may require a lower dose of Factor VIII to be administered. Optionally, a patient who is partially or fully treated may not require the on-going administration of FVIII. Optionally, the method may further comprise a step of assessing whether a patient who has been partially or fully treated for haemophilia no longer needs ongoing treatment or needs a reduced level of ongoing treatment with Factor VIII, and adjusting the patient's treatment regimen appropriately, e.g. to reduce the dose or dose frequency of the Factor VIII that is to be administered, or to halt the administration of the Factor VIII.

A “therapeutically effective amount” refers to an amount effective, at dosages and for periods of time necessary, to achieve the desired therapeutic result, such as raising the level of functional factor VIII in a subject (so as to lead to functional factor VIII production at a level sufficient to ameliorate the symptoms of haemophilia A).

Optionally, the construct/viral particle is administered at a dose of less than 1×10¹¹, less than 1×10¹², less than 5×10¹², less than 2×10¹², less than 1.5×10¹², less than 3×10¹², less than 1×10¹³, less than 2×10¹³, or less than 3×10¹³ construct genomes per kg of weight of patient. Optionally, the polynucleotide, viral particle or composition is administered in a dose of at least 4.5×10¹¹, or between 4.5×10¹¹ and 1×10¹² construct genomes per kg of weight of patient (vg/kg). Optionally, the polynucleotide, viral particle or composition is administered in a dose of less than 5×10¹¹ vg/kg. Optionally, the polynucleotide, viral particle or composition is administered in a dose of between 4.5×10¹¹ vg/kg and 5×10¹¹ vg/kg, or between 4.5×10¹¹ vg/kg and 4.9×10¹¹ vg/kg. Optionally, the polynucleotide or viral particle that is administered is produced from a mammalian cell, and/or possesses characteristics which result from use of mammalian viral construct production cells and distinguish from constructs produced in insect viral construct production cells (e.g. baculovirus system).

Optionally, administration of a given dose of constructs/viral particles —quantified in terms of the number of construct genomes—is achieved using qPCR to titrate construct genomes.

The transgene cassette is also sometimes known as a transgene expression cassette.

Methods of performing qPCR are known to those of skill in the art. Using a real-time PCR cycler and DNA-binding dye such as SYBR Green (ThermoFisher Scientific) the amplification of nascent double-stranded amplicons can be detected in real time. Known quantities of the qPCR template genetic material (e.g. the promoter region) may then be serially diluted to create a standard curve, and sample construct genome titre interpolated from the standard curve.

### EXAMPLES

### Example 1—General Materials and Methods

FVIII Constructs

The cDNA of human FVIII-SQ (encoding FVIII containing a 14 amino acid linker region in place of the B domain, (Lind et al. 1995. Eur J Biochem 232, 19-27) was cloned into a liver-specific promoter-driven adeno-associated virus (AAV) vector (FLJ-PL207).

Different elements of the AAV construct were optimised to improve expression. 21 different codon optimised FVIII variants were designed and all were based on FVIII-SQ. 21 FVIII-SQ sequences were codon optimised on the basis of highly liver-expressed proteins, and tested for improved expression relative to wild type FVIII coding sequence. Over ten signal peptides derived from liver-expressed plasma proteins were tested in conjunction with the FVIII-SQ transgenes. To reduce the AAV recombinant genome size, a number of small liver-specific transcription regulatory elements including truncated HLP2-derived promoters were designed and tested with the FVIII-SQ transgenes.

Numerous FVIII-SQ internal deletion variants were generated by site-directed mutagenesis of the AAV-FVIII-SQ template using the Q5 Site-Directed Mutagenesis kit (NEB) following manufacturer's instructions, and sequenced for confirmation.

Generation of AAV Vectors

AAV particles were produced by triple plasmid transfection of HEK293T cells with plasmids encoding the AAV Rep and Cap functions; adenoviral helper functions; and the recombinant genome containing the FVIII expression cassette flanked by AAV2 ITRs. Cell pellet and supernatant were harvested 72 hours post-transfection and AAV particles purified by affinity chromatography using resins such as POROS Capture Select and AVB Sepharose. AAV was then dialysed into PBS overnight, stored at 4° C. and titred by qPCR.

Assays

FVIII Chromogenic Activity Assay

The Biophen FVIII:C chromogenic assay (Hyphen BioMed, ref 221406) measures the cofactor activity of FVIII (FVIII:C).

Through thrombin activation, the FVIII:C polypeptide forms a complex with human Factor IXa, phospholipids and calcium. Under these conditions, Factor X, provided in this assay at a specific concentration and in excess, is converted into Factor Xa (activated). This Factor Xa produced is directly proportional to FVIII:C, the limiting factor. Factor Xa is directly measured by a chromogenic substrate, Sxa-11. Factor Xa cleaves the chromogenic substrate and releases pNA. Production of pNA is proportional to Factor Xa activity, which is directly related to FVIII:C activity. The level of pNA released can be determined by measuring colour development at 405 nm, and this is relative to the amount of the Factor Xa polypeptide generated by Factor VIII:C in the sample, which is proportional to the activity of FVIII:C in the sample.

The assay is performed according to manufacturer's instructions. Briefly, to a microplate well, preincubated at 37° C., 50 μl of calibrator plasmas, diluted (in reagent R4) test plasmas or cell supernatants/lysates or controls, is added, followed by 50 μl each of reagent R1 and R2, which are reconstituted with 6 mL of distilled water and prewarmed to 37° C. After mixing, these components form a 150 μl reaction that is allowed to incubate for 5 min at 37° C. Subsequently, the reaction is supplemented with reagent R3, which is itself resuspended in 6mL of distilled water and prewarmed to 37° C., and the 200 μL mix is allowed to incubate for a further 5 min at 37° C. The reaction is stopped by adding 50 μl of 20% acetic acid or citric acid (20 g/l), before measuring the absorbance of the resulting 250 μl mixture at 405 nm.

Reagents:

R1—Human Factor X, lyophilized in presence of a fibrin polymerization inhibitor.

R2—Activation Reagent-Factor IXa (human), at a constant and optimized concentration, containing human thrombin, calcium and synthetic phospholipids, lyophilized.

R3 —SXa-11-Chromogenic substrate, specific for Factor Xa, lyophilized, with a thrombin inhibitor.

R4—Tris-BSA Buffer. Contains 1% BSA, PEG, FVIII:C Stabilizer and sodium azide (0.9g/L).

In relation to the readout from the chromogenic activity assay, the “% FVIII activity” (also referred to as “% FVIII:C”) is the “% normal” which means, for example in the context of expressing a FVIII expression cassette in HuH-7 cells, that relative to a human plasma sample having 100% FVIII activity, the FVIII activity detected in a supernatant following expression of a FVIII expression cassette in HuH-7 cells is a specified % of the FVIII activity detected in said human plasma sample having 100% FVIII activity.

FVIII Sandwich ELISA Antigen Assay

The Asserachrom VIII:Ag kit (Stago Diagnostica, ref 00280) is an antigenic assay for quantification of FVIII in plasma by enzyme-linked immunosorbent assay (ELISA). FVIII in assayed samples is captured by a mouse-monoclonal anti-human VIII:Ag antibody, pre-coating the walls of a plastic microplate well. Following sufficient incubation and washing to reduce non-specific binding, mouse anti-human FVIII antibodies, coupled to peroxidase, bind to the remaining free antigenic determinants of the captured FVIII. The bound peroxidase is then revealed by TMB substrate. The colour development induced by TMB is halted by the addition of a strong acid. The intensity of the colour development is directly proportional to the FVIII concentration in the assayed sample, determined by measuring the absorbance at 450 nm.

The readout from this assay may be expressed as “% normal” which means, for example in the context of expressing FVIII constructs in mice, that relative to a human plasma sample having 100% FVIII activity, the number of FVIII molecules (strictly, epitopes) detected in a mouse plasma sample is a specified % of the number of FVIII molecules/epitopes detected in said human plasma sample having 100% FVIII activity.

In both the activity and antigen assays described above, FVIII (activity or antigen levels) is quantified in mouse, or human cell supernatant, samples using the manufacturer recommended or included lyophilized human plasma samples of known FVIII activity or antigen (as appropriate), calibrated against the WHO International Standard (NIBSC code 07/316).

### Example 2—Codon-Optimisation of FVIII-SQ Coding Sequence

In an attempt to optimise expression of the FVIII-SQ protein in human liver cells, various codon-optimisation algorithms were applied to the ‘wild type’ coding sequence for FVIII-SQ (i.e. the wild type sequence encoding human FVIII, but with internal deletion necessary to encode FVIII-SQ). Codon optimisation algorithms were given codon bias inputs based on particular genes known to express highly into plasma from the liver; in some cases multiple gene sequences were used as inputs.

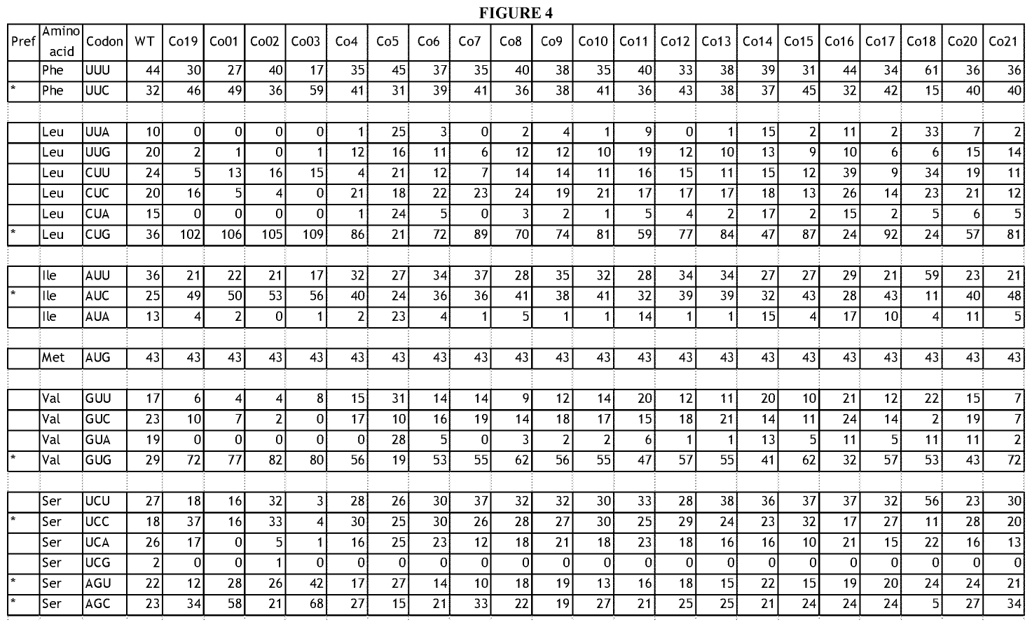

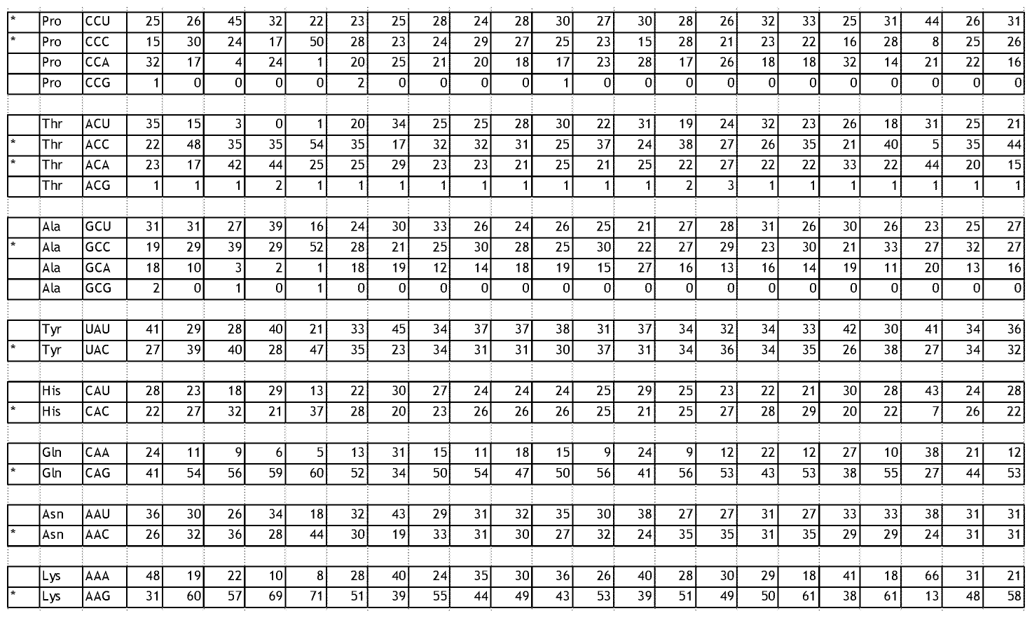

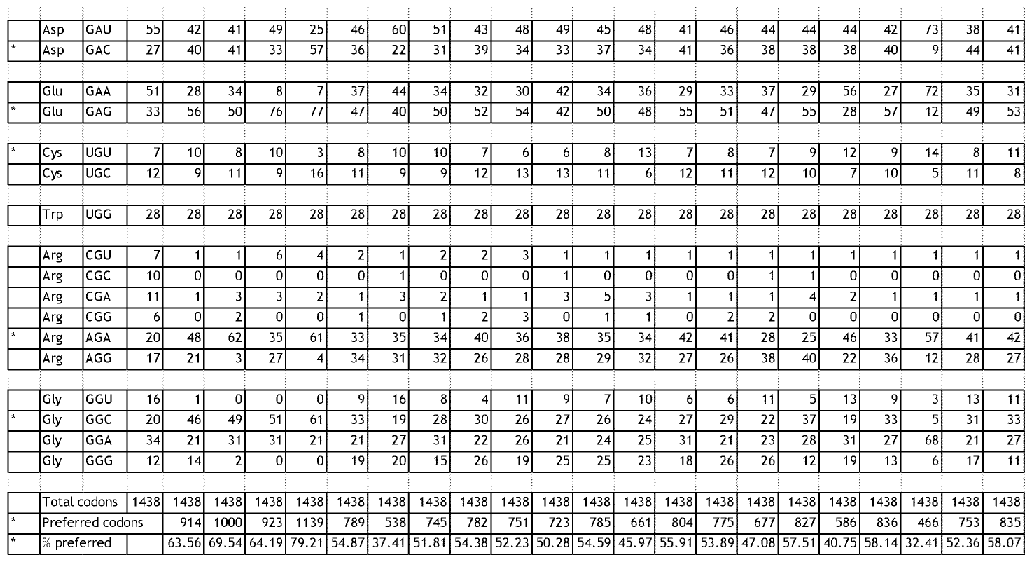

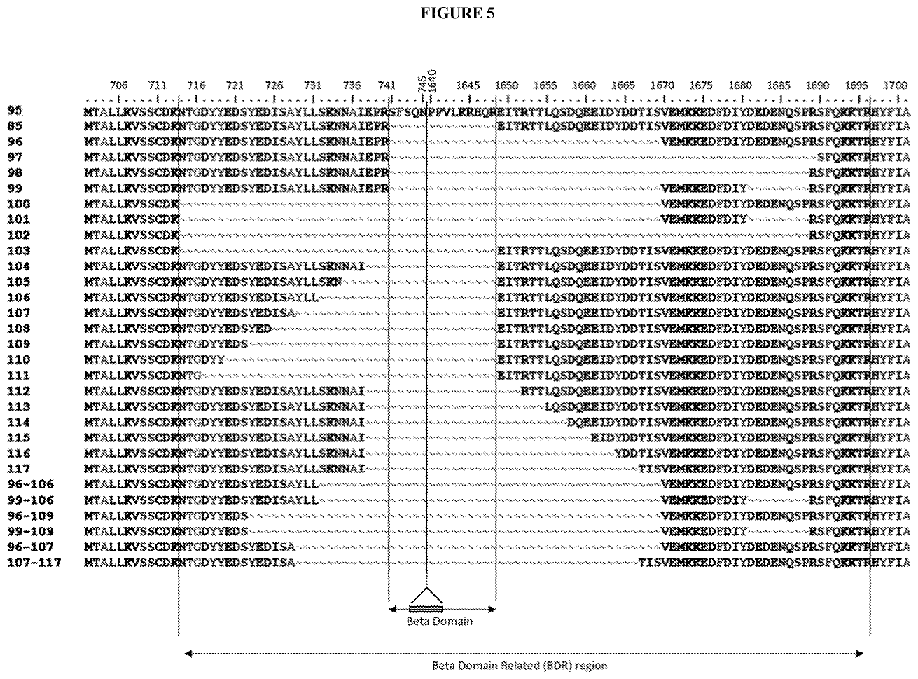

The output sequences were analysed and, where possible, CpG dinucleotides were removed (in some cases a CpG position was retained, for example if removing it would generate a potential cryptic splice set; sequences were also examined for the presence of these). A summary of the codon usage of the sequences is shown in FIG. 4.

The 21 codon-optimised FVIII-SQ sequences (named “Co1”, “Co2” etc.) were synthesised and cloned into a plasmid between the HLP2 promoter and bovine growth hormone poly A (BgpA, SEQ ID NO:66).

In Vitro Evaluation of Codon-Optimised FVIII-SQ Coding Sequences

Huh7 cells were transfected with the plasmids containing the respective codon-optimised FVIII-SQ sequences, using FuGENE® HD (Promega) transfection reagent and following the manufacturer's protocol.

To monitor the transfection efficiency, a plasmid expressing green fluorescent protein (GFP) from the strong, ubiquitously-expressed CMV promoter (CMV-GFP)—included as 10% of the total plasmid amount—was included in each case. The activity of secreted FVIII in the supernatant was measured at day 3 post-plasmid transfection by using a chromogenic assay (Biophen FVIII:C chromogenic assay —see Example 1), following manufacturer's instructions for end point quantification. GFP expression in the cells was measured using a FACS LSRFortessa X-20. GFP expression was used as internal control to normalise the FVIII activity.

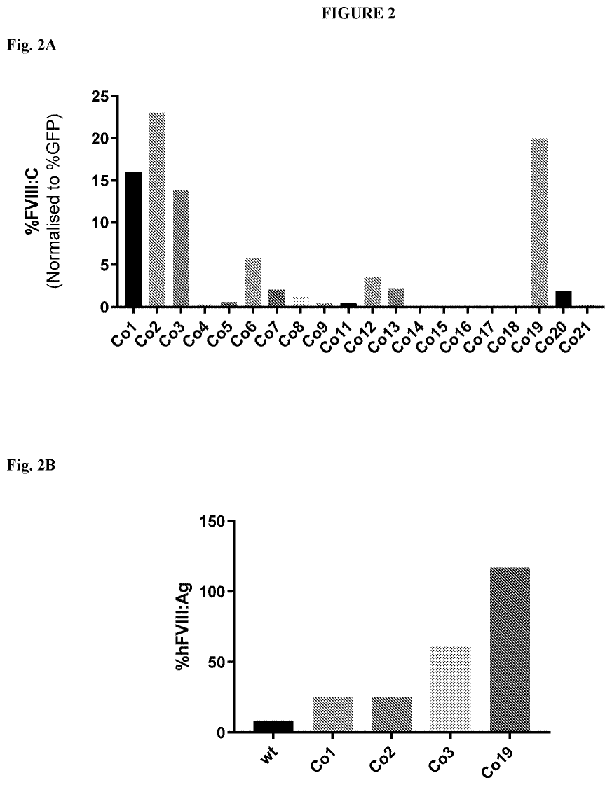

FIG. 2A shows that following plasmid transfection (and normalisation of FVIII activity to GFP expression) a high level of FVIII activity was detected from FVIII-SQ-col (SEQ ID NO:4), FVIII-SQ-cot (SEQ ID NO:5), FVIII-SQ-co3 (SEQ ID NO:6) and FVIII-SQ-co 19 (SEQ ID NO:3), compared to the other codon-optimised FVIII-SQ constructs. Accordingly, these constructs were selected for further characterisation in vivo.

In Vivo Evaluation of Codon-Optimised FVIII-SQ Coding Sequences

Based on the observation in vitro that FVIII-SQ-col, 2, 3 and 19 appeared to show the highest activity values, this experiment compared the level of FVIII-SQ antigen (i.e. expressed protein) expressed from these four codon-optimised sequences in vivo.

To assess FVIII-SQ antigen level in vivo, C57B1/6 mice were injected with single stranded AAV8 vectors containing the FVIII-SQ codon-optimised genes cloned between FRE1 promoter and BgpA poly A, i.e. AAV-FRE1-FVIII-SQ-col-BgpA, AAV-FRE1-FVIII-SQ-co2-BgpA, AAV-FRE1-FVIII-SQ-co3-BgpA and AAV-FRE1-FVIII-SQ-co19-BgpA constructs.

All AAV8 packaged viruses for this study were titered at the same time by qPCR method. 6-8 week old C57B1/6 mice were injected with 1.5×10¹²vg/kg of one of the single stranded AAV8 vectors described above. As a control, 1.5×10¹²vg/kg of ssAAV-FRE1-FVIII-SQ wt-BgpA (wild type coding sequence for FVIII-SQ (SEQ ID NO: 7)) was also administered intravenously. 5 mice were injected per vector.

Four weeks post-injection, blood was collected into citrate anticoagulant and FVIII-SQ antigen level was analysed by Asserachrom VIII:Ag ELISA kit (see Example 1) following manufacturer's instructions. As shown in FIG. 2B, the FVIII-SQ antigen data demonstrated higher FVIII-SQs levels from co3, and in particular col9 (all four codon-optimised sequences were improved relative to wild type). FVIII-SQ-col9 was selected as the codon-optimisation for use in the final FVIII construct.

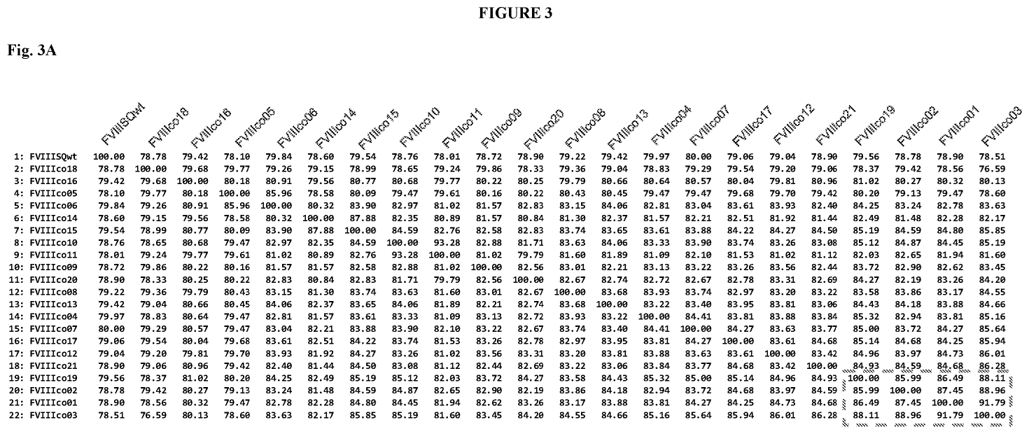

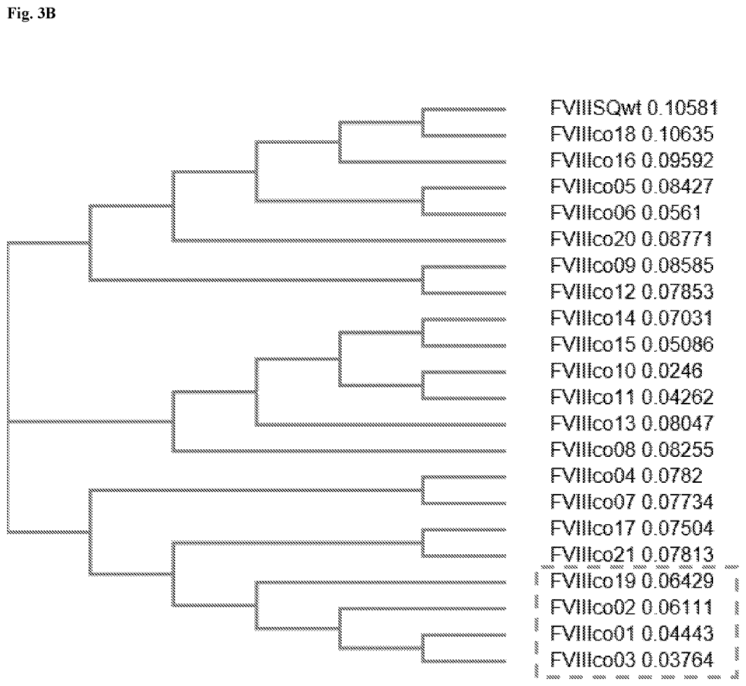

Interestingly, when the 21 codon-optimised sequences are multiply aligned using Clustalw (FIG. 3A), the four highest-expressing sequences are clustered by the program (FIG. 3A), reflecting that these sequences are a distinct group (boxed area in FIG. 3B) within the complete set of sequences on account of the higher degree of sequence identity within the group than between members of the group and sequences not in the group (FIG. 3A).

### Example 3 —internally-deleted FVIII variants to reduce size of FVIII transgene

Using the FVIII-SQ-cot codon-optimised sequence as a starting point, numerous shorter FVIII variant coding sequences were produced, wherein the encoded variants lacked the SQ linker as well as lacking additional stretches of sequence of various lengths immediately N-terminal, or C-terminal, or both, of the SQ linker, and within the region defined herein as the Beta Domain Related region (BDR region). FIG. 5 schematically depicts the deleted regions for each of the numbered variant sequences (variant ‘95’ is FVIII-SQ, while variant ‘85’ is FVIII RE (Lind et al. supra)), and also shows a number of ‘compound deletions’ comprising a combination of individual deleted stretches (e.g. the ‘96-106’ variant consisting of the deleted regions from the ‘96’ and ‘106’ variants).

To assess the impact of these deletions on the resulting shorter FVIII variant proteins, the variant coding sequences were cloned into commercially available expression vector pcDNA5-FRT (Invitrogen). Plasmid DNA was transfected into expi293 suspension cells (Invitrogen) in 96 Deep-well plates according to the manufacturer's protocol and incubated in a humidified 8% CO2 incubator at 37° C., shaking at 400rpm for 5 days. Cell cultures were then centrifuged at 1000×g for 5min at 4° C. and the supernatants collected for immediate testing in chromogenic activity, and antigen quantification (ELISA), assays described in Example 1.

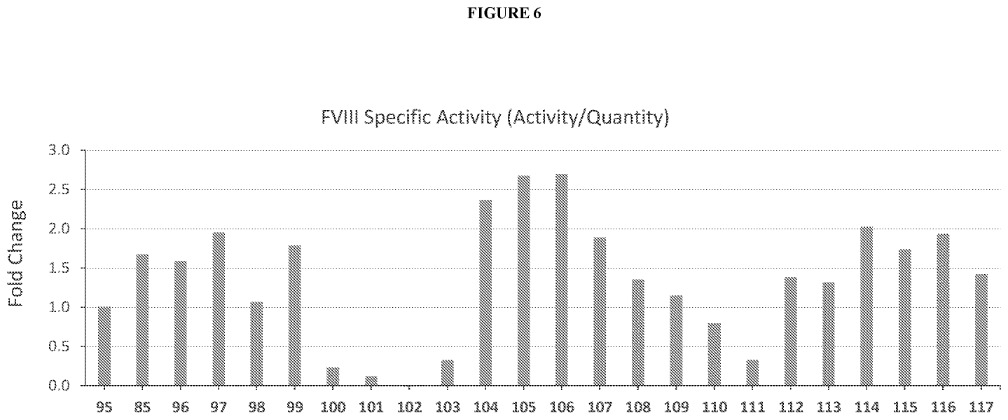

FVIII specific activity was calculated as the ratio: FVIII activity divided by FVIII quantity (antigen). FIG. 6 shows the fold-change in specific activity exhibited by the variants 96 to 117 relative to the FVIII-SQ (‘95’) starting point.

### Example 4—Shorter, Heterologous Alternatives to Native FVIII Signal Peptide

To investigate whether the FVIII transgene could be further shortened, and/or expression enhanced, a number of alternative signal peptides (SPs)—derived from highly-secreted liver proteins—were tested in the place of the native FVIII signal peptide (which is 57 base pairs in length).

Details of the signal peptides are provided in the below table:

| SP | Encoding sequence length |
|---|---|
| number | (base pairs) |
| SP2 | 54 |
| SP3 | 72 |
| SP4 | 54 |
| SP5 | 54 |
| SP6 | 57 |
| SP7 | 66 |
| SP8 | 72 |
| SP9 | 57 |
| SP10 | 54 |
| SP11 | 84 |
| SP12 | 84 |
| SP13 | 60 |
| SP14 | 57 |

In Vitro Evaluation of Heterologous Signal Peptides

FVIII-SQ-co2 sequences respectively comprising the various heterologous signal peptides (encoded by native nucleotide sequences) instead of the native FVIII signal peptides were synthesised and cloned into a plasmid between the FREI promoter and bovine growth hormone poly A (BgpA).

Huh7 cells were transfected with the plasmids using FuGENE® HD (Promega) transfection reagent and following the manufacturer's protocol.

To monitor the transfection efficiency, a plasmid expressing green fluorescent protein (GFP) from the strong, ubiquitously-expressed CMV promoter (CMV-GFP)—included as 10% of the total plasmid amount—was included in each case. The activity of secreted FVIII in the supernatant was measured at day 3 post-plasmid transfection by using a chromogenic assay (Biophen FVIII:C chromogenic assay —see Example 1), following manufacturer's instructions for end point quantification (data not shown). GFP expression in the cells was measured using a FACS LSRFortessa X-20. GFP expression was used as internal control to normalise the FVIII activity.

On the basis of relative activity levels and the lengths of the signal peptides, a subset of signal peptides including SPs 5 (amino acid sequence MKLLAATVLLLTICSLEG, SEQ ID NO:56), 8 (sequence MPSSVSWGILLLAGLCCLVPVSLA, SEQ ID NO:60) and 10 (sequence MKWVWALLLLAALGSGRA, SEQ ID NO:58) were tested in vivo.

In Vivo Evaluation of Heterologous Signal Peptides

From the preceding in vitro work, a subset of signal peptides were selected for in vivo testing, again using FVIII-SQ antigen quantity as the readout.

C57Bl/6 mice were injected intravenously with single stranded AAV8 vectors containing FVIII-SQ-cot sequences with the native FVIII signal peptide-encoding sequence (codon-optimised sequence), or the respective heterologous signal peptide-encoding sequences (native sequences) positioned immediately 5′ in place of the native FVIII signal peptide-encoding sequence. The resulting FVIII-SQ genes were cloned between HLP2 promoter and BgpA poly A.

All AAV8 packaged viruses for this study were titered at the same time by qPCR method.

6-8 week old C57Bl/6 mice were injected with 1.5×10¹²vg/kg of one of the single stranded AAV8 vectors described above. 5 mice were injected per vector.

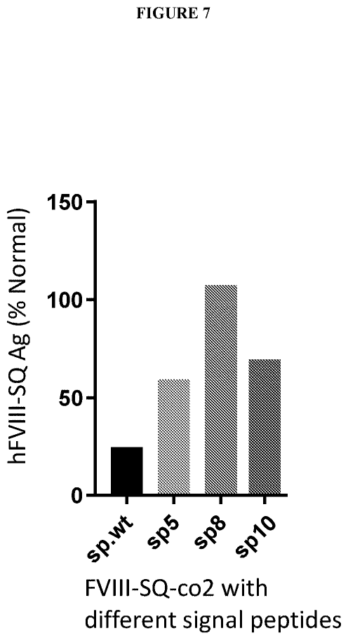

Four weeks post-injection, blood was collected into citrate anticoagulant and FVIII-SQ antigen level was analysed by Asserachrom VIII:Ag ELISA kit (see Example 1) following manufacturer's instructions. FIG. 7 reveals that each of SPs 5, 8 and 10 resulted in elevated FVIII antigen levels relative to the native FVIII SP. Taking into account the short length of SP5 and the good performance in human cells in vitro, SP5 was selected for use in the final FVIII construct.

Further In Vitro Evaluation of SP5

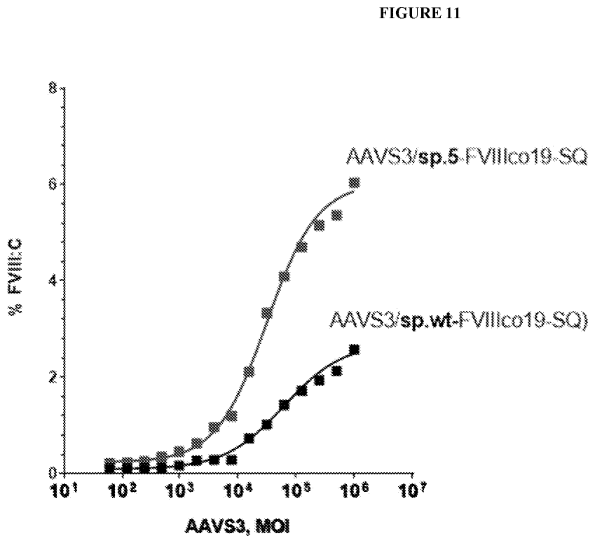

The activity of secreted FVIII in the supernatant was measured day 3 post-AAV transduction by using a chromogenic assay (Biophen FVIII:C chromogenic assay—see Example 1), following manufacturer's instructions for end point quantification. Huh7 cells were transduced with an AAVS3 expressing FVIIIco19-SQ coding sequences having a wild-type (native FVIII) signal peptide (codon optimised coding sequence) or signal peptide 5 (SP5) (native coding sequence). The AAV construct also comprised AAV2 ITRs, the FRE72 TRE and synthentic polyA (SpA). The activity was measured at different doses of the vector. As shown in FIG. 11, there was a higher level of FVIII secretion detected at various MOI when using SP5 compared to the wild-type signal peptide.

### Example 5—Additional Materials and Methods for Examples 6-8

FVIII constructs

The cDNA of human FVIII-SQ (encoding FVIII containing a 14 amino acid linker region in place of the B domain, as discussed above) was cloned into a liver-specific promoter-driven adeno-associated virus (AAV) vector.

Two different codon optimised FVIII variants (termed “co02” (SEQ ID NO: 5) and “col9” (SEQ ID NO: 3)) were used. To reduce the AAV recombinant genome size, a number of small liver-specific promoters were designed as set out below.

Generation of AAV Vectors

AAV particles were produced by triple plasmid transfection of HEK293T cells with plasmids encoding the AAV Rep and Cap functions; adenoviral helper functions; and the recombinant genome containing the FVIII expression cassette flanked by AAV2 ITRs. Cell pellet and supernatant were harvested 72 hours post-transfection and AAV particles purified by affinity chromatography using resins such as POROS Capture Select and AVB Sepharose. AAV was then dialysed into PBS overnight, stored at 4° C. and titred by qPCR.

### Example 6—Design and Selection of Small Liver-Specific Transcription Regulatory Elements

A number of different TREs were designed based on the HLP2 TRE and selected based on overall length. Deletions to the TRE were applied, based on looking at conserved regions in the related alpha-1-antitrypsin TRE in various vertebrates. It was surprisingly found that TREs could be made which are significantly shorter than the HLP2 TRE but which retain a degree of functionality. In some cases the level of activity is at least comparable to the HLP2 TRE, as shown in the examples below.

The transcription regulatory element FRE72 (SEQ ID NO: 51) was designed and compared to the HLP2 TRE.

In order to determine the minimum length required to obtain a transcription regulatory element having at least a basic level of functionality, three further transcription regulatory elements were designed and tested:
FRE46 consists of the “core nucleotide sequence” defined by SEQ ID NO: 43. FRE46 therefore corresponds to nucleotides 170-242 of SEQ ID NO:52 and is 73 nucleotides in length;
FRE47 consists of the “core nucleotide sequence” defined by SEQ ID NO: 43 together with a TSS sequence defined by SEQ ID NO: 44 located 3′ to the “core nucleotide sequence”. FRE47 therefore corresponds to nucleotides 170-242 plus nucleotides 297-302 of SEQ ID NO: 52 and is 79 nucleotides in length;
FRE48 consists of the “extended core nucleotide sequence” defined by SEQ ID NO: 50 together with a TSS sequence defined by SEQ ID NO: 44 located 3′ to the “extended core nucleotide sequence”. FRE48 therefore corresponds to nucleotides 163-242 plus nucleotides 297-302 of SEQ ID NO: 52 and is 86 nucleotides in length.

### Example 7—In Vitro Assessment

To evaluate the activity of the designed transcription regulatory elements (TREs) in vitro, hepatocyte derived cellular carcinoma cell line Huh7 was transiently transfected with candidate plasmids comprising the TREs (either HLP2 (for comparison purposes) or one of those defined in Example 6 above) positioned upstream of a designated “co02” codon-optimised transgene for a human clotting factor VIII variant (FVIII-SQ). The “co02” sequence is provided as SEQ ID NO: 5. The transgene and transcription regulatory element were flanked by ITRs from AAV2. In total 2.5×10⁵ Huh7 cells were seeded per well of a 12 well plate in DM EM low glucose +10% FBS +glutamax (D10 medium). Experiments were performed in triplicate.

For plasmid transient transfection, following FuGENE® HD Transfection protocol, 24 hours post cell seeding, 1.8 μg of the candidate plasmid and 0.2 μg CMV-Luciferase plasmids were mixed and added to FuGENE HD Reagent (8 _{f}iL). CMV-Luciferase plasmid (10% of the total plasmid) was included in each transfection in order to monitor the transfection efficiency.

Post transfection (around 18 hours later), the medium was replaced with 500 μl fresh DM EM low glucose+10% FBS+glutamax (D10 medium). 24 h later, medium was replaced by fresh DMEM low glucose+glutamax (D0 media). Cells and medium were harvested the following day, i.e. at day 3 post-transfection.

FVIII activity was assessed using the BIOPHEN FVIII:C(6) (ref. 221406) kit. The absorbance was measured on SpectraMax i3. In parallel, cells were lysed (Promega E397A lysis buffer) and subjected to a Luciferase assay (Promega E1501) to measure the Luciferase expression. Luciferase expression was used as an internal control to normalise the FVIII activity. Analysis was performed using the software Graphpad Prism v7.

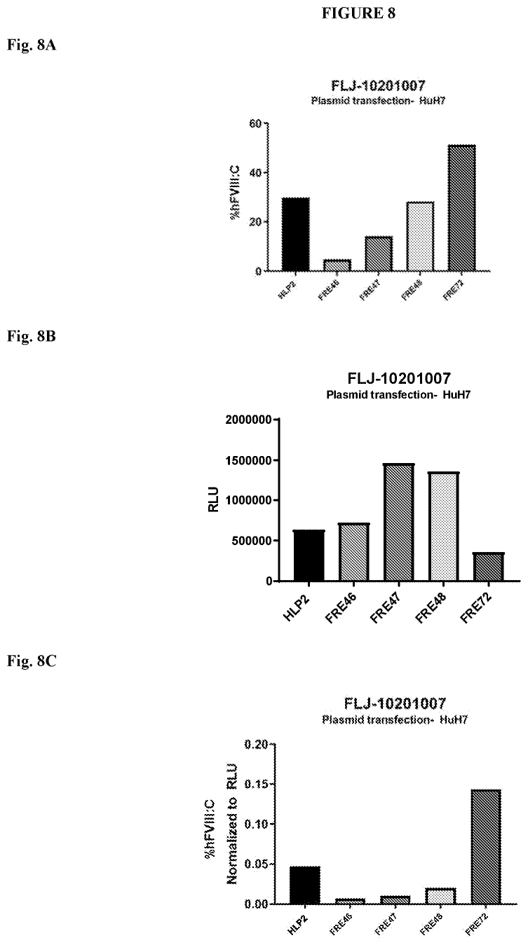

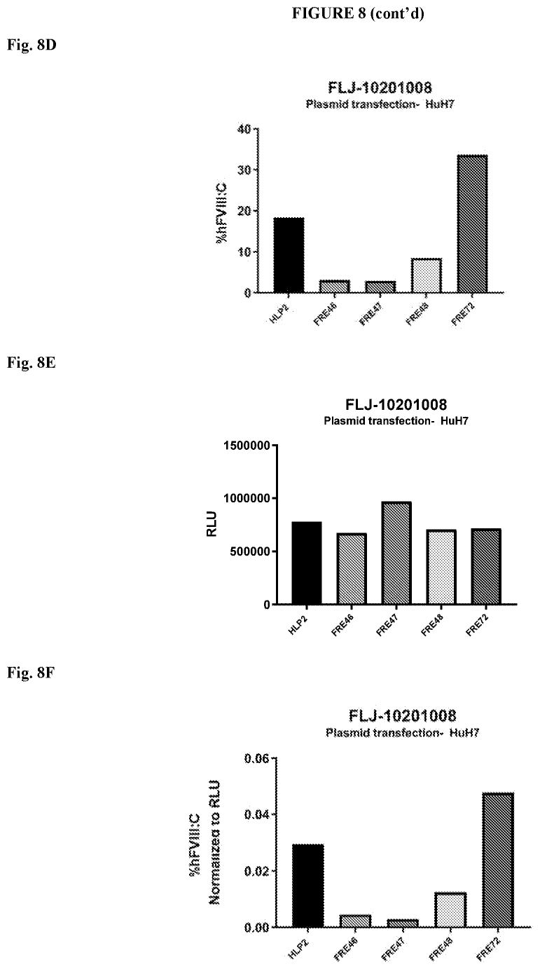

The results of the in vitro experiments are shown in FIG. 8 (panels A-C and D-F). The most relevant panels are FIG. 8C and 8F, which show the relative mean FVIII expression achieved by the various TREs when normalised to the level of transfection.

### Example 8—In Vivo Assessment

AAV particles were produced as described above having a genome comprising a codon-optimised nucleotide sequence (designated “col9”) encoding a human FVIII-SQ (i.e. a FVIII containing a 14bp linker region in place of the B domain, as described e.g. in Lind et al. 1995 supra) encapsidated by an AAV8 capsid. The “co 19” sequence is provided as SEQ ID NO: 3. AAV particles were produced as described above by triple plasmid transfection of HEK293T cells with plasmids encoding the AAV Rep and Cap functions; an adenoviral helper function;

and the recombinant genome containing the respective TRE to be tested, positioned upstream of the “co19” FVIII-SQ expression cassette, with the genome being flanked by AAV2 ITRs. Cell pellet and supernatant were harvested 72 hours post-transfection and AAV particles purified by affinity chromatography using resins such as POROS Capture Select and AVB Sepharose. AAV was then dialysed into PBS overnight, stored at 4° C. and titered by qPCR.

6-8-week-old male C57BL/6 mice were intravenously injected with 2×10¹² vg/kg viral vector. Six mice were injected per construct. On day 28 post-injection the mice were culled, and blood harvested into citrate anticoagulant. Blood and murine liver were provided for analysis.

Blood was used to carry out FVIII analysis while liver biopsies were used to calculate the vector genome.

To determine the number of vector genomes per liver cell post-AAV injection, DNA was isolated from frozen liver samples, approximately 40 mg, using QIAGEN DNeasy Blood and Tissue Kit (QIAGEN) following manufacturers' instructions. Quantitative real-time PCR (q-PCR) amplification was carried out using the PowerUp SYBR Green Master mix (Applied Biosystems) according to the manufacturer's instructions. q-PCR was performed on a QuantStudio™ instrument (Applied Biosystems). The primer sets are designed to quantify the transgene, allowing an estimation of AAV copy number. Genome copy number was calculated from the standard curve and after normalization to mouse GAPDH quantified by qPCR.

To determine the levels of FVIII protein post-AAV injection, FVIII antigen level from citrated plasma was measured by Asserachrom VIII:Ag ELISA kit (Diagnostica Stago) following manufacturer's instructions. Further dilutions were performed when deemed necessary.

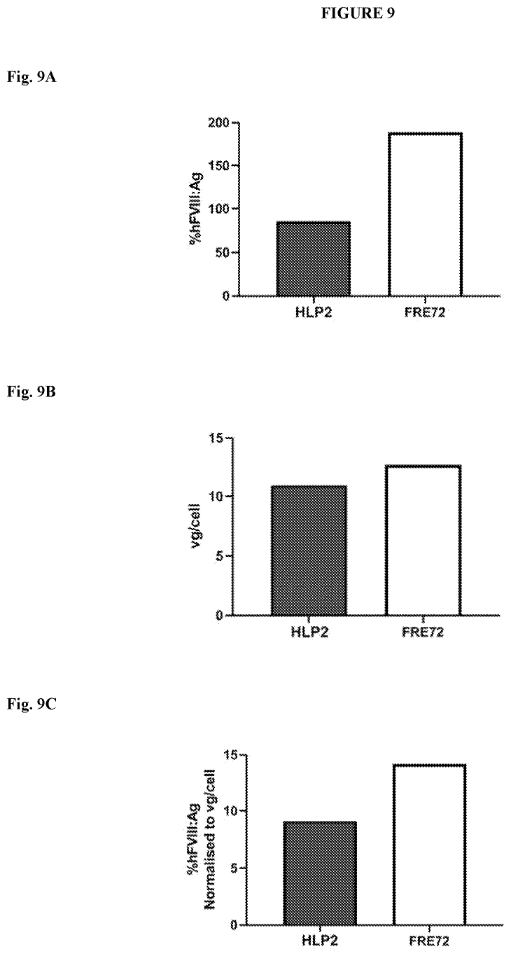

Results are shown in FIG. 9. The most relevant panel is FIG. 9C, which shows the relative FVIII level, normalised to the viral genome level per cell.

### Example 9—In Vivo Evaluation of Internally-Deleted FVIII Transgene Construct

As a rapid way to comparatively assess construct performance in vivo, hydrodynamic tail vein plasmid injection (HDTVI) was used to compare FVIII variant ‘96-106’, carrying a deletion of 132 base pairs relative to FVIII-SQ, against FVIII-SQ, in terms of FVIII activity. These FVIII variant coding sequences, comprising non-codon-optimised sequences encoding heterologous signal peptide 10 (SP10) in place of the native FVIII signal peptide, were cloned (without promoter), into plasmid pLIVE under the control of mouse albumin promoter.

Sp10-FVIIIco19-SQ, Sp10-FVIIIco19-(96-106) and comparator FVIIIco-SQ sequences were cloned into pLIVE plasmid, under the control of albumin promoter, upstream of the polyadenylation site of pLIVE plasmid.

On day 0, 8-16 weeks old C7BL/6 Factor VIII-knockout (FVIII-KO) mice were weighed in order to calculate an accurate dose. Plasmid DNA was diluted to a total of 25 μg in a volume of isotonic dosing solution volume equal to 10% of body weight (v/w) and warmed to 25-37° C. prior to injection. (For example, 2.5 ml injection volume for 25 g mouse.) Plasmid DNA was diluted immediately before dosing as this is known to improve expression.

Dosing solution was drawn into a sterile 5 ml syringe, clearing any air bubbles. Mice were warmed in a heat box set to 38° C. for 10 min prior to administering isoflurane anaesthesia in order to dilate the tail vein. Whilst maintaining anaesthesia via a nose cone, mice were injected via the tail vein with dosing solution at a defined pressure over a period of 4-8 s. Heart rate and oxygen saturations were recorded throughout using a portable oxygen saturation monitor to enable monitoring of the expected changes in these parameters. Once the needle was removed, slight pressure was applied to stop any bleeding. Isoflurane anaesthesia was removed and the animal allowed to recover in a warm environment.

The plasmids injected included:
pLIVE-SP10-FVIIIco19-SQ
pLIVE-SP10FVIIIco19-(96-106)
pLIVE-comparator-FVIIIco-SQ

pLIVE-[SP10-FVIIIco19-SQ] contains the FVIII-SQ variant, encoded by the ‘co19’ codon-optimisation, with (non-codon-optimised) SP10 signal peptide. The transgene is under the control of the mouse albumin promoter and utilises the pLIVE polyA sequence. pLIVE-[SP10-FVIIIco19-(96-106)] is identical except for the encoded FVIII variant contains the 96-106 deletion. pLIVE[comparator-FVIIIco-SQ] contains a codon-optimised FVIII-SQ with native signal peptide corresponding to SEQ ID NO: 54, and is encoded by SEQ ID NO:37. 3 days post injection, between 100 and 200 μl of blood was collected from each mouse by retro-orbital puncture with non-heparinised blue capillary tubes on citrate (1:9 ratio of the total volume of blood). Plasma was prepared by centrifugation for 20 min at 4000 rpm.

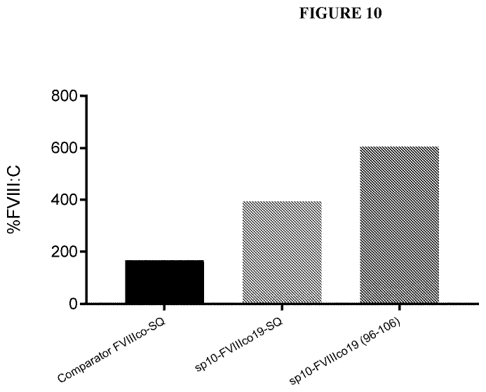

The plasma from animals injected with the above plasmids was analysed for FVIII activity by chromogenic assay described in Example 1. FVIII:C activity for each construct is shown in FIG. 10.

### Example 10—Assessing Tissue Specificity of FRE72

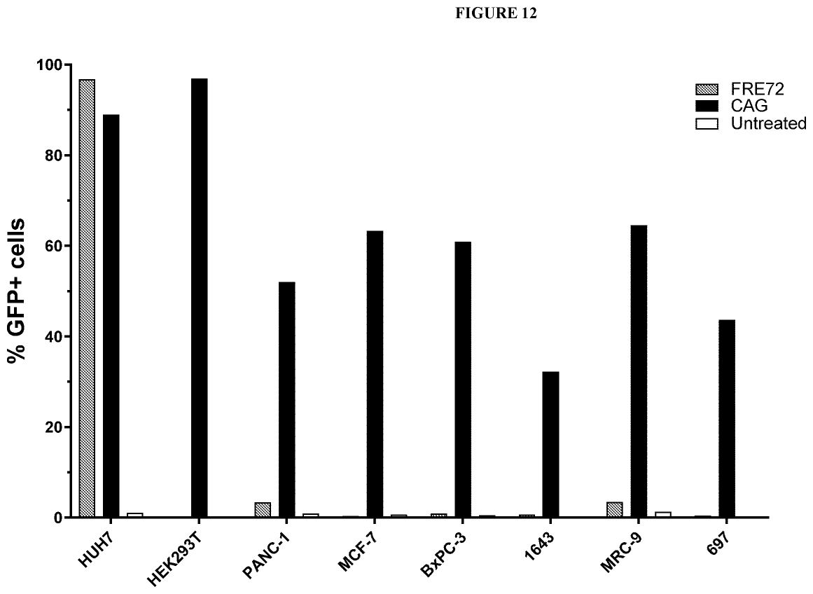

FIG. 12 shows the results of in vitro studies into the promoter fidelity of FRE72. FRE72 promoter fidelity was assessed in cell lines from a range of different tissues; Huh7: liver. HEK293T: kidney. PANC1: pancreas. BxPC-3: pancreas. MCF7: breast. 1643: neuroblastoma. MRC-9: lung. 697: early B cell. Cells were transduced with the control vector AAVS3.CAG.GFP or AAVS3.FRE72.GFP or untreated at MOI of 1×10⁵. FIG. 12 shows three columns for each cell type; the left hand column for each cell type (grey) relates to cells transduced with AAVS3.FRE72.GFP; the central column for each cell type (black) relates to cells transduced with the control vector; and the right hand column for each cell type (white) relates to untreated cells. For the HEK293T and MCF-7 cells the left hand (“grey”) column is so small that it is not visible in FIG. 12; similarly for the HEK293T, 1643 and 697 cells the right hand (“white”) columns is so small that it is not visible in FIG. 12.

### Example 11—In Vitro Evaluation of Relative Specific Activity of FVIII Substitution Mutation Variants

In silico modelling was used to predict single amino acid substitutions, and pairs of cysteine substitutions, on non-surface-exposed inter-domain surfaces, which might increase the stability of FVIII. However, any impact on stability does not necessarily equate to a beneficial effect on activity, and in some cases increased stability would have a deleterious effect, e.g. a substitution increasing stability but not allowing the necessary FVIII domain rearrangement into an active form. Numerous such substitution variants were tested in vitro as described below.

Codon-optimised nucleotide sequences encoding FVIII-SQ variants comprising one or more such substitutions were gene-synthesised and cloned into the commercially available expression vector pcDNA5-FRT (Invitrogen). Plasmid DNA was transfected into expi293 suspension cells (Invitrogen) in 96 Deepwell plates according to the manufacturer's protocol, and incubated in a humidified 8% CO₂ incubator at 37° C. shaking at 400rpm for 5 days. Cell cultures were centrifuged at 1000×g for 5min at 4° C. and the supernatants filtered through 0.2 μl filter for immediate testing in chromogenic assay and antigen quantification as described in Example 1.

Following blank subtraction, the FVIII activity readout from the chromogenic assay was divided by the FVIII antigen quantification (ELISA) readout to obtain the specific activity (SA) value.

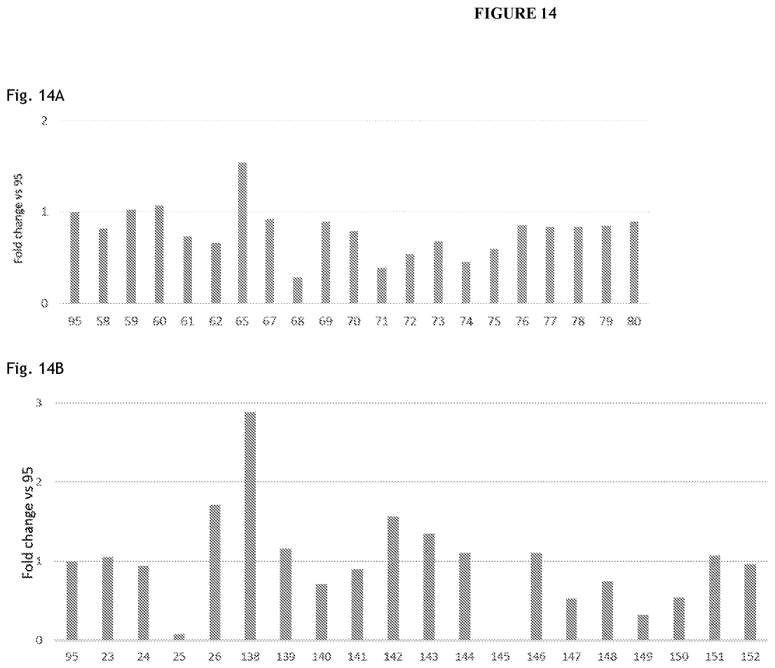

FIG. 14A shows the fold-change in SA, relative to the FVIII-SQ (‘95’) control lacking any substitution mutations, for several different single amino-acid substitution variants, including a number of alternative substituted residues for each variant. The following table shows the identity of the substitutions corresponding to each of the numbered constructs. Variant 65 (H693W) exhibits an elevation in SA relative to 95.

| Construct no. | 58 | 59 | 60 | 61 | 62 | 65 | 67 | 68 |
|---|---|---|---|---|---|---|---|---|
| Position | L687 | | H693 | | | | N694 | |
| Substituted residue | R | W | R | I | K | W | E | W |

| Construct no. | 69 | 70 | 71 | 72 | 73 | 74 | 75 | 76 | 77 | 78 | 79 | 80 |
|---|---|---|---|---|---|---|---|---|---|---|---|---|
| Position | S695 | | | | | | | D696 | | | | |
| Substituted | R | I | L | M | F | W | Y | R | Q | F | W | K |
| residue | | | | | | | | | | | | |

In another experiment, the substitution M662W (called ‘26’) had demonstrated a marked increase in SA relative to ‘95’. For position M662 it was decided to screen the SA of all alternative substitutions. The results are shown in FIG. 14B, which also includes a double substitution, combining M662W with H693W (65 from FIG. 14A). Again see below table for the identity of the substitutions. Substitutions at position M662 with C and E appear to give some uplift in SA relative to 95. The greatest increase in SA was observed for the M662W+H693W combination.

| Construct no. | 23 | 24 | 25 | 26 | 138 | 139 | 140 | 141 | 142 | 143 |
|---|---|---|---|---|---|---|---|---|---|---|
| Position | M662 | | | | | | | | | |
| Substituted | N | Q | I | W | W + | A | R | D | C | E |
| residue | | | | | H693W | | | | | |

| Construct no. | 144 | 145 | 146 | 147 | 148 | 149 | 150 | 151 | 152 |
|---|---|---|---|---|---|---|---|---|---|
| Position | M662 | | | | | | | | |
| Substituted | G | P | S | Y | H | L | K | F | T |
| residue | | | | | | | | | |

### Example 12—In Vitro Evaluation of Relative Specific Activity of Additional FVIII Substitution Mutation Variants Predicted to Form Disulphide Bridges

Based on the in silico prediction work in Example 11, a number of pairs of cysteine substitutions were tested using the methodology described in Example 11.

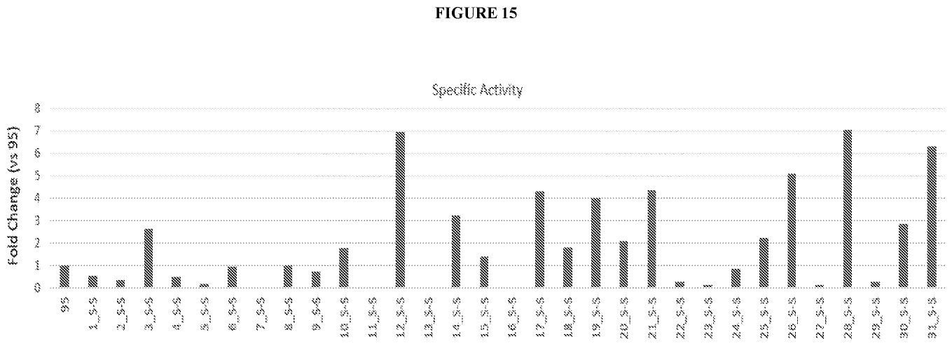

The results—again expressed as fold-change in specific activity relative to FVIII-SQ (‘95’) (SEQ ID NO:3)—are shown in FIG. 15. The below table indicates the substitution pairs corresponding to the construct numbers shown in FIG. 15. Several of the C-C substitution variants show several-fold increases in specific activity relative to control.

| Construct no. | 1_S-S | 2_S-S | 3_S-S | 4_S-S | 5_S-S | 6_S-S | 7_S-S | 8_S-S | 9_S-S |
|---|---|---|---|---|---|---|---|---|---|
| C | S285C | E287C | S289C | T646C | D647C | K659C | Y664C | T667C | T667C |
| substitution | E676C | F673C | N1977C | N1950C | Y1979C | M1823C | K1967C | S1788C | A1836C |
| pair | | | | | | | | | |

| Construct no. | 10 S-S | 11 S-S | 12 S-S | 13 S-S | 14 S-S | 15 S-S | 16 S-S | 17 S-S | 18 S-S |
|---|---|---|---|---|---|---|---|---|---|
| C substitution | T669C | F671C | L687C | W688C | I689C | F697C | G102C | A108C | T118C |
| pair | V1982C | Y1979C | A1800C | S710C | G1799C | S1949C | A1974C | Q2329C | N2172C |

| Construct no. | 19_S-S | 20_S-S | 21_S-S | 22_S-S | 23_S-S | 24_S-S | 25_S-S | 26_S-S | 27_S-S | 28_S-S |
|---|---|---|---|---|---|---|---|---|---|---|
| C | V137C | M147C | S149C | P264C | I291C | N280C | T667C | T669C | S268C | N684C |
| substitution | Y2332C | E1970C | E1969C | E1951C | S1955C | S524C | G1981C | Y1979C | P672C | S1791C |
| pair | | | | | | | | | | |

| Construct no. | 29 S-S | 30 S-S | 31 S-S |
|---|---|---|---|
| C substitution | G686C | L687C | S695C |
| pair | R1803C | R1803C | E1844C |

### Example 13—Confirmatory In Vitro Testing of Particular Promising Substitution Variants

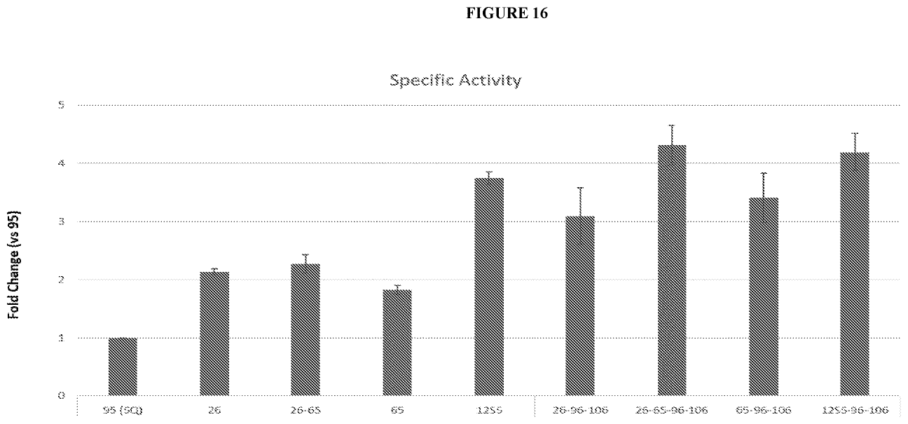

FIG. 16 confirms, for a subset of tested substitution variants of Examples 11 and 12, the fold-increase in SA relative to the FVIII-SQ (‘95’) control. The substitutions were evaluated in two FVIII ‘backgrounds’, the first being FVIII-SQ as in the above Examples:
FVIII-SQ-M662W (‘26’)
FVIII-SQ-H693W (‘65’)
FVIII-SQ-M662W-H693W (‘26-65’)
FVIII-SQ-L687C-A1800C (‘12SS’)

The second FVIII background differs from FVIII-SQ in that it contains a more extensive internal deletion encompassing, and extending on either side of, the B domain. The deleted region corresponds to amino acids positions 732-1669, and the variant protein is referred to as FVIII-(96-106).
FVIII-(96-106)-M662W (‘26-96-106’)
FVIII-(96-106)-H693W (‘65-96-106’)
FVIII-(96-106)-M662W-H693W (‘26-65-96-106’)
FVIII-(96-106)-L687C-A1800C (‘12S S-96-106’)

As FIG. 16 shows, each of the above subset of tested constructs showed an approximately 2 or more fold increase in specific activity relative to FVIII-SQ control (95).

### Example 14—In Vivo Evaluation of Internally-Deleted FVIII Transgene Construct Containing Stabilising Substitution Mutation

In this study, AAV8 vectors were made from the following constructs:
FRE72-SP5-FVIIICo19 (26-96-106)-SpA (SEQ ID NO:71)
FRE72-SP5-FVIIICo19-SQ-SpA (SEQ ID NO:73)

The above constructs contain the FRE72 promoter, signal peptide 5, and synthetic polyA. They differ in that one encodes FVIII-SQ, whilst the other encodes the shorter variant with the “96-106” internal deletion, as well as the substitution mutation “26” (=M662W).
Comparator FVIIIco-SQ

The above construct comprises a codon-optimised FVIII-SQ-encoding sequence with native FVIII signal peptide, SpA, and a liver-specific promoter. The construct has the sequence of SEQ ID NO: 72.

The AAV8-Comparator FVIIIco-SQ was respectively compared against AAV8-FRE72-5P5-FVIIICo19 (26-96-106)-SpA and AAV8-FRE72-SP5-FVIIICo19-SQ-SpA in separate experiments.

6-8 week old C7BL/6 Factor VIII-knockout (FVIII-KO) mice were intravenously injected with 2×10¹² vg/kg of one of each of the above vectors. All AAV8 vectors for this study were titered at the same time by qPCR method.

At 6 weeks post injection, between 100 and 200 μl of blood was collected from each mouse by retro-orbital puncture with non-heparinised blue capillary tubes on citrate (1:10 dilution). Plasma was prepared by centrifugation for 20 min at 4000 rpm.

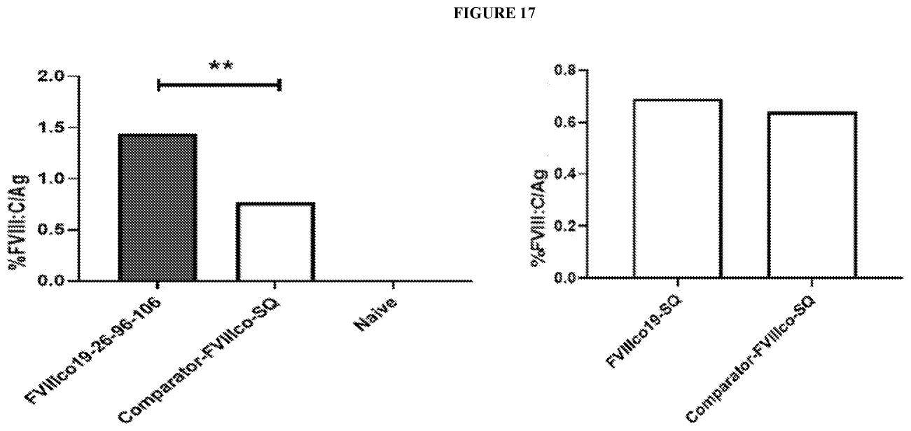

The plasma from injected and naïve animals were analysed for FVIII activity and human FVIII antigen level (assays as described in Example 1), and the ratio of FVIII activity to antigen level (i.e. specific activity) was calculated (FIG. 17).

### Example 15—Comparative Evaluation of Manufacturability and Product Quality of Internally-Deleted FVIII Transgene Construct

The impact of the minimised 4,713 nucleotide vector genome length enabled by the FRE72-SP5-EVIIICo19 (26-96-106)-SpA construct on manufacturing and quality parameters was assessed and compared against the 4,970 nucleotide comparator FVIIIco-SQ vector genome described in Example 14.

Cell Cultivation

HEK293T cells were maintained in adherent culture under standard conditions at 37° C., 95% relative humidity, and 5% v/v CO₂ in Dulbecco's Modified Eagle's Medium (DMEM) supplemented with 10% Fetal Bovine Serum (FBS) and 1% GlutaMai^{rm} (L-alanine-L-glutamine dipeptide). Cellular confluence during passaging ranged from 40-95%.

Transfection of HEK293T Cells and Preparation of Cell Lysates

HEK293T cells were transfected with helper plasmid (comprising the adenoviral helper functions and AAV2 rep gene) and vector plasmid (comprising an engineered cap gene (encoding SEQ ID NO: 68), and FRE72-5P5-EVIIICo19 (26-96-106)-SpA or comparator FVIIIco-SQ vector cassettes; see Example 14) using molar plasmid ratios of 1:3 (helper plasmid:vector plasmid) while maintaining the total plasmid DNA amount for all setups. The trans-split two plasmid system is described in WO 2020/208379. 6.0×10⁴ viable cells per cm² culture area were seeded in 15-cm dishes in a volume of 22 ml DM EM containing 10% FBS and 1% GlutaMax™ the day before transfection resulting in approximately 80% confluency on the day of transfection. PEI-DNA complexes were prepared in DMEM without supplements using the linear polyethylenimine transfection reagent PEIpro™ (Polyplus) according to the manufacturer's manual. An amount of 42 μg total plasmid DNA and a PEI-to-DNA ratio of 2:1 was maintained independent of the applied plasmids. Cells were cultured until day 3 post-transfection, harvested in the medium and lysed by three freeze-thaw cycles. Cell debris was removed by centrifugation at 3,700×g for 30 min.

Quantification of rAAV Vector Genomes

The AAV vector genome assay is based on a quantitative polymerase chain reaction (qPCR) specific for a sequence of the rAAV expression cassette.

Cell lysate test samples were subjected to a nuclease treatment procedure in order to remove non-packed vector genomes prior to performing the qPCR. To that aim the samples were pre-diluted 1:250 in nuclease-free water containing 0.124% Pluronic F-68. 25 μl of the pre-dilution were used for the digest with 2 units of Turbo DNase (ThermoFisher Scientific, Waltham, USA) and 1× Turbo DNase reaction buffer, resulting in a total reaction volume of 29 μl. Incubation was performed for 1 h at 37° C. Afterwards, 1 volume of 0.4 M NaOH was added and the samples were incubated for 45 min at 65° C. 1035 μl nuclease-free water supplemented with 0.1% Pluronic F-68 were added together with 30 μl 0.4 M HCl. To control for the quality of the Turbo DNase digest, a trending control containing unpurified cell lysate with a known AAV vector genome titre and spike-in controls using plasmid DNA carrying the transgene sequence were measured in parallel.

Per sample, 12.5 μl QuantiFast SYBR Green PCR Master Mix (Qiagen, Venlo, Netherlands) were mixed with 0.75 μl of qPCR primer working stock solution (containing 10 μM of each primer) and filled up to a volume of 20 μl with nuclease-free water. 5 μl of Turbo DNase treated cell lysate or purified virus test sample were added to the mix (total reaction volume 25 μl, final primer concentration in the reaction 300 nM each) and qPCR was performed in a CFX 96 Touch Real Time PCR cycler (Bio-Rad Laboratories Inc., Hercules, USA) with the following program steps: 95° C. 5 mM; 39 cycles (95° C. 10 s, 60° C. 30 s, plate read); 95° C. 10 sec; 60-95° C. (+0.5° C./step), 10 sec; plate read. To control for the quality of the qPCR, a trending control with known AAV vector genome titre was measured in parallel. To check for contaminants, a no template control (NTC, 5 μl H₂O) was also included. Standard row, test samples and controls were measured in triplicate for each dilution. Purified virus test samples and trending controls were generally measured in 3 different dilutions in EB buffer (10 mM Tris-Cl, pH 8.5). Turbo DNase treated cell lysate test samples were directly used in the qPCR without any further dilution. Data were analysed using the CFX Maestro Software 1.1 (Bio-Rad Laboratories Inc.).

Melting curve analysis confirmed the presence of only one amplicon. Amplification results in nascent double stranded DNA amplicons detected with the fluorescent intercalator SYBR Green to monitor the PCR reaction in real time. Known quantities of the genetic material, in the form of a linearized plasmid, were serially diluted to create a standard curve and sample vector genome titre was interpolated from the standard curve.

Quantification of rAAV Particles (Capsids)

The AAV2 Titration ELISA method is a measure of total AAV particles (capsids) and is based on a commercially available kit (Progen™, Heidelberg, Germany; catalogue number PRATV). This sandwich immunometric technique utilises monoclonal antibody A20 (Wobus et al (2000), J Virol, 74:9281-9293) for both capture and detection. The antibody is specific for a conformational epitope present on assembled capsids of serotypes AAV2, AAV3, and the engineered capsid used in these experiments.

The AAV2 Titration ELISA kit was used to quantify total AAV particles in cell lysates and purified virus preparations according to the manufacturer's instructions. In brief, 100 μl diluted AAV2 Kit Control, test samples, or trending control of engineered capsid with known total particle titre were added per well of a microtiter plate coated with monoclonal antibody A20 and incubated for 1 h at 37° C. Standard row, test samples and controls were measured in duplicates for each dilution. In a second step, 100 μl of pre-diluted biotin-conjugated monoclonal antibody A20 (1:20 in Assay Buffer [ASSB]) were added and incubated for 1 h at 37° C. Then, 100 μl of a pre-diluted streptavidin peroxidase conjugate (1:20 in ASSB) were added and incubated for 1 h at 37° C. 100 μl substrate solution (TMB [Tetramethylbenzidine]) were added and after incubation for 15 min, the reaction was stopped using 100 μl stop solution. The absorbance was measured photochemically at 450 nm using the SpectraMax M3 microplate reader (Molecular Devices, San Jose, USA). Data was analysed with the SoftMax Pro 7.0 Software (Molecular Devices).

The test samples were diluted into the assay range and AAV total particle concentrations were determined by interpolation using the standard curve which was prepared using the provided AAV2 Kit Control. ASSB was used as blank.

Vector Genome to Total Particle Ratio

The ratio of vector genomes to total AAV particles is calculated as a percentage. This is based on the vector genome titre (determined by qPCR, as described above) and the number of total AAV particles (determined by the capsid ELISA, as described above).

Quick Purification of rAAV Particles for Impurity Quantification

Clarified freeze-thaw lysates were nuclease treated to remove any non-packaged DNA followed by a one-step affinity chromatography step using AVB Sepharose HP (GE Lifesciences, Chicago, Ill.).

Quantification of Encapsidated Non-Vector Nucleic Acids

Prokaryotic DNA sequences, such as antibiotic resistance genes or parts of them originating from the bacterial backbone of the producer plasmids, can be packaged into the rAAV particles, constituting product-related impurities. Plasmid-derived impurity quantification is based on qPCRs specific for sequences of the kanamycin resistance gene (kanR), present on both helper and vector plasmid, and the AAV cap gene, which is present on the vector plasmid. Plasmid-derived impurity qPCRs were performed on the purified material from the preceding step according to the method for quantification of rAAV vector genomes outlined above with appropriate modifications to reflect the impurity templates, such as the primer pairs, standards and controls. Specifically, modifications were made to the impurity specific primer pairs, the linearized plasmid standards, the trending controls and the annealing temperatures (57° C.) in the qPCR setup.

Determination of Vector Integrity

Vector integrity, i.e. packaging of full-length vector genomes as well as homogeneity of the vector preparation (mispackaging of vector fragments or non-vector nucleic acids) was analyzed by alkaline gel electrophoresis. This assay is performed under alkaline conditions in the presence of sodium dodecyl sulfate, which frees viral genomes from AAV particles and keeps them in single stranded DNA conformation during electrophoretic separation. These DNA strands then migrate through the gel as a function of their respective size. For this assay, 1% agarose gel was prepared by heating lg of agarose in 100 ml of autoclaved deionized water. After cooling to 50° C., 2 ml of 50× alkaline electrophoresis buffer were added and the gel was cast into a 15×15 cm gel form with a thickness of approximately 0.5 to 0.7 mm and equilibrated for 15 —30 min at 4° C. Test samples were diluted to a final concentration of 1.7×10¹¹ vg/ml with nuclease-free water. For 10 μl of each sample, 2 μl of loading buffer containing 15 ng of a 1252 bp long, linear fragment of pUC19 vector as a loading control, were added and 12 μl of this mixture were loaded onto the gel. To allow size determination of nucleic acids from 200 bp to 10 kb, a molecular weight marker (Hyperladder, Bioline, London, UK) was included in the gel electrophoresis. The gel was run for 6 hat 60 V and 4° C. (2.7 V/cm) towards the cathode. After the run, the gel was equilibrated in 0.5 M Tris-HCl buffer at pH 7.9 for 15 min and then stained in 0.1 M Tris-HCl staining buffer containing 1× SybrGold (Life Technologies, Carlsbad, Calif.) for 90 min. Image acquisition of the stained gel was done on a FusionFX-7-826.EPI/SuperBright Advanced system (Vilber, Collégien, FR) with an excitation wavelength of 312 nm using the associated Fusion Capt Advance software (Vilber) for acquisition. The Bio 1D software (Vilber) was used for image processing and analysis.

Results

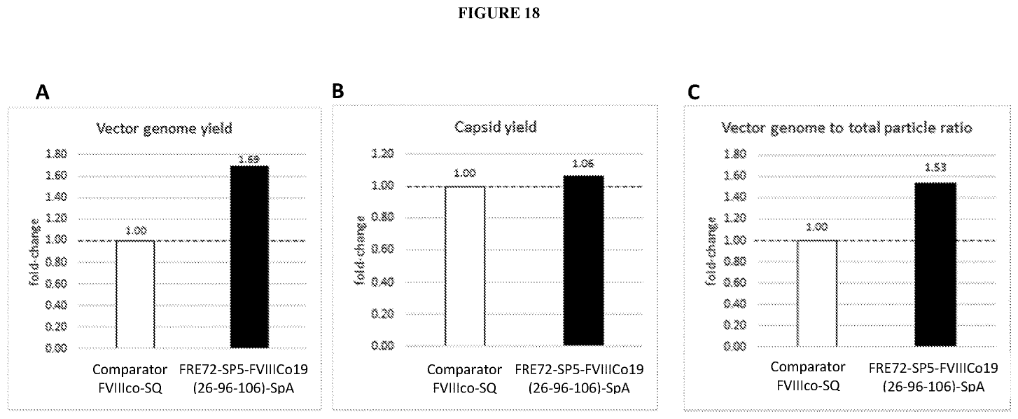

As is apparent from FIG. 18 the FRE72-SP5-FVIIICo19 (26-96-106)-SpA construct results in higher vector (FIG. 18A) and capsid (FIG. 18B) yields in comparison to a comparator FVIIIco-SQ construct of larger size, when the two constructs are manufactured in the same trans-split, two plasmid manufacturing system. The relative increase of vector genomes in comparison to particle (capsid) yields was more pronounced (1.69-fold vs 1.06-fold), resulting in a higher vector genome to total particle ratio (FIG. 18C) for the FRE72-SP5-FVIIICo19 (26-96-106)-SpA construct (1.53-fold).

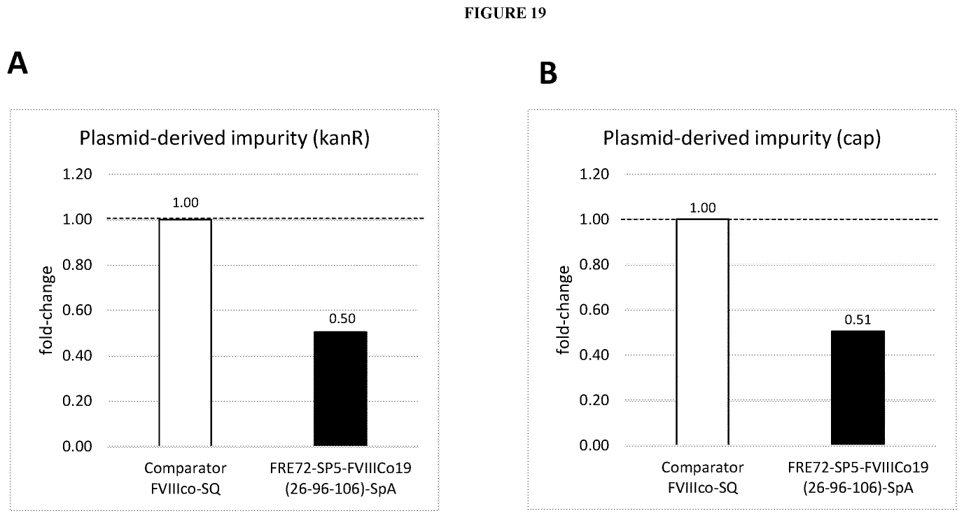

Quantification of packaged plasmid-derived impurities, originating from the producer plasmids, demonstrated reduced mispackaging for the FRE72-SP5-FVIIICo19 (26-96-106)-SpA construct in comparison to the comparator FVIIIco-SQ construct independent of the impurity marker analyzed (FIGS. 19A and B). For mispackaged sequences originating from the kanamycin antibiotics resistance gene (kanR), a reduction to 50% of mispackaged sequences was shown for the FRE72-SP5-FVIIICo19 (26-96-106)-SpA construct in comparison to the comparator FVIIIco-SQ construct (FIG. 19A). A comparable reduction was demonstrated for rAAV capsid (cap)-derived sequences (reduction to 51%, FIG. 19B). Reductions indicate an improved quality profile of the FRE72-SP5-FVIIICo19 (26-96-106)-SpA construct compared to the comparator FVIIIco-SQ construct.

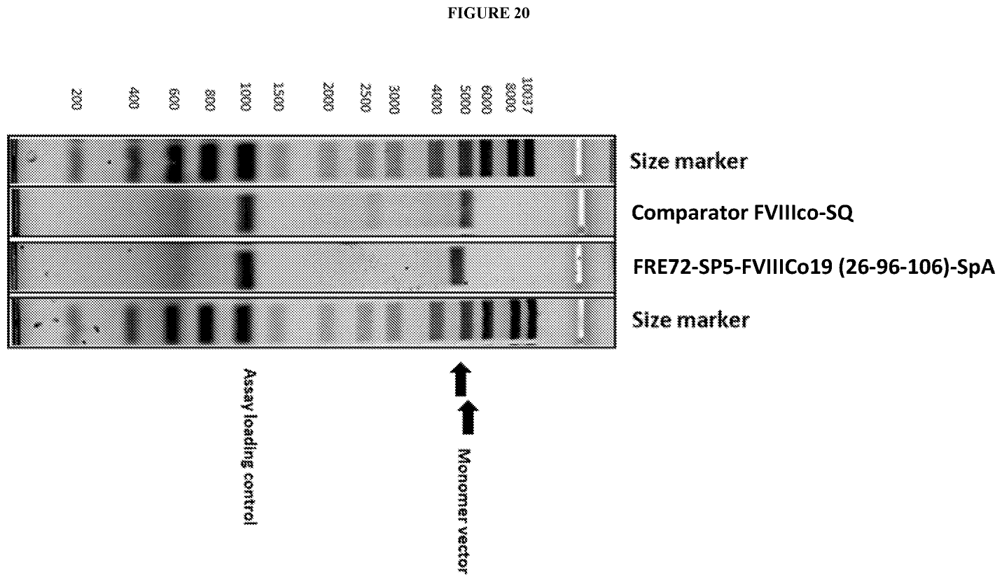

Predominant packaging of full-length vector genome sized molecules was demonstrated by alkaline gel electrophoresis (4,970 bases for comparator FVIIIco-SQ and 4,713 bases for FRE72-SP5-FVIIICo19 (26-96-106)-SpA) (FIG. 20). However, alkaline gel electrophoresis revealed an additional band between 2.5 and 3 kb for the comparator FVIIIco-SQ, which is not present in FRE72-SP5-FVIIICo19 (26-96-106)-SpA . In addition, increased background with possible additional defined signals were observed for the comparator FVIIIco-SQ in comparison to the FRE72-SP5-FVIIICo19 (26-96-106)-SpA construct. Thus increased integrity and homogeneity of the FRE72-SP5-FVIIICo19 (26-96-106)-SpA construct in comparison to the comparator FVIIIco-SQ was demonstrated by alkaline gel electrophoresis.

### Example 16—the FRE72 Promoter Provides Long-Term Expression In Vivo

An AAV vector comprising a FVIII-SQ transgene (designated FVIIIco19-SQ) under the transcriptional control of the FRE72 promotor was pseudotyped with AAV8 capsid. The overall vector genome, including ITRs, promoter and transgene, was 4845 bp long (SEQ ID NO: 73).

The resulting AAV8 vector was administered into the tail vein of C57BL6 wild type mice at 6-8 weeks of age. Vectors were stored at 4° C. prior to injection. Original viral suspensions were diluted in sterile X-vivo 10 (Lonza, BE04-380Q) in order to obtain an adequate inoculum yielding a dose of 2×10¹² vg/kg.

At days 31, 56 and 104 post-injection, a blood sample of 100u1 was taken from the lateral tail vein of each of the mice. At day 230 post-injection, terminal bleeding was performed and maximum volume (approx. 1 ml) blood sample was taken via cardiac puncture from heavily anaesthetised animals which were culled following the blood sample. Collected blood was diluted with citrate anticoagulant (1:10 dilution) and centrifuged at 5000 rpm for 5 minutes.

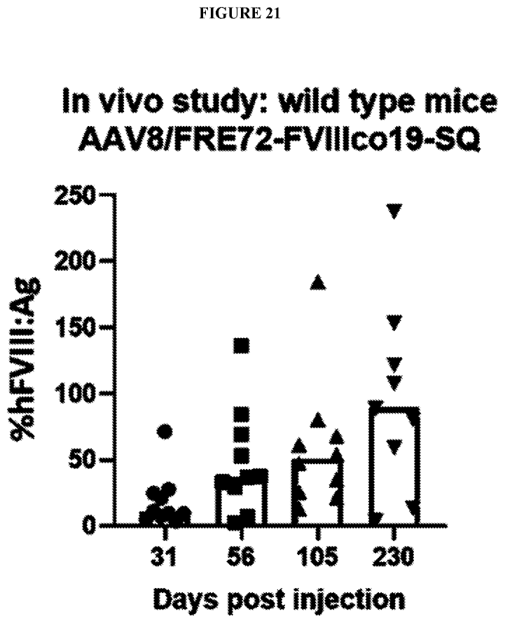

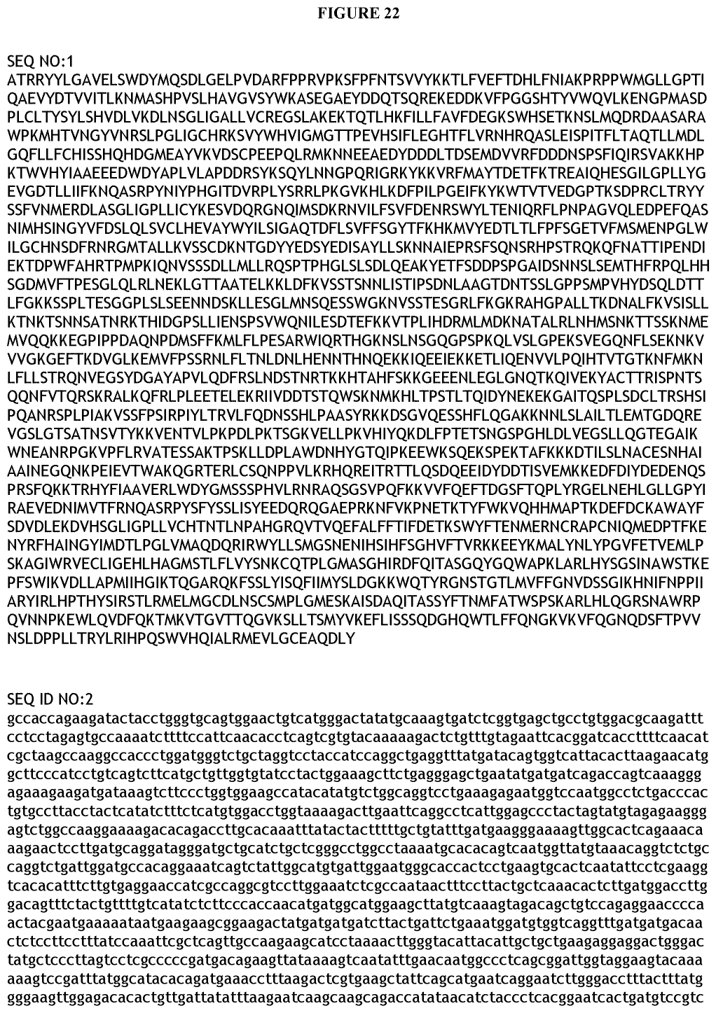

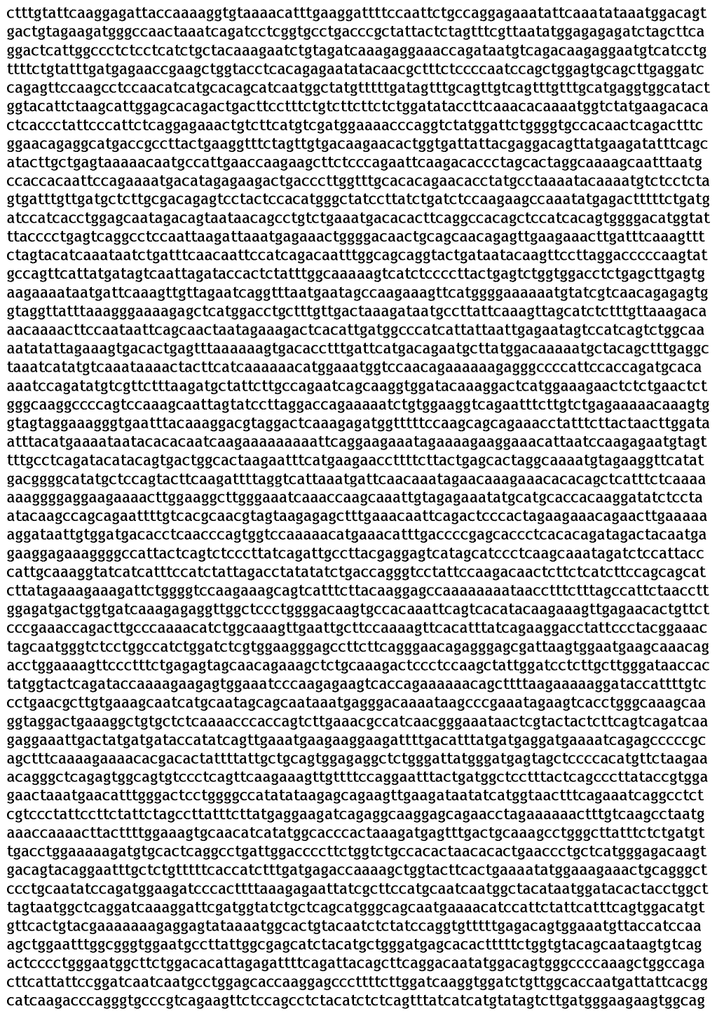

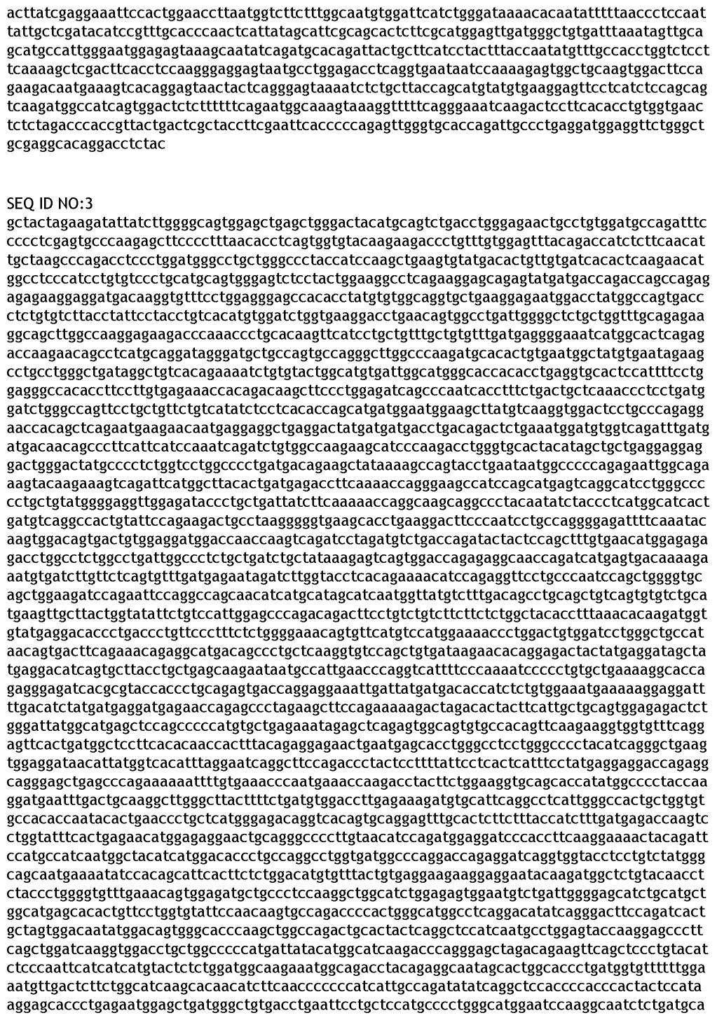

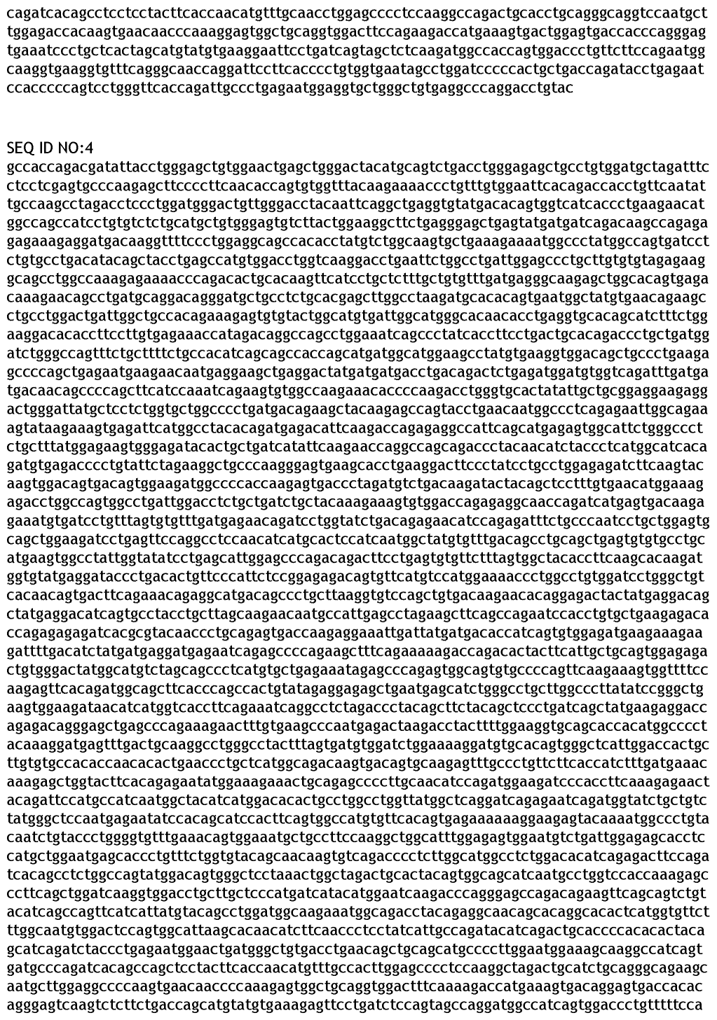

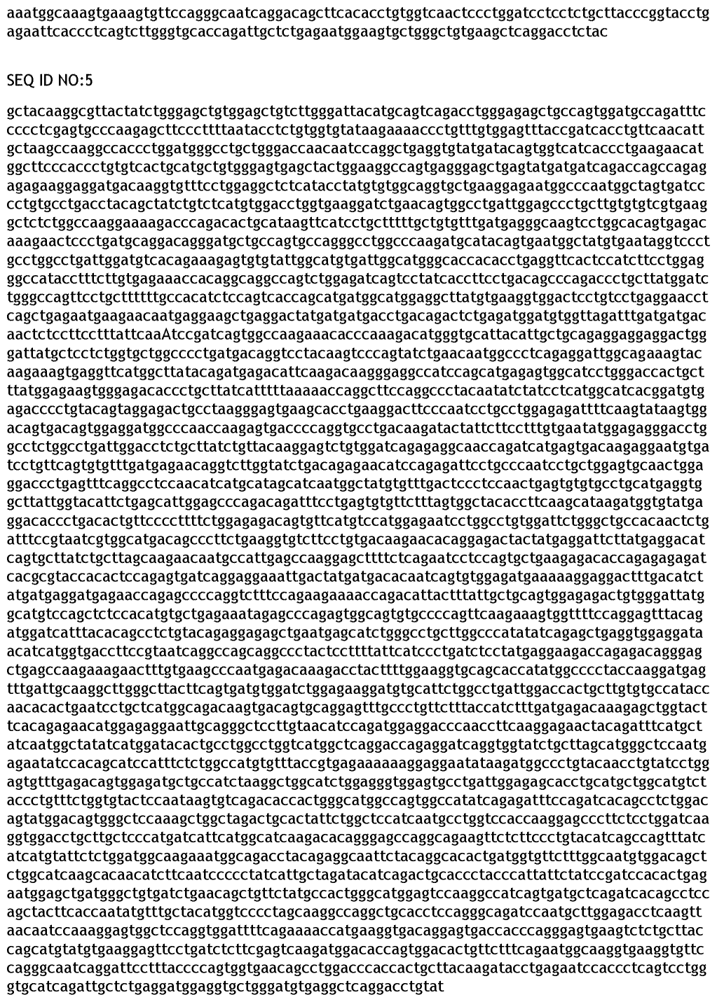

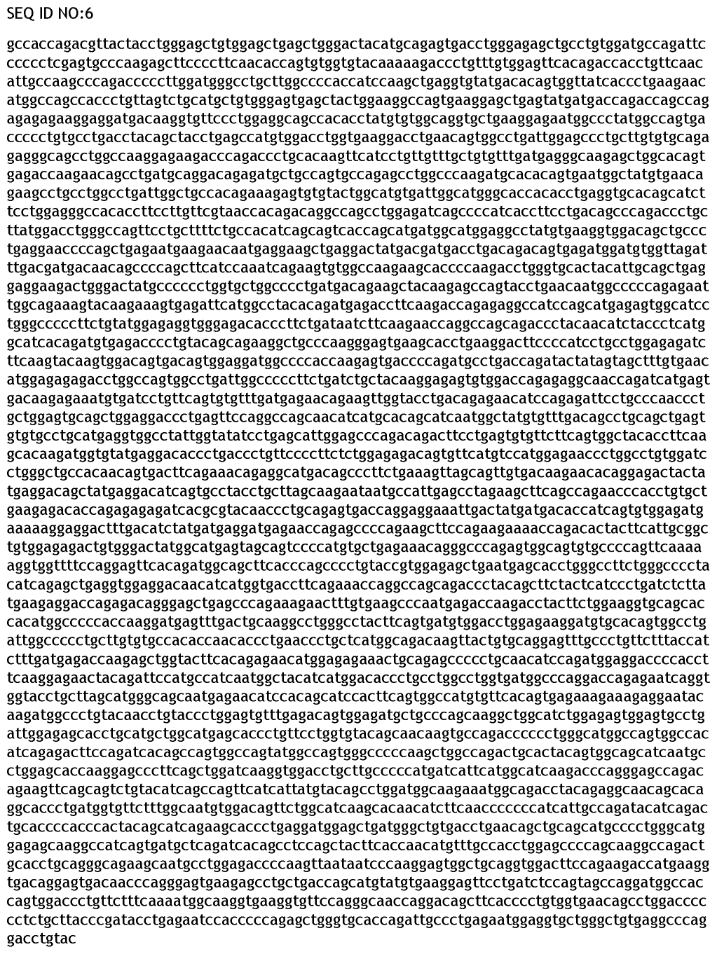

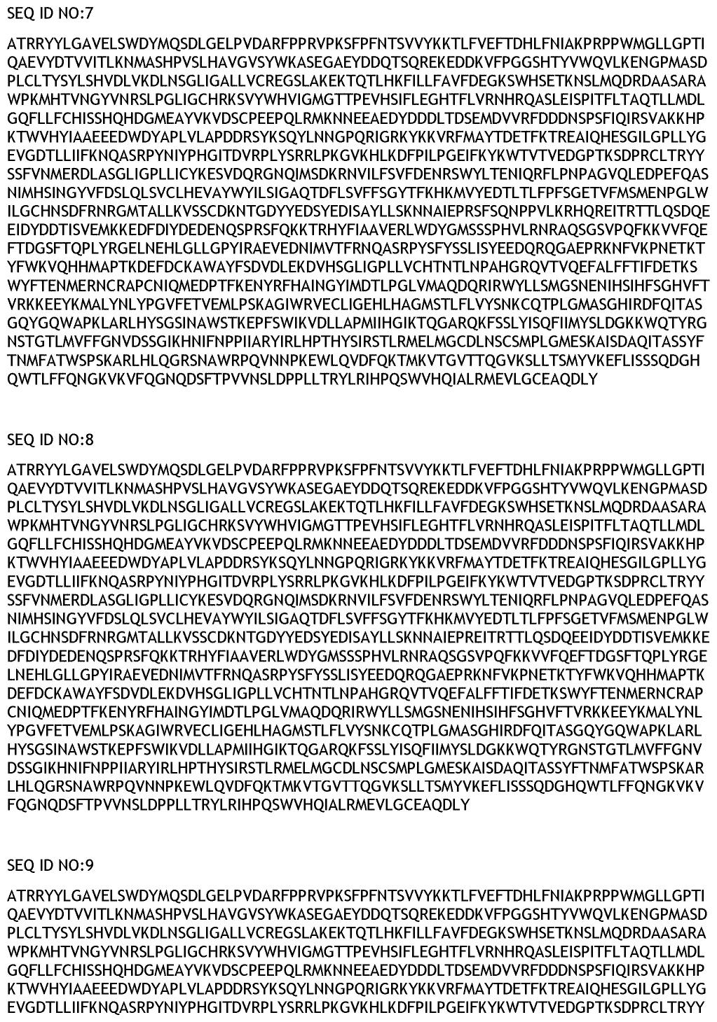

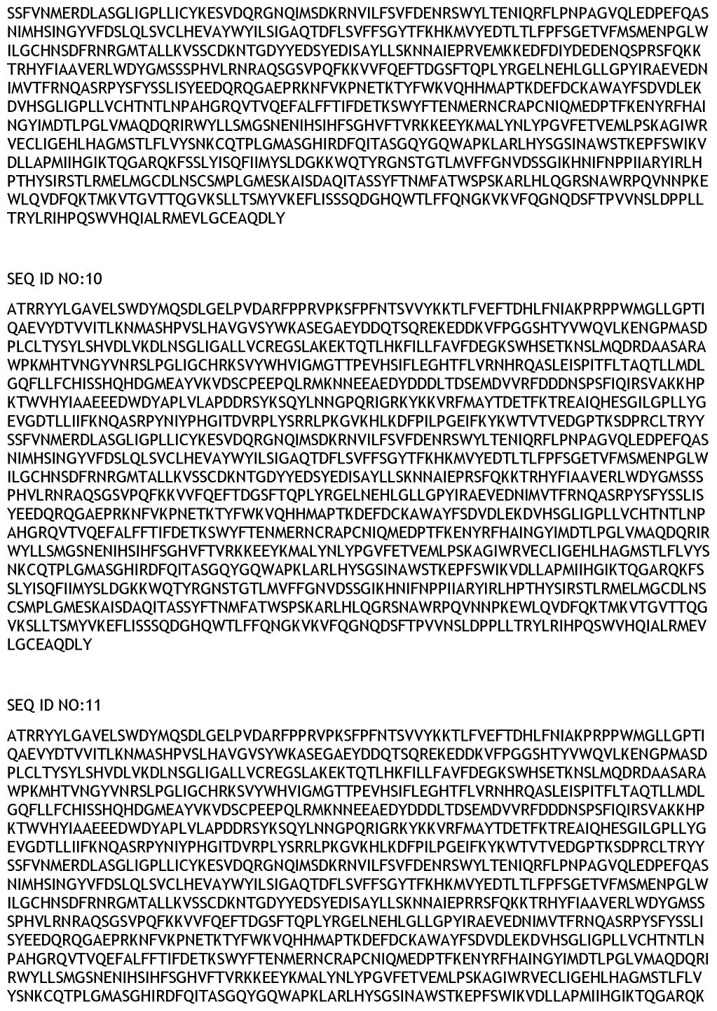

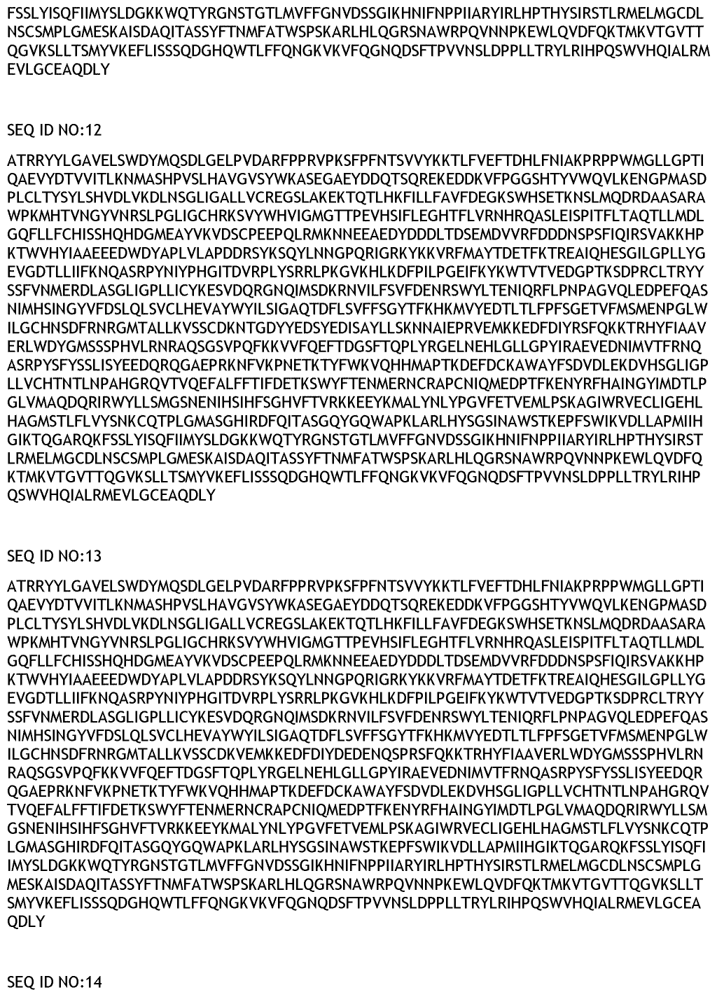

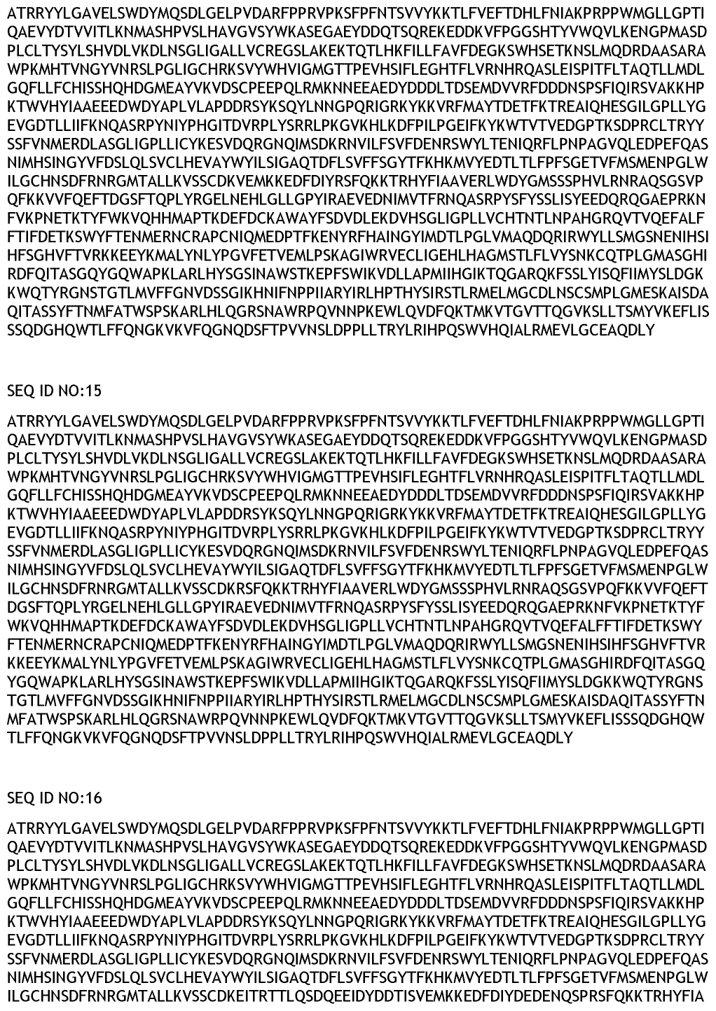

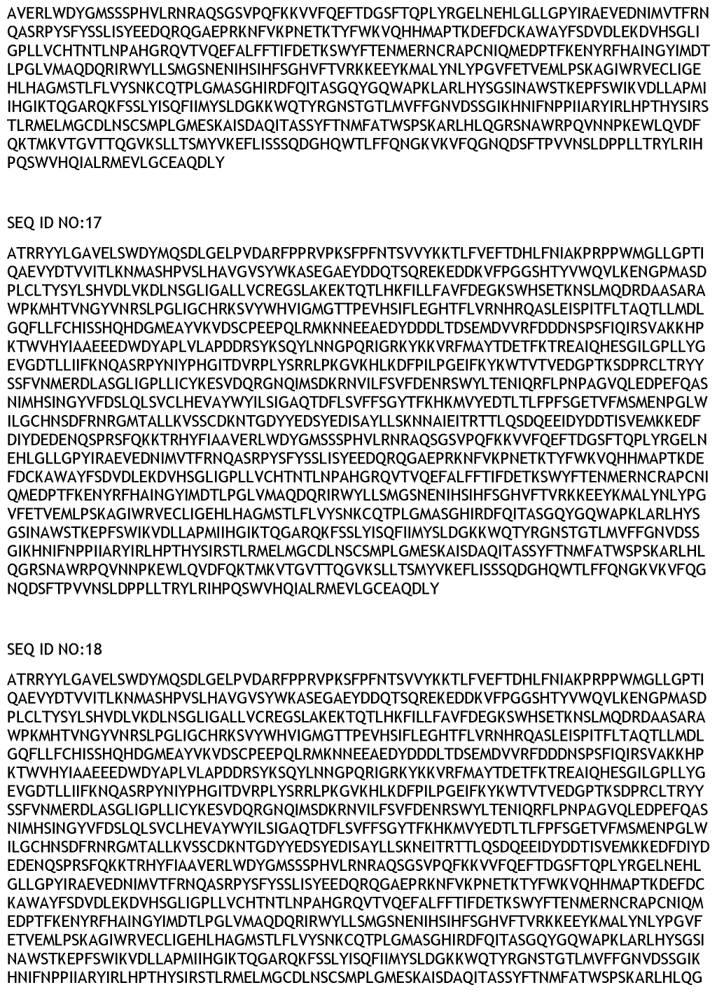

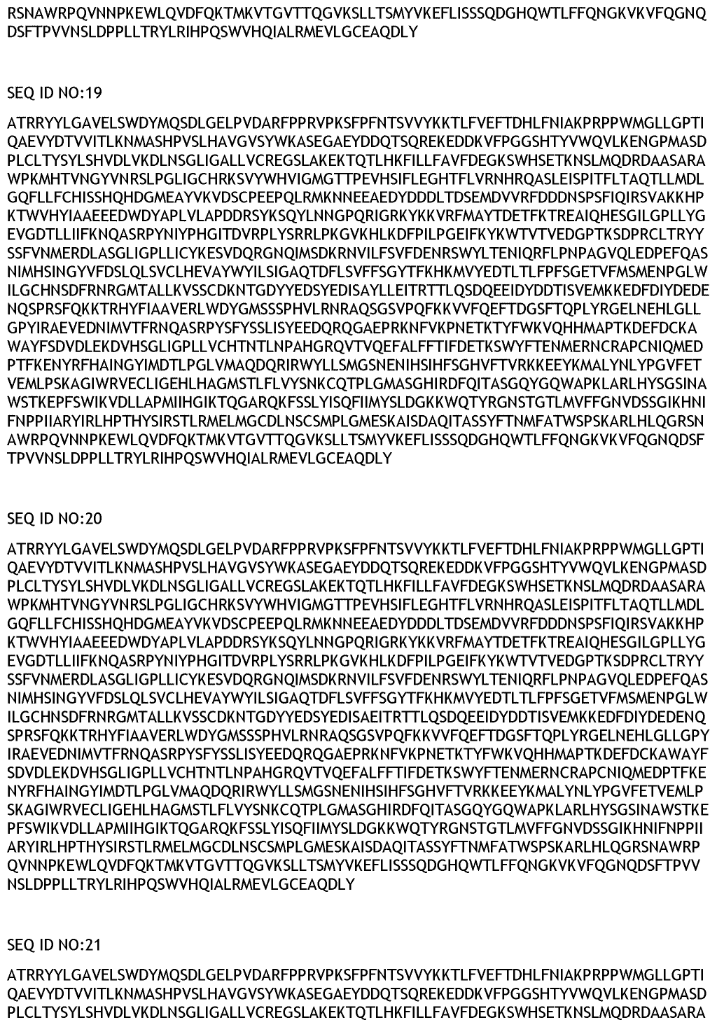

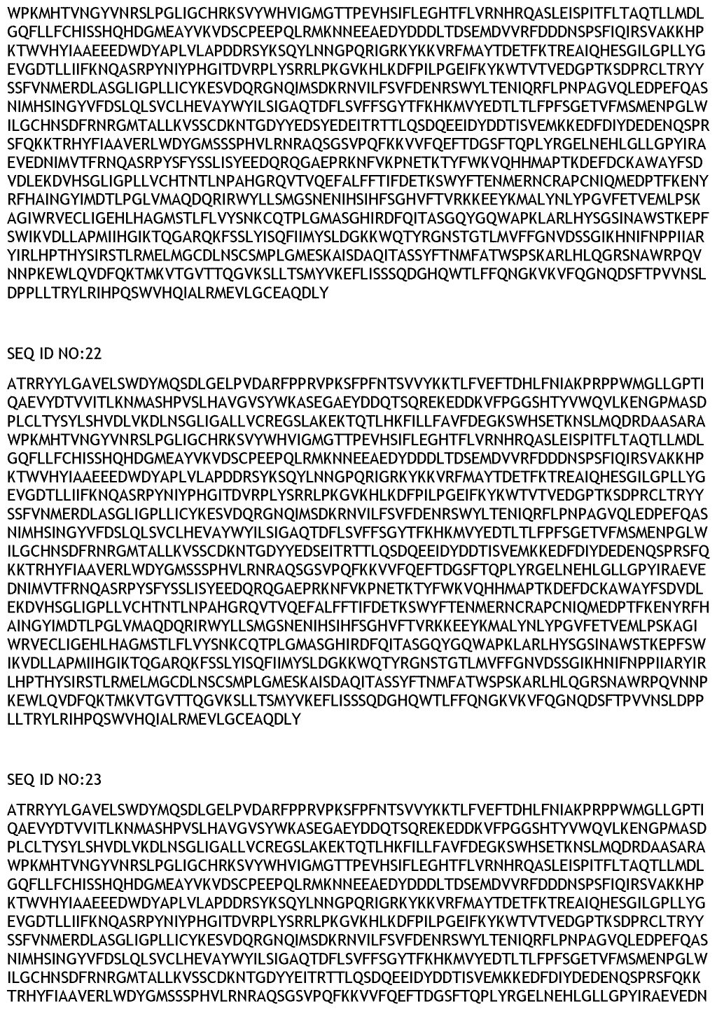

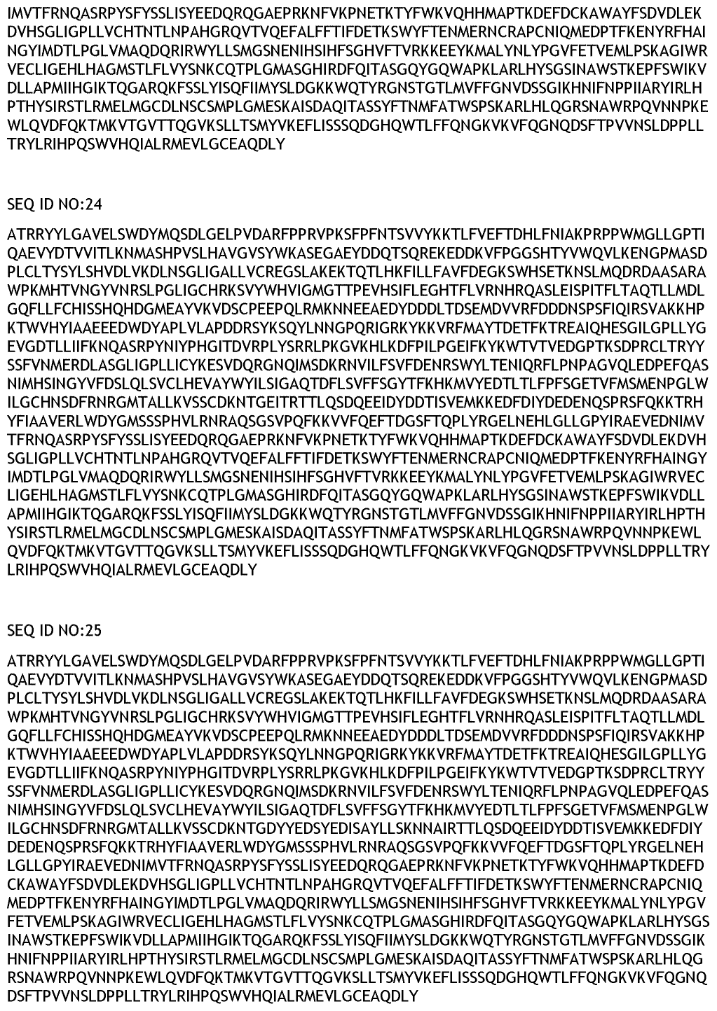

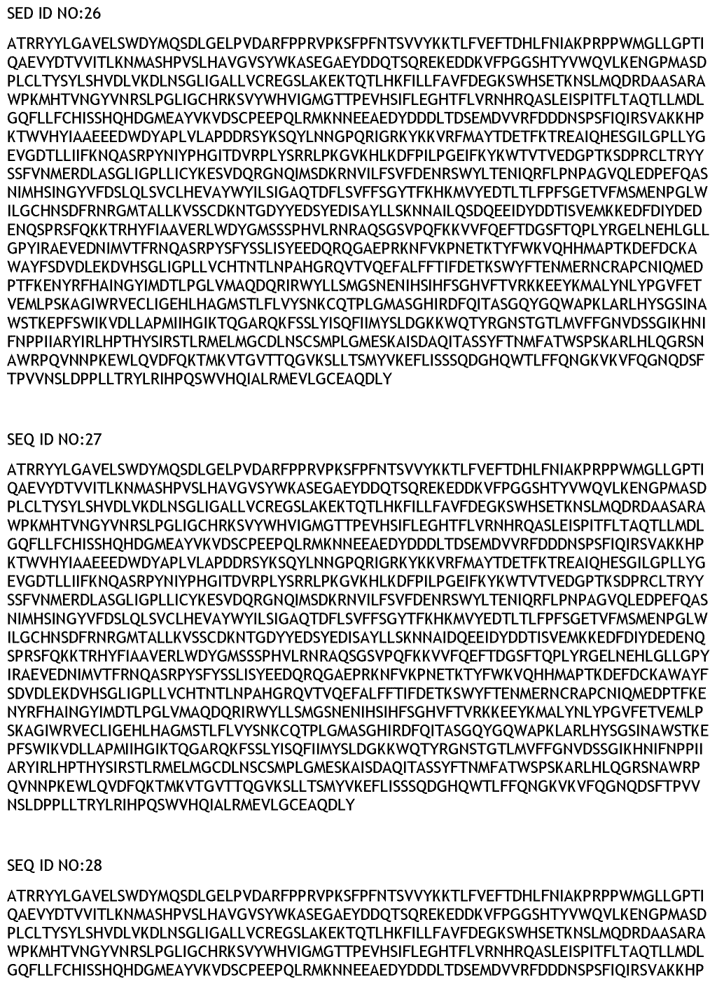

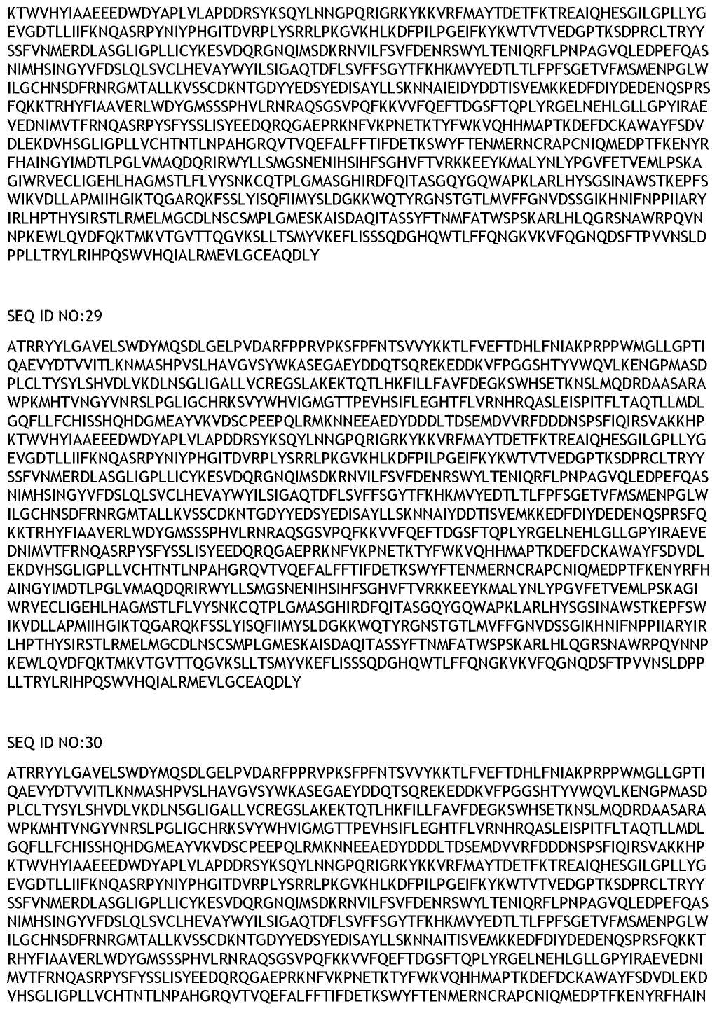

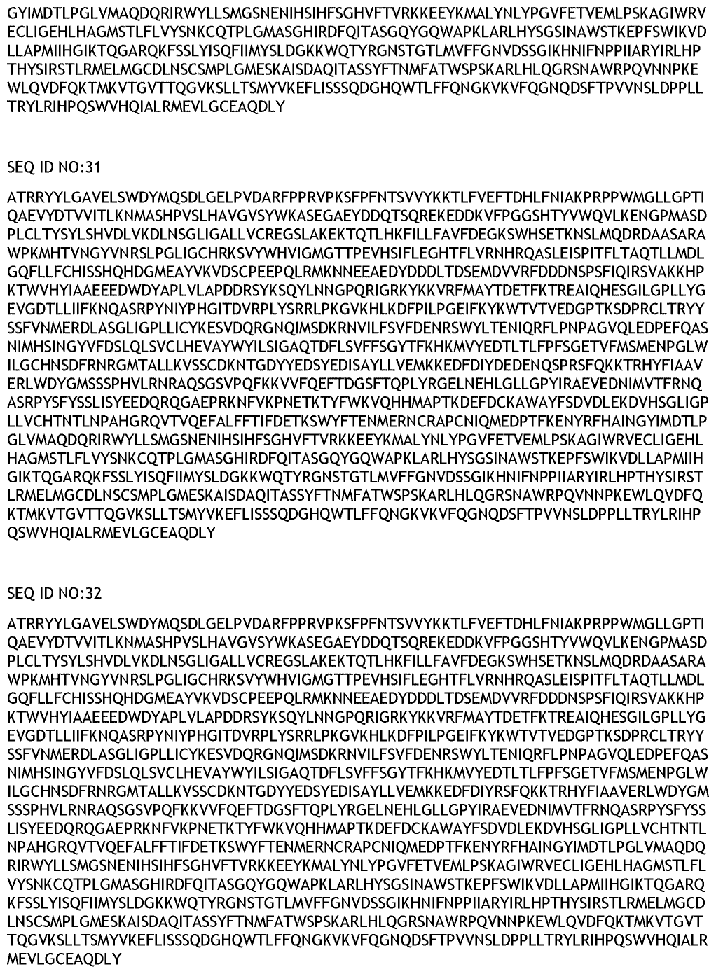

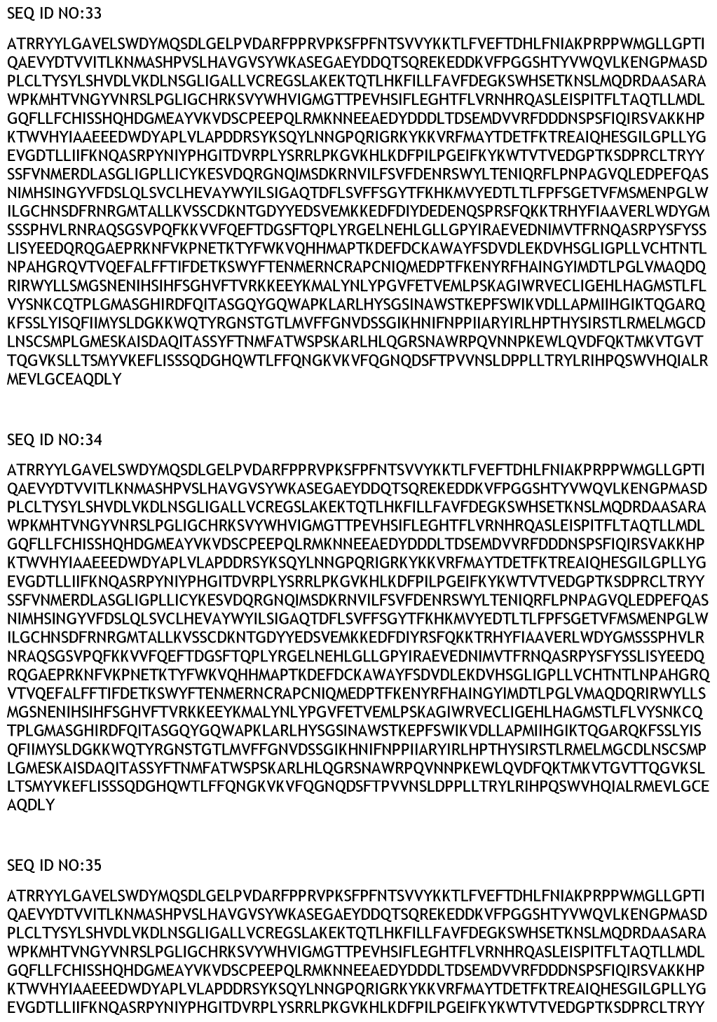

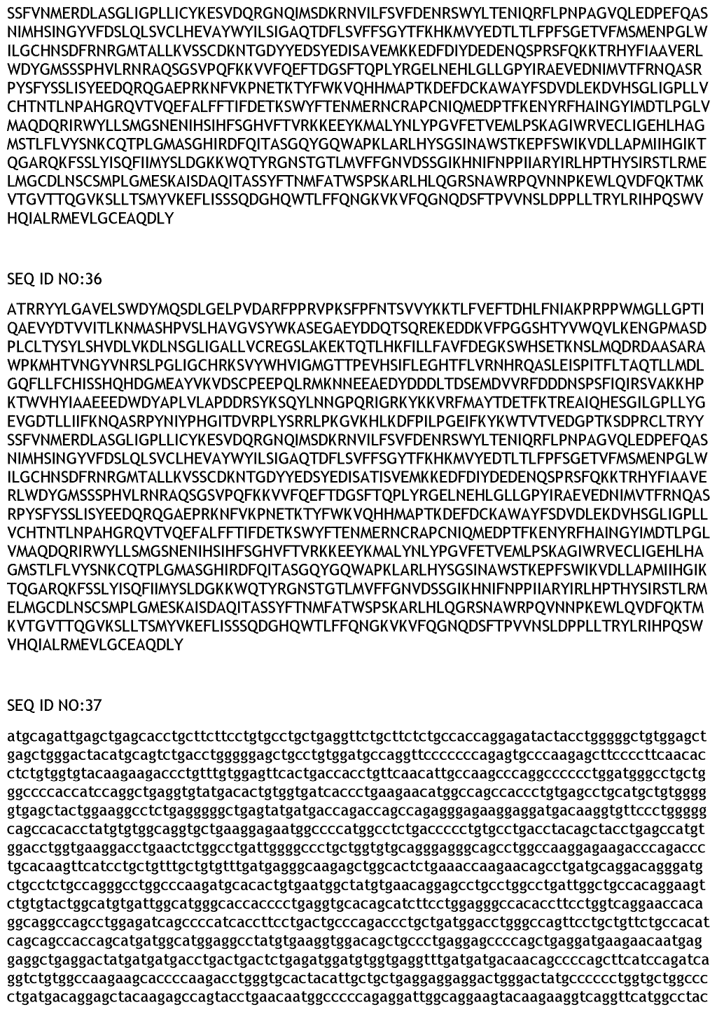

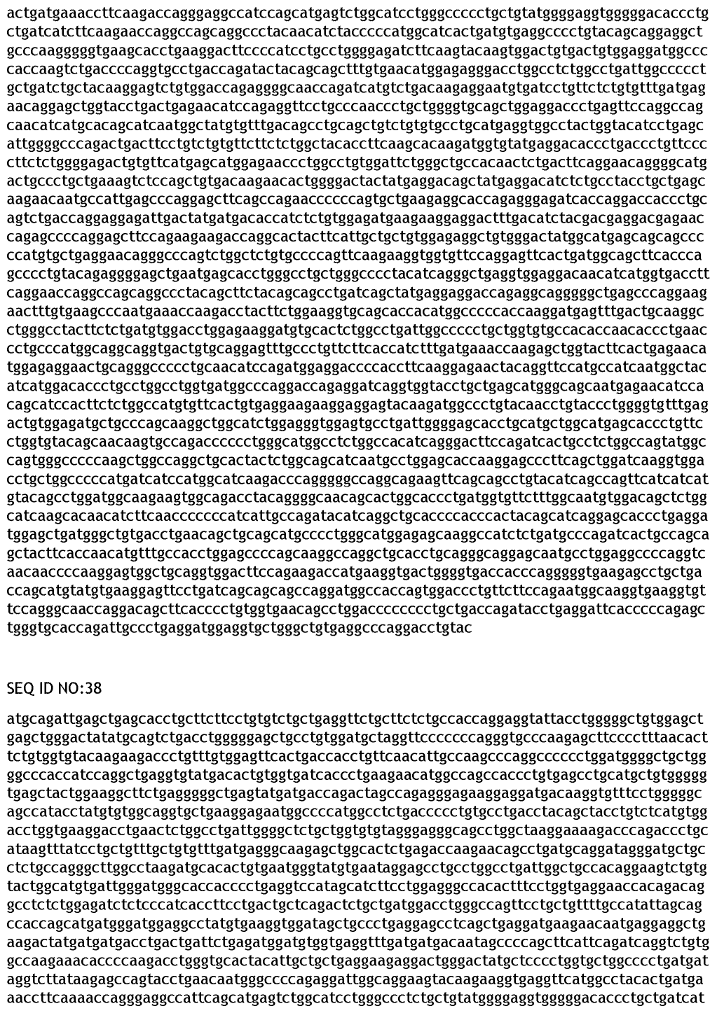

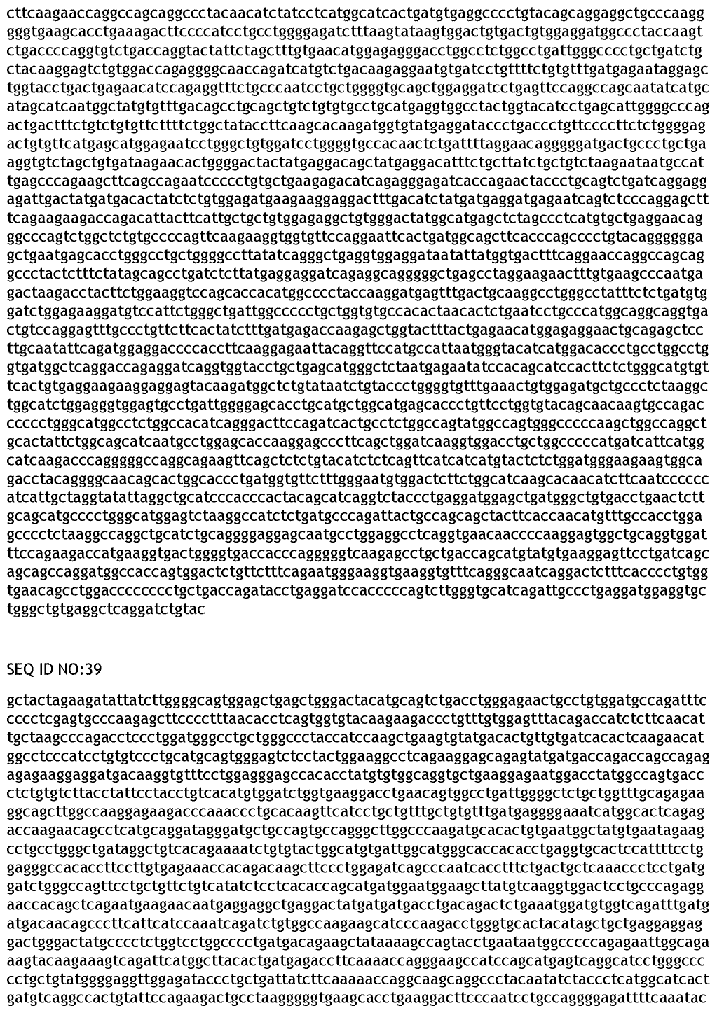

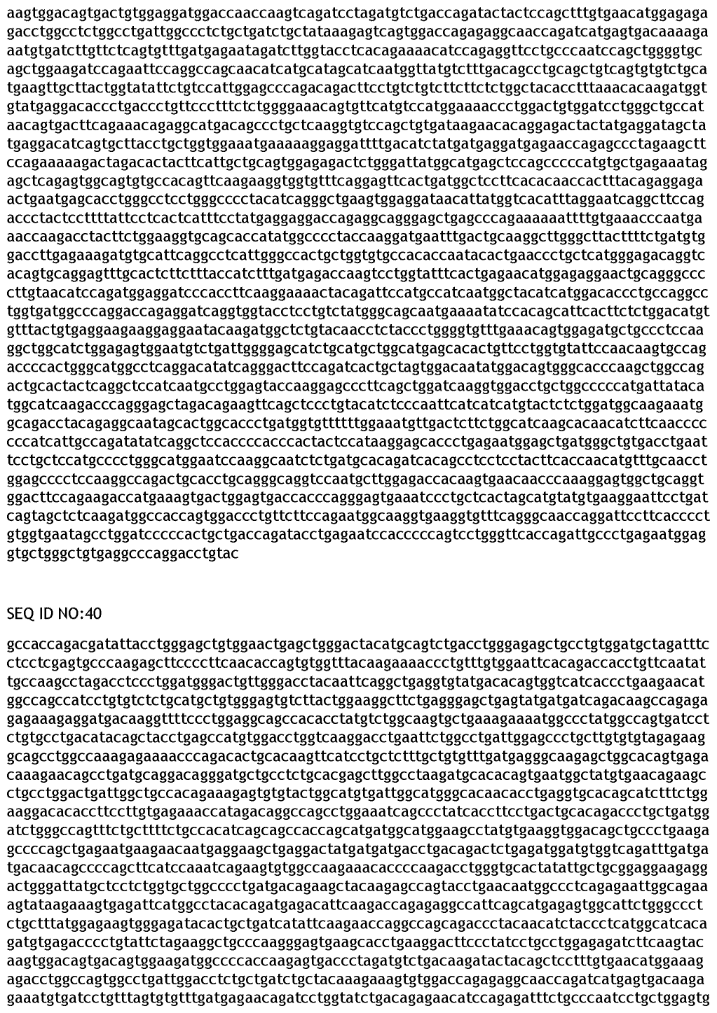

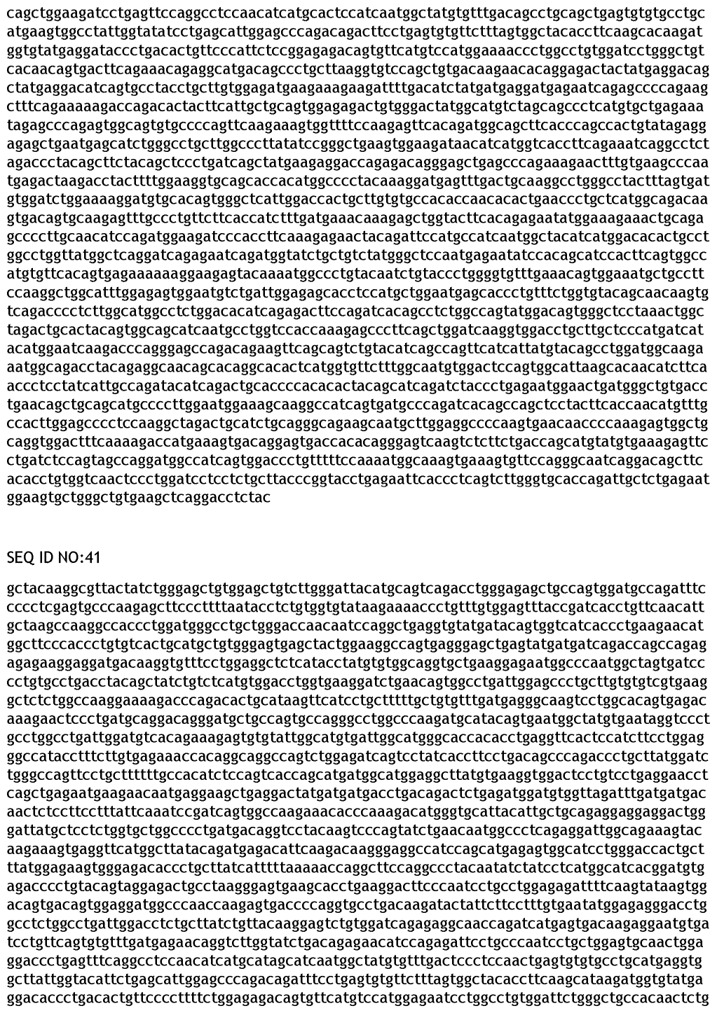

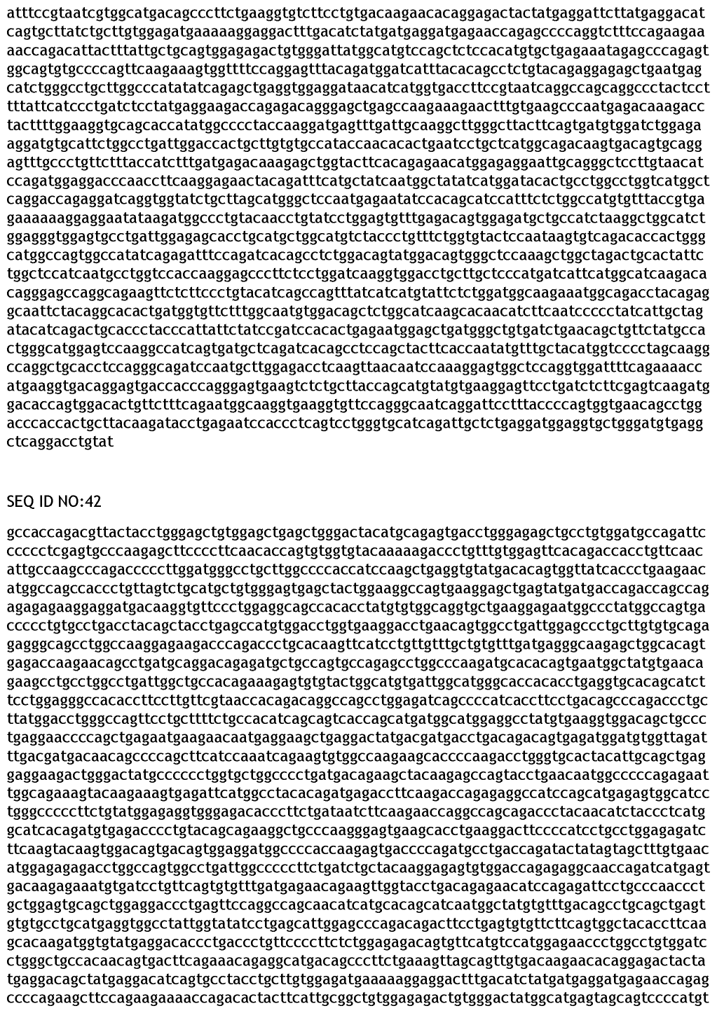

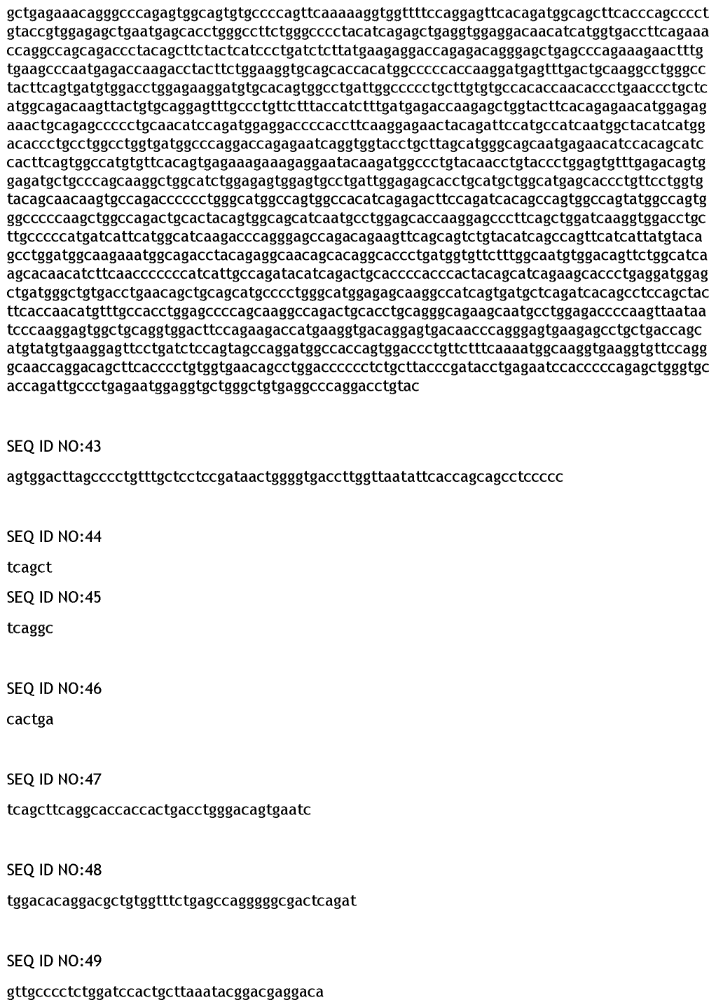

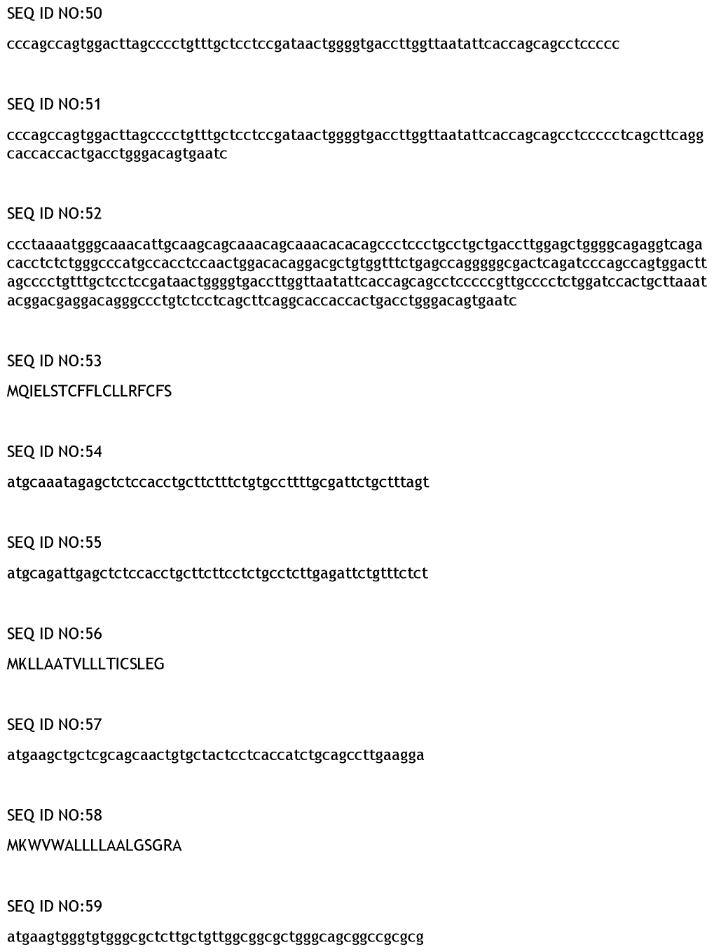

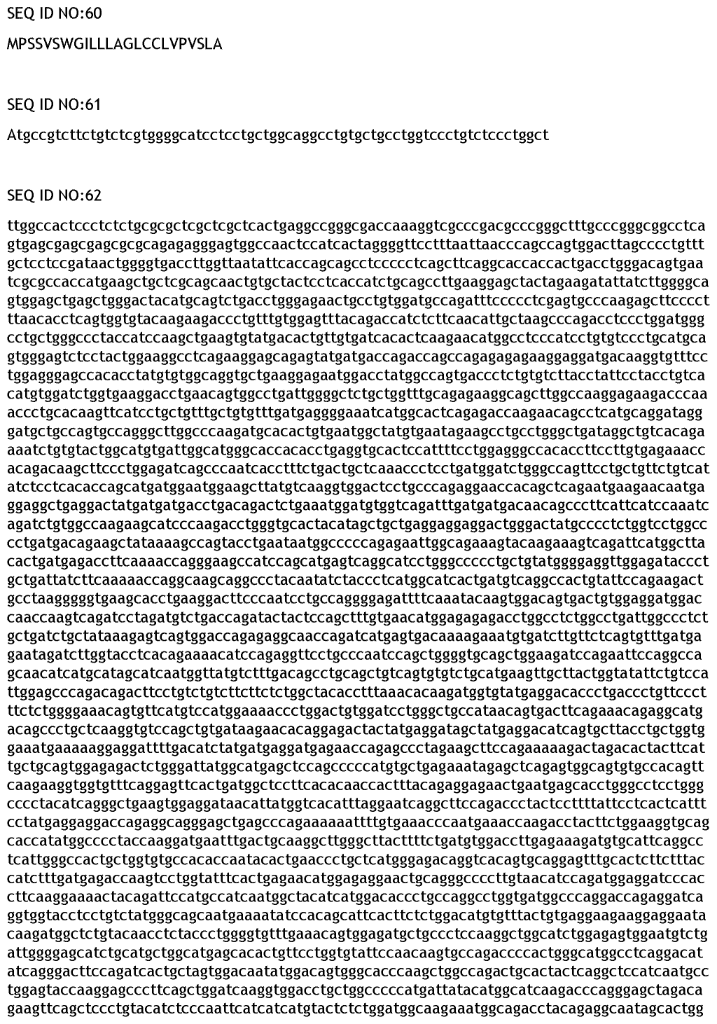

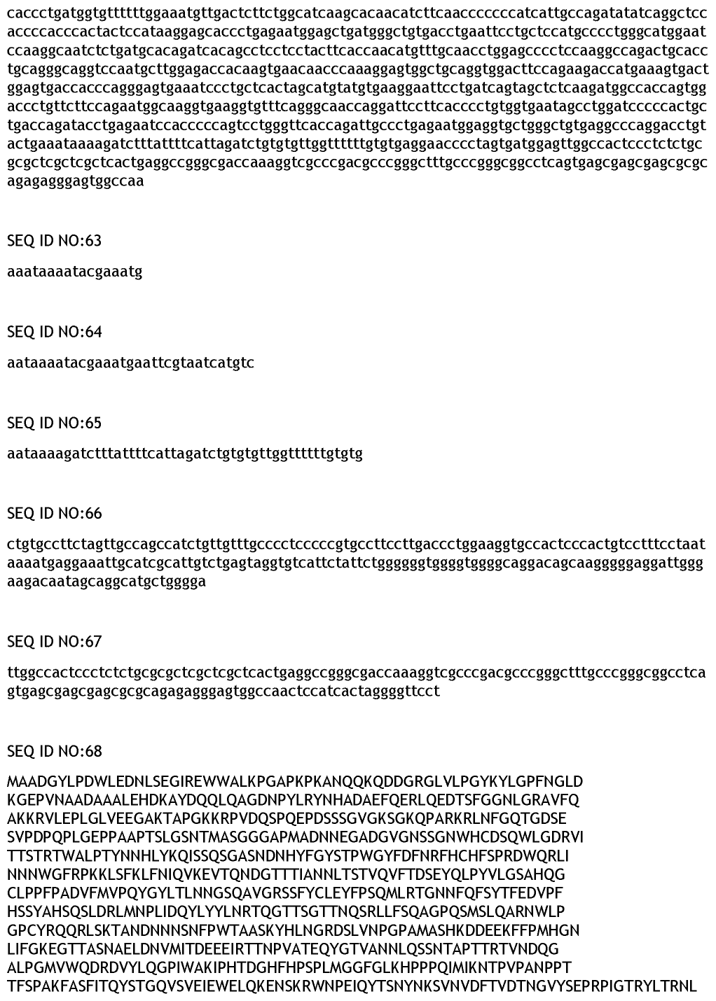

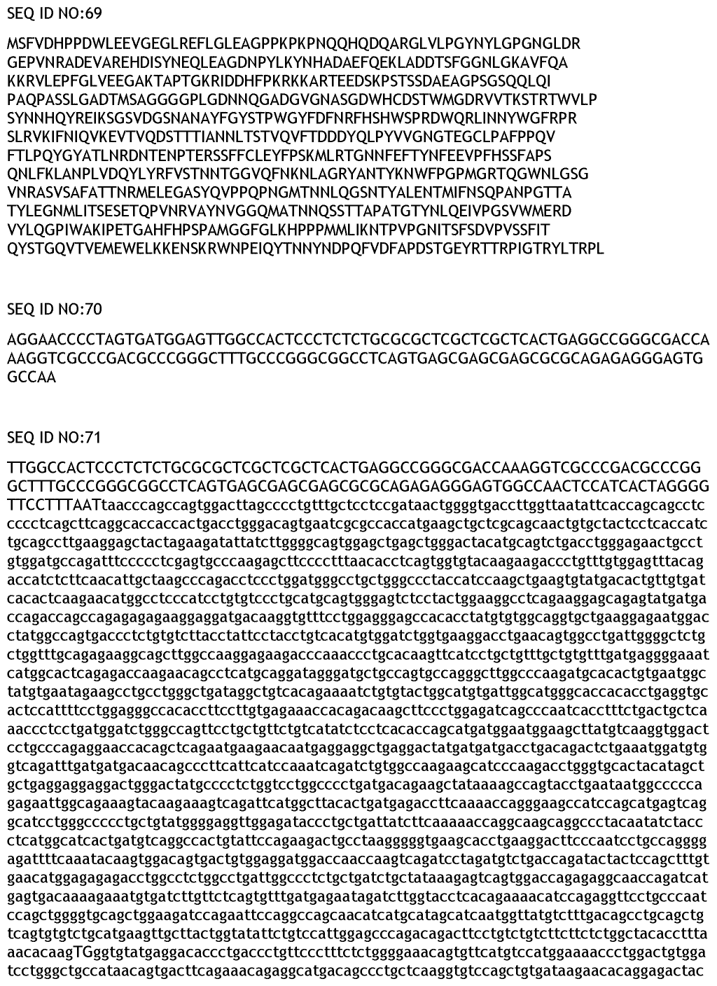

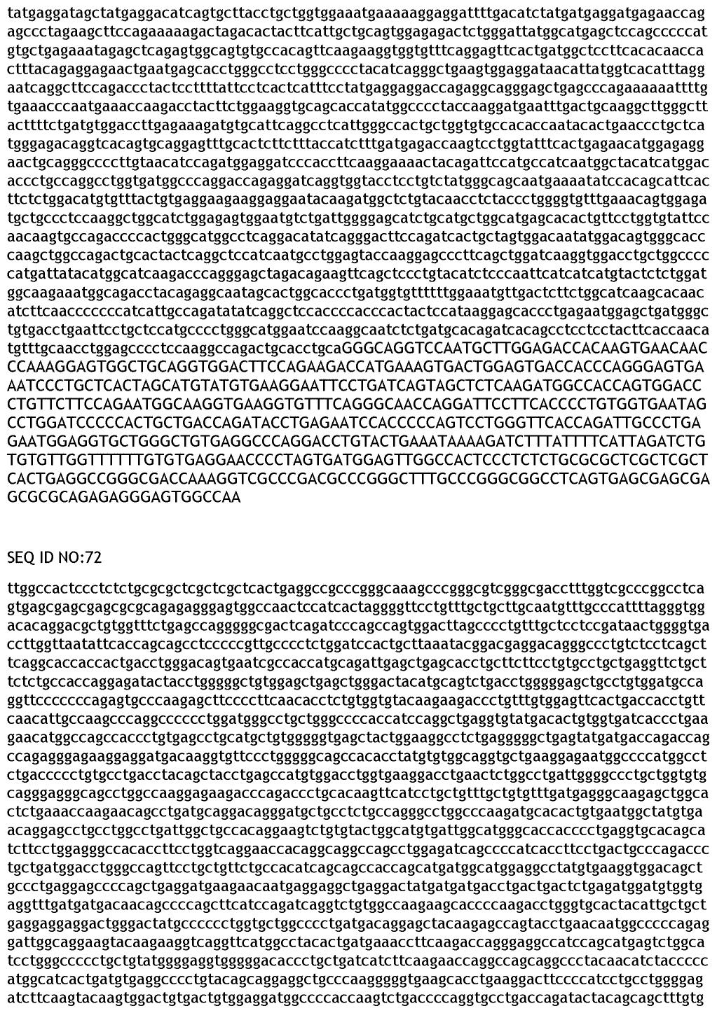

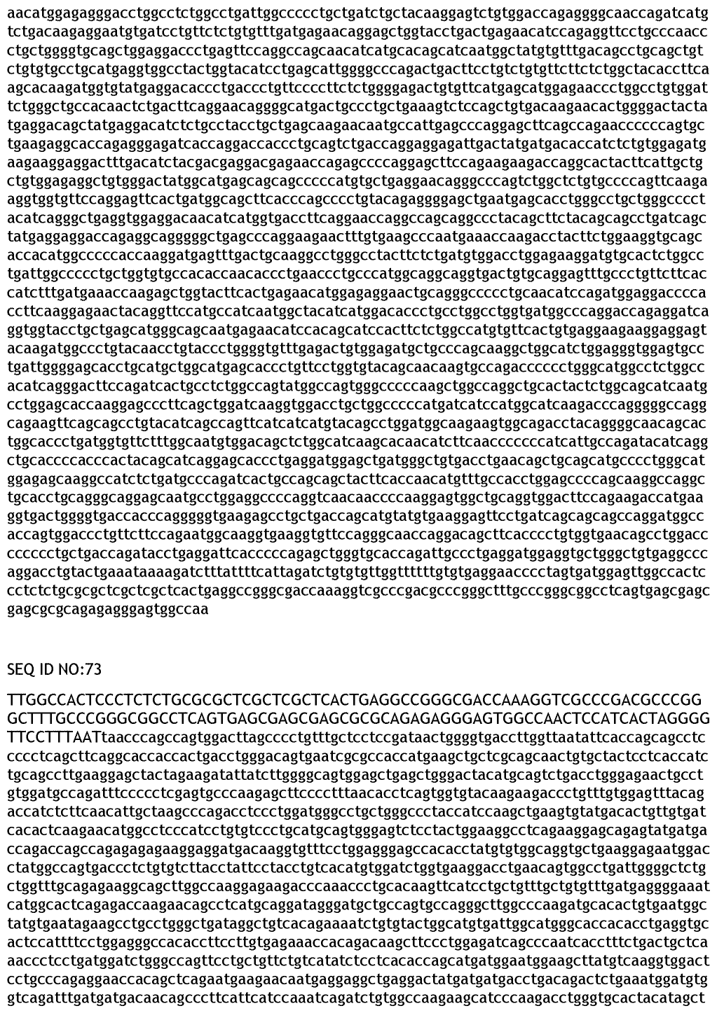

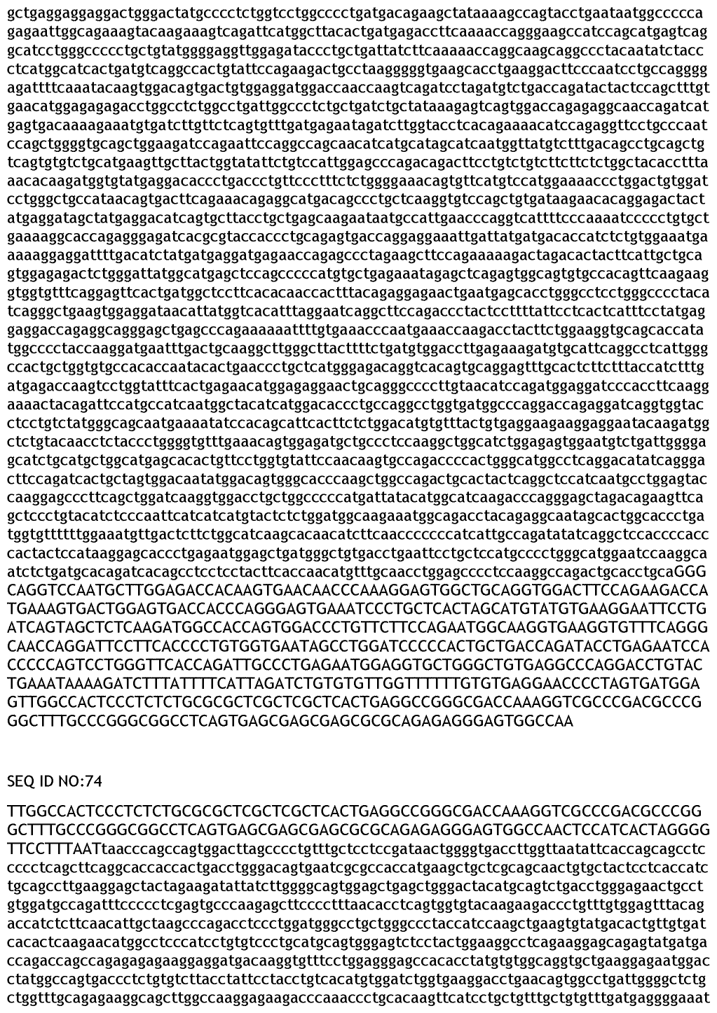

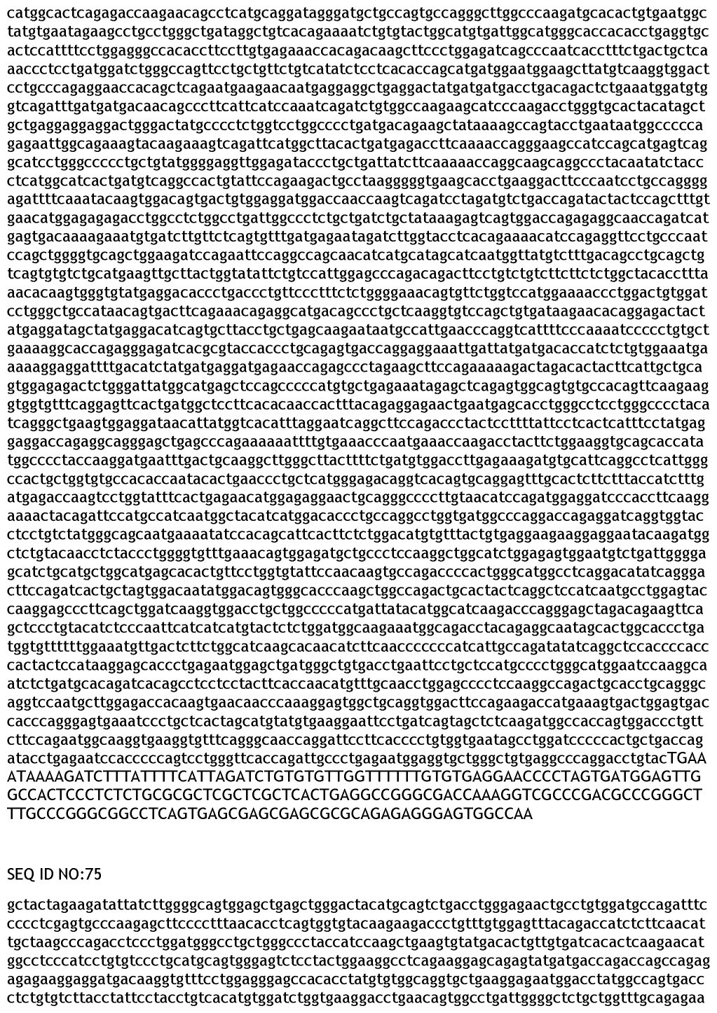

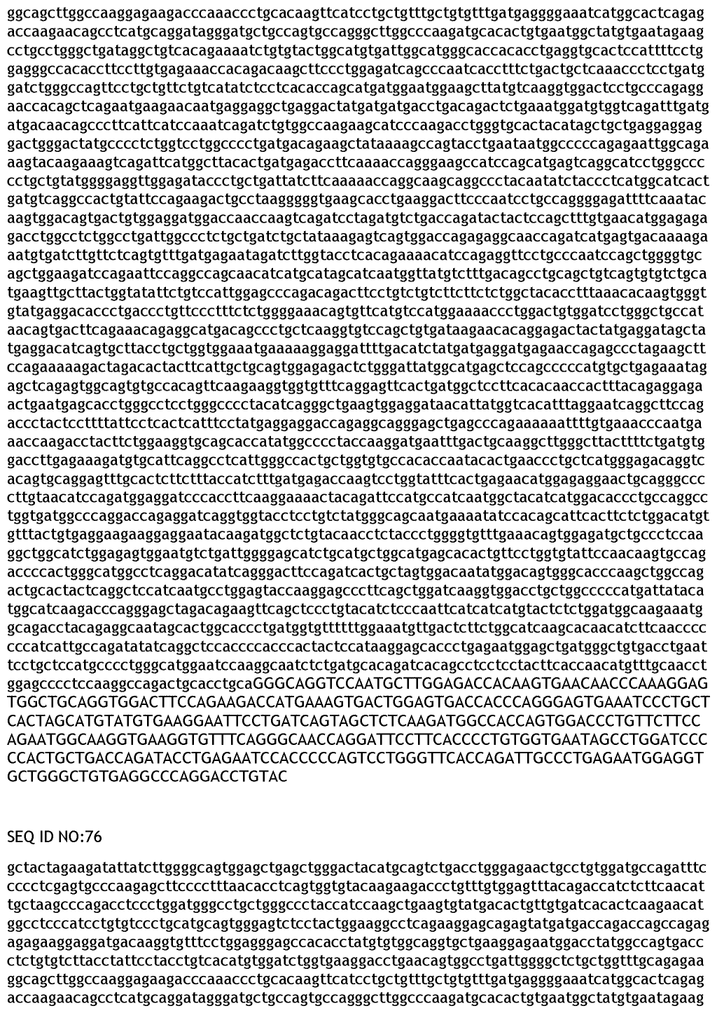

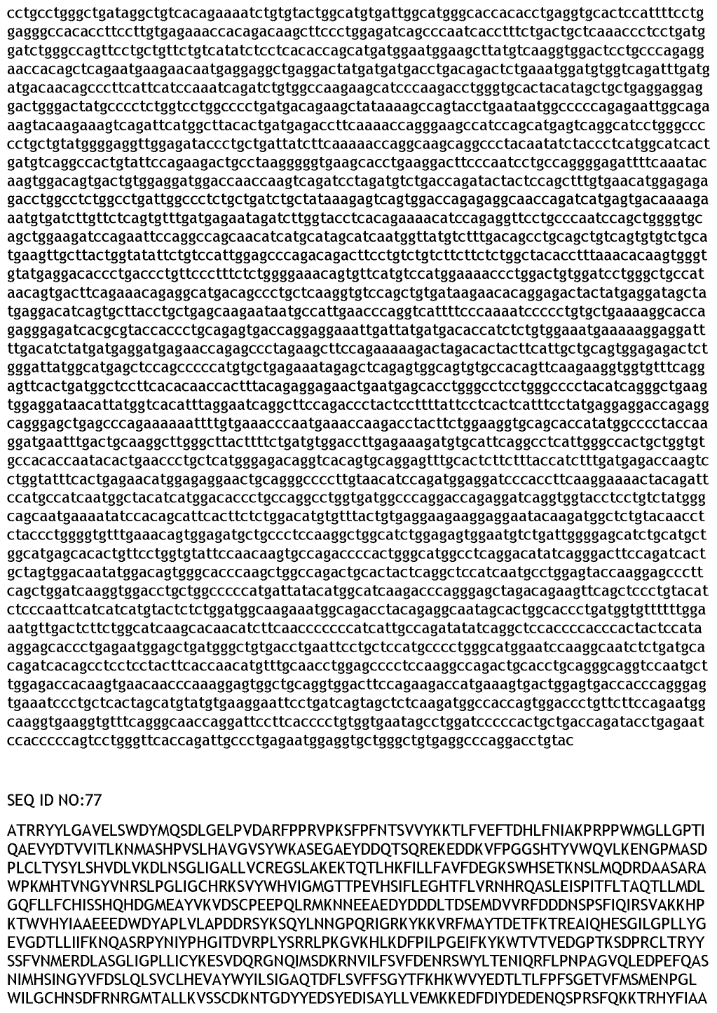

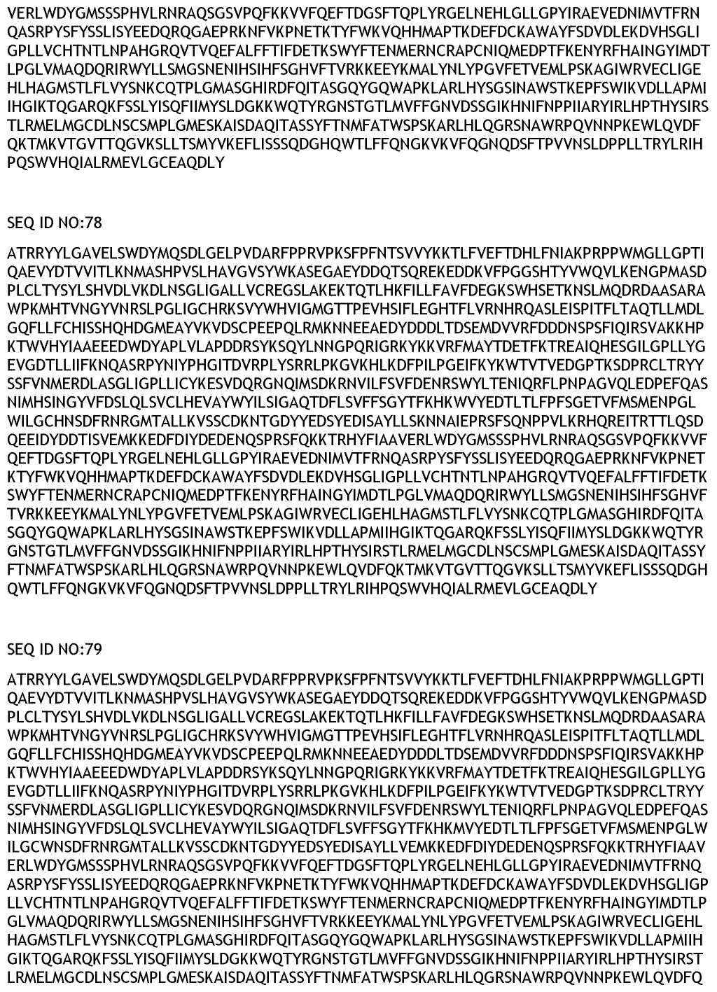

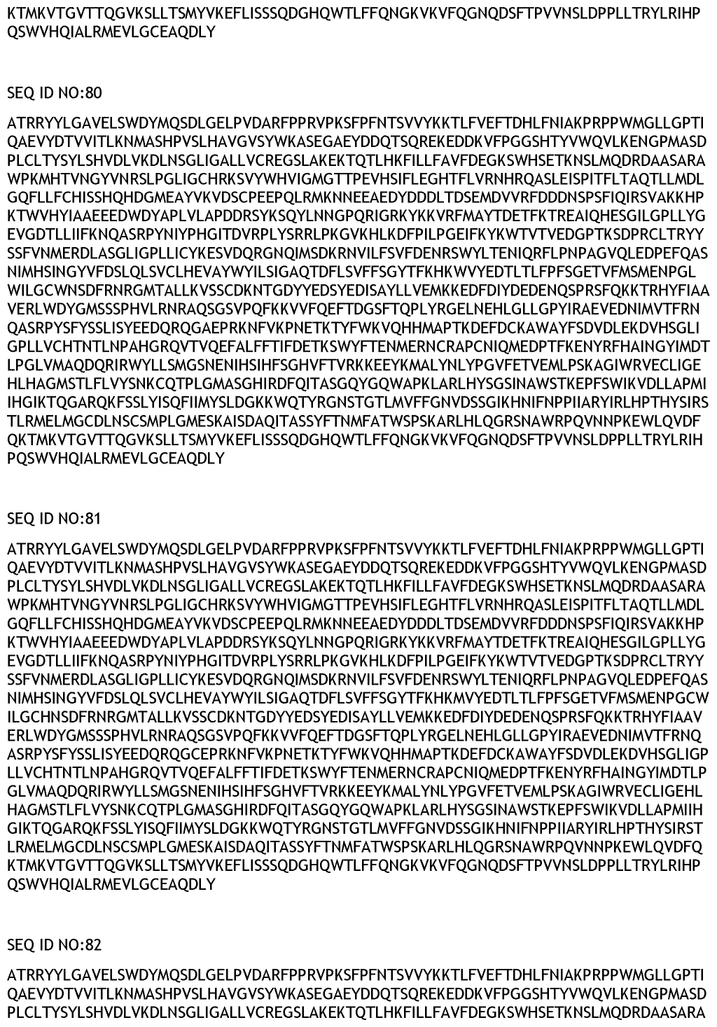

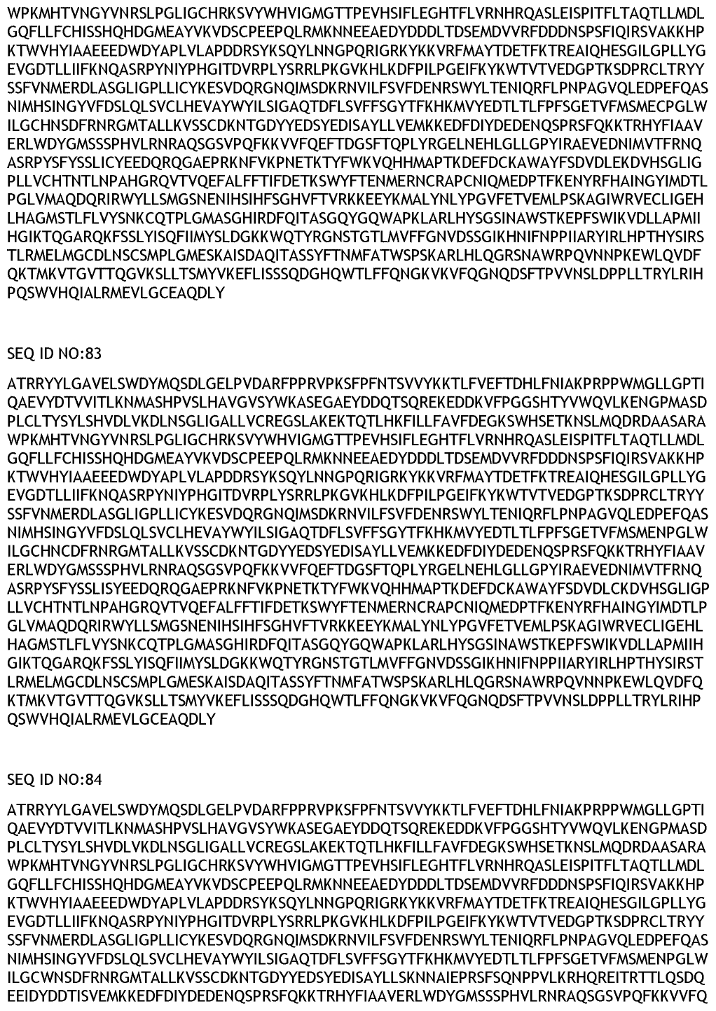

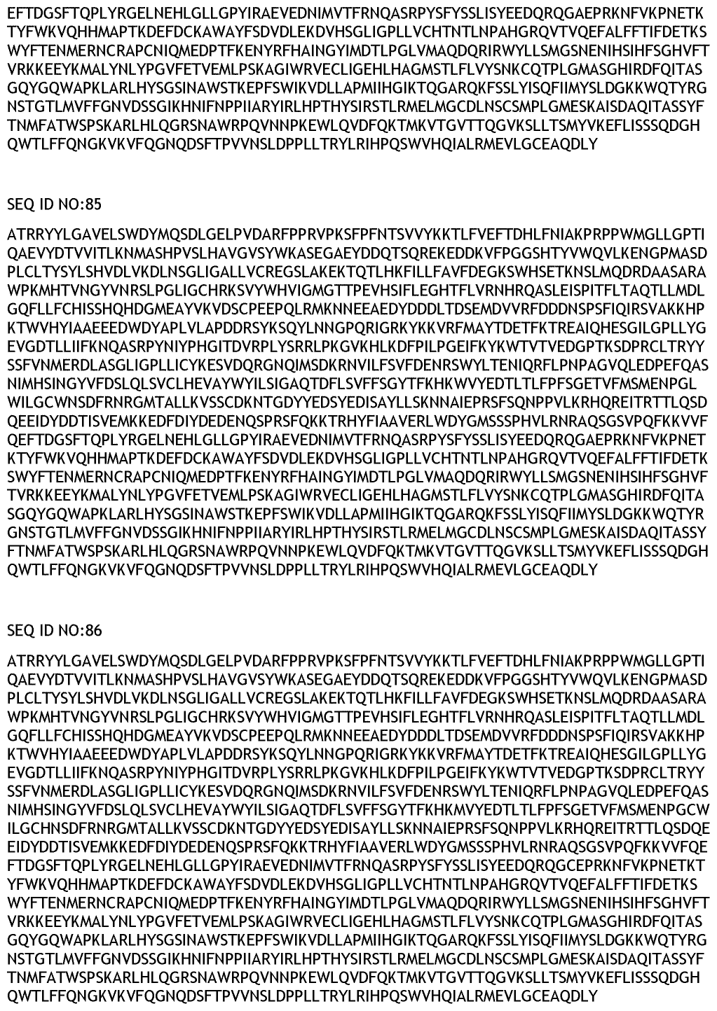

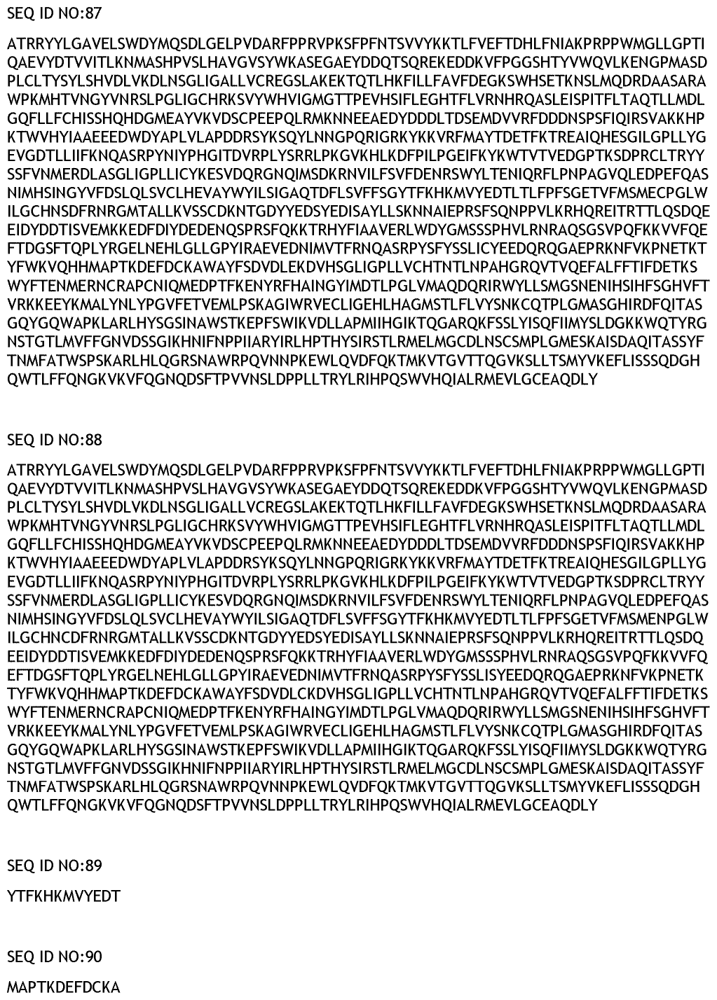

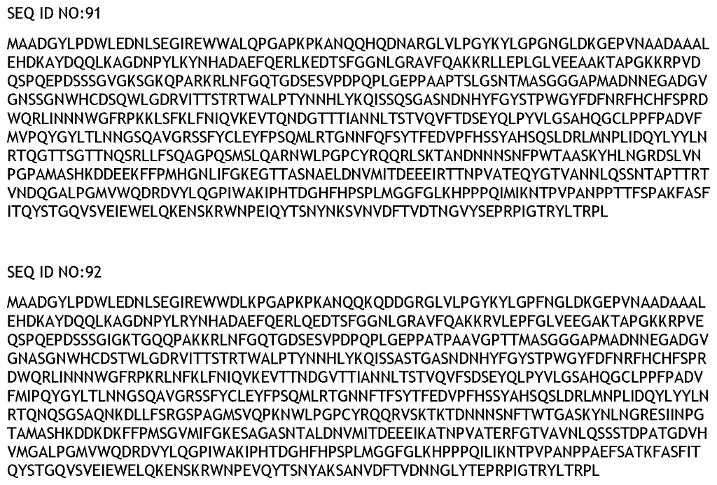

Plasma samples were analysed for FVIII antigen level using an FVIII sandwich ELISA antigen assay as described above in Materials and Methods. The results are shown in FIG. 21. The bars represent median values.

Numbered Aspects of the Invention

1. A Factor VIII polypeptide comprising a Factor VIII amino acid sequence wherein the Factor VIII amino acid sequence comprises a modified beta domain related (BDR) region which is modified relative to wild-type BDR region, which wild-type BDR region corresponds to the region between positions 713 and 1697 of SEQ ID NO: 1, wherein:
(i) the modified BDR region comprises a maximum of 88 amino acids, and the Factor VIII polypeptide has a specific activity which is higher than the specific activity of a polypeptide of SEQ ID NO: 7; and/or
(ii) the modified BDR region comprises a maximum of 74 amino acids.

2. The Factor VIII polypeptide according to aspect 1, wherein the Factor VIII polypeptide has a specific activity which is at least 50%, at least 60%, at least 70%, at least 75%, at least 80%, at least 85%, at least 90%, at least 95%, at least 98%, at least 99%, or at least 100% of the specific activity of a reference wild-type Factor VIII polypeptide.

3. The Factor VIII polypeptide according to aspect 1 or 2, wherein the Factor VIII polypeptide has a specific activity which is higher than the specific activity of a reference wild-type Factor VIII polypeptide.

4. The Factor VIII polypeptide according to any one of the preceding aspects, wherein the Factor VIII polypeptide has a specific activity which is at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of a reference wild-type Factor VIII.

5. The Factor VIII polypeptide according to any one of aspects 2 to 4, wherein the reference wild-type Factor VIII sequence is the Factor VIII polypeptide of SEQ ID NO: 1.

6. The Factor VIII polypeptide according to any one of the preceding aspects, wherein the Factor VIII polypeptide has a specific activity which is at least 50%, at least 60%, at least 70%, at least 75%, at least 80%, at least 85%, at least 90%, at least 95%, at least 98%, at least 99%, or at least 100% of the specific activity of the Factor VIII polypeptide of SEQ ID NO: 7.

7. The Factor VIII polypeptide according to any one of the preceding aspects, wherein the Factor VIII polypeptide has a specific activity which is higher than the specific activity of the Factor VIII polypeptide of SEQ ID NO: 7.

8. The Factor VIII polypeptide according to any one of the preceding aspects, wherein the Factor VIII polypeptide has a specific activity which is at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of the Factor VIII polypeptide of SEQ ID NO: 7.

9. The Factor VIII polypeptide according to any one of the preceding aspects, wherein the Factor VIII polypeptide has a specific activity which is at least 50%, at least 60%, at least 70%, at least 75%, at least 80%, at least 85%, at least 90%, at least 95%, at least 98%, at least 99%, or at least 100% of the specific activity of the Factor VIII polypeptide of SEQ ID NO: 8.

10. The Factor VIII polypeptide according to any one of the preceding aspects, wherein the Factor VIII polypeptide has a specific activity which is higher than the specific activity of the Factor VIII polypeptide of SEQ ID NO: 8.

11. The Factor VIII polypeptide according to any one of the preceding aspects, wherein the Factor VIII polypeptide has a specific activity which is at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of the Factor VIII polypeptide of SEQ ID NO: 8.

12. The Factor VIII polypeptide according to any one of the preceding aspects, wherein the Factor VIII polypeptide has a specific activity which is higher than the specific activity of the Factor VIII polypeptide encoded by SEQ ID NO: 37.

13. The Factor VIII polypeptide according to any one of the preceding aspects, wherein the Factor VIII polypeptide has a specific activity which is higher than the specific activity of the Factor VIII polypeptide encoded by SEQ ID NO: 38.

14. The Factor VIII polypeptide according to any one of the preceding aspects, wherein the specific activity is measured using a chromogenic assay.

15. The Factor VIII polypeptide according to any one of the preceding aspects, wherein the specific activity is measured using a clotting assay, optionally a one-stage clotting assay.

16. The Factor VIII polypeptide according to any one of the preceding aspects, wherein the modified BDR region comprises a maximum of 87, 85, 80, 75, 70, 65, 60, 55, 50, or 45 amino acids.

17. The Factor VIII polypeptide according to any one of the preceding aspects, wherein the modified BDR region comprises a maximum of 74 amino acids.

18. The Factor VIII polypeptide according to any one of the preceding aspects, wherein the modified BDR region comprises a maximum of 47 amino acids.

19. The Factor VIII polypeptide according to any one of the preceding aspects, wherein the modified BDR region comprises a maximum of 45 amino acids.

20. The Factor VIII polypeptide according to any one of the preceding aspects, wherein the modified BDR region comprises at least 20, at least 25, at least 28, at least 30, at least 35, at least 40, at least 45, at least 50, at least 54, at least 55, at least 57, at least 58, at least 60, or at least 65 amino acids.

21. The Factor VIII polypeptide according to aspect 20, wherein the modified BDR region comprises at least 28 amino acids.

22. The Factor VIII polypeptide according to aspect 20, wherein the modified BDR region comprises at least 30 amino acids.

23. The Factor VIII polypeptide according to any one of the preceding aspects, wherein the modified BDR region consists of between 20 and 89, between 25 and 89, between 27 and 89, between 27 and 80, between 27 and 75, between 27 and 70, between 27 and 65, between 27 and 60, between 27 and 55, or between 27 and 50 amino acids.

24. The Factor VIII polypeptide according to any one of aspects 1 to 22, wherein the modified BDR region consists of between 30 and 89, between 30 and 89, between 30 and 80, between 30 and 75, between 30 and 70, between 30 and 65, between 30 and 60, between 30 and 55, or between 30 and 50 amino acids.

25. The Factor VIII polypeptide according to any one of aspects 1 to 22, wherein the modified BDR region consists of between 27 and 48, between 30 and 48, between 33 and 48, between 35 and 48, between 40 and 48, or between 42 and 48 amino acids.

26. The Factor VIII polypeptide according to any one of aspects 1 to 22, wherein the modified BDR region consists of between 28 and 48 amino acids.

27. The Factor VIII polypeptide according to any one of aspects 1 to 22, wherein the modified BDR region consists of between 30 and 48 amino acids.

28. The Factor VIII polypeptide according to any one of aspects 1 to 22, wherein the modified BDR region consists of around 45 amino acids.

29. The Factor VIII polypeptide according to any one of aspects 1 to 22, wherein the modified BDR region consists of between 40 and 75, between 40 and 70, between 40 and 65, or between 40 and 60 amino acids.

30. The Factor VIII polypeptide according to any one of aspects 1 to 22, wherein the modified BDR region consists of between 53 and 89, between 53 and 75, between 56 and 75, between 57 and 75, or between 60 and 75 amino acids.

31. The Factor VIII polypeptide according to aspect 29, wherein the modified BDR region consists of between 40 and 60 amino acids.

32. The Factor VIII polypeptide according to any one of aspects 1 to 27 or 29 to 31, wherein the modified BDR region consists of around 54 amino acids.

33. The Factor VIII polypeptide according to any one of the preceding aspects, wherein the Factor VIII amino acid sequence does not comprise at least 806, at least 810, at least 820, at least 835, at least 850, at least 875, or 894 amino acids corresponding to positions 746 to 1639 of SEQ ID NO: 1.

34. The Factor VIII polypeptide according to any one of the preceding aspects, wherein the Factor VIII amino acid sequence does not comprise at least 820, at least 825, at least 834, at least 850, at least 875, or 908 amino acids corresponding to positions 741 to 1648 of SEQ ID NO: 1.

35. The Factor VIII polypeptide according to any one of the preceding aspects, wherein the Factor VIII amino acid sequence comprises at least 650, at least 700, at least 710, or 713 amino acids corresponding to positions 1 to 713 of SEQ ID NO: 1.

36. The Factor VIII polypeptide according to any one of the preceding aspects, wherein the Factor VIII amino acid sequence comprises at least 550, at least 600, at least 620, or 636 amino acids corresponding to positions 1697 to 2332 of SEQ ID NO: 1.

37. The Factor VIII polypeptide according to any one of the preceding aspects, wherein the Factor VIII amino acid sequence does not comprise at least one amino acid(s) corresponding to positions 724 to 740 of SEQ ID NO: 1.

38. The Factor VIII polypeptide according to any one of the preceding aspects, wherein the Factor VIII amino acid sequence does not comprise at least 2, at least 3, at least 4, at least 5, at least 6, at least 7, at least 8, at least 9, at least 10, at least 11, at least 12, at least 13, at least 14, at least 15, at least 16, or 17 amino acids corresponding to positions 724 to 740 of SEQ ID NO: 1.

39. The Factor VIII polypeptide according to any one of the preceding aspects, wherein the Factor VIII amino acid sequence does not comprise 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, or 2 or fewer amino acids corresponding to positions 724 to 740 of SEQ ID NO: 1.

40. The Factor VIII polypeptide according to any one of the preceding aspects, wherein the Factor VIII amino acid sequence does not comprise between 2 and 18, between 5 and 16, or between 8 and 13 amino acids corresponding to positions 724 to 740 of SEQ ID NO: 1.

41. The Factor VIII polypeptide according to any one of the preceding aspects, wherein the Factor VIII amino acid sequence does not comprise at least 2 contiguous amino acids corresponding to positions 724 to 740 of SEQ ID NO: 1.

42. The Factor VIII polypeptide according to any one of the preceding aspects, wherein the Factor VIII amino acid sequence does not comprise at least 3, at least 4, at least 5, at least 6, at least 7, at least 8, at least 9, at least 10, at least 11, at least 12, at least 13, at least 14, at least 15, at least 16, or 17 contiguous amino acids corresponding to positions 724 to 740 of SEQ ID NO: 1.

43. The Factor VIII polypeptide according to any one of the preceding aspects, wherein the Factor VIII amino acid sequence does not comprise amino acids corresponding to positions 739 to 740, 738 to 740, 737 to 740, 736 to 740, 735 to 740, 734 to 740, 733 to 740, 732 to 740, 731 to 740, 730 to 740, 729 to 740, 728 to 740, 727 to 740, 726 to 740, 725 to 740, or 724 to 740 of SEQ ID NO: 1.

44. The Factor VIII polypeptide according to any one of the preceding aspects, wherein the Factor VIII amino acid sequence does not comprise amino acids corresponding to positions 738 to 740 of SEQ ID NO: 1.

45. The Factor VIII polypeptide according to any one of the preceding aspects, wherein the Factor VIII amino acid sequence does not comprise amino acids corresponding to positions 735 to 740 of SEQ ID NO: 1.

46. The Factor VIII polypeptide according to any one of the preceding aspects, wherein the Factor VIII amino acid sequence does not comprise amino acids corresponding to positions 732 to 740 of SEQ ID NO: 1.

47. The Factor VIII polypeptide according to any one of the preceding aspects, wherein the Factor VIII amino acid sequence does not comprise amino acids corresponding to positions 729 to 740 of SEQ ID NO: 1.

48. The Factor VIII polypeptide according to any one of the preceding aspects, wherein the Factor VIII amino acid sequence does not comprise amino acids corresponding to positions 726 to 740 of SEQ ID NO: 1.

49. The Factor VIII polypeptide according to any one of the preceding aspects, wherein the Factor VIII amino acid sequence does not comprise amino acids corresponding to positions 724 to 740 of SEQ ID NO: 1.

50. The Factor VIII polypeptide according to any one of aspects 1 to 48, wherein the Factor VIII amino acid sequence comprises at least one amino acid(s) corresponding to positions 724 to 740 of SEQ ID NO: 1.

51. The Factor VIII polypeptide according to any one of aspects 1 to 48 or 50, wherein the Factor VIII amino acid sequence comprises at least 2, at least 3, at least 4, at least 5, at least 6, at least 7, at least 8, at least 9, at least 10, at least 11, at least 12, at least 13, at least 14, at least 15, at least 16, or 17 amino acids corresponding to positions 724 to 740 of SEQ ID NO: 1.

52. The Factor VIII polypeptide according to any one of aspects 1 to 48 or 50 to 51, wherein the Factor VIII amino acid sequence comprises 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, or 2 or fewer amino acids corresponding to positions 724 to 740 of SEQ ID NO: 1.

53. The Factor VIII polypeptide according to any one of aspects 50 to 52, wherein the Factor VIII amino acid sequence comprises between 1 and 18, between 4 and 15, between 4 and 12, or between 4 and 9 amino acids corresponding to positions 724 to 740 of SEQ ID NO: 1.

54. The Factor VIII polypeptide according to any one of aspects 50 to 53, wherein the Factor VIII amino acid sequence comprises at least 2 contiguous amino acids corresponding to positions 724 to 740 of SEQ ID NO: 1.

55. The Factor VIII polypeptide according to any one of aspects 50 to 54, wherein the Factor VIII amino acid sequence comprises at least 3, at least 4, at least 5, at least 6, at least 7, at least 8, at least 9, at least 10, at least 11, at least 12, at least 13, at least 14, at least 15, at least 16, or 17 contiguous amino acids corresponding to positions 724 to 740 of SEQ ID NO: 1.

56. The Factor VIII polypeptide according to any one of aspects 50 to 55, wherein the Factor VIII amino acid sequence comprises amino acids corresponding to positions 724 to 725, 724 to 726, 724 to 727, 724 to 728, 724 to 729, 724 to 730, 724 to 731, 724 to 732, 724 to 733, 724 to 734, 724 to 735, 724 to 736, 724 to 737, 724 to 738, 724 to 739, or 724 to 740 of SEQ ID NO: 1.

57. The Factor VIII polypeptide according to any one of aspects 50 to 56, wherein the Factor VIII amino acid sequence comprises amino acids corresponding to positions 724 to 725 of SEQ ID NO: 1.

58. The Factor VIII polypeptide according to any one of aspects 50 to 57, wherein the Factor VIII amino acid sequence comprises amino acids corresponding to positions 724 to 728 of SEQ ID NO: 1.

59. The Factor VIII polypeptide according to any one of aspects 50 to 58, wherein the Factor VIII amino acid sequence comprises amino acids corresponding to positions 724 to 731 of SEQ ID NO: 1.

60. The Factor VIII polypeptide according to any one of aspects 50 to 59, wherein the Factor VIII amino acid sequence comprises amino acids corresponding to positions 724 to 734 of SEQ ID NO: 1.

61. The Factor VIII polypeptide according to any one of aspects 50 to 60, wherein the

Factor VIII amino acid sequence comprises amino acids corresponding to positions 724 to 737 of SEQ ID NO: 1.

62. The Factor VIII polypeptide according to any one of aspects 50 to 61, wherein the Factor VIII amino acid sequence comprises amino acids corresponding to positions 724 to 740 of SEQ ID NO: 1.

63. The Factor VIII polypeptide according to any one of the preceding aspects, wherein the Factor VIII amino acid sequence comprises at least one of the tyrosine amino acids corresponding to positions 718, 719 and 723 of SEQ ID NO: 1.

64. The Factor VIII polypeptide according to aspect 63, wherein the Factor VIII amino acid sequence comprises the tyrosine amino acid corresponding to position 718 of SEQ ID NO: 1.

65. The Factor VIII polypeptide according to aspect 63 or 64, wherein the Factor VIII amino acid sequence comprises the tyrosine amino acid corresponding to position 719 of SEQ ID NO: 1.

66. The Factor VIII polypeptide according to any one of aspects 63 to 65, wherein the Factor VIII amino acid sequence comprises the tyrosine amino acid corresponding to position 723 of SEQ ID NO: 1.

67. The Factor VIII polypeptide according to any one of aspects 63 to 66, wherein the Factor VIII amino acid sequence comprises the tyrosine amino acids corresponding to positions 718, 719 and 723 of SEQ ID NO: 1.

68. The Factor VIII polypeptide according to any one of the preceding aspects, wherein the Factor VIII amino acid sequence comprises at least one of the amino acid(s) corresponding to positions 714 to 723 of SEQ ID NO: 1.

69. The Factor VIII polypeptide according to any one of the preceding aspects, wherein the Factor VIII amino acid sequence comprises at least 2, at least 3, at least 4, at least 5, at least 6, at least 7, at least 8, at least 9, or 10 amino acids corresponding to positions 714 to 723 of SEQ ID NO: 1.

70. The Factor VIII polypeptide according to any one of the preceding aspects, wherein the Factor VIII amino acid sequence comprises 10 or fewer, 9 or fewer, 8 or fewer, 7 or fewer, 6 or fewer, 5 or fewer, 4 or fewer, 3 or fewer, or 2 or fewer amino acids corresponding to positions 714 to 723 of SEQ ID NO: 1.

71. The Factor VIII polypeptide according to any one of the preceding aspects, wherein the Factor VIII amino acid sequence comprises between 2 and 11, between 5 and 11, or between 7 and 11 amino acids corresponding to positions 714 to 723 of SEQ ID NO: 1.

72. The Factor VIII polypeptide according to any one of the preceding aspects, wherein the Factor VIII amino acid sequence comprises at least 2 contiguous amino acids corresponding to positions 714 to 723 of SEQ ID NO: 1.

73. The Factor VIII polypeptide according to any one of the preceding aspects, wherein the Factor VIII amino acid sequence comprises at least 3, at least 4, at least 5, at least 6, at least 7, at least 8, at least 9, or 10 contiguous amino acids corresponding to positions 714 to 723 of SEQ ID NO: 1.

74. The Factor VIII polypeptide according to any one of the preceding aspects, wherein the Factor VIII amino acid sequence comprises amino acids corresponding to positions 714 to 715, 714 to 716, 714 to 717, 714 to 718, 714 to 719, 714 to 720, 714 to 721, 714 to 722, or 714 to 723 of SEQ ID NO: 1.

75. The Factor VIII polypeptide according to any one of the preceding aspects, wherein the Factor VIII amino acid sequence comprises amino acids corresponding to positions 714 to 719 of SEQ ID NO: 1.

76. The Factor VIII polypeptide according to any one of the preceding aspects, wherein the Factor VIII amino acid sequence comprises amino acids corresponding to positions 714 to 722 of SEQ ID NO: 1.

77. The Factor VIII polypeptide according to any one of the preceding aspects, wherein the Factor VIII amino acid sequence comprises amino acids corresponding to positions 714 to 723 of SEQ ID NO: 1.

78. The Factor VIII polypeptide according to any one of the preceding aspects, wherein the Factor VIII amino acid sequence does not comprise at least one amino acid(s) corresponding to positions 741 to 745 of SEQ ID NO: 1.

79. The Factor VIII polypeptide according to any one of the preceding aspects, wherein the Factor VIII amino acid sequence does not comprise at least 2, at least 3, at least 4, or 5 amino acids corresponding to positions 741 to 745 of SEQ ID NO: 1.

80. The Factor VIII polypeptide according to any one of the preceding aspects, wherein the Factor VIII amino acid sequence does not comprise 5 or fewer, 4 or fewer, 3 or fewer, or 2 or fewer amino acids corresponding to positions 741 to 745 of SEQ ID NO: 1.

81. The Factor VIII polypeptide according to any one of the preceding aspects, wherein the Factor VIII amino acid sequence does not comprise between 1 and 6, or between 3 and 6 amino acids corresponding to positions 741 to 745 of SEQ ID NO: 1.

82. The Factor VIII polypeptide according to any one of the preceding aspects, wherein the Factor VIII amino acid sequence does not comprise at least 2 contiguous amino acids corresponding to positions 741 to 745 of SEQ ID NO: 1.

83. The Factor VIII polypeptide according to any one of the preceding aspects, wherein the Factor VIII amino acid sequence does not comprise at least 3, at least 4, or 5 contiguous amino acids corresponding to positions 741 to 745 of SEQ ID NO: 1.

84. The Factor VIII polypeptide according to any one of the preceding aspects, wherein the Factor VIII amino acid sequence does not comprise amino acids corresponding to positions 744 to 745, 743 to 745, 742 to 745, or 741 to 745 of SEQ ID NO: 1.

85. The Factor VIII polypeptide according to any one of the preceding aspects, wherein the Factor VIII amino acid sequence does not comprise amino acids corresponding to positions 741 to 745 of SEQ ID NO: 1.

86. The Factor VIII polypeptide according to any one of the preceding aspects, wherein the Factor VIII amino acid sequence does not comprise at least one amino acid(s) corresponding to positions 1640 to 1648 of SEQ ID NO: 1.

87. The Factor VIII polypeptide according to any one of the preceding aspects, wherein the Factor VIII amino acid sequence does not comprise at least 2, at least 3, at least 4, at least 5, at least 6, at least 7, at least 8, or 9 amino acids corresponding to positions 1640 to 1648 of SEQ ID NO: 1.

88. The Factor VIII polypeptide according to any one of the preceding aspects, wherein the Factor VIII amino acid sequence does not comprise 9 or fewer, 8 or fewer, 7 or fewer, 6 or fewer, 5 or fewer, 4 or fewer, 3 or fewer, or 2 or fewer amino acids corresponding to positions 1640 to 1648 of SEQ ID NO: 1.

89. The Factor VIII polypeptide according to any one of the preceding aspects, wherein the Factor VIII amino acid sequence does not comprise between 1 and 10, between 4 and 10, or between 6 and 10 amino acids corresponding to positions 1640 to 1648 of SEQ ID NO: 1.

90. The Factor VIII polypeptide according to any one of the preceding aspects, wherein the Factor VIII amino acid sequence does not comprise at least 2 contiguous amino acids corresponding to positions 1640 to 1648 of SEQ ID NO: 1.

91. The Factor VIII polypeptide according to any one of the preceding aspects, wherein the Factor VIII amino acid sequence does not comprise at least 3, at least 4, at least 5, at least 6, at least 7, at least 8, or 9 contiguous amino acids corresponding to positions 1640 to 1648 of SEQ ID NO: 1.

92. The Factor VIII polypeptide according to any one of the preceding aspects, wherein the Factor VIII amino acid sequence does not comprise amino acids corresponding to positions 1640 to 1641, 1640 to 1642, 1640 to 1643, 1640 to 1644, 1640 to 1645, 1640 to 1646, 1640 to 1647, or 1640 to 1648 of SEQ ID NO: 1.

93. The Factor VIII polypeptide according to any one of the preceding aspects, wherein the Factor VIII amino acid sequence does not comprise amino acids corresponding to positions 1640 to 1648 of SEQ ID NO: 1.

94. The Factor VIII polypeptide according to any one of aspects 1 to 92, wherein the Factor VIII amino acid sequence comprises amino acids corresponding to positions 1645 to 1648 of SEQ ID NO: 1, preferably wherein:
the amino acid at the position corresponding to position 1645 of SEQ ID NO: 1 is arginine;
the amino acid at the position corresponding to position 1646 of SEQ ID NO: 1 is histidine;
the amino acid at the position corresponding to position 1647 of SEQ ID NO: 1 is glutamine; and
the amino acid at the position corresponding to position 1648 of SEQ ID NO: 1 is arginine.

95. The Factor VIII polypeptide according to any one of the preceding aspects, wherein the Factor VIII amino acid sequence does not comprise at least one amino acid(s) corresponding to positions 1649 to 1669 of SEQ ID NO: 1.

96. The Factor VIII polypeptide according to any one of the preceding aspects, wherein the Factor VIII amino acid sequence does not comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or 21 amino acids corresponding to positions 1649 to 1669 of SEQ ID NO: 1.

97. The Factor VIII polypeptide according to any one of the preceding aspects, wherein the Factor VIII amino acid sequence does not comprise 21 or fewer, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, or 2 or fewer amino acids corresponding to positions 1649 to 1669 of SEQ ID NO: 1.

98. The Factor VIII polypeptide according to any one of the preceding aspects, wherein the Factor VIII amino acid sequence does not comprise between 1 and 22, between 5 and 22, between 8 and 22, between 11 and 22, between 14 and 22, between 17 and 22, or between 19 and 22 amino acids corresponding to positions 1649 to 1669 of SEQ ID NO: 1.

99. The Factor VIII polypeptide according to any one of the preceding aspects, wherein the Factor VIII amino acid sequence does not comprise at least 2 contiguous amino acids corresponding to positions 1649 to 1669 of SEQ ID NO: 1.

100. The Factor VIII polypeptide according to any one of the preceding aspects, wherein the Factor VIII amino acid sequence does not comprise at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or 21 contiguous amino acids corresponding to positions 1649 to 1669 of SEQ ID NO: 1.

101. The Factor VIII polypeptide according to any one of the preceding aspects, wherein the Factor VIII amino acid sequence does not comprise amino acids corresponding to positions 1649 to 1650, 1649 to 1651, 1649 to 1652, 1649 to 1653, 1649 to 1654, 1649 to 1655, 1649 to 1656, 1649 to 1657, 1649 to 1658, 1649 to 1659, 1649 to 1660, 1649 to 1661, 1649 to 1662, 1649 to 1663, 1649 to 1664, 1649 to 1665, 1649 to 1666, 1649 to 1667, 1649 to 1668, or 1649 to 1669 of SEQ ID NO: 1.

102. The Factor VIII polypeptide according to any one of the preceding aspects, wherein the Factor VIII amino acid sequence does not comprise amino acids corresponding to positions 1649 to 1651 of SEQ ID NO: 1.

103. The Factor VIII polypeptide according to any one of the preceding aspects, wherein the Factor VIII amino acid sequence does not comprise amino acids corresponding to positions 1649 to 1654 of SEQ ID NO: 1.

104. The Factor VIII polypeptide according to any one of the preceding aspects, wherein the Factor VIII amino acid sequence does not comprise amino acids corresponding to positions 1649 to 1657 of SEQ ID NO: 1.

105. The Factor VIII polypeptide according to any one of the preceding aspects, wherein the Factor VIII amino acid sequence does not comprise amino acids corresponding to positions 1649 to 1660 of SEQ ID NO: 1.

106. The Factor VIII polypeptide according to any one of the preceding aspects, wherein the Factor VIII amino acid sequence does not comprise amino acids corresponding to positions 1649 to 1663 of SEQ ID NO: 1.

107. The Factor VIII polypeptide according to any one of the preceding aspects, wherein the Factor VIII amino acid sequence does not comprise amino acids corresponding to positions 1649 to 1664 of SEQ ID NO: 1.

108. The Factor VIII polypeptide according to any one of the preceding aspects, wherein the Factor VIII amino acid sequence does not comprise amino acids corresponding to positions 1649 to 1666 of SEQ ID NO: 1.

109. The Factor VIII polypeptide according to any one of the preceding aspects, wherein the Factor VIII amino acid sequence does not comprise amino acids corresponding to positions 1649 to 1667 of SEQ ID NO: 1.

110. The Factor VIII polypeptide according to any one of the preceding aspects, wherein the Factor VIII amino acid sequence does not comprise amino acids corresponding to positions 1649 to 1669 of SEQ ID NO: 1.

111. The Factor VIII polypeptide according to any one of aspects 1 to 109, wherein the Factor VIII amino acid sequence comprises at least one of the amino acid(s) corresponding to positions 1649 to 1669 of SEQ ID NO: 1.

112. The Factor VIII polypeptide according to aspect 111, wherein the Factor VIII amino acid sequence comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or 21 amino acids corresponding to positions 1649 to 1669 of SEQ ID NO: 1.

113. The Factor VIII polypeptide according to aspect 111 or 112, wherein the Factor VIII amino acid sequence comprises 21 or fewer, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, or 2 or fewer amino acids corresponding to positions 1649 to 1669 of SEQ ID NO: 1.

114. The Factor VIII polypeptide according to any one of aspects 111 to 113, wherein the Factor VIII amino acid sequence comprises between 1 and 22, between 1 and 19, between 1 and 16, between 1 and 12, between 1 and 9, between 1 and 6, or between 1 and 4 amino acids corresponding to positions 1649 to 1669 of SEQ ID NO: 1.

115. The Factor VIII polypeptide according to any one of aspects 111 to 114, wherein the Factor VIII amino acid sequence comprises at least 2 contiguous amino acids corresponding to positions 1649 to 1669 of SEQ ID NO: 1.

116. The Factor VIII polypeptide according to any one of aspects 111 to 115, wherein the Factor VIII amino acid sequence comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or 21 contiguous amino acids corresponding to positions 1649 to 1669 of SEQ ID NO: 1.

117. The Factor VIII polypeptide according to any one of aspects 111 to 116, wherein the Factor VIII amino acid sequence comprises amino acids corresponding to positions 1668 to 1669, 1667 to 1669, 1666 to 1669, 1665 to 1669, 1664 to 1669, 1663 to 1669, 1662 to 1669, 1661 to 1669, 1660 to 1669, 1659 to 1669, 1658 to 1669, 1657 to 1669, 1656 to 1669, 1655 to 1669, 1654 to 1669, 1653 to 1669, 1652 to 1669, 1651 to 1669, 1650 to 1669, or 1649 to 1669 of SEQ ID NO: 1.

118. The Factor VIII polypeptide according to any one of aspects 111 to 117, wherein the Factor VIII amino acid sequence comprises an amino acid corresponding to position 1664 of SEQ ID NO: 1, preferably wherein the amino acid is a tyrosine.

119. The Factor VIII polypeptide according to any one of aspects 111 to 118, wherein the Factor VIII amino acid sequence comprises amino acids corresponding to positions 1668 and/or 1669 of SEQ ID NO: 1.

120. The Factor VIII polypeptide according to any one of aspects 111 to 119, wherein the Factor VIII amino acid sequence comprises amino acids corresponding to positions 1668 to 1669 of SEQ ID NO: 1.

121. The Factor VIII polypeptide according to any one of aspects 111 to 120, wherein the Factor VIII amino acid sequence comprises amino acids corresponding to positions 1667 to 1669 of SEQ ID NO: 1.

122. The Factor VIII polypeptide according to any one of aspects 111 to 121, wherein the Factor VIII amino acid sequence comprises amino acids corresponding to positions 1664 to 1669 of SEQ ID NO: 1.

123. The Factor VIII polypeptide according to any one of aspects 111 to 122, wherein the Factor VIII amino acid sequence comprises amino acids corresponding to positions 1661 to 1669 of SEQ ID NO: 1.

124. The Factor VIII polypeptide according to any one of aspects 111 to 123, wherein the Factor VIII amino acid sequence comprises amino acids corresponding to positions 1658 to 1669 of SEQ ID NO: 1.

125. The Factor VIII polypeptide according to any one of aspects 111 to 124, wherein the Factor VIII amino acid sequence comprises amino acids corresponding to positions 1655 to 1669 of SEQ ID NO: 1.

126. The Factor VIII polypeptide according to any one of aspects 111 to 125, wherein the Factor VIII amino acid sequence comprises amino acids corresponding to positions 1652 to 1669 of SEQ ID NO: 1.

127. The Factor VIII polypeptide according to any one of aspects 111 to 126, wherein the Factor VIII amino acid sequence comprises amino acids corresponding to positions 1649 to 1669 of SEQ ID NO: 1.

128. The Factor VIII polypeptide according to any one of the preceding aspects, wherein the Factor VIII amino acid sequence comprises at least one amino acid(s) corresponding to positions 1670 to 1678 of SEQ ID NO: 1.

129. The Factor VIII polypeptide according to any one of the preceding aspects, wherein the Factor VIII amino acid sequence comprises at least 2, at least 3, at least 4, at least 5, at least 6, at least 7, at least 8, or 9 amino acids corresponding to positions 1670 to 1678 of SEQ ID NO: 1.

130. The Factor VIII polypeptide according to any one of the preceding aspects, wherein the Factor VIII amino acid sequence comprises 9 or fewer, 8 or fewer, 7 or fewer, 6 or fewer, 5 or fewer, 4 or fewer, 3 or fewer, or 2 or fewer amino acids corresponding to positions 1670 to 1678 of SEQ ID NO: 1.

131. The Factor VIII polypeptide according to any one of the preceding aspects, wherein the Factor VIII amino acid sequence comprises between 1 and 10, between 4 and 10, or between 7 and 10 amino acids corresponding to positions 1670 to 1678 of SEQ ID NO: 1.

132. The Factor VIII polypeptide according to any one of the preceding aspects, wherein the Factor VIII amino acid sequence comprises at least 2 contiguous amino acids corresponding to positions 1670 to 1678 of SEQ ID NO: 1.

133. The Factor VIII polypeptide according to any one of the preceding aspects, wherein the Factor VIII amino acid sequence comprises at least 3, at least 4, at least 5, at least 6, at least 7, at least 8, or 9 contiguous amino acids corresponding to positions 1670 to 1678 of SEQ ID NO: 1.

134. The Factor VIII polypeptide according to any one of the preceding aspects, wherein the Factor VIII amino acid sequence comprises amino acids corresponding to positions 1670 to 1678 of SEQ ID NO: 1.

135. The Factor VIII polypeptide according to any one of the preceding aspects, wherein the Factor VIII polypeptide can bind to a von Willebrand factor.

136. The Factor VIII polypeptide according to any one of the preceding aspects, wherein the Factor VIII amino acid sequence comprises a von Willebrand factor binding site.

137. The Factor VIII polypeptide according to aspect 135 or 136, wherein the ability of the Factor VIII polypeptide to bind to a von Willebrand factor is measured by ELISA or wherein the Factor VIII amino acid sequence comprises a von Willebrand factor binding site if it can bind to a von Willebrand factor as measured by an ELISA.

138. The Factor VIII polypeptide according to any one of the preceding aspects, wherein the Factor VIII amino acid sequence comprises an amino acid corresponding to position 1679 of SEQ ID NO: 1.

139. The Factor VIII polypeptide according to any one of the preceding aspects, wherein the Factor VIII amino acid sequence comprises an amino acid corresponding to position 1680 of SEQ ID NO: 1, preferably wherein the amino acid is a tyrosine.

140. The Factor VIII polypeptide according to any one of the preceding aspects, wherein the Factor VIII amino acid sequence comprises amino acids corresponding to positions 1679 and 1680 of SEQ ID NO: 1, preferably wherein the amino acid corresponding to position 1680 is a tyrosine.

141. The Factor VIII polypeptide according to any one of aspects 1 to 137 or 139, wherein the Factor VIII amino acid sequence does not comprise an amino acid corresponding to position 1679 of SEQ ID NO: 1.

142. The Factor VIII polypeptide according to any one of aspects 1 to 138 or 141, wherein the Factor VIII amino acid sequence does not comprise an amino acid corresponding to position 1680 of SEQ ID NO: 1.

143. The Factor VIII polypeptide according to aspect 141 or 142, wherein the Factor VIII amino acid sequence does not comprise amino acids corresponding to positions 1679 and 1680 of SEQ ID NO: 1.

144. The Factor VIII polypeptide according to any one of the preceding aspects, wherein the Factor VIII amino acid sequence does not comprise at least one amino acid(s) corresponding to positions 1681 to 1689 of SEQ ID NO: 1.

145. The Factor VIII polypeptide according to any one of the preceding aspects, wherein the Factor VIII amino acid sequence does not comprise at least 2, at least 3, at least 4, at least 5, at least 6, at least 7, at least 8, or 9 amino acids corresponding to positions 1681 to 1689 of SEQ ID NO: 1.

146. The Factor VIII polypeptide according to any one of the preceding aspects, wherein the Factor VIII amino acid sequence does not comprise 9 or fewer, 8 or fewer, 7 or fewer, 6 or fewer, 5 or fewer, 4 or fewer, 3 or fewer, or 2 or fewer amino acids corresponding to positions 1681 to 1689 of SEQ ID NO: 1.

147. The Factor VIII polypeptide according to any one of the preceding aspects, wherein the Factor VIII amino acid sequence does not comprise between 1 and 10, between 4 and 10, or between 6 and 10 amino acids corresponding to positions 1681 to 1689 of SEQ ID NO: 1.

148. The Factor VIII polypeptide according to any one of the preceding aspects, wherein the Factor VIII amino acid sequence does not comprise at least 2 contiguous amino acids corresponding to positions 1681 to 1689 of SEQ ID NO: 1.

149. The Factor VIII polypeptide according to any one of the preceding aspects, wherein the Factor VIII amino acid sequence does not comprise at least 3, at least 4, at least 5, at least 6, at least 7, at least 8, or 9 contiguous amino acids corresponding to positions 1681 to 1689 of SEQ ID NO: 1.

150. The Factor VIII polypeptide according to any one of the preceding aspects, wherein the Factor VIII amino acid sequence does not comprise amino acids corresponding to positions 1681 to 1682, 1681 to 1683, 1681 to 1684, 1681 to 1685, 1681 to 1686, 1681 to 1687, 1681 to 1688, or 1681 to 1689 of SEQ ID NO: 1.

151. The Factor VIII polypeptide according to any one of the preceding aspects, wherein the Factor VIII amino acid sequence does not comprise amino acids corresponding to positions 1681 to 1683 of SEQ ID NO: 1.

152. The Factor VIII polypeptide according to any one of the preceding aspects, wherein the Factor VIII amino acid sequence does not comprise amino acids corresponding to positions 1681 to 1686 of SEQ ID NO: 1.

153. The Factor VIII polypeptide according to any one of the preceding aspects, wherein the Factor VIII amino acid sequence does not comprise amino acids corresponding to positions 1681 to 1688 of SEQ ID NO: 1.

154. The Factor VIII polypeptide according to any one of the preceding aspects, wherein the Factor VIII amino acid sequence does not comprise amino acids corresponding to positions 1681 to 1689 of SEQ ID NO: 1.

155. The Factor VIII polypeptide according to any one of aspects 1 to 153, wherein the Factor VIII amino acid sequence comprises at least one of the amino acid(s) corresponding to positions 1681 to 1689 of SEQ ID NO: 1.

156. The Factor VIII polypeptide according to aspect 155, wherein the Factor VIII amino acid sequence comprises at least 2, at least 3, at least 4, at least 5, at least 6, at least 7, at least 8, or 9 amino acids corresponding to positions 1681 to 1689 of SEQ ID NO: 1.

157. The Factor VIII polypeptide according to aspect 155 or 156, wherein the Factor VIII amino acid sequence comprises 9 or fewer, 8 or fewer, 7 or fewer, 6 or fewer, 5 or fewer, 4 or fewer, 3 or fewer, or 2 or fewer amino acids corresponding to positions 1681 to 1689 of SEQ ID NO: 1.

158. The Factor VIII polypeptide according to any one of aspects 155 to 157, wherein the Factor VIII amino acid sequence comprises between 1 and 10, between 4 and 10, or between 6 and 10 amino acids corresponding to positions 1681 to 1689 of SEQ ID NO: 1.

159. The Factor VIII polypeptide according to any one of aspects 155 to 158, wherein the Factor VIII amino acid sequence comprises at least 2 contiguous amino acids corresponding to positions 1681 to 1689 of SEQ ID NO: 1.

160. The Factor VIII polypeptide according to any one of aspects 155 to 159, wherein the Factor VIII amino acid sequence comprises at least 3, at least 4, at least 5, at least 6, at least 7, at least 8, or 9 contiguous amino acids corresponding to positions 1681 to 1689 of SEQ ID NO: 1.

161. The Factor VIII polypeptide according to any one of aspects 155 to 160, wherein the Factor VIII amino acid sequence comprises amino acids corresponding to positions 1688 to 1689, 1687 to 1689, 1686 to 1689, 1685 to 1689, 1684 to 1689, 1683 to 1689, 1682 to 1689, or 1681 to 1689 of SEQ ID NO: 1.

162. The Factor VIII polypeptide according to any one of aspects 155 to 161, wherein the Factor VIII amino acid sequence comprises an amino acid corresponding to position 1689 of SEQ ID NO: 1.

163. The Factor VIII polypeptide according to any one of aspects 155 to 162, wherein the Factor VIII amino acid sequence comprises amino acids corresponding to positions 1687 to 1689 of SEQ ID NO: 1.

164. The Factor VIII polypeptide according to any one of aspects 155 to 163, wherein the Factor VIII amino acid sequence comprises amino acids corresponding to positions 1684 to 1689 of SEQ ID NO: 1.

165. The Factor VIII polypeptide according to any one of aspects 155 to 164, wherein the Factor VIII amino acid sequence comprises amino acids corresponding to positions 1681 to 1689 of SEQ ID NO: 1.

166. The Factor VIII polypeptide according to any one of the preceding aspects, wherein the Factor VIII amino acid sequence comprises at least one of the amino acid(s) corresponding to positions 1690 to 1696 of SEQ ID NO: 1.

167. The Factor VIII polypeptide according to any one of the preceding aspects, wherein the Factor VIII amino acid sequence comprises at least 2, at least 3, at least 4, at least 5, at least 6, or 7 amino acids corresponding to positions 1690 to 1696 of SEQ ID NO: 1.

168. The Factor VIII polypeptide according to any one of the preceding aspects, wherein the Factor VIII amino acid sequence comprises 7 or fewer, 6 or fewer, 5 or fewer, 4 or fewer, 3 or fewer, or 2 or fewer amino acids corresponding to positions 1690 to 1696 of SEQ ID NO: 1.

169. The Factor VIII polypeptide according to any one of the preceding aspects, wherein the Factor VIII amino acid sequence comprises between 2 and 8, or between 5 and 8 amino acids corresponding to positions 1690 to 1696 of SEQ ID NO: 1.

170. The Factor VIII polypeptide according to any one of the preceding aspects, wherein the Factor VIII amino acid sequence comprises at least 2 contiguous amino acids corresponding to positions 1690 to 1696 of SEQ ID NO: 1.

171. The Factor VIII polypeptide according to any one of the preceding aspects, wherein the Factor VIII amino acid sequence comprises at least 3, at least 4, at least 5, at least 6, or 7 contiguous amino acids corresponding to positions 1690 to 1696 of SEQ ID NO: 1.

172. The Factor VIII polypeptide according to any one of the preceding aspects, wherein the Factor VIII amino acid sequence comprises amino acids corresponding to positions 1695 to 1696, 1694 to 1696, 1693 to 1696, 1692 to 1696, 1691 to 1696, or 1690 to 1696 of SEQ ID NO: 1.

173. The Factor VIII polypeptide according to any one of the preceding aspects, wherein the Factor VIII amino acid sequence comprises amino acids corresponding to positions 1690 to 1696 of SEQ ID NO: 1.

174. The Factor VIII polypeptide according to any one of aspects 1 to 35, 37 to 94, 95 to 110 or 128 to 173, wherein the Factor VIII amino acid sequence does not comprise amino acids corresponding to positions 741 to 1669 of SEQ ID NO: 1.

175. The Factor VIII polypeptide according to aspect 174, wherein the Factor VIII amino acid sequence does not comprise amino acids corresponding to positions 741 to 1669 of SEQ ID NO: 1, and the Factor VIII amino acid sequence comprises amino acids corresponding to positions 724 to 740 of SEQ ID NO: 1.

176. The Factor VIII polypeptide according to aspect 174 or 175, wherein the Factor VIII amino acid sequence does not comprise amino acids corresponding to positions 741 to 1669 of SEQ ID NO: 1, and the Factor VIII amino acid sequence comprises amino acids corresponding to positions 1670 to 1689 of SEQ ID NO: 1.

177. The Factor VIII polypeptide according to any one of aspects 174 to 176, wherein the Factor VIII amino acid sequence does not comprise amino acids corresponding to positions 741 to 1669 of SEQ ID NO: 1, and the Factor VIII amino acid sequence comprises amino acids corresponding to positions 724 to 740 and 1670 to 1689 of SEQ ID NO: 1.

178. The Factor VIII polypeptide according to any one of aspects 174 to 177, wherein the Factor VIII amino acid sequence does not comprise amino acids corresponding to positions 741 to 1669 of SEQ ID NO: 1, and the Factor VIII amino acid sequence comprises amino acids corresponding to positions 724 to 740 and 1670 to 1689 of SEQ ID NO: 1 and the tyrosine amino acids corresponding to positions 718, 719 and 723 of SEQ ID NO: 1.

179. The Factor VIII polypeptide according to any one of aspects 174 to 178, wherein the Factor VIII amino acid sequence does not comprise amino acids corresponding to positions 741 to 1669 of SEQ ID NO: 1, and the Factor VIII amino acid sequence comprises amino acids corresponding to positions 714 to 740 and 1670 to 1689 of SEQ ID NO: 1.

180. The Factor VIII polypeptide according to any one of aspects 174 to 179, wherein the Factor VIII amino acid sequence does not comprise amino acids corresponding to positions 741 to 1669 of SEQ ID NO: 1, and the Factor VIII amino acid sequence comprises amino acids corresponding to positions 1 to 740 and 1670 to 2332 of SEQ ID NO: 1.

181. The Factor VIII polypeptide according to aspect 180, wherein the Factor VIII amino acid sequence comprises SEQ ID NO: 9.

182. The Factor VIII polypeptide according to any one of aspects 1 to 35, 59 to 109, or 128 to 180, wherein the Factor VIII amino acid sequence does not comprise amino acids corresponding to positions 732 to 1669 of SEQ ID NO: 1.

183. The Factor VIII polypeptide according to aspect 182, wherein the Factor VIII amino acid sequence does not comprise amino acids corresponding to positions 732 to 1669 of SEQ ID NO: 1, and the Factor VIII amino acid sequence comprises amino acids corresponding to positions 724 to 731 of SEQ ID NO: 1.

184. The Factor VIII polypeptide according to aspect 182 or 183, wherein the Factor VIII amino acid sequence does not comprise amino acids corresponding to positions 732 to 1669 of SEQ ID NO: 1, and the Factor VIII amino acid sequence comprises amino acids corresponding to positions 1670 to 1689 of SEQ ID NO: 1.

185. The Factor VIII polypeptide according to any one of aspects 182 to 184, wherein the Factor VIII amino acid sequence does not comprise amino acids corresponding to positions 732 to 1669 of SEQ ID NO: 1, and the Factor VIII amino acid sequence comprises amino acids corresponding to positions 724 to 731 and 1670 to 1689 of SEQ ID NO: 1.

186. The Factor VIII polypeptide according to any one of aspects 182 to 185, wherein the Factor VIII amino acid sequence does not comprise amino acids corresponding to positions 732 to 1669 of SEQ ID NO: 1, and the Factor VIII amino acid sequence comprises amino acids corresponding to positions 724 to 731 and 1670 to 1689 of SEQ ID NO: 1 and the tyrosine amino acids corresponding to positions 718, 719 and 723 of SEQ ID NO: 1.

187. The Factor VIII polypeptide according to any one of aspects 182 to 186, wherein the Factor VIII amino acid sequence does not comprise amino acids corresponding to positions 732 to 1669 of SEQ ID NO: 1, and the Factor VIII amino acid sequence comprises amino acids corresponding to positions 714 to 731 and 1670 to 1689 of SEQ ID NO: 1.

188. The Factor VIII polypeptide according to any one of aspects 182 to 187, wherein the Factor VIII amino acid sequence does not comprise amino acids corresponding to positions 732 to 1669 of SEQ ID NO: 1, and the Factor VIII amino acid sequence comprises amino acids corresponding to positions 1 to 731 and 1670 to 2332 of SEQ ID NO: 1.

189. The Factor VIII polypeptide according to any one of aspects 182 to 188, wherein the Factor VIII amino acid sequence comprises SEQ ID NO: 31.

190. The Factor VIII polypeptide according to any one of the preceding aspects, wherein the Factor VIII amino acid sequence comprises any one of SEQ ID NOs: 9-36.

191. The Factor VIII polypeptide according to any one of aspects 1 to 190, wherein the Factor VIII amino acid sequence comprises one or more substitution mutations at an inter-domain interface selected from the group consisting of:
a. the A1/A3 domain interface;
b. the A2/A3 domain interface; or
c. the A1/C2 domain interface,
   wherein:
(i) the one or more substitution mutations comprises substitution of an amino acid with a more hydrophobic amino acid; or
(ii) the one or more substitution mutations comprises substitution of a pair of amino acids in the respective domains with cysteine residues;
   and wherein the Factor VIII polypeptide has higher specific activity than a reference wild-type Factor VIII polypeptide.

192. The Factor VIII polypeptide according to any one of aspects 1 to 190, wherein the Factor VIII amino acid sequence comprises one or more substitution mutations at an inter-domain interface selected from the group consisting of:
a. the A1/A3 domain interface;
b. the A2/A3 domain interface; or
c. the A1/C2 domain interface
   wherein:
(i) the one or more substitution mutations comprises substitution of an amino acid with a more hydrophobic amino acid; or
(ii) the one or more substitution mutations comprises substitution of a pair of amino acids in respective domains with cysteine residues;
   and wherein the Factor VIII polypeptide has higher stability than a reference wild-type Factor VIII polypeptide.

193. The Factor VIII polypeptide according to any one aspects 1 to 190, wherein the Factor VIII amino acid sequence comprises one or more substitution mutations at an inter-domain interface selected from the group consisting of:
a. the A1/A3 domain interface;
b. the A2/A3 domain interface; or
c. the A1/C2 domain interface
   wherein:
(i) the one or more substitution mutations comprises substitution of an amino acid with a more hydrophobic amino acid; or
(ii) the one or more substitution mutations comprises substitution of a pair of amino acids in respective domains with cysteine residues;
   and wherein the Factor VIII polypeptide is expressed at a higher level in a host cell than a reference wild-type Factor VIII polypeptide.

194. The Factor VIII polypeptide according to any one of aspects 1 to 190, wherein the Factor VIII amino acid sequence comprises a one or more substitution mutations selected from the group consisting of:
a. a substitution of an amino acid corresponding to M662 or H693 of SEQ ID NO: 1; or
b. a substitution of a pair of amino acids comprising a first amino acid and a second amino acid with cysteine residues, wherein:
   1. the first amino acid corresponds to M147, S149 or S289 of SEQ ID NO: 1 and the second amino acid corresponds to E1969, E1970 or N1977 of SEQ ID NO: 1;
   2. the first amino acid corresponds to T667, T669, N684, L687, I689, S695 or F697 of SEQ ID NO: 1 and the second amino acid corresponds to S1791, G1799, A1800, R1803, E1844, S1949, G1981, V1982, or Y1979 of SEQ ID NO: 1; or
   3. the first amino acid corresponds to A108, T118 or V137 of SEQ ID NO: 1 and the second amino acid corresponds to N2172, Q2329 or Y2332 of SEQ ID NO: 1.

195. The Factor VIII polypeptide according to any one of aspects 191 to 194, wherein the Factor VIII polypeptide has higher specific activity relative to a reference wild-type Factor VIII polypeptide.

196. The Factor VIII polypeptide according to aspect 191 or 195, wherein the Factor VIII polypeptide has a specific activity which is at least 1.1 fold, at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of the reference Factor VIII polypeptide.

197. The Factor VIII polypeptide according to any one of aspects 191 or 193 to 196, wherein the Factor VIII polypeptide has higher stability relative to a reference wild-type Factor VIII polypeptide.

198. The Factor VIII polypeptide according to aspect 192 or 197, wherein the Factor VIII polypeptide has a longer half-life relative to the reference wild-type Factor VIII polypeptide, optionally wherein the Factor VIII polypeptide has a longer half-life relative to the reference wild-type Factor VIII polypeptide when activated.

199. The Factor VIII polypeptide according to aspect 198, wherein the Factor VIII polypeptide has a longer half-life which is at least 1.1, at least 1.2, at least 1.5, at least 1.7, at least 1.8, at least 2, at least 2.2, at least 2.5, at least 2.8 or at least 3 times the half-life of a reference wild-type Factor VIII polypeptide, and/or wherein the Factor VIII polypeptide has a half-life when activated which is at least 1.1, at least 1.2, at least 1.5, at least 1.7, at least 1.8, at least 2, at least 2.2, at least 2.5, at least 2.8 or at least 3 times the half-life of a reference wild-type Factor VIII polypeptide when activated.

200. The Factor VIII polypeptide according to aspect 198 or 199, wherein the longer half-life is longer half-life in plasma.

201. The Factor VIII polypeptide according to aspect 191, 192 or 194 to 196, wherein the Factor VIII polypeptide is expressed at a higher level in a host cell than a reference wild-type Factor VIII polypeptide.

202. The Factor VIII polypeptide according to aspect 201, wherein the level of Factor VIII polypeptide that is expressed is the level of FVIII polypeptide secreted by a host cell.

203. The Factor VIII polypeptide according to aspect 201 or 202, wherein the host cell is a human liver cell.

204. The Factor VIII polypeptide according to aspect 203, wherein the human liver cell is an Huh7 cell.

205. The Factor VIII polypeptide according to aspect 201, wherein the level of Factor VIII polypeptide that is expressed is in vivo expression.

206. The Factor VIII polypeptide according to any one of aspects 191 to 205, wherein the Factor VIII polypeptide has higher specific activity and/or higher stability and/or is expressed at a higher level in a host cell than a reference Factor VIII polypeptide which comprises the Factor VIII amino acid sequence of the Factor VIII polypeptide but which does not comprise the one or more substitution mutations.

207. The Factor VIII polypeptide according to aspect 206, wherein the reference Factor VIII polypeptide is the Factor VIII polypeptide of SEQ ID NO: 1, 3 or 5.

208. The Factor VIII polypeptide according to any one of aspects 191 to 207, wherein the one or more substitution mutations at the inter-domain interface stabilise the interaction of the respective domains of the Factor VIII polypeptide when activated.

209. The Factor VIII polypeptide according to aspect 208, wherein at least one of the one or more acid substitution mutations increases pi-stacking interactions between amino acid side chains of the respective domains.

210. The Factor VIII polypeptide according to aspect 208 or 209, wherein at least one of the one or more substitution mutations increases hydrophobic packing between the amino acid side-chains of the respective domains.

211. The Factor VIII polypeptide according to any one of aspects 208 to 210, wherein at least one of the one or more substitution mutations reduces steric clashing and/or unfavourable electrostatic interactions between amino acid side chains of the respective domains.

212. The Factor VIII polypeptide according to any one of aspects 191 to 193 or 195 to 211, wherein the one or more substitution mutations comprise a substitution of one or more surface-inaccessible amino acids at an inter-domain interface.

213. The Factor VIII polypeptide according to any one of aspects 191 to 193 or 195 to 212, wherein the amino acid substituted with a more hydrophobic amino acid is methionine or histidine.

214. The Factor VIII polypeptide according to any one of aspects 191 to 193 or 195 to 213, wherein the amino acid substituted with a more hydrophobic amino acid is methionine corresponding to the amino acid at position 662 of SEQ ID NO: 1 or histidine corresponding to the amino acid at position 693 of SEQ ID NO: 1.

215. The Factor VIII polypeptide according to any one of aspects 191 to 214, wherein the one or more substitution mutation does not comprise the M662C substitution.

216. The Factor VIII polypeptide according to any one of aspects 191 to 215, wherein:
a. the one or more substitution mutations comprises substitution of methionine with tyrosine, isoleucine, leucine, phenylalanine or tryptophan;
b. the one or more substitution mutations comprises substitution of histidine with glutamate, cysteine, valine, methionine, tyrosine, isoleucine, leucine, phenylalanine or tryptophan.

217. The Factor VIII polypeptide according to any one of aspects 191 to 216, wherein the one or more substitution mutations comprises substitution of an amino acid with an aromatic amino acid.

218. The Factor VIII polypeptide according to any one of aspects 191 to 217, wherein the one or more substitution mutations comprises the M662W substitution.

219. The Factor VIII polypeptide according to any one of aspects 191 to 218, wherein the one or more substitution mutations comprises the H693W substitution.

220. The Factor VIII polypeptide according to any one of aspects 191 to 219, wherein the one or more substitution mutations comprises the M662W and H693W substitutions.

221. The Factor VIII polypeptide according to any one of aspects 191 to 220, wherein the one or more substitution mutations comprises substitution of a pair of amino acids in the respective domains with cysteine residues, wherein the cysteine residues form a disulphide bond between the respective domains.

222. The Factor VIII polypeptide according to aspect 221, wherein the pair of amino acids are in the A1 and A3 domains.

223. The Factor VIII polypeptide according to aspect 221 or 222, wherein the pair of amino acids comprises a first amino acid and a second amino acid, wherein the first amino acid corresponds to M147, S149 or S289 of SEQ ID NO: 1 and the second amino acid corresponds to E1969, E1970 or N1977 of SEQ ID NO: 1.

224. The Factor VIII polypeptide according to any one of aspects 221 to 223, wherein the one or more substitution mutations comprises a pair of substitution mutations selected from the list consisting of (i) S289C and N1977C, (ii) M147C and E1970C, and (iii) S149C and E1969C.

225. The Factor VIII polypeptide according to aspect 221, wherein the pair of amino acids are in the A2 and A3 domains.

226. The Factor VIII polypeptide according to aspect 221 or 225, wherein the pair of amino acids comprises a first amino acid and a second amino acid, wherein the first amino acid corresponds to T667, T669, N684, L687, 1689, S695 or F697 of SEQ ID NO: 1 and the second amino acid corresponds to S1791, G1799, A1800, R1803, E1844, S1949, G1981, V1982, or Y1979 of SEQ ID NO: 1.

227. The Factor VIII polypeptide according to any one of aspects 221, 225 or 226, wherein the one or more substitution mutations comprises a pair of substitution mutations selected from the list consisting of (i) T669C and V1982C, (ii) L687C and A1800C, (iii) I689C and G1799C, (iv) F697C and 51949C, (v) T667C and G1981C, (vi) T669C and Y1979C, (vii) N684C and 51791C, (viii) L687C and R1803C, and (ix) S695C and E1844C.

228. The Factor VIII polypeptide according to aspect 221, wherein the pair of amino acids are in the A1 and C2 domains.

229. The Factor VIII polypeptide according to aspect 221 or 228, wherein the pair of amino acids comprises a first amino acid and a second amino acid, wherein the first amino acid corresponds to A108, T118 or V137 of SEQ ID NO: 1 and the second amino acid corresponds to N2172, Q2329 or Y2332 of SEQ ID NO: 1.

230. The Factor VIII polypeptide according to any one of aspects 221, 228 or 229, wherein the one or more substitution mutations comprises a pair of substitution mutations selected from the list consisting of (i) A108C and Q2329C, (ii) T118C and N2172C, and (iii) V137C and Y2332C.

231. The Factor VIII polypeptide according to any one of aspects 191 to 230, wherein the one or more substitution mutations do not inhibit activation of the Factor VIII polypeptide.

232. The Factor VIII polypeptide according to any one of aspects 1 to 231, wherein the Factor VIII amino acid sequence comprises the amino acid sequence set forth in SEQ ID NO: 77 , or an amino acid sequence that is at least 90%, at least 95%, at least 96%, at least 97%, at least 98%, at least 99% or 100% identical to the amino acid sequence set forth in SEQ ID NO: 77.

233. A polynucleotide comprising a Factor VIII nucleotide sequence, wherein the Factor VIII nucleotide sequence encodes a Factor VIII polypeptide and wherein at least a portion of the Factor VIII nucleotide sequence is not wild-type.

234. The polynucleotide of aspect 233, wherein the Factor VIII polypeptide comprises a Factor VIII amino acid sequence.

235. The polynucleotide according to aspect 233 or 234, wherein the Factor VIII nucleotide sequence encodes the Factor VIII polypeptide according to any one of aspects 1 to 232.

236. The polynucleotide of any one of aspects 233 to 235, wherein the Factor VIII polypeptide comprises a Factor VIII amino acid sequence and wherein the Factor VIII amino acid sequence comprises a modified beta domain related (BDR) region which is modified relative to wild-type BDR region, which wild-type BDR region corresponds to the region between positions 713 and 1697 of SEQ ID NO: 1, wherein:
(i) the modified BDR region comprises a maximum of 88 amino acids, and the Factor VIII polypeptide has a specific activity which is higher than the specific activity of a polypeptide of SEQ ID NO: 7; and/or
   the modified BDR region comprises a maximum of 74 amino acids.

237. The polynucleotide according to any one of aspects 233 to 236, wherein the portion of the Factor VIII nucleotide sequence that is not wild-type is codon-optimised.

238. The polynucleotide according to aspect 237, wherein the portion of the Factor VIII nucleotide sequence that is codon-optimised is codon-optimised for expression in human liver cells.

239. The polynucleotide according to aspect 237 or 238, wherein the portion of the Factor VIII nucleotide sequence that is codon-optimised comprises at least two contiguous portions.

240. The polynucleotide according to any one of aspects 237 to 239, wherein the portion of the Factor VIII nucleotide sequence that is codon-optimised does not encode amino acids corresponding to positions 746 to 1639 of SEQ ID NO: 1.

241. The polynucleotide according to any one of aspects 237 to 240, wherein the portion of the Factor VIII nucleotide sequence that is codon-optimised does not encode the amino acid sequence of the modified BDR region or the wild-type BDR region.

242. The polynucleotide according to any one of aspects 233 to 241, wherein the Factor VIII nucleotide sequence encodes at least one amino acid corresponding to positions 746 to 1639 of SEQ ID NO: 1.

243. The polynucleotide according to any one of aspects 234 to 241, wherein at least a portion of the Factor VIII nucleotide sequence encodes a region of the Factor VIII amino acid sequence which does not comprise amino acids corresponding to positions 746 to 1639 of SEQ ID NO: 1.

244. The polynucleotide according to aspect 243, wherein the portion of the Factor VIII nucleotide sequence is at least 85.5%, at least 86.0%, at least 88%, at least 90%, at least 92%, at least 95%, at least 98%, at least 99%, or 100% identical to the portion of SEQ ID NO: 3 which encodes the region of the Factor VIII amino acid sequence.

245. The polynucleotide according to aspect 243 or 244, wherein the portion of the Factor VIII nucleotide sequence is at least 85.0%, at least 85.5%, at least 86.0%, at least 88%, at least 90%, at least 92%, at least 95%, at least 98%, at least 99%, or 100% identical to the portion of SEQ ID NO: 4 which encodes the region of the Factor VIII amino acid sequence.

246. The polynucleotide according to any one of aspects 243 to 245, wherein the portion of the Factor VIII nucleotide sequence is at least 85.0%, at least 85.5%, at least 86.0%, at least 88%, at least 90%, at least 92%, at least 95%, at least 98%, at least 99%, or 100% identical to the portion of SEQ ID NO: 5 which encodes the region of the Factor VIII amino acid sequence.

247. The polynucleotide according to any one of aspects 243 to 246, wherein the portion of the Factor VIII nucleotide sequence is at least 86.5%, at least 87.0%, at least 87.5%, at least 88%, at least 90%, at least 92%, at least 95%, at least 98%, at least 99%, or 100% identical to the portion of SEQ ID NO: 6 which encodes the region of the Factor VIII amino acid sequence.

248. The polynucleotide according to any one of aspects 243 to 247, wherein the region of the Factor VIII amino acid sequence comprises at least 650, at least 700, at least 720, or 745 amino acids corresponding to positions 1 to 745 of SEQ ID NO: 1.

249. The polynucleotide according to any one of aspects 243 to 248, wherein the region of the Factor VIII amino acid sequence comprises at least 550, at least 600, at least 650, at least 700, or 712 amino acids corresponding to positions 1640 to 2332 of SEQ ID NO: 1.

250. The polynucleotide according to any one of aspects 243 to 249, wherein the region of the Factor VIII amino acid sequence comprises at least 1200, at least 1300, at least 1400, at least 1450, or 1457 amino acids corresponding to positions 1 to 745 and 1640 to 2332 of SEQ ID NO: 1.

251. The polynucleotide according to any one of aspects 243 to 250, wherein the region of the Factor VIII amino acid sequence comprises amino acids corresponding to positions 714 to 745 of SEQ ID NO: 1 or a fragment thereof.

252. The polynucleotide according to aspect 251, wherein the region of the Factor VIII amino acid sequence comprises at least 10, at least 15, at least 20, at least 25, at least 30, or 32 amino acids corresponding to positions 714 to 745 of SEQ ID NO: 1.

253. The polynucleotide according to any one of aspects 243 to 252, wherein the region of the Factor VIII amino acid sequence comprises amino acids corresponding to positions 1640 to 1696 of SEQ ID NO: 1 or a fragment thereof.

254. The polynucleotide according to aspect 253, wherein the region of the Factor VIII amino acid sequence comprises at least 20, at least 25, at least 30, at least 35, at least 40, at least 45, at least 50, at least 55, or 57 amino acids corresponding to positions 1640 to 1696 of SEQ ID NO: 1.

255. The polynucleotide according to any one of aspects 243 to 254, wherein the region of the Factor VIII amino acid sequence comprises amino acids corresponding to positions 714 to 745 and 1640 to 1696 of SEQ ID NO: 1 or a fragment thereof.

256. The polynucleotide according to aspect 255, wherein the region of the Factor VIII amino acid sequence comprises at least 30, at least 35, at least 40, at least 45, at least 50, at least 55, at least 60, at least 65, at least 70, at least 75, at least 80, at least 85, or 89 amino acids corresponding to positions 714 to 745 and 1640 to 1696 of SEQ ID NO: 1.

257. The polynucleotide according to any one of aspects 243 to 256, wherein the region of the Factor VIII amino acid sequence does not comprise the modified BDR region.

258. The polynucleotide according to aspect 257, wherein the region of the Factor VIII amino acid sequence comprises at least 650, at least 700, at least 710 or 713 amino acids corresponding to positions 1 to 713 of SEQ ID NO: 1.

259. The polynucleotide according to any one of aspects 243 to 258, wherein the region of the Factor VIII amino acid sequence comprises at least 550, at least 600, at least 650, or 655 amino acids corresponding to positions 1697 to 2332 of SEQ ID NO: 1.

260. The polynucleotide according to any one of aspects 243 to 259, wherein the region of the Factor VIII amino acid sequence comprises at least 1200, at least 1300, at least 1350 or 1368 amino acids corresponding to positions 1 to 713 and 1697 to 2332 of SEQ ID NO: 1.

261. The polynucleotide according to any one of aspects 237 to 260, wherein the portion of the Factor VIII nucleotide sequence that is codon-optimised is at least 1500, at least 2000, at least 2500, at least 3000, at least 3500, at least 4000, at least 4100, at least 4200, at least 4300, at least 4350, 4371 or fewer, between 1500 and 4372, between 2500 and 4372, between 3500 and 4372, between 4000 and 4372, or around 4371 nucleotides in length.

262. The polynucleotide according to any one of aspects 237 to 261, wherein the portion of the Factor VIII nucleotide sequence that is codon-optimised is at least 1500, at least 2000, at least 2500, at least 3000, at least 3500, at least 4000, at least 4100, 4104 or fewer, between 1500 and 4105, between 2500 and 4105, between 3500 and 4105, between 4000 and 4105, or around 4104 nucleotides in length.

263. The polynucleotide according to any one of aspects 237 to 262, wherein the portion of the Factor VIII nucleotide sequence that is codon-optimised encodes a Factor VIII amino acid sequence which corresponds to a mature Factor VIII polypeptide.

264. The polynucleotide according to any one of aspects 237 to 263, wherein the portion of the Factor VIII nucleotide sequence that is codon-optimised does not encode all or a portion of a signal peptide.

265. The polynucleotide according to any one of aspects 237 to 263, wherein the portion of the Factor VIII nucleotide sequence that is codon-optimised encodes all or a portion of a signal peptide.

266. The polynucleotide according to any one of aspects 237 to 265, wherein, in the portion of the Factor VIII amino acid sequence that is codon-optimised, at least 50%, at least 55%, at least 60%, at least 61%, at least 62%, at least 63%, at least 64%, at least 65%, at least 69%, at least 70%, at least 75%, or at least 78% of the codons are selected from the group consisting of: TTC, CTG, ATC, GTG, TCC, AGT, AGC, CCT, CCC, ACC, ACA, GCC, TAC, CAC, CAG, AAC, AAG, GAC, GAG, TGT, AGA, and GGC.

267. The polynucleotide according to aspect 266, wherein, in the portion of the Factor VIII amino acid sequence that is codon-optimised, at least 60% of the codons are selected from the group consisting of: TTC, CTG, ATC, GTG, TCC, AGT, AGC, CCT, CCC, ACC, ACA, GCC, TAC, CAC, CAG, AAC, AAG, GAC, GAG, TGT, AGA, and GGC.

268. The polynucleotide according to any one of aspects 233 to 267, wherein the Factor VIII polypeptide encoded by the Factor VIII nucleotide sequence is expressed in human liver cells at higher levels compared to a reference wild-type Factor VIII nucleotide sequence.

269. The polynucleotide according to any one of aspects 233 to 268, wherein the Factor VIII polypeptide encoded by the Factor VIII nucleotide sequence is expressed in human liver cells at least 1.1 fold, at least 1.2 fold, at least 1.5 fold, at least 1.8 fold, at least 2 fold, at least 5 fold, at least 10 fold, at least 20 fold, at least 30 fold, at least 40 fold, or at least 50 fold higher compared to a reference wild-type Factor VIII nucleotide sequence.

270. The polynucleotide according to aspect 268 or 269, wherein the reference wild-type Factor VIII nucleotide sequence is the Factor VIII nucleotide sequence of SEQ ID NO: 2.

271. The polynucleotide according to any one of aspects 233 to 270, wherein the Factor VIII polypeptide encoded by the Factor VIII nucleotide sequence is expressed in human liver cells at at least 50%, at least 60%, at least 70%, at least 75%, at least 80%, at least 85%, at least 90%, at least 95%, at least 98%, at least 99%, or at least 100% of the level expressed by a polypeptide encoded by the polynucleotide of SEQ ID NO: 3.

272. The polynucleotide according to any one of aspects 233 to 271, wherein the Factor VIII polypeptide encoded by the Factor VIII nucleotide sequence is expressed in human liver cells at at least 50%, at least 60%, at least 70%, at least 75%, at least 80%, at least 85%, at least 90%, at least 95%, at least 98%, at least 99%, or at least 100% of the level expressed by a polypeptide encoded by the polynucleotide of SEQ ID NO: 4.

273. The polynucleotide according to any one of aspects 233 to 272, wherein the Factor VIII polypeptide encoded by the Factor VIII nucleotide sequence is expressed in human liver cells at at least 50%, at least 60%, at least 70%, at least 75%, at least 80%, at least 85%, at least 90%, at least 95%, at least 98%, at least 99%, or at least 100% of the level expressed by a polypeptide encoded by the polynucleotide of SEQ ID NO: 5.

274. The polynucleotide according to any one of aspects 233 to 273, wherein the Factor VIII polypeptide encoded by the Factor VIII nucleotide sequence is expressed in human liver cells at at least 50%, at least 60%, at least 70%, at least 75%, at least 80%, at least 85%, at least 90%, at least 95%, at least 98%, at least 99%, or at least 100% of the level expressed by a polypeptide encoded by the polynucleotide of SEQ ID NO: 6.

275. The polynucleotide according to any one of aspects 234 to 274, wherein the Factor VIII nucleotide sequence encoding the Factor VIII amino acid sequence comprises a sequence that is at least 85.5%, at least 86.0%, at least 88%, at least 90%, at least 92%, at least 95%, at least 98%, at least 99% or 100% identical to the portion of SEQ ID NO: 3 which encodes the Factor VIII amino acid sequence.

276. The polynucleotide according to aspect 275, wherein the Factor VIII nucleotide sequence encoding the Factor VIII amino acid sequence comprises a sequence that is at least 85.5%, at least 86.0%, at least 88%, at least 90%, at least 92%, at least 95%, at least 98%, at least 99% or 100% identical to SEQ ID NO: 3.

277. The polynucleotide according to aspect 275 or 276, wherein the Factor VIII nucleotide sequence encoding the Factor VIII amino acid sequence comprises the sequence of SEQ ID NO: 3.

278. The polynucleotide according to any one of aspects 234 to 277, wherein the Factor VIII nucleotide sequence encoding the Factor VIII amino acid sequence comprises a sequence that is at least 85.0%, at least 85.5%, at least 86.0%, at least 88%, at least 90%, at least 92%, at least 95%, at least 98%, at least 99% or 100% identical to the portion of SEQ ID NO: 4 which encodes the Factor VIII amino acid sequence.

279. The polynucleotide according to aspect 278, wherein the Factor VIII nucleotide sequence encoding the Factor VIII amino acid sequence comprises a sequence that is at least 85.0%, at least 85.5%, at least 86.0%, at least 88%, at least 90%, at least 92%, at least 95%, at least 98%, at least 99% or 100% identical to SEQ ID NO: 4.

280. The polynucleotide according to aspect 278 or 279, wherein the Factor VIII nucleotide sequence encoding the Factor VIII amino acid sequence comprises the sequence of SEQ ID NO: 4.

281. The polynucleotide according to any one of aspects 234 to 280, wherein the Factor VIII nucleotide sequence encoding the Factor VIII amino acid sequence comprises a sequence that is at least 85.0%, at least 85.5%, at least 86.0%, at least 88%, at least 90%, at least 92%, at least 95%, at least 98%, at least 99% or 100% identical to the portion of SEQ ID NO: 5 which encodes the Factor VIII amino acid sequence.

282. The polynucleotide according to aspect 281, wherein the Factor VIII nucleotide sequence encoding the Factor VIII amino acid sequence comprises a sequence that is at least 85.0%, at least 85.5%, at least 86.0%, at least 88%, at least 90%, at least 92%, at least 95%, at least 98%, at least 99% or 100% identical to SEQ ID NO: 5.

283. The polynucleotide according to aspect 281 or 282, wherein the Factor VIII nucleotide sequence encoding the Factor VIII amino acid sequence comprises the sequence of SEQ ID NO: 5.

284. The polynucleotide according to any one of aspects 234 to 283, wherein the Factor VIII nucleotide sequence encoding the Factor VIII amino acid sequence comprises a sequence that is at least 86.5%, at least 87.0%, at least 87.5%, at least 88.0%, at least 90%, at least 92%, at least 95%, at least 98%, at least 99% or 100% identical to the portion of SEQ ID NO: 6 which encodes the Factor VIII amino acid sequence.

285. The polynucleotide according to aspect 284, wherein the Factor VIII nucleotide sequence encoding the Factor VIII amino acid sequence comprises a sequence that is at least 86.5%, at least 87.0%, at least 87.5%, at least 88.0%, at least 90%, at least 92%, at least 95%, at least 98%, at least 99% or 100% identical to SEQ ID NO: 6.

286. The polynucleotide according to aspect 284 or 285, wherein the Factor VIII nucleotide sequence encoding the Factor VIII amino acid sequence comprises the sequence of SEQ ID NO: 6.

287. The polynucleotide according to any one of aspects 234 to 275, wherein the Factor VIII nucleotide sequence encoding the Factor VIII amino acid sequence comprises the sequence of SEQ ID NO: 39, SEQ ID NO: 40, SEQ ID NO: 41, or SEQ ID NO: 42, preferably SEQ ID NO: 39.

288. The polynucleotide according to any one of aspects 233 to 287, wherein the Factor VIII nucleotide encodes a Factor VIII amino acid sequence as defined in any one of aspects 191 to 232.

289. The polynucleotide according to aspect 288, wherein the Factor VIII nucleotide sequence encoding the Factor VIII amino acid sequence comprises a nucleotide sequence that is at least 85%, at least 90%, at least 95%, at least 96%, at least 97%, at least 98%, at least 99% or 100% identical to the nucleotide sequence set forth in SEQ ID NO: 76, or a nucleotide sequence that is at least 85%, at least 90%, at least 95%, at least 96%, at least 97%, at least 98%, at least 99% or 100% identical to a nucleotide sequence comprising at least 4047 nucleotides of SEQ ID NO: 76.

290. The polynucleotide according aspect 288 or 289, wherein the Factor VIII nucleotide sequence encoding the Factor VIII amino acid sequence comprises the nucleotide sequence set forth in SEQ ID NO:75 or a nucleotide sequence that is at least 85%, at least 90%, at least 95%, at least 96%, at least 97%, at least 98%, at least 99% or 100% identical to the nucleotide sequence set forth in SEQ ID NO:75.

291. A recombinant AAV construct which comprises a polynucleotide comprising a Factor VIII nucleotide sequence, wherein the Factor VIII nucleotide sequence encodes a Factor VIII polypeptide comprising a Factor VIII amino acid sequence.

292. The recombinant AAV construct according to aspect 291 which is less than 4900 nucleotides in length.

293. The recombinant AAV construct according to aspect 291 or 292, which is between 4700 and 4900, between 4850 and 4900, or around 4713 nucleotides in length.

294. The recombinant AAV construct according to any one of aspects 291 to 293, which is less than 4850, less than 4800, or less than 4750 nucleotides in length.

295. The recombinant AAV construct according to any one of aspects 291 to 294, which is between 4700 and 4900, between 4700 and 4850, between 4700 and 4800, between 4700 and 4750 or around 4713 nucleotides in length.

296. The recombinant AAV construct according to any one of aspects 291 to 295, wherein the Factor VIII polypeptide is the Factor VIII polypeptide according to any one of aspects 1 to 232.

297. The recombinant AAV construct according to any one of aspects 291 to 296, wherein the Factor VIII amino acid sequence does not comprise amino acids corresponding to positions 746 to 1639 of SEQ ID NO: 1.

298. The recombinant AAV construct according to any one of aspects 291 to 297, wherein the Factor VIII amino acid sequence is SEQ ID NO: 7.

299. The recombinant AAV construct according to any one of aspects 291 to 297, wherein the Factor VIII amino acid sequence is SEQ ID NO: 8.

300. The recombinant AAV construct according to any one of aspects 291 to 299, wherein the polynucleotide is the polynucleotide according to any one of aspects 233-290.

301. The recombinant AAV construct according to any one of aspects 291 to 300, wherein the recombinant AAV construct is single-stranded.

302. The recombinant AAV construct according to any one of aspects 291 to 301, further comprising a transcriptional regulatory element.

303. The recombinant AAV construct according to aspect 302, wherein the transcription regulatory element comprises a liver-specific promoter.

304. The recombinant AAV construct according to aspect 302 or 303, wherein the transcription regulatory element is fewer than 270 nucleotides in length.

305. The recombinant AAV construct according to any one of aspects 302 to 304, wherein the transcription regulatory element comprises a core nucleotide sequence which comprises or consists of a nucleotide sequence having at least 95% identity to SEQ ID NO: 43, or a sequence which differs from SEQ ID NO: 43 by a single nucleotide, and wherein the transcription regulatory element is between 80 and 280 nucleotides in length; optionally wherein the transcription regulatory element is between 80 and 225 nucleotides in length.

306. The recombinant AAV construct according to any one of aspects 302 to 305, wherein the transcription regulatory element comprises a core nucleotide sequence which comprises or consists of a sequence having at least 95% identity to SEQ ID NO: 43, or a sequence which differs from SEQ ID NO: 43 by a single nucleotide, wherein the transcription regulatory element:
(a) does not comprise at least 20, at least 30 or at least 40 consecutive nucleotides of SEQ ID NO: 48 and/or
(b) does not comprise at least 20, at least 30 or at least 40 consecutive nucleotides of SEQ ID NO: 49;
and wherein the transcription regulatory element is between 80 and 280 nucleotides in length.

307. The recombinant AAV construct according to aspect 305 or 306, wherein the transcription regulatory element further comprises a nucleotide sequence located 3′ to the core nucleotide sequence, and the nucleotide sequence located 3′ to the core nucleotide sequence comprises or consists of a nucleotide sequence:
a. of SEQ ID NO: 44, or a sequence which differs from SEQ ID NO: 44 by a single nucleotide;
b. of SEQ ID NO: 45, or a sequence which differs from SEQ ID NO: 45 by a single nucleotide;
c. of SEQ ID NO: 46, or a sequence which differs from SEQ ID NO: 46 by a single nucleotide;
d. having at least 95% identity to SEQ ID NO: 47, or a sequence which differs from SEQ ID NO: 47 by a single nucleotide.

308. The recombinant AAV construct according to aspect 307, wherein the nucleotide sequence located 3′ to the core nucleotide sequence is shorter than 50 nucleotides; optionally is shorter than 40 nucleotides; and optionally is shorter than 30 nucleotides.

309. The recombinant AAV construct according to any one of aspects 305 to 308, wherein the transcription regulatory element:

a. does not comprise a nucleotide sequence according to SEQ ID NO: 48, or does not comprise at least 20, at least 30 or at least 40 consecutive nucleotide of SEQ ID NO: 48; and/or
b. does not comprise a nucleotide sequence according to SEQ ID NO: 49 or does not comprise at least 20, at least 30 or at least 40 consecutive nucleotide of SEQ ID NO: 49.

310. The recombinant AAV construct according to aspect 309, wherein the transcription regulatory element:
a. does not comprise a sequence having at least 90% or at least 95% identity to SEQ ID NO: 48; and/or
b. does not comprise a sequence having at least 90% or at least 95% identity to SEQ ID NO: 49.

311. The recombinant AAV construct according to any one of aspects 305 to 310, wherein the transcription regulatory element is shorter than 200 nucleotides, optionally shorter than 150 nucleotides, optionally shorter than 125 nucleotides.

312. The recombinant AAV construct according to any one of aspects 305 to 311, wherein the transcription regulatory element is at least 85 nucleotides in length, optionally at least 100 nucleotides in length, optionally at least 110 nucleotides in length.

313. The recombinant AAV construct according to any one of aspects 305 to 312, wherein the transcription regulatory element terminates in a ten-nucleotide sequence selected from:
a. acagtgaatc; or
b. ctcctcagct.

314. The recombinant AAV construct according to any one of aspects 305 to 313, wherein the core nucleotide sequence is 73-80 nucleotides in length.

315. The recombinant AAV construct according to any one of aspects 305 to 314, wherein the core nucleotide sequence has at least 95% identity, and optionally at least 98% identity, to SEQ ID NO: 50.

316. The recombinant AAV construct according to any one of aspects 305 to 315, wherein the transcription regulatory element comprises or consists of a sequence which has at least 90% identity, optionally at least 95% identity, or optionally at least 98% identity to SEQ ID NO: 51.

317. The recombinant AAV construct of aspect 316, wherein the transcription regulatory element comprises or consists of a sequence of SEQ ID NO: 51.

318. The recombinant AAV construct according to any one of aspects 302 to 317, wherein the transcription regulatory element is operably linked to the Factor VIII nucleotide sequence.

319. The recombinant AAV construct according to aspect 318, wherein the transcription regulatory element expresses the Factor VIII nucleotide sequence at 50% or better, 80% or better or 100% or better compared to a transcription regulatory element defined by SEQ ID NO: 52 optionally wherein expression of the Factor VIII nucleotide sequence is determined in vitro in Huh7 cells.

320. The recombinant AAV construct according to any one of aspects 291 to 319, further comprising a nucleotide sequence encoding a signal peptide.

321. The recombinant AAV construct according to aspect 320, wherein the signal peptide is a wild-type Factor VIII signal peptide.

322. The recombinant AAV construct according to aspect 320 or 321, wherein the signal peptide comprises SEQ ID NO: 53 or wherein the nucleotide sequence encoding the signal peptide comprises SEQ ID NO: 54 or 55.

323. The recombinant AAV construct according to aspect 320, wherein the signal peptide is not a wild-type Factor VIII signal peptide.

324. The recombinant AAV construct according to aspect 323, wherein the Factor VIII polypeptide or fragment thereof encoded by the Factor VIII nucleotide sequence is expressed in plasma at a higher level following administration of the recombinant AAV construct compared to the administration of an equivalent dose of an equivalent recombinant AAV construct which comprises a wild-type Factor VIII signal peptide.

325. The recombinant AAV construct according to aspect 323 or 324, wherein the Factor VIII polypeptide or fragment thereof encoded by the Factor VIII nucleotide sequence is expressed in plasma at least 1.1 fold, at least 1.2 fold, at least 1.5 fold, at least 1.8 fold, at least 2 fold, at least 5 fold higher compared to the equivalent recombinant AAV construct which comprises a wild-type Factor VIII signal peptide.

326. The recombinant AAV construct according to any one of aspects 323 to 325, wherein the signal peptide comprises a sequence that is at least 90%, at least 92%, at least 95%, at least 98%, at least 99% or 100% identical to the sequence of any one of SEQ ID NOs: 56, 58 or 60.

327. The recombinant AAV construct according to aspect 326, wherein the signal peptide comprises SEQ ID NO: 56 or wherein the nucleotide sequence encoding the signal peptide comprises SEQ ID NO: 57.

328. The recombinant AAV construct according to aspect 326, wherein the signal peptide comprises SEQ ID NO: 60 or wherein the nucleotide sequence encoding the signal peptide comprises SEQ ID NO: 61.

329. The recombinant AAV construct according to aspect 326, wherein the signal peptide comprises SEQ ID NO: 58 or wherein the nucleotide sequence encoding the signal peptide comprises SEQ ID NO: 59.

330. The recombinant AAV construct according to any one of aspects 320, 323 to 327 or 329, wherein the nucleotide sequence encoding the signal peptide is fewer than 57 nucleotides in length.

331. The recombinant AAV construct according to aspect 320, 323 to 327, 329 or 330, wherein the nucleotide sequence encoding the signal peptide is around 54 nucleotides in length.

332. The recombinant AAV construct according to any one of aspects 291 to 331, further comprising a polyA nucleotide sequence.

333. The recombinant AAV construct according to aspect 332, wherein the polyA nucleotide sequence comprises the nucleotide sequence of any one of SEQ ID NOs: 63 to 66.

334. The recombinant AAV construct according to aspect 332 or 333, wherein the polyA nucleotide sequence is fewer than 50, or around 49 nucleotides in length.

335. The recombinant AAV construct according to aspect 333 or 334, wherein the polyA nucleotide sequence comprises the nucleotide sequence of SEQ ID NO: 65.

336. The recombinant AAV construct according to any one of aspects 291 to 335, further comprising one or two ITR(s).

337. The recombinant AAV construct according to aspect 336, wherein the nucleotide sequence of the or each ITR is fewer than 157, fewer than 154, or around 145 nucleotides in length.

338. The recombinant AAV construct according to aspect 336 or 337, wherein the or each ITR is a wild-type ITR.

339. The recombinant AAV construct according to any one of aspects 336 to 338, wherein the or each ITR is an AAV2 ITR.

340. The recombinant AAV construct according to any one of aspects 336 to 339, wherein the nucleotide sequence of the or each ITR comprises a nucleotide sequence of SEQ ID NO: 67 or SEQ ID NO: 70.

341. The recombinant AAV construct according to any one of aspects 291 to 340, wherein the AAV construct is an AAV genome.

342. The recombinant AAV construct according to any one of aspects 291 to 297, 300 to 320, 323 to 326, 330 to 341, wherein the Factor VIII nucleotide sequence encoding the Factor VIII amino acid sequence comprises the sequence of SEQ ID NO: 39 or SEQ ID NO: 75 and the nucleotide sequence encoding the signal peptide comprises the nucleotide sequence of SEQ ID NO: 57.

343. The recombinant AAV construct according to aspect 342, wherein the recombinant AAV construct comprises a transcriptional regulatory element which is a liver-specific promoter, and the liver-specific promoter comprises the nucleotide sequence of SEQ ID NO: 51.

344. The recombinant AAV construct according to aspect 343, wherein the recombinant AAV construct comprises two ITRs and a polyA nucleotide sequence, wherein the nucleotide sequence of each ITR is SEQ ID NO: 67 and/or SEQ ID NO: 70 and the polyA nucleotide sequence comprises the nucleotide sequence of SEQ ID NO: 65.

345. The recombinant AAV construct according to any one of aspects 291 to 344, wherein the recombinant AAV construct comprises or consists of SEQ ID NO:71.

346. The recombinant AAV construct according to any one of aspects 292 to 345, wherein vector genome yield is increased when said recombinant AAV construct is used to produce AAV viral particles when compared to the vector genome yield obtained when a comparator recombinant AAV construct which is more than 4900 nucleotides in length is used.

347. The recombinant AAV construct according to aspect 346, wherein the vector genome yield is at least 1.5, at least 2, at least 2.25, at least 2.5, at least 2.75, at least 3, at least 4, or at least 5 fold higher than the vector genome yield obtained when the comparator recombinant AAV construct is used, optionally wherein the vector genome yield is between 1.25 and 3 fold higher, between 1.5 and 3 fold higher, or between 2 and 3 fold higher than the vector genome yield obtained when the comparator recombinant AAV construct is used.

348. The recombinant AAV construct according to any one of aspects 292 to 347, wherein vector genome to total particle ratio is increased when said recombinant AAV construct is used to produce AAV viral particles when compared to the vector genome to total particle ratio obtained when a comparator recombinant AAV construct which is more than 4900 nucleotides in length is used.

349. The recombinant AAV construct according to aspect 348, wherein the vector genome to total particle ratio is at least 1.25, at least 1.5, at least 1.75, at least 2, at least 2.25, at least 2.5, at least 2.75, at least 3, at least 3.25, at least 3.5, at least 4, or at least 5 fold higher than the vector genome to total particle ratio obtained when the comparator recombinant AAV construct is used, optionally wherein the vector genome to total particle ratio is between 1.25 and 4 fold higher, or between 1.5 and 3.5 fold higher than the vector genome to total particle ratio obtained when the comparator recombinant AAV construct is used.

350. The recombinant AAV construct according to any one of aspects 292 to 349, wherein nucleic acid impurity level, optionally plasmid-derived impurity level, is decreased when said recombinant AAV construct is used to produce AAV viral particles when compared to the impurity level obtained when a comparator recombinant AAV construct which is more than 4900 nucleotides in length is used.

351. The recombinant AAV construct according to aspect 350, wherein the nucleic acid impurity level is 85% or lower, 75% or lower, 60% or lower, 50% or lower, 40% or lower, 30% or lower, or 20% or lower than the nucleic acid impurity level obtained when the comparator recombinant AAV construct is used, optionally wherein the nucleic acid impurity level is between 40% and 80%, or between 50% and 70%, or between 15% and 55% of the nucleic acid impurity level obtained when the comparator recombinant AAV construct is used.

352. The recombinant AAV construct according to aspect 350 or 351, wherein the nucleic acid impurity is an AAV cap-derived nucleic acid.

353. The recombinant AAV construct according to any one of aspects 346 to 352, wherein when the recombinant AAV construct and the comparator recombinant AAV construct are used to produce AAV viral particles, the same AAV production system is used in each case, optionally wherein said AAV production system is a mammalian production system, optionally wherein said AAV production system is a two-plasmid system consisting of a helper plasmid comprising sequences encoding AAV Rep functions and a vector plasmid comprising sequences encoding AAV Cap functions.

354. The recombinant AAV construct according aspect 353, wherein the AAV production system is a two-plasmid system consisting of a helper plasmid comprising AAV Rep functions and a vector plasmid comprising AAV Cap functions, and wherein the molar ratio of helper plasmid to vector plasmid is between about 12:1 and 1:12, between about 1:10 and 10:1, between about 1:5 and 5:1, between about 1:3 and 4:3 or between about 1:2 and 1:4, optionally wherein the molar ratio of helper plasmid to vector plasmid is around 4:3 or around 1:3.

355. The recombinant AAV construct according to any one of aspects 346 to 354, wherein the recombinant AAV construct is between 4500 and 4900, between 4500 and 4850, between 4500 and 4750, around 4715, or around 4713 nucleotides in length.

356. The recombinant AAV construct according to any one of aspects 346 to 355, wherein the recombinant AAV construct is 4713 nucleotides in length.

357. The recombinant AAV construct according to any one of aspects 346 to 356, wherein the comparator recombinant AAV construct is more than 4910, more than 4920, more than 4930, more than 4940, more than 4950, more than 4960, more than 4970, more than 4980, more than 4990, or more than 5000 nucleotides in length, optionally wherein the comparator recombinant AAV construct comprises or consists of SEQ ID NO: 72.

358. An AAV viral particle comprising the recombinant AAV construct according to any one of aspects 291 to 357.

359. The AAV viral particle according to aspect 358, wherein the viral particle comprises a capsid.

360. The AAV viral particle according to aspect 359, wherein the capsid is selected from the group consisting of:
(i) a capsid comprising a sequence which is at least 96%, at least 98%, at least 99%, at least 99.5%, at least 99.8% or 100% identical to SEQ ID NO: 68;
(ii) a capsid comprising a sequence which is at least 96%, at least 98%, at least 99%, at least 99.5%, at least 99.8%, or 100% identical to SEQ ID NO: 69;
(iii) a liver-tropic capsid; and
(iv) an AAVS capsid.

361. The AAV viral particle according to aspect 359, wherein the capsid is selected from the group consisting of:
(i) a capsid comprising a sequence which is at least 96%, at least 98%, at least 99%, at least 99.5%, at least 99.8% or 100% identical to SEQ ID NO: 91;
(ii) a capsid comprising a sequence which is at least 96%, at least 98%, at least 99%, at least 99.5%, at least 99.8%, or 100% identical to SEQ ID NO: 92; and
(iii) an AAV6 capsid.

362. The recombinant AAV construct or AAV viral particle according to any one of aspects 291 to 361, wherein the activity of the polypeptide encoded by the Factor VIII nucleotide sequence achieved is higher following administration of the recombinant AAV construct or AAV viral particle compared to the administration of an equivalent dose of an equivalent recombinant AAV construct or AAV viral particle which comprises the AAV construct of SEQ ID NO: 1 of WO 2017/053677 (SEQ ID NO: 72).

363. The recombinant AAV construct or AAV viral particle according to aspect 362, wherein the activity of the polypeptide encoded by the Factor VIII nucleotide sequence achieved is at least 1.1 fold, at least 1.2 fold, at least 1.5 fold, at least 1.8 fold, at least 2 fold, or at least 5 fold higher compared to the equivalent recombinant AAV construct or AAV viral particle which comprises the AAV construct of SEQ ID NO: 1 of WO 2017/053677 (SEQ ID NO: 72).

364. A Factor VIII polypeptide encoded by the polynucleotide or recombinant AAV construct according to any one of aspects 233 to 357.

365. A composition comprising the Factor VIII polypeptide, polynucleotide, recombinant AAV construct or AAV viral particle according to any one of aspects 1 to 364, and a pharmaceutically acceptable excipient.

366. The Factor VIII polypeptide, polynucleotide, recombinant AAV construct, AAV viral particle or composition according to any one of aspects 1 to 365 for use in a method of treatment.

367. The Factor VIII polypeptide, polynucleotide, recombinant AAV construct, AAV viral particle or composition for use according to aspect 366, wherein the method of treatment comprises administering an effective amount of the Factor VIII polypeptide, polynucleotide, recombinant AAV construct or AAV viral particle of any one of aspects 1 to 365 to a patient.

368. A method of treatment comprising administering an effective amount of the Factor VIII polypeptide, polynucleotide, recombinant AAV construct, AAV viral particle or composition according to any one of aspects 1 to 365 to a patient.

369. Use of the Factor VIII polypeptide, polynucleotide, recombinant AAV construct, AAV viral particle or composition according to any one of aspects 1 to 365 in the manufacture of a medicament for use in a method of treatment.

370. The use according to aspect 369 wherein the method of treatment comprises administering an effective amount of the Factor VIII polypeptide, polynucleotide, recombinant AAV construct, AAV viral particle or composition according to any one of aspects 1 to 365 to a patient.

371. The Factor VIII polypeptide, polynucleotide, recombinant AAV construct, AAV viral particle, composition, method or use according to any one of aspects 366 to 370, wherein the method of treatment is a method of treating haemophilia.

372. The Factor VIII polypeptide, polynucleotide, recombinant AAV construct, AAV viral particle, composition, method or use according to any one of aspects 366 to 371, wherein the method of treatment is a method of treating haemophilia A.

373. Use of the recombinant AAV construct as defined in any one of aspects 291-357 for producing a population of AAV viral particles.

374. The use according to aspect 373, wherein the AAV construct is less than 4900 nucleotides in length, wherein the population of AAV viral particles:
a) has an increased vector genome yield when compared to the vector genome yield obtained when a comparator recombinant AAV construct is used;
b) has an increased vector genome to total particle ratio when compared to the vector genome to total particle ratio obtained when a comparator recombinant AAV construct is used; and/or
c) has a decreased nucleic acid impurity level when compared to the nucleic acid impurity level obtained when a comparator recombinant AAV construct is used; wherein the comparator recombinant AAV construct is more than 4900 nucleotides in length.

375. Use of a recombinant AAV construct as defined in any one of aspects 292-357 for:
a) increasing the vector genome yield during AAV viral particle production when compared to the vector genome yield obtained when a comparator recombinant AAV construct is used;
b) increasing the vector genome to total particle ratio during AAV viral particle production when compared to the vector genome to total particle ratio obtained when a comparator recombinant AAV construct is used; and/or
c) decreasing the nucleic acid impurity level during AAV viral particle production when compared to the nucleic acid impurity level obtained when a comparator recombinant AAV construct is used;
   wherein the comparator recombinant AAV construct is more than 4900 nucleotides in length.

376. The use according to any one of aspects 373 to 375, wherein the use comprises transfecting a host cell with the recombinant AAV construct of any one of aspects 291-357 and culturing the host cell under conditions suitable for AAV viral particle production.

377. The use according to any one of aspects 374 to 376, wherein the vector genome yield is at least 1.5, at least 2, at least 2.25, at least 2.5, at least 2.75, at least 3, at least 4, or at least 5 fold higher than the vector genome yield obtained when the comparator recombinant AAV construct is used, optionally wherein the vector genome yield is between 1.25 and 3 fold higher, between 1.5 and 3 fold higher, or between 2 and 3 fold higher than the vector genome yield obtained when the comparator recombinant AAV construct is used.

378. The use according to any one of aspects 374 to 377, wherein the vector genome to total particle ratio is at least 1.25, at least 1.5, at least 1.75, at least 2, at least 2.25, at least 2.5, at least 2.75, at least 3, at least 3.25, at least 3.5, at least 4, or at least 5 fold higher than the vector genome to total particle ratio obtained when the comparator recombinant AAV construct is used, optionally wherein the vector genome to total particle ratio is between 1.25 and 4 fold higher, or between 1.5 and 3.5 fold higher than the vector genome to total particle ratio obtained when the comparator recombinant AAV construct is used.

379. The use according to any one of aspects 374 to 378, wherein the nucleic acid impurity level is 85% or lower, 75% or lower, 60% or lower, 50% or lower, 40% or lower, 30% or lower, or 20% or lower than the nucleic acid impurity level obtained when the comparator recombinant AAV construct is used, optionally wherein the nucleic acid impurity level is between 40% and 80%, between 50% and 70%, or between 15% and 55% of the nucleic acid impurity level obtained when the comparator recombinant AAV construct is used.

380. A method for producing a population of AAV viral particles comprising:
a) obtaining the recombinant AAV construct as defined in any one of aspects 291-357;
b) transfecting a host cell with the said recombinant AAV construct; and
c) culturing the host cell under conditions suitable for AAV viral particle production.

381. A method for increasing the vector genome yield during the production of a population of AAV viral particles, comprising:
a) obtaining the recombinant AAV construct as defined in any one of aspects 292-357;
b) transfecting a host cell with the said recombinant AAV construct; and
c) culturing the host cell under conditions suitable for AAV viral particle production;
   wherein the vector genome yield is increased when compared to the vector genome yield obtained when a comparator recombinant AAV construct of more than 4900 nucleotides in length is used.

382. The method according to aspect 381, wherein the vector genome yield is at least 1.5, at least 2, at least 2.25, at least 2.5, at least 2.75, at least 3, at least 4, or at least 5 fold higher than the vector genome yield obtained when the comparator recombinant AAV construct is used, optionally wherein the vector genome yield is between 1.25 and 3 fold higher, between 1.5 and 3 fold higher, or between 2 and 3 fold higher than the vector genome yield obtained when the comparator recombinant AAV construct is used.

383. A method for increasing the vector genome to total particle ratio during the production of a population of AAV viral particles, comprising:
a) obtaining the recombinant AAV construct as defined in any one of aspects 292-357;
b) transfecting a host cell with the said recombinant AAV construct; and
c) culturing the host cell under conditions suitable for AAV viral particle production;
   wherein the vector genome to total particle ratio is increased when compared to the vector genome to total particle ratio obtained when a comparator recombinant AAV construct of more than 4900 nucleotides in length is used.

384. The method according to aspect 383, wherein the vector genome to total particle ratio is at least 1.25, at least 1.5, at least 1.75, at least 2, at least 2.25, at least 2.5, at least 2.75, at least 3, at least 3.25, at least 3.5, at least 4, or at least 5 fold higher than the vector genome to total particle ratio obtained when the comparator recombinant AAV construct is used, optionally wherein the vector genome to total particle ratio is between 1.25 and 4 fold higher, or between 1.5 and 3.5 fold higher than the vector genome to total particle ratio obtained when the comparator recombinant AAV construct is used.

385. A method for decreasing the nucleic acid impurity level during the production of a population of AAV viral particles, comprising:
a) obtaining the recombinant AAV construct as defined in any one of aspects 292-357;
b) transfecting a host cell with the said recombinant AAV construct; and
c) culturing the host cell under conditions suitable for AAV viral particle production;
   wherein the nucleic acid impurity level is decreased when compared to the nucleic acid impurity level obtained when a comparator recombinant AAV construct of more than 4900 nucleotides in length is used.

386. The method according to aspect 385, wherein the nucleic acid impurity level is 85% or lower, 75% or lower, 60% or lower, 50% or lower, 40% or lower, 30% or lower, or 20% or lower than the nucleic acid impurity level obtained when the comparator recombinant AAV construct is used, optionally wherein the nucleic acid impurity level is between 40% and 80%, between 50% and 70%, or between 15% and 55% of the nucleic acid impurity level obtained when the comparator recombinant AAV construct is used.

387. The method according to any one of aspects 376 to 386, further comprising a step of harvesting the AAV viral particles to provide a population of AAV viral particles.

388. The method or use according to any one of aspects 376 to 387, wherein the host cell is selected from the group consisting of a HEK293T cell, a HEK293 cell, a HEK293EBNA cell, a CAP cell, a CAP-T cell, an AGE1.CR cell, a PerC6 cell, a C139 cell, and an EB66 cell.

389. The method or use according to any one of aspects 376 to 388, further comprising a step of purifying the AAV viral particles.

390. The method or use according to aspect 389, where the step of purifying the AAV viral particles is carried out using a technique selected from the group consisting of gradient density centrifugation (such as CsCl or Iodixanol gradient density centrifugation), filtration, ion exchange chromatography, size exclusion chromatography, affinity chromatography and hydrophobic interaction chromatography.

391. The method or use according to aspect 389 or 390, comprising further concentrating the AAV viral particles using ultracentrifugation, tangential flow filtration, or gel filtration.

392. The method or use according to any one of aspects 376 to 391, comprising formulating the AAV viral particles with a pharmaceutically acceptable excipient.

393. The method or use according to any one of aspects 374 to 379 or 381 to 392, wherein the comparator is more than 4910, more than 4920, more than 4930, more than 4940, more than 4950, more than 4960, more than 4970, more than 4980, more than 4990, or more than 5000 nucleotides in length, optionally wherein the comparator comprises or consists of SEQ ID NO: 72.

394. A population of AAV viral particles obtained or obtainable by the method of any one of aspects 380 to 393.

1. A recombinant AAV construct which is less than 4900 nucleotides in length and which comprises a polynucleotide comprising a Factor VIII nucleotide sequence, wherein the Factor VIII nucleotide sequence encodes a Factor VIII polypeptide comprising a Factor VIII amino acid sequence.

2. The recombinant AAV construct according to claim 1, which is:
(i) between 4700 and 4900, between 4850 and 4900, or around 4713 nucleotides in length; and/or
(ii) less than 4850, less than 4800, or less than 4750 nucleotides in length; and/or
(iii) between 4700 and 4900, between 4700 and 4850, between 4700 and 4800, between 4700 and 4750 or around 4713 nucleotides in length.

3. The recombinant AAV construct according to claim 1 or 2, wherein the Factor VIII amino acid sequence:
(i) does not comprise amino acids corresponding to positions 746 to 1639 of SEQ ID NO: 1; and/or
(ii) is SEQ ID NO: 7 or SEQ ID NO: 8.

4. The recombinant AAV construct according to any one of claims 1 to 3, further comprising a transcriptional regulatory element, optionally wherein the transcription regulatory element:
(i) comprises a liver-specific promoter; and/or
(ii) is fewer than 270 nucleotides in length; and/or
(iii) comprises a core nucleotide sequence which comprises or consists of a nucleotide sequence having at least 95% identity to SEQ ID NO: 43, or a sequence which differs from SEQ ID NO: 43 by a single nucleotide, and wherein the transcription regulatory element is between 80 and 280 nucleotides in length; optionally wherein the transcription regulatory element is between 80 and 225 nucleotides in length; and/or
(iv) comprises a core nucleotide sequence which comprises or consists of a sequence having at least 95% identity to SEQ ID NO: 43, or a sequence which differs from SEQ ID NO: 43 by a single nucleotide, wherein the transcription regulatory element:
(a) does not comprise at least 20, at least 30 or at least 40 consecutive nucleotides of SEQ ID NO: 48 and/or
(b) does not comprise at least 20, at least 30 or at least 40 consecutive nucleotides of SEQ ID NO: 49;
and wherein the transcription regulatory element is between 80 and 280 nucleotides in length; and/or
(v) comprises or consists of a sequence which has at least 90% identity, optionally at least 95% identity, or optionally at least 98% identity to SEQ ID NO: 51; and/or
(vi) comprises or consists of a sequence of SEQ ID NO: 51; and/or
(vii) is operably linked to the Factor VIII nucleotide sequence; and/or
(viii) is operably linked to the Factor VIII nucleotide sequence, and wherein the transcription regulatory element expresses the Factor VIII nucleotide sequence at 50% or better, 80% or better or 100% or better compared to a transcription regulatory element defined by SEQ ID NO: 52, optionally wherein expression of the Factor VIII nucleotide sequence is determined in vitro in Huh7 cells.

5. The recombinant AAV construct according to any one of claims 1 to 4, further comprising a nucleotide sequence encoding a signal peptide, optionally wherein:
(I) (i) the signal peptide is a wild-type Factor VIII signal peptide; and/or
(ii) the signal peptide comprises SEQ ID NO: 53 or wherein the nucleotide sequence encoding the signal peptide comprises SEQ ID NO: 54 or 55; or
(II) (i) the signal peptide is not a wild-type Factor VIII signal peptide; and/or
(ii) the Factor VIII polypeptide or fragment thereof encoded by the Factor VIII nucleotide sequence is expressed in plasma at a higher level following administration of the recombinant AAV construct compared to the administration of an equivalent dose of an equivalent recombinant AAV construct which comprises a wild-type Factor VIII signal peptide; and/or
(iii) the Factor VIII polypeptide or fragment thereof encoded by the Factor VIII nucleotide sequence is expressed in plasma at least 1.1 fold, at least 1.2 fold, at least 1.5 fold, at least 1.8 fold, at least 2 fold, at least 5 fold higher compared to the equivalent recombinant AAV construct which comprises a wild-type Factor VIII signal peptide; and/or
(iv) the signal peptide comprises a sequence that is at least 90%, at least 92%, at least 95%, at least 98%, at least 99% or 100% identical to the sequence of any one of SEQ ID NOs: 56, 58 or 60.

6. The recombinant AAV construct according to claim 5, wherein:
(a) the signal peptide comprises SEQ ID NO: 56 or wherein the nucleotide sequence encoding the signal peptide comprises SEQ ID NO: 57; or
(b) the signal peptide comprises SEQ ID NO: 60 or wherein the nucleotide sequence encoding the signal peptide comprises SEQ ID NO: 61; or
(c) the signal peptide comprises SEQ ID NO: 58 or wherein the nucleotide sequence encoding the signal peptide comprises SEQ ID NO: 59.

7. The recombinant AAV construct according to claim 5 or 6, wherein the nucleotide sequence encoding the signal peptide is fewer than 57 nucleotides in length or around 54 nucleotides in length.

8. The recombinant AAV construct according to any one of the preceding claims, further comprising a polyA nucleotide sequence, optionally wherein the polyA nucleotide sequence:
(i) comprises the nucleotide sequence of any one of SEQ ID NOs: 63 to 66; and/or
(ii) is fewer than 50, or around 49 nucleotides in length; and/or
(iii) comprises the nucleotide sequence of SEQ ID NO: 65.

9. The recombinant AAV construct according to any one of the preceding claims, further comprising one or two ITR(s), optionally wherein:
(i) the nucleotide sequence of the or each ITR is fewer than 157, fewer than 154, or around 145 nucleotides in length; and/or
(ii) the or each ITR is a wild-type ITR; and/or
(iii) the or each ITR is an AAV2 ITR; and/or
(iv) the nucleotide sequence of the or each ITR comprises a nucleotide sequence of SEQ ID NO: 67 or SEQ ID NO: 70.

10. The recombinant AAV construct according to any one of the preceding claims, wherein:
(i) the Factor VIII nucleotide sequence encoding the Factor VIII amino acid sequence comprises the sequence of SEQ ID NO: 39 or SEQ ID NO: 75 and the nucleotide sequence encoding the signal peptide comprises the nucleotide sequence of SEQ ID NO: 57; and
(ii) the recombinant AAV construct comprises a transcriptional regulatory element which is a liver-specific promoter, and the liver-specific promoter comprises the nucleotide sequence of SEQ ID NO: 51; and
(iii) the recombinant AAV construct comprises two ITRs and a polyA nucleotide sequence, wherein the nucleotide sequence of each ITR is SEQ ID NO: 67 and/or SEQ ID NO: 70 and the polyA nucleotide sequence comprises the nucleotide sequence of SEQ ID NO: 65.

11. The recombinant AAV construct according to any one of the preceding claims, wherein the recombinant AAV construct comprises or consists of SEQ ID NO:71.

12. The recombinant AAV construct according to any one of the preceding claims, wherein:
(i) vector genome yield is increased when said recombinant AAV construct is used to produce AAV viral particles when compared to the vector genome yield obtained when a comparator recombinant AAV construct which is more than 4900 nucleotides in length is used; and/or
(ii) vector genome yield is increased when said recombinant AAV construct is used to produce AAV viral particles when compared to the vector genome yield obtained when a comparator recombinant AAV construct which is more than 4900 nucleotides in length is used, and the vector genome yield is at least 1.5, at least 2, at least 2.25, at least 2.5, at least 2.75, at least 3, at least 4, or at least 5 fold higher than the vector genome yield obtained when the comparator recombinant AAV construct is used, optionally wherein the vector genome yield is between 1.25 and 3 fold higher, between 1.5 and 3 fold higher, or between 2 and 3 fold higher than the vector genome yield obtained when the comparator recombinant AAV construct is used.

13. The recombinant AAV construct according to any one of the preceding claims, wherein:
(i) vector genome to total particle ratio is increased when said recombinant AAV construct is used to produce AAV viral particles when compared to the vector genome to total particle ratio obtained when a comparator recombinant AAV construct which is more than 4900 nucleotides in length is used; and/or
(ii) vector genome to total particle ratio is increased when said recombinant AAV construct is used to produce AAV viral particles when compared to the vector genome to total particle ratio obtained when a comparator recombinant AAV construct which is more than 4900 nucleotides in length is used, and the vector genome to total particle ratio is at least 1.25, at least 1.5, at least 1.75, at least 2, at least 2.25, at least 2.5, at least 2.75, at least 3, at least 3.25, at least 3.5, at least 4, or at least 5 fold higher than the vector genome to total particle ratio obtained when the comparator recombinant AAV construct is used, optionally wherein the vector genome to total particle ratio is between 1.25 and 4 fold higher, or between 1.5 and 3.5 fold higher than the vector genome to total particle ratio obtained when the comparator recombinant AAV construct is used.

14. The recombinant AAV construct according to any one of the preceding claims, wherein:
(i) nucleic acid impurity level, optionally plasmid-derived impurity level, is decreased when said recombinant AAV construct is used to produce AAV viral particles when compared to the impurity level obtained when a comparator recombinant AAV construct which is more than 4900 nucleotides in length is used; and/or
(ii) nucleic acid impurity level, optionally plasmid-derived impurity level, is decreased when said recombinant AAV construct is used to produce AAV viral particles when compared to the impurity level obtained when a comparator recombinant AAV construct which is more than 4900 nucleotides in length is used, and the nucleic acid impurity level is 85% or lower, 75% or lower, 60% or lower, 50% or lower, 40% or lower, 30% or lower, or 20% or lower than the nucleic acid impurity level obtained when the comparator recombinant AAV construct is used, optionally wherein the nucleic acid impurity level is between 40% and 80%, or between 50% and 70%, or between 15% and 55% of the nucleic acid impurity level obtained when the comparator recombinant AAV construct is used.

15. The recombinant AAV construct according to any one of claims 12 to 14, wherein the comparator recombinant AAV construct is more than 4910, more than 4920, more than 4930, more than 4940, more than 4950, more than 4960, more than 4970, more than 4980, more than 4990, or more than 5000 nucleotides in length, optionally wherein the comparator recombinant AAV construct comprises or consists of SEQ ID NO: 72.

16. A Factor VIII polypeptide comprising a Factor VIII amino acid sequence wherein the Factor VIII amino acid sequence comprises a modified beta domain related (BDR) region which is modified relative to wild-type BDR region, which wild-type BDR region corresponds to the region between positions 713 and 1697 of SEQ ID NO: 1, wherein:
(iii) the modified BDR region comprises a maximum of 88 amino acids, and the Factor VIII polypeptide has a specific activity which is higher than the specific activity of a polypeptide of SEQ ID NO: 7; and/or
(iv) the modified BDR region comprises a maximum of 74 amino acids.

17. The Factor VIII polypeptide according to claim 16, wherein:
(i) the Factor VIII polypeptide has a specific activity which is at least 50%, at least 60%, at least 70%, at least 75%, at least 80%, at least 85%, at least 90%, at least 95%, at least 98%, at least 99%, or at least 100% of the specific activity of a reference wild-type Factor VIII polypeptide, optionally the Factor VIII polypeptide of SEQ ID NO: 1; and/or
(ii) the Factor VIII polypeptide has a specific activity which is higher than the specific activity of a reference wild-type Factor VIII polypeptide, optionally the Factor VIII polypeptide of SEQ ID NO: 1; and/or
(iii) the Factor VIII polypeptide has a specific activity which is at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of a reference wild-type Factor VIII, optionally the Factor VIII polypeptide of SEQ ID NO: 1; and/or
(iv) the Factor VIII polypeptide has a specific activity which is at least 50%, at least 60%, at least 70%, at least 75%, at least 80%, at least 85%, at least 90%, at least 95%, at least 98%, at least 99%, or at least 100% of the specific activity of the Factor VIII polypeptide of SEQ ID NO: 7; and/or
(v) the Factor VIII polypeptide has a specific activity which is higher than the specific activity of the Factor VIII polypeptide of SEQ ID NO: 7; and/or
(vi) the Factor VIII polypeptide has a specific activity which is at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of the Factor VIII polypeptide of SEQ ID NO: 7; and/or
(vii) the Factor VIII polypeptide has a specific activity which is at least 50%, at least 60%, at least 70%, at least 75%, at least 80%, at least 85%, at least 90%, at least 95%, at least 98%, at least 99%, or at least 100% of the specific activity of the Factor VIII polypeptide of SEQ ID NO: 8; and/or
(viii) the Factor VIII polypeptide has a specific activity which is higher than the specific activity of the Factor VIII polypeptide of SEQ ID NO: 8; and/or
(ix) the Factor VIII polypeptide has a specific activity which is at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of the Factor VIII polypeptide of SEQ ID NO: 8; and/or
(x) the Factor VIII polypeptide has a specific activity which is higher than the specific activity of the Factor VIII polypeptide encoded by SEQ ID NO: 37; and/or
(xi) the Factor VIII polypeptide has a specific activity which is higher than the specific activity of the Factor VIII polypeptide encoded by SEQ ID NO: 38; and/or
(xii) the specific activity is measured using a chromogenic assay; and/or
(xiii) the specific activity is measured using a clotting assay, optionally a one-stage clotting assay; and/or
(xiv) the modified BDR region comprises a maximum of 87, 85, 80, 75, 70, 65, 60, 55, 50, or 45 amino acids; and/or
(xv) the modified BDR region comprises a maximum of 74 amino acids; and/or
(xvi) the modified BDR region comprises a maximum of 47 amino acids; and/or
(xvii) the modified BDR region comprises a maximum of 45 amino acids; and/or
(xviii) the modified BDR region consists of between 28 and 48 amino acids; and/or
(xix) the modified BDR region consists of between 30 and 48 amino acids; and/or
(xx) the modified BDR region consists of around 45 amino acids.

18. The Factor VIII polypeptide according to claim 16 or 17, wherein the Factor VIII amino acid sequence:
(i) does not comprise amino acids corresponding to positions 732 to 1669 of SEQ ID NO: 1; and/or
(ii) does not comprise amino acids corresponding to positions 732 to 1669 of SEQ ID NO: 1, and the Factor VIII amino acid sequence comprises amino acids corresponding to positions 724 to 731 of SEQ ID NO: 1; and/or
(iii) does not comprise amino acids corresponding to positions 732 to 1669 of SEQ ID NO: 1, and the Factor VIII amino acid sequence comprises amino acids corresponding to positions 1670 to 1689 of SEQ ID NO: 1; and/or
(iv) does not comprise amino acids corresponding to positions 732 to 1669 of SEQ ID NO: 1, and the Factor VIII amino acid sequence comprises amino acids corresponding to positions 724 to 731 and 1670 to 1689 of SEQ ID NO: 1; and/or
(v) does not comprise amino acids corresponding to positions 732 to 1669 of SEQ ID NO: 1, and the Factor VIII amino acid sequence comprises amino acids corresponding to positions 724 to 731 and 1670 to 1689 of SEQ ID NO: 1 and the tyrosine amino acids corresponding to positions 718, 719 and 723 of SEQ ID NO: 1; and/or
(vi) does not comprise amino acids corresponding to positions 732 to 1669 of SEQ ID NO: 1, and the Factor VIII amino acid sequence comprises amino acids corresponding to positions 714 to 731 and 1670 to 1689 of SEQ ID NO: 1; and/or
(vii) does not comprise amino acids corresponding to positions 732 to 1669 of SEQ ID NO: 1, and the Factor VIII amino acid sequence comprises amino acids corresponding to positions 1 to 731 and 1670 to 2332 of SEQ ID NO: 1; and/or
(viii) comprises SEQ ID NO: 31; and/or
(ix) comprises any one of SEQ ID NOs: 9-36.

19. The Factor VIII polypeptide according to any one of claims 16 to 18, wherein the Factor VIII amino acid sequence comprises one or more substitution mutations at an inter-domain interface selected from the group consisting of:
d. the A1/A3 domain interface;
e. the A2/A3 domain interface; or
f. the A1/C2 domain interface,
wherein:
(iii) the one or more substitution mutations comprises substitution of an amino acid with a more hydrophobic amino acid; or
(iv) the one or more substitution mutations comprises substitution of a pair of amino acids in the respective domains with cysteine residues;
and wherein
(a) the Factor VIII polypeptide has higher specific activity than a reference wild-type Factor VIII polypeptide; and/or
(b) the Factor VIII polypeptide has higher stability than a reference wild-type Factor VIII polypeptide; and/or
(c) the Factor VIII polypeptide is expressed at a higher level in a host cell than a reference wild-type Factor VIII polypeptide.

20. The Factor VIII polypeptide according to any one of claims 16 to 18, wherein the Factor VIII amino acid sequence comprises a one or more substitution mutations selected from the group consisting of:
c. a substitution of an amino acid corresponding to M662 or H693 of SEQ ID NO: 1; or
d. a substitution of a pair of amino acids comprising a first amino acid and a second amino acid with cysteine residues, wherein:
1. the first amino acid corresponds to M147, S149 or S289 of SEQ ID NO: 1 and the second amino acid corresponds to E1969, E1970 or N1977 of SEQ ID NO: 1;
2. the first amino acid corresponds to T667, T669, N684, L687, I689, S695 or F697 of SEQ ID NO: 1 and the second amino acid corresponds to S1791, G1799, A1800, R1803, E1844, S1949, G1981, V1982, or Y1979 of SEQ ID NO: 1; or
3. the first amino acid corresponds to A108, T118 or V137 of SEQ ID NO: 1 and the second amino acid corresponds to N2172, Q2329 or Y2332 of SEQ ID NO: 1.

21. The Factor VIII polypeptide according to claim 19 or 20, wherein the Factor VIII polypeptide:
(i) has higher specific activity relative to a reference wild-type Factor VIII polypeptide; and/or
(ii) has higher stability relative to a reference wild-type Factor VIII polypeptide; and/or
(iii) has higher stability relative to a reference wild-type Factor VIII polypeptide, and the Factor VIII polypeptide has a longer half-life relative to the reference wild-type Factor VIII polypeptide, optionally wherein the Factor VIII polypeptide has a longer half-life relative to the reference wild-type Factor VIII polypeptide when activated; and/or
(iv) is expressed at a higher level in a host cell than a reference wild-type Factor VIII polypeptide.

22. The Factor VIII polypeptide according to any one of claims 19 to 21, wherein the Factor VIII polypeptide has higher specific activity and/or higher stability and/or is expressed at a higher level in a host cell than a reference Factor VIII polypeptide which comprises the Factor VIII amino acid sequence of the Factor VIII polypeptide but which does not comprise the one or more substitution mutations, optionally wherein the reference Factor VIII polypeptide is the Factor VIII polypeptide of SEQ ID NO: 1, 3 or 5.

23. The Factor VIII polypeptide according to any one of claim 19, 21 or 22, wherein the amino acid substituted with a more hydrophobic amino acid is methionine corresponding to the amino acid at position 662 of SEQ ID NO: 1 or histidine corresponding to the amino acid at position 693 of SEQ ID NO: 1.

24. The Factor VIII polypeptide according to any one of claims 19 to 24, wherein:
(i) the one or more substitution mutation does not comprise the M662C substitution; and/or
(ii) a. the one or more substitution mutations comprises substitution of methionine with tyrosine, isoleucine, leucine, phenylalanine or tryptophan; and/or b. the one or more substitution mutations comprises substitution of histidine with glutamate, cysteine, valine, methionine, tyrosine, isoleucine, leucine, phenylalanine or tryptophan; and/or
(iii) the one or more substitution mutations comprises substitution of an amino acid with an aromatic amino acid; and/or
(iv) the one or more substitution mutations comprises the M662W substitution; and/or
(v) the one or more substitution mutations comprises the H693W or H693Y substitution; and/or
(vi) the one or more substitution mutations comprises the M662W and H693W substitutions; and/or
(vii) the one or more substitution mutations comprises substitution of a pair of amino acids in the respective domains with cysteine residues, wherein the cysteine residues form a disulphide bond between the respective domains.

25. The Factor VIII polypeptide according to any one of claims 16 to 24, wherein the Factor VIII amino acid sequence comprises the amino acid sequence set forth in SEQ ID NO: 77, or an amino acid sequence that is at least 90%, at least 95%, at least 96%, at least 97%, at least 98%, at least 99% or 100% identical to the amino acid sequence set forth in SEQ ID NO: 77.

26. A polynucleotide comprising a Factor VIII nucleotide sequence, wherein the Factor VIII nucleotide sequence encodes a Factor VIII polypeptide and wherein at least a portion of the Factor VIII nucleotide sequence is not wild-type.

27. The polynucleotide of claim 26, wherein the Factor VIII polypeptide comprises a Factor VIII amino acid sequence.

28. The polynucleotide according to claim 26 or 27, wherein the Factor VIII nucleotide sequence encodes the Factor VIII polypeptide according to any one of claims 16 to 25.

29. The polynucleotide according to any one of claims 26 to 28, wherein the portion of the Factor VIII nucleotide sequence that is not wild-type is codon-optimised.

30. The polynucleotide according to claim 29, wherein, in the portion of the Factor VIII amino acid sequence that is codon-optimised, at least 50%, at least 55%, at least 60%, at least 61%, at least 62%, at least 63%, at least 64%, at least 65%, at least 69%, at least 70%, at least 75%, or at least 78% of the codons are selected from the group consisting of: TTC, CTG, ATC, GTG, TCC, AGT, AGC, CCT, CCC, ACC, ACA, GCC, TAC, CAC, CAG, AAC, AAG, GAC, GAG, TGT, AGA, and GGC.

31. The polynucleotide according to any one of claims 26 to 30, wherein:
(i) the Factor VIII polypeptide encoded by the Factor VIII nucleotide sequence is expressed in human liver cells at higher levels compared to a reference wild-type Factor VIII nucleotide sequence, optionally wherein the reference wild-type Factor VIII nucleotide sequence is the Factor VIII nucleotide sequence of SEQ ID NO: 2; and/or
(ii) the Factor VIII polypeptide encoded by the Factor VIII nucleotide sequence is expressed in human liver cells at least 1.1 fold, at least 1.2 fold, at least 1.5 fold, at least 1.8 fold, at least 2 fold, at least 5 fold, at least 10 fold, at least 20 fold, at least 30 fold, at least 40 fold, or at least 50 fold higher compared to a reference wild-type Factor VIII nucleotide sequence, optionally wherein the reference wild-type Factor VIII nucleotide sequence is the Factor VIII nucleotide sequence of SEQ ID NO: 2.

32. The polynucleotide according to any one of claims 26 to 31, wherein the Factor VIII polypeptide encoded by the Factor VIII nucleotide sequence is expressed in human liver cells at at least 50%, at least 60%, at least 70%, at least 75%, at least 80%, at least 85%, at least 90%, at least 95%, at least 98%, at least 99%, or at least 100% of the level expressed by a polypeptide encoded by the polynucleotide of:
(i) SEQ ID NO: 3; and/or.
(ii) SEQ ID NO: 4; and/or
(iii) SEQ ID NO: 5; and/or
(iv) SEQ ID NO: 6.

33. The polynucleotide according to any one of claims 27 to 32, wherein the Factor VIII nucleotide sequence encoding the Factor VIII amino acid sequence comprises:
(i) a sequence that is at least 85.5%, at least 86.0%, at least 88%, at least 90%, at least 92%, at least 95%, at least 98%, at least 99% or 100% identical to the portion of SEQ ID NO: 3 which encodes the Factor VIII amino acid sequence; and/or
(ii) a sequence that is at least 85.5%, at least 86.0%, at least 88%, at least 90%, at least 92%, at least 95%, at least 98%, at least 99% or 100% identical to SEQ ID NO: 3; and/or
(iii) the sequence of SEQ ID NO: 3; and/or
(iv) a sequence that is at least 85.0%, at least 85.5%, at least 86.0%, at least 88%, at least 90%, at least 92%, at least 95%, at least 98%, at least 99% or 100% identical to the portion of SEQ ID NO: 4 which encodes the Factor VIII amino acid sequence; and/or
(v) a sequence that is at least 85.0%, at least 85.5%, at least 86.0%, at least 88%, at least 90%, at least 92%, at least 95%, at least 98%, at least 99% or 100% identical to SEQ ID NO: 4; and/or
(vi) the sequence of SEQ ID NO: 4; and/or
(vii) a sequence that is at least 85.0%, at least 85.5%, at least 86.0%, at least 88%, at least 90%, at least 92%, at least 95%, at least 98%, at least 99% or 100% identical to the portion of SEQ ID NO: 5 which encodes the Factor VIII amino acid sequence; and/or
(viii) a sequence that is at least 85.0%, at least 85.5%, at least 86.0%, at least 88%, at least 90%, at least 92%, at least 95%, at least 98%, at least 99% or 100% identical to SEQ ID NO: 5; and/or
(ix) the sequence of SEQ ID NO: 5; and/or
(x) a sequence that is at least 86.5%, at least 87.0%, at least 87.5%, at least 88.0%, at least 90%, at least 92%, at least 95%, at least 98%, at least 99% or 100% identical to the portion of SEQ ID NO: 6 which encodes the Factor VIII amino acid sequence; and/or
(xi) a sequence that is at least 86.5%, at least 87.0%, at least 87.5%, at least 88.0%, at least 90%, at least 92%, at least 95%, at least 98%, at least 99% or 100% identical to SEQ ID NO: 6; and/or
(xii) the sequence of SEQ ID NO: 6.

34. The polynucleotide according to any one of claims 27 to 33, wherein the Factor VIII nucleotide sequence encoding the Factor VIII amino acid sequence comprises the sequence of SEQ ID NO: 39, SEQ ID NO: 40, SEQ ID NO: 41, or SEQ ID NO: 42, preferably SEQ ID NO: 39.

35. The polynucleotide according to any one of claims 26 to 34, wherein the Factor VIII nucleotide encodes a Factor VIII amino acid sequence as defined in any one of claims 19 to 25.

36. The polynucleotide according to claim 35, wherein the Factor VIII nucleotide sequence encoding the Factor VIII amino acid sequence comprises:
(i) a nucleotide sequence that is at least 85%, at least 90%, at least 95%, at least 96%, at least 97%, at least 98%, at least 99% or 100% identical to the nucleotide sequence set forth in SEQ ID NO: 76, or a nucleotide sequence that is at least 85%, at least 90%, at least 95%, at least 96%, at least 97%, at least 98%, at least 99% or 100% identical to a nucleotide sequence comprising at least 4047 nucleotides of SEQ ID NO: 76; and/or
(ii) the nucleotide sequence set forth in SEQ ID NO:75 or a nucleotide sequence that is at least 85%, at least 90%, at least 95%, at least 96%, at least 97%, at least 98%, at least 99% or 100% identical to the nucleotide sequence set forth in SEQ ID NO:75.

37. A recombinant AAV construct which comprises a polynucleotide comprising a Factor VIII nucleotide sequence, wherein the Factor VIII nucleotide sequence encodes a Factor VIII polypeptide comprising a Factor VIII amino acid sequence.

38. The recombinant AAV construct according to any one of claim 1 to 15 or 37, wherein the Factor VIII polypeptide is the Factor VIII polypeptide according to any one of claims 16 to 25.

39. The recombinant AAV construct according to any one of claim 1 to 15, 37 or 38, wherein the polynucleotide is the polynucleotide according to any one of claims 26 to 36.

40. A Factor VIII polypeptide encoded by the polynucleotide or recombinant AAV construct according to any one of claims 1 to 15 or 26 to 39.

41. An AAV viral particle comprising the recombinant AAV construct according to any one of claims 1 to 15 or 37 to 39.

42. The AAV viral particle according to claim 41, wherein the viral particle comprises a capsid, optionally wherein the capsid is selected from the group consisting of:
(i) a capsid comprising a sequence which is at least 96%, at least 98%, at least 99%, at least 99.5%, at least 99.8% or 100% identity to SEQ ID NO: 68;
(ii) a capsid comprising a sequence which is at least 96%, at least 98%, at least 99%, at least 99.5%, at least 99.8%, or 100% identity to SEQ ID NO: 69;
(iii) a liver-tropic capsid; and
(iv) an AAVS capsid.

43. The recombinant AAV construct or AAV viral particle according to any one of claims 1 to 15 or 37 to 42, wherein the activity of the polypeptide encoded by the Factor VIII nucleotide sequence achieved:
(i) is higher following administration of the recombinant AAV construct or AAV viral particle compared to the administration of an equivalent dose of an equivalent recombinant AAV construct or AAV viral particle which comprises the AAV construct of SEQ ID NO: 1 of WO 2017/053677 (SEQ ID NO: 72); and/or
(ii) is at least 1.1 fold, at least 1.2 fold, at least 1.5 fold, at least 1.8 fold, at least 2 fold, or at least 5 fold higher compared to the equivalent recombinant AAV construct or AAV viral particle which comprises the AAV construct of SEQ ID NO: 1 of WO 2017/053677 (SEQ ID NO: 72).

44. A composition comprising the Factor VIII polypeptide, polynucleotide, recombinant AAV construct or AAV viral particle according to any one of claims 1 to 43, and a pharmaceutically acceptable excipient.

45. The Factor VIII polypeptide, polynucleotide, recombinant AAV construct, AAV viral particle or composition according to any one of claims 1 to 44 for use in a method of treatment.

46. The Factor VIII polypeptide, polynucleotide, recombinant AAV construct, AAV viral particle or composition for use according to claim 45, wherein the method of treatment comprises administering an effective amount of the Factor VIII polypeptide, polynucleotide, recombinant AAV construct or AAV viral particle of any one of claims 1 to 44 to a patient.

47. A method of treatment comprising administering an effective amount of the Factor VIII polypeptide, polynucleotide, recombinant AAV construct, AAV viral particle or composition according to any one of claims 1 to 44 to a patient.

48. Use of the Factor VIII polypeptide, polynucleotide, recombinant AAV construct, AAV viral particle or composition according to any one of claims 1 to 44 in the manufacture of a medicament for use in a method of treatment.

49. The use according to claim 48 wherein the method of treatment comprises administering an effective amount of the Factor VIII polypeptide, polynucleotide, recombinant AAV construct, AAV viral particle or composition according to any one of claims 1 to 44 to a patient.

50. The Factor VIII polypeptide, polynucleotide, recombinant AAV construct, AAV viral particle, composition, method or use according to any one of claims 45 to 49, wherein the method of treatment is a method of treating haemophilia, optionally wherein the haemophilia is haemophilia A.

51. Use of the recombinant AAV construct as defined in any one of claims 1 to 15 or 37 to 39 for producing a population of AAV viral particles.

52. The use according to claim 51, wherein the AAV construct is less than 4900 nucleotides in length, wherein the population of AAV viral particles:
d) has an increased vector genome yield when compared to the vector genome yield obtained when a comparator recombinant AAV construct is used;
e) has an increased vector genome to total particle ratio when compared to the vector genome to total particle ratio obtained when a comparator recombinant AAV construct is used; and/or
f) has a decreased nucleic acid impurity level when compared to the nucleic acid impurity level obtained when a comparator recombinant AAV construct is used;
wherein the comparator recombinant AAV construct is more than 4900 nucleotides in length.

53. Use of a recombinant AAV construct as defined in any one of claim 1 to 15, 38 or 39 for:
d) increasing the vector genome yield during AAV viral particle production when compared to the vector genome yield obtained when a comparator recombinant AAV construct is used;
e) increasing the vector genome to total particle ratio during AAV viral particle production when compared to the vector genome to total particle ratio obtained when a comparator recombinant AAV construct is used; and/or
f) decreasing the nucleic acid impurity level during AAV viral particle production when compared to the nucleic acid impurity level obtained when a comparator recombinant AAV construct is used;
wherein the comparator recombinant AAV construct is more than 4900 nucleotides in length.

54. The use according to claim 52 or 53, wherein:
(i) the vector genome yield is at least 1.5, at least 2, at least 2.25, at least 2.5, at least 2.75, at least 3, at least 4, or at least 5 fold higher than the vector genome yield obtained when the comparator recombinant AAV construct is used, optionally wherein the vector genome yield is between 1.25 and 3 fold higher, between 1.5 and 3 fold higher, or between 2 and 3 fold higher than the vector genome yield obtained when the comparator recombinant AAV construct is used; and/or
(ii) the vector genome to total particle ratio is at least 1.25, at least 1.5, at least 1.75, at least 2, at least 2.25, at least 2.5, at least 2.75, at least 3, at least 3.25, at least 3.5, at least 4, or at least 5 fold higher than the vector genome to total particle ratio obtained when the comparator recombinant AAV construct is used, optionally wherein the vector genome to total particle ratio is between 1.25 and 4 fold higher, or between 1.5 and 3.5 fold higher than the vector genome to total particle ratio obtained when the comparator recombinant AAV construct is used; and/or
(iii) nucleic acid impurity level is 85% or lower, 75% or lower, 60% or lower, 50% or lower, 40% or lower, 30% or lower, or 20% or lower than the nucleic acid impurity level obtained when the comparator recombinant AAV construct is used, optionally wherein the nucleic acid impurity level is between 40% and 80%, between 50% and 70%, or between 15% and 55% of the nucleic acid impurity level obtained when the comparator recombinant AAV construct is used.

55. A method for producing a population of AAV viral particles comprising:
a) obtaining the recombinant AAV construct as defined in any one of claims 1 to 15 or 37 to 39;
b) transfecting a host cell with the said recombinant AAV construct; and
c) culturing the host cell under conditions suitable for AAV viral particle production.

56. A method for increasing the vector genome yield during the production of a population of AAV viral particles, comprising:
a) obtaining the recombinant AAV construct as defined in any one of claim 1 to 15, 38 or 39;
b) transfecting a host cell with the said recombinant AAV construct; and
c) culturing the host cell under conditions suitable for AAV viral particle production;
wherein the vector genome yield is increased when compared to the vector genome yield obtained when a comparator recombinant AAV construct of more than 4900 nucleotides in length is used.

57. A method for increasing the vector genome to total particle ratio during the production of a population of AAV viral particles, comprising:
a) obtaining the recombinant AAV construct as defined in any one of claim 1 to 15, 38 or 39;
b) transfecting a host cell with the said recombinant AAV construct; and
c) culturing the host cell under conditions suitable for AAV viral particle production;
wherein the vector genome to total particle ratio is increased when compared to the vector genome to total particle ratio obtained when a comparator recombinant AAV construct of more than 4900 nucleotides in length is used.

58. A method for decreasing the nucleic acid impurity level during the production of a population of AAV viral particles, comprising:
a) obtaining the recombinant AAV construct as defined in any one of claim 1 to 15, 38 or 39;
b) transfecting a host cell with the said recombinant AAV construct; and
c) culturing the host cell under conditions suitable for AAV viral particle production;
wherein the nucleic acid impurity level is decreased when compared to the nucleic acid impurity level obtained when a comparator recombinant AAV construct of more than 4900 nucleotides in length is used.

59. The method or use according to any one of claims 52 to 54 or 56 to 58, wherein the comparator is more than 4910, more than 4920, more than 4930, more than 4940, more than 4950, more than 4960, more than 4970, more than 4980, more than 4990, or more than 5000 nucleotides in length, optionally wherein the comparator comprises or consists of SEQ ID NO: 72.

60. A population of AAV viral particles obtained or obtainable by the method of any one of claims 55 to 59.
